(19) United States
(12) Patent Application Publication
(10) Pub. No.: US 2013/0237445 A1
(43) Pub. Date: Sep. 12, 2013

(54) METHODS AND KITS FOR DETECTING MELANOMA

(71) Applicants: Nancy Thomas, Durham, NC (US); Kathleen Dorsey, Pittsboro, NC (US); Sharon Edmiston, Chapel Hill, NC (US); Pamela Groben, Mebane, NC (US)

(72) Inventors: Nancy Thomas, Durham, NC (US); Kathleen Dorsey, Pittsboro, NC (US); Sharon Edmiston, Chapel Hill, NC (US); Pamela Groben, Mebane, NC (US)

(73) Assignee: THE UNIVERSITY OF NORTH CAROLINA AT CHAPEL HILL, CHAPEL HILL, NC (US)

(21) Appl. No.: 13/823,056

(22) PCT Filed: Sep. 13, 2011
(86) PCT No.: PCT/US2011/051401

§ 371 Date: May 29, 2013

Related U.S. Application Data
(60) Provisional application No. 61/382,623, filed on Sep. 14, 2010.

(51) Int. Cl. C12Q 1/68; G01N 33/68

(52) U.S. Cl. CPC C12Q 1/6886; G01N 33/6881
USPC 506/9; 435/6.11

(57) ABSTRACT

This invention is directed to a method for detecting melanoma in a tissue sample by measuring a level of methylation of one or more regulatory elements differentially methylated in melanoma and benign nevi. The invention provides methods for detecting melanoma, related kits, and methods of screening for compounds to prevent or treat melanoma.

### 1. RELATED APPLICATION

This application claims the benefit of U.S. Provisional Patent Application No. 61/382,623, filed Sep. 14, 2010 entitled “Methods and Kits for Detecting Melanoma” naming Nancy Thomas et al. as inventors with Attorney Docket No. UNC10001USV. The entire contents of which are hereby incorporated by reference including all text, tables, and drawings.

### STATEMENT REGARDING FEDERALLY SPONSORED RESEARCH OR DEVELOPMENT

This invention was made at least in part with government support under grant number 1R21CA134368-01 awarded by the National Cancer Institute. The United States Government has certain rights in the invention.

### 2. FIELD OF THE INVENTION

This invention relates generally to the discovery of novel differentially methylated regulatory elements associated with melanoma. The invention provides methods for detecting melanoma, related kits, and methods of screening for compounds to prevent or treat melanoma.

### BACKGROUND OF THE INVENTION

### 2.1. Skin Cancer and Melanoma

Skin cancer is the most common form of cancer. There are two major types of skin cancer, keratinocyte cancers (basal and squamous cell carcinomas) and melanoma. Though melanoma is less than five percent of the skin cancers, it is the seventh most common malignancy in the U.S. and is responsible for most of the skin cancer related deaths. Specifically, the American Cancer Society estimates that in the U.S. 114,000 new cases of melanoma, including 68,000 invasive and 46,000 noninvasive melanomas, will be diagnosed in 2010 and almost 9,000 people will die of melanoma (Jemal et al., CA Cancer J. Clin. 2010 July 7 [Epub ahead of print]). The WHO estimates that 48,000 people die worldwide of melanoma every year (Lucas, R., Global Burden of Disease of Solar Ultraviolet Radiation, Environmental Burden of Disease Series, Jul. 25, 2006; No. 13. News release, World Health Organization).

As with many cancers, the clinical outcome for melanoma depends on the stage at the time of the initial diagnosis. When melanoma is diagnosed early, the prognosis is good. However, if diagnosed in late stages, it is a deadly disease. In particular in 2010 the ACS reports that the 5-year survival rate is 92% for melanoma diagnosed when small and localized, stage IA or IB. However, when the melanoma has spread beyond the original area of skin and nearby lymph nodes, the 5-year survival rate drops to 15-20% for distant metastatic disease, or stage 1V melanoma. It is therefore imperative to diagnose melanoma in its earliest form. In addition, interventions for melanoma such as use of cytotoxic chemotherapy and other available agents, rarely impact the course of disease (Avril et al., 2004, J. Clin. Oncol. 15, 1118-1125; Middleton et al., 2000, J. Clin. Oncol. 18, 158-166).

### 2.2. Issues with Melanoma Diagnosis

Early diagnosis is difficult due to the overlap in clinical and histopathological features of early melanomas and benign nevi, especially benign atypical nevi (Strauss et al., 2007, Br. J. Dermatol. 157, 758-764). Moreover, there is a sizeable disagreement amongst pathologists regarding the diagnosis of melanoma and benign diseases such as compound melanocytic nevi or Spitz nevi. One study reported a 15% discordance (Shoo et al. 2010, J. Am. Acad. Dermatol. 62(5), 751-756). An earlier study of over 1000 melanocytic lesions reported that an expert panel found a 14% rate of false positives, misclassifying benign lesions as invasive melanoma; and a 17% rate of false negatives, misclassifying malignant melanoma as benign (Veenhuizen et al. 1997, J. Pathol. 182, 266-272). In one study where an expert panel interpreted lesions as melanoma, a group of general pathologists mistakenly diagnosed dysplastic nevi in 12% of the readings (Brochez et al., 2002, J. Pathol. 196, 459-466). In fact, many nevi, especially atypical or dysplastic nevi, are difficult to distinguish from melanoma, even by expert pathologists (Farmer et al., 1996, Hum. Pathol. 27, 528-531). This results in a quandary for clinicians who not only biopsy but re-excise with margins large numbers of benign atypical nevi in the population (Fung, 2003, Arch. Dermatol. 139, 1374-1375), at least, in part, due to lack of confidence in the histopathologic diagnosis. The numbers involved are substantial in the U.S. alone. One study estimated that with 1,500,000 to 4,500,000 annual biopsies of melanocytic neoplasms, 200,000 to 650,000 discordant cases would result annually (Shoo et al. 2010, J. Am. Acad. Dermatol. 62(5), 751-756). This high rate of misdiagnosis is problematic on many levels. The false positives lead to unnecessary costly medical interventions, e.g., overly large excisions, high-dose interleukin-2 or interferon alpha, and needless stress for the patients. The false negatives mean increased likelihood of a presentation with more severe disease, which as discussed above, dramatically increases the risk of a poor clinical outcome and risk of death.

Furthermore, current guidelines recommend wide excisional biopsy with 0.5 to 2.0 cm margins for patients presenting with primary melanoma (NCCN, Clin. Pract. Guidelines in Oncology—v.2.2010: Melanoma, Mar. 17, 2010, page ME-B). However, excisional biopsy with such broad margins may not be appropriate for sites such as the face, ears, fingers, palms, or soles of the feet. Better histopathology will improve the ability for doctors to choose the appropriate intervention, such as margin controlled surgery (Mohs surgery) with 0.2 cm margins.

### 2.3. Standard of Care for Melanoma

For suspicious pigmented lesions current guidelines recommend excisional biopsy with 1-3 mm margins and rebiopsy if the sample is inadequate for diagnosis or microstaging. Pathologists typically assess Breslow's depth or thickness, ulceration, mitotic rate, margin status and Clark's level (based on the skin layer penetrated). A positive diagnosis for melanoma may lead to an evaluation for potential spread to the lymph nodes or other organs. Patients with stage I or II melanoma are further staged with sentinel lymph node (SLN) biopsy including immunohistochemical (IHC) staining. IHC is often used as an adjunct to the standard histopathologic examination (hematoxylin and eosin (H&E) staining, etc.) for melanocytic lesions or to determine the tumor of origin. Antibodies such as S100, HMB-45, Ki-67 (MIB1), MITF and MART-1/Melan-A or cocktails of several may be used for staining (Ivan & Prieto, 2010, Future Oncol. 6(7), 1163-1175; Linos et al., 2011, Biomarkers Med. 5(3) 333-360). In a literature review Rothberg et al. report that melanoma cell adhesion molecule (MCAM)/MUC18, matrix metalloproteinase-2, Ki-67, proliferating cell nuclear antigen (PCNA) and p16/INK4A are predictive of either all-cause mortality or melanoma specific mortality (Rothberg et al., 2009 J. Nat. Canc. Inst. 101(7) 452-474). Rothberg et al. also note that these and other “molecular prognostic markers have largely failed to be incorporated into guidelines, staging systems, or the standard of care for melanoma patients.”

Follow up may include cross sectional imaging (CT, MRI, PET). For patients suspected with stage III disease, with clinically positive lymph nodes, guidelines recommend fine needle aspiration or open biopsy of the enlarged lymph node. For patients with distant metastases, stage 1V, serum lactate dehydrogenase (LDH) may have a prognostic role (NCCN Guidelines).

As discussed above, wide excision is recommended for primary melanoma. For patients with lymph node involvement, stage III, complete lymph dissection may be indicated. For patients with resected stage IIB or III melanoma, some studies have shown that adjuvant interferon alfa has led to longer disease free survival. For first- or second-line stage III and IV melanoma systemic treatments include: carboplatin, cisplatin, dacarbazine, interferon alfa, high-dose interleukin-2, paclitaxel, temozolomide, vinblastine or combinations thereof (NCCN Guidelines, ME-D, MS-9-13). Recently, the FDA approved Zelboraf™ (vemurafenib, also known as INN, PLX4032, RG7204 or R05185426) for unresectable or metastatic melanoma with the BRAF V600E mutation (Bollag et al., 2010, Nature 467, 596-599, Chapman et al., 2011, New Eng. J. Med. 364 2507-2516). Another recently approved drug for unresectable or metastatic melanoma is Yervoy® (ipilimumab) an antibody which binds to cytotoxic T-lymphocyte-associated antigen 4 (CTLA-4) (Hodi et al., 2010, New Eng. J. Med. 363 711-723). Others recently reported that patients with KIT receptor activating mutations or over-expression responded to Gleevac® (imatinib mesylate) (Carvajal et al., 2011, JAMA 305(22) 2327-2334).

### 2.4. Emerging Molecular Diagnostic Tools

Ivan and Prieto review recent reports of antibodies associated with melanoma pathogenesis but their prognostic significance is unclear. Specifically, they discuss work with adhesion molecules (catenins, claudins), apoptosis inhibitors (survivin), cell cycle regulators (cyclins, HDM2, Ki67), growth factors and receptors (c-Kit/SCF, KIT, VEGF, VEGF R3), signaling molecules (Akt), transcription factors (ATF-1), and tumor suppressors (p53, PTEN). Others have reported use of a tissue microarray to predict melanoma progression and in particular found that Ki67, p16^{INK4a}, p21^{CIP1} and Bcl-6 correlated with metastatic disease (Alonso et al., 2004, Am. J. Pathol. 164(1) 193-203).

In a study of melanoma progression, Haqq et al. show gene expression patterns associated with metastatic melanomas (Haqq et al., 2005, Proc. Nat. Acad. Sci. USA, 102(17), 6092-6097). The value of these markers is uncertain because the researchers used a very small sample set melanoma (N=6) and moles (N=9). Riker et al. report gene expression profiles of primary and metastatic melanomas (Riker et al., 2008, BMC Med. Genomics, 1, 13, pub. 28 Apr. 2008). Limited numbers of frozen melanomas and nevi have been profiled using 19K-41K gene expression arrays (Haqq et al., 2005; Scatolini et al., 2010, Int. J. Cancer 126:1869-81; Talantov et al., 2005, Clin. Cancer Res. 11:7234-42). Upon further investigation of candidate markers on an FFPE training set, Kashani-Sabet et al. achieved a 91% sensitivity and 95% specificity using a 5-marker IHC panel analyzed with a composite diagnostic algorithm that takes into account the distribution of staining from top-to-bottom of the specimen (Kashani-Sabet et al., 2009, Proc. Nat. Acad. Sci. USA, 106:6268-72). Alexandrescu et al. found that, using RT-PCR for unequivocal melanoma vs. benign nevi, candidate markers SILV, GDF15, and L1CAM normalized to TYR gave areas under the curve (AUC) of 0.94, 0.67, and 0.5, respectively, while SILV, the best marker, gave an AUC of 0.74 for differentiating melanoma from atypical nevi (Alexandrescu et al., 2010, J. Invest. Dermatol. 130:1887-92). In a different study, candidate gene expression differences were selected for FFPE primary cutaneous melanomas (N=38) vs. conventional nevi (N=48) using a custom gene expression array probing 1,100 unique genes (Koh et al., 2009, Mod. Pathol. 22:538-46). A ‘leave-one-out’ cross-validation using a 100 probe qPCR-based classifier incorporating candidate markers showed concordance of 89% between gene classification and histopathologic diagnosis for all samples (N=120 melanomas and nevi) (Koh et al., 2009).

Others have studied both proteins and nucleic acids associated with melanocytes transforming into melanomas (Hoek et al., 2004, Can. Res. 64, 5270-5282). Bastian et al. describe comparative genomic hybridization (CGH) as a means to find patterns of chromosomal aberrations associated with melanoma (Bastian et al., 2003, Am. J. Pathol. 163(5), 1765-1770). The utility of CGH in a clinical setting is limited because it currently requires approximately a microgram of DNA and about a month for results. Gerami et al. report a fluorescence in situ hybridization (FISH) panel of 4 probes, chromosome 6p25, 6 centromere, 6q23 and 11q13 showed a 86.7% sensitivity and 95.4% specificity (Gerami et al., 2009, Am. J. Surg. Pathol. 33(8) 1146-1156). FISH for melanoma has shown promise in the clinic and healthcare providers currently reimburse such tests. However, FISH is better for detecting amplifications than deletions so some information from CGH is lost.

Recent studies show that activating mutations in the BRAF or NRAS oncogenes occur in approximately 50% (Thomas et al., 2004, J. Invest Dermatol. 122, 1245-1250; Edlundh-Rose et al., 2006, Melanoma Res. 16, 471-478; Thomas et al., 2007, Cancer Epidemiol. Biomarkers Prev. 16, 991-977) and 20% (Edlundh-Rose et al., 2006; Thomas et al., 2007) of primary cutaneous melanomas, respectively. However, the majority of nevi also contain these mutations (Pollock et al., 2003, Nat. Genet. 33, 19-20; reviewed in Thomas et al., 2006, Melanoma Res. 16, 97-103, Uribe et al. 2006, Am. J. Dermatopathol. 25, 365-370; Poynter et al., 2006, Melanoma Res. 16, 267-273; Wu et al., 2007, J. Dermatopathol. 29, 534-537), which limits their usefulness for melanoma diagnosis. As mentioned above, Zelboraf™ (vemurafenib) has been approved for patients with the BRAF V600E mutation. As a companion diagnostic, the FDA approved the Roche Cobas® 4800 V600 BRAF Mutation Test for use on formalin-fixed paraffin-embedded (FFPE) samples.

DNA methylation may provide a tool, in conjunction with histopathology, for the molecular diagnostics of melanoma. DNA methylation is an epigenetic chemical modification that does not alter the sequence code, but can be heritable, and is involved in the regulation of gene expression (Plass, 2002, Hum. Mol. Genet. 11, 2479-2488). The most common methylation site in mammals is a cytosine located next to a guanosine (CpG). Clusters of CpGs, referred to as islands, are found in the 5′ regulatory and promoter regions of genes (Antequera and Bird, 1993, Proc. Natl. Acad. Sci. USA, 90, 11995-11999). Hypermethylation of CpG islands in promoter regions is a common mechanism of tumor suppressor gene silencing in cancer (Balmain et al., 2003, Nat. Genet. 33 Suppl, 238-244; Baylin and Herman, 2000, Trends Genet. 16, 168-174; Feinberg and Tycko, 2004, Nat. Rev. Cancer 4, 143-153; Plass, 2002). Aberrant promoter methylation with silencing of tumor suppressor genes has been shown to occur widely in human melanomas (Furuta et al., 2004, Cancer Sci. 95, 962-968; Hoon et al., 2004, Oncogene 23, 4014-4022; Bonazzi et al., 2008, Genes Chromosomes Cancer, 48, 10-21), and in histologically pre-malignant lesions associated with a variety of cancer types (Fackler et al., 2003, Int. J. Cancer, 107, 970-975). These studies suggest methylation may be useful as an early diagnostic marker for melanoma. However much of the work to date has been performed with passaged cells or cell lines rather than actual tissue samples. Changes associated with passaging and/or immortalization create artifacts that reduce their usefulness (Staveren et al., 2009, Biochim. Biophys. Acta Rev. Cancer, 1795 (2) 92-103).

Molecular diagnosis of melanoma holds promise but, due to the small size of melanocytic lesions which are typically submitted in entirety for diagnosis, any new diagnostic tests need to be valid and reproducible in FFPE tissues. Previously, gene expression arrays were used to identify markers of melanoma heterogeneity using cell lines and a few frozen and FFPE melanomas, but found that only 24% of unselected FFPE samples produced RNA of sufficient quality for microarray analysis (Penland et al., 2007, Lab. Invest. 87, 383-391). Improvements in melanoma diagnosis could be accelerated by the use of molecular assays that are less sensitive to tissue fixation than RNA-based assays. Moreover, there is an unmet medical need for improved melanoma diagnosis. The invention described herein provides a solution.

### 3. SUMMARY OF THE INVENTION

In particular non-limiting embodiments, the present invention provides a method for detecting melanoma in a tissue sample which comprises: (a) measuring a level of methylation of one or more regulatory elements differentially methylated in melanoma and benign nevi; and (b) determining whether melanoma is present or absent in the tissue sample. The methylation may be measured at single CpG site resolution. The tissue sample may be a common nevi, a dysplastic nevi, or a benign atypical nevi sample, or a melanocytic lesion of unknown potential. The sample may be prepared in a variety of ways including, but not limited to, a formalin-fixed, paraffin-embedded (FFPE) sample, a fresh-frozen sample, or a fresh tissue sample. There are many sources for the samples, including but not limited to, dissected tissue, an excision biopsy, a needle biopsy, a punch biopsy, a shave biopsy, a tape biopsy, or a skin biopsy. Alternatively, the sample may be from a lymph node biopsy, a sentinel lymph node, or a cancer metastasis.

In particular non-limiting embodiments, the present invention provides that the differentially methylatated regulatory elements are elements associated with immune response/inflammatory pathway genes, hormonal regulation genes, or cell growth/cell adhesion/apoptosis genes. The regulatory elements may be associated with a gene encoding CARD15, CCL3, CD2, EMR3, EVI2A, FRZB, GSTM2, HLA-DPA1, IFNG, ITK, KCNK4, KLK10, LAT, MPO, NPR2, OSM, PSCA, PTHLH, PTHR1, RUNX3, TNFSF8 or TRIP6. In one non-limiting embodiment, hypermethylation of the regulatory elements associated with a gene encoding FRZB, GSTM2, KCNK4, NPR2, or TRIP6 is indicative of melanoma. In another non-limiting embodiment, hypomethylation of the regulatory elements associated with a gene encoding CARD15, CCL3, CD2, EMR3, EVI2A, HLA-DPA1, IFNG, ITK, KLK10, LAT, MPO, OSM, PSCA, PTHLH, PTHR1, RUNX3 or TNFSF8 is indicative of melanoma. In one non-limiting embodiment, a panel of 22 genes is used. In another non-limiting embodiment a panel of 14 genes is used. The level of methylation may be measured using a variety of methods including, but not limited to, assays based on bisulfate conversion-based microarray, differential hybridization, methylated DNA immunoprecipitation, methylated CpG island recovery (MIRA), methylation specific polymerase chain reaction (MSP), or methylation-sensitive high resolution melting (MS-HRM). The detection of the differentially methylated elements may also be by microarray or mass spectrometry. The differentially methylated elements may be amplified by pyrosequencing, invasive cleavage amplification, sequencing by ligation, or emulsion-based PCR.

In non-limiting embodiments, the regulatory element differentially methylated has a sensitivity analysis area under the curve of greater than 0.70, 0.75, 0.8, 0.85, 0.9, 0.95, 0.98, or 0.99. The levels of methylation for 4 or more regulatory elements may be measured. Alternatively, 8 or 12 or more regulatory elements are measured.

In non-limiting embodiments, the method further comprises evaluating the quality of the sample by measuring the levels of skin specific markers using antibody staining, differential methylation, expression analysis, or fluorescence in situ hybridization (FISH). The methods of the present invention may also include staining the tissue sample with one or more antibodies specific for melanoma. The antibody may be 5100, gp100 (HMB-45 antibody), MART-1/Melan-A, MITF, or tyrosinase antibodies, or a cocktail of all three antibodies. Alternatively, the methods may further comprise fluorescence in situ hybridization (FISH), comparative genomic hybridization (CGH), or gene expression analysis.

Moreover, the invention also includes measuring transcription of genes or the translation of proteins that are indirectly or directly under the influence of a gene hyper- or hypomethylated in melanoma. Specifically, the invention includes using antibodies or probes or primers to measure FRZB, GSTM2, KCNK4, NPR2, or TRIP6 proteins or nucleic acids, wherein reduced levels are indicative of melanoma. The levels relative to a benign control may be about 80%, preferably 50%, more preferably 25-0%. Alternatively, antibodies or probes or primers to measure CARD15, CCL3, CD2, EMR3, EVI2A, HLA-DPA1, IFNG, ITK, KLK10, LAT, MPO, OSM, PSCA, PTHLH, PTHR1, RUNX3, or TNFSF8 proteins or nucleic acids, wherein elevated levels are is indicative of melanoma. The levels relative to a benign control may be 110%, more preferably 150%, more preferably 200-500% (i.e., two to five fold higher relative to the control), more preferably 1000-3000% higher.

In other non-limiting embodiments, the present invention provides a kit comprising: (a) at least one reagent selected from the group consisting of: (i) a nucleic acid probe capable of specifically hybridizing with a regulatory element differentially methylated in melanoma and benign nevi; (ii) a pair of nucleic acid primers capable of PCR amplification of a regulatory element differentially methylated in melanoma and benign nevi; and (iii) a methylation specific antibody and a probe capable of specifically hybridizing with a regulatory element differentially methylated in melanoma and benign nevi; and (b) instructions for use in measuring a level of methylation of at least one regulatory element in a tissue sample from a subject suspected of having melanoma.

In other non-limiting embodiments, the present invention provides a method of identifying a compound that prevents or treats melanoma progression, the method comprising the steps of: (a) contacting a compound with a sample comprising a cell or a tissue; (b) measuring a level of methylation of one or more regulatory elements differentially methylated in melanoma and benign nevi; and (c) determining a functional effect of the compound on the level of methylation; thereby identifying a compound that prevents or treats melanoma.

### 4. BRIEF DESCRIPTION OF THE FIGURES

FIGS. 1A-1I show correlation curves showing the reproducibility and effects of formalin fixation and normal cell contamination on melanocytic methylation profiles obtained with the Illumina GoldenGate methylation array. FIGS. 1A-1C show the reproducibility and effects of formalin fixation on methylation profile. Shown are non-fixed duplicates of the MCF-7 breast cancer cell line (r2=0.98) (FIG. 1A), duplicates of the MeI-505 melanoma cell line (r2=0.99) (FIG. 1B), and comparison of formalin-fixed, paraffin-embedded Mel-505 with non-fixed Mel-505 cells (r2=0.99) (FIG. 1C). FIGS. 1D-1I show the effect of contamination with increasing proportions of normal peripheral blood leukocyte (PBL) DNA on the Mel-505 melanoma cell methylation profile. Shown are Mel-505 cells that were mixed with PBL DNA in the following proportions: 100% Mel-505, (FIG. 1D); 90% Mel-505/10% PBL (FIG. 1E); 80% Mel-505/20% PBL (FIG. 1F); 70% Mel-505/30% PBL (FIG. 1G); 60% Mel-505/40% PBL (FIG. 1H); and 50% Mel-505/50% PBL (FIG. 1I).

FIG. 2 shows the hierarchical clustering of methylation β values using the Illumina GoldenGate Cancer Panel I array in FFPE benign nevi and malignant melanomas. DNA methylation profiles for 22 melanomas and 27 nevi are shown. Columns represent tissue samples; rows represent CpG loci. The methylation levels (β) range from 0 (very light grey/unmethylated) to 1 (dark grey/highly methylated). Missing values are shown in white. FIG. 2 displays clusters based on the 29 CpG sites/genes showing significantly different methylation β levels between moles and melanomas after adjustment for age and sex and Bonferroni correction for multiple comparisons. The upper portion of the heatmap shows 7 CpG loci in 6 genes exhibiting hypermethylation and 22 CpG loci in 18 genes exhibiting hypomethylation in melanomas compared with moles.

FIGS. 3A-3L show box plots of methylation β levels in the 12 CpG loci identified by PAM analysis that predict melanoma. The loci shown differed by >0.2 mean β between melanomas and moles, except for ITK_P114_F. Each box plot shows the mean β value (dark bar within box), the standard deviation (outer boundaries of box), and the range of β values (broken line) within the melanomas or nevus groups. Additional information on mean β values for nevi and melanomas, differences in mean β values, and p-values adjusted for age, sex, and multiple comparisons through Bonferroni correction are given in Table 3A.

FIG. 4A-4O show ROC curves showing the sensitivity and specificity of selected CpG loci to distinguish melanomas from benign nevi based on methylation level. The area under the curve (AUC) is presented, showing sensitivity and specificity of melanoma diagnosis for CpG sites that exhibited either significant hypomethylation (n=22) or hypermethylation (n=7) in melanomas compared with benign nevi after adjustment for age, sex and multiple comparisons. Sensitivity, or the frequency of detection of true positives (melanoma vs nevus), is shown along the y axis, while specificity, or the frequency of false positives, is shown along the x axis. The calculated AUC is given for each plot.

FIG. 5 shows a Venn diagram of CpG sites that significantly differentiate non-dysplastic and dysplastic nevi from primary melanomas or metastases.

### 5. DETAILED DESCRIPTION OF THE INVENTION

### 5.1. Definitions

The term “melanoma” refers to malignant neoplasms of melanocytes, which are pigment cells present normally in the epidermis, in adnexal structures including hair follicles, and sometimes in the dermis, as well as extracutaneous sites such as the mucosa, meninx, conjuctiva, and uvea. Sometimes it is referred to as “cutaneous melanoma” or “malignant melanoma.” There are at least four types of cutaneous melanoma: lentigo maligna melanoma (LMM), superficial spreading melanoma (SSM), nodular melanoma (NM), and acral lentiginous melanoma (ALM). Cutaneous melanoma typically starts as a proliferation of single melanocytes, e.g., at the junction of the epidermis and the dermis. The cells first grow in a horizontal manner and settle in an area of the skin that can vary from a few millimeters to several centimeters. As noted above, in most instances the transformed melanocytes produce increased amounts of pigment so that the area involved can be seen by the clinician.

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., messenger RNA (mRNA), short inhibitory RNA (siRNA), ribosomal RNA (rRNA), tRNA, microRNA, RNA highly expressed by the melanoma or nevi, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. Examples of nucleic acids are SEQ ID Nos. 1-75 shown in Table 4A and Table 4B; SEQ ID Nos. 76-93 in Table 7A and 7B; SEQ ID Nos. 94-265 in Table 9D; SEQ ID Nos. 266-283 in Table 13; SEQ ID Nos. 284-339 in Table 14; and SEQ ID Nos. 340-353 in Table 15, which may be methylated or unmethylated at any CpG site present in the sequence, including the CpG sites shown in brackets on some sequences. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses methylated forms, conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil.

A “methylated regulatory element” as used herein refers to a segment of DNA sequence at a defined location in the genome of an individual. Typically, a “methylated regulatory element” is at least 15 nucleotides in length and contains at least one cytosine. It may be at least 18, 20, 25, 30, 50, 80, 100, 150, 200, 250, or 300 nucleotides in length and contain 1 or 2, 5, 10, 15, 20, 25, or 30 cytosines. For any one “methylated regulatory element” at a given location, e.g., within a region centering around a given genetic locus, nucleotide sequence variations may exist from individual to individual and from allele to allele even for the same individual. Typically, such a region centering around a defined genetic locus (e.g., a CpG island) contains the locus as well as upstream and/or downstream sequences. Each of the upstream or downstream sequence (counting from the 5′ or 3′ boundary of the genetic locus, respectively) can be as long as 10 kb, in other cases may be as long as 5 kb, 2 kb, 1 kb, 500 bp, 200 bp, or 100 bp. Furthermore, a “methylated regulatory element” may modulate expression of a nucleotide sequence transcribed into a protein or not transcribed for protein production (such as a non-coding mRNA). The “methylated regulatory element” may be an inter-gene sequence, intra-gene sequence (intron), protein-coding sequence (exon), a non protein-coding sequence (such as a transcription promoter or enhancer), or a combination thereof.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has, or has the possibility of occurring. For example a location containing CpG is a methylation site wherein the cytosine may or may not be methylated.

As used herein, a “CpG site” or “methylation site” is a nucleotide within a nucleic acid that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

The term “epigenetic state” or “epigenetic status” as used herein refers to any structural feature at a molecular level of a nucleic acid (e.g., DNA or RNA) other than the primary nucleotide sequence. For instance, the epigenetic state of a genomic DNA may include its secondary or tertiary structure determined or influenced by, e.g., its methylation pattern or its association with cellular proteins.

The term “methylation profile” “methylation state” or “methylation status,” as used herein to describe the state of methylation of a genomic sequence, refers to the characteristics of a DNA segment at a particular genomic locus relevant to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles. The term “methylation” profile” or “methylation status” also refers to the relative or absolute concentration of methylated C or unmethylated C at any particular stretch of residues in a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., sample nucleic acid) are methylated as compared to another sequence from a different region or from a different individual (e.g., relative to normal nucleic acid), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”, and more specifically, when the methylation status differs between melanoma and benign or healthy moles, the sequences are considered “differentially methylated in melanoma and benign nevi”. Measurement of the levels of differential methylation may be done by a variety of ways known to those skilled in the art. One method is to measure the ratio of methylated to unmethylated alleles or β-value (see section 6.5 below). The difference in the ratios between methylated and unmethylated sequences in melanoma and benign nevi may be 0.1, 0.15, 0.2, 0.25, 0.3, 0.4, 0.5, 0.55, 0.6, 0.65, 0.7, 0.8, or 0.9. In non-limiting embodiments, the difference in the ratios is between 0.2 and 0.65, or between 0.2 and 0.4.

The term “agent that binds to methylated nucleotides” as used herein refers to a substance that is capable of binding to methylated nucleic acid. The agent may be naturally-occurring or synthetic, and may be modified or unmodified. In one embodiment, the agent allows for the separation of different nucleic acid species according to their respective methylation states. An example of an agent that binds to methylated nucleotides is described in PCT Pub. No. WO 2006/056480 A2 (Rehli), hereby incorporated by reference in its entirety. The described agent is a bifunctional polypeptide comprising the DNA-binding domain of a protein belonging to the family of Methyl-CpG binding proteins (MBDs) and an Fc portion of an antibody. The recombinant methyl-CpG-binding, antibody-like protein can preferably bind CpG methylated DNA in an antibody-like manner. That means, the methyl-CpG-binding, antibody-like protein has a high affinity and high avidity to its “antigen”, which is preferably DNA that is methylated at CpG dinucleotides. The agent may also be a multivalent MBD.

The term “bisulfite” as used herein encompasses any suitable type of bisulfite, such as sodium bisulfite, or other chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C→U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

The terms “non-bisulfite-based method” and “non-bisulfite-based quantitative method” as used herein refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

The term “target nucleic acid” as used herein refers to a nucleic acid examined using the methods disclosed herein to determine if the nucleic acid is melanoma associated. The term “control nucleic acid” as used herein refers to a nucleic acid used as a reference nucleic acid according to the methods disclosed herein to determine if the nucleic acid is associated with melanoma. The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (PCR), to amplify a nucleotide sequence based on the polynucleotide sequence corresponding to a particular genomic sequence, e.g., one specific for a particular CpG site. At least one of the PCR primers for amplification of a polynucleotide sequence is sequence-specific for the sequence.

The term “template” refers to any nucleic acid molecule that can be used for amplification in the technology. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In embodiments, the method of amplification includes, but is not limited to, polymerase chain reaction, self-sustained sequence reaction, ligase chain reaction, rapid amplification of cDNA ends, polymerase chain reaction and ligase chain reaction, Q-β replicase amplification, strand displacement amplification, rolling circle amplification, or splice overlap extension polymerase chain reaction. In some embodiments, a single molecule of nucleic acid may be amplified.

The term “sensitivity” as used herein refers to the number of true positives divided by the number of true positives plus the number of false negatives, where sensitivity (sens) may be within the range of 0<sens<1. Ideally, method embodiments herein have the number of false negatives equaling zero or close to equaling zero, so that no subject is wrongly identified as not having melanoma when they indeed have melanoma. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0<spec<1. Ideally, the methods described herein have the number of false positives equaling zero or close to equaling zero, so that no subject is wrongly identified as having melanoma when they do not in fact have melanoma. Hence, a method that has both sensitivity and specificity equaling one, or 100%, is preferred.

“RNAi molecule” or “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

An “antisense” polynucleotide is a polynucleotide that is substantially complementary to a target polynucleotide and has the ability to specifically hybridize to the target polynucleotide. Ribozymes are enzymatic RNA molecules capable of catalyzing specific cleavage of RNA. The composition of ribozyme molecules preferably includes one or more sequences complementary to a target mRNA, and the well-known catalytic sequence responsible for mRNA cleavage or a functionally equivalent sequence (see, e.g., U.S. Pat. Nos. 5,093,246 (Cech et al.); 5,766,942 (Haseloff et al.); 5,856,188 (Hampel et al.) which are incorporated herein by reference in their entirety). Ribozyme molecules designed to catalytically cleave target mRNA transcripts can also be used to prevent translation of genes associated with the progression of melanoma. These genes may be genes found to be hypomethylated in melanoma.

The phrase “functional effects” in the context of assays for testing means compounds that modulate a methylation of a regulatory region of a gene associated with melanoma. This may also be a chemical or phenotypic effect such as altered transcriptional activity of a gene hyper- or hypomethylated in melanoma, or altered activities and the downstream effects of proteins encoded by these genes. A functional effect may include transcriptional activation or repression, the ability of cells to proliferate, expression in cells during melanoma progression, and other characteristics of melanoma cells. “Functional effects” include in vitro, in vivo, and ex vivo activities. By “determining the functional effect” is meant assaying for a compound that increases or decreases the transcription of genes or the translation of proteins that are indirectly or directly under the influence of a gene hyper- or hypomethylated in melanoma. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. Validation the functional effect of a compound on melanoma progression can also be performed using assays known to those of skill in the art such as metastasis of melanoma cells by tail vein injection of melanoma cells in mice. The functional effects can be evaluated by many means known to those skilled in the art, e.g., microscopy for quantitative or qualitative measures of alterations in morphological features, measurement of changes in RNA or protein levels for other genes expressed in melanoma cells, measurement of RNA stability, identification of downstream or reporter gene expression (CAT, luciferase, β-gal, GFP and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of the methylation state, the expression of genes hyper- or hypomethylated in melanoma or the translation proteins encoded thereby Inhibitors, activators, or modulators also include naturally occurring and synthetic ligands, antagonists, agonists, antibodies, peptides, cyclic peptides, nucleic acids, antisense molecules, ribozymes, RNAi molecules, small organic molecules and the like. Such assays for inhibitors and activators include, e.g., (1)(a) measuring methylation states, (b) the mRNA expression, or (c) proteins expressed by genes hyper- or hypomethylated in melanoma in vitro, in cells, or cell extracts; (2) applying putative modulator compounds; and (3) determining the functional effects on activity, as described above.

Samples or assays comprising genes hyper- or hypomethylated in melanoma are treated with a potential activator, inhibitor, or modulator are compared to control samples without the inhibitor, activator, or modulator to examine the extent of inhibition. Control samples (untreated with inhibitors) are assigned a relative activity value of 100%. Inhibition of methylation, expression, or proteins encoded by genes hyper- or hypomethylated in melanoma is achieved when the activity value relative to the control is about 80%, preferably 50%, more preferably 25-0%. Activation of methylation, expression, or proteins encoded by genes hyper- or hypomethylated in melanoma is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide, small organic molecule, polysaccharide, peptide, circular peptide, lipid, fatty acid, siRNA, polynucleotide, oligonucleotide, etc., to be tested for the capacity to directly or indirectly modulate genes hyper- or hypomethylated in melanoma. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis. The compound may be “small organic molecule” that is an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

### 5.2. Tissue Samples

The tissue sample may be from a patient suspected of having melanoma or from a patient diagnosed with melanoma, e.g., for confirmation of diagnosis or establishing a clear margin or for the detection of melanoma cells in other tissues such as lymph nodes. The biological sample may also be from a subject with an ambiguous diagnosis in order to clarify the diagnosis. The sample may be obtained for the purpose of differential diagnosis, e.g., a subject with a histopathologically benign lesion to confirm the diagnosis. The sample may also be obtained for the purpose of prognosis, i.e., determining the course of the disease and selecting primary treatment options. Tumor staging and grading are examples of prognosis. The sample may also be evaluated to select or monitor therapy, selecting likely responders in advance from non-responders or monitoring response in the course of therapy. In addition, the sample may be evaluated as part of post-treatment ongoing surveillance of patients who have had melanoma. The sample may also be obtained to differentiate dysplastic nevi from other benign nevi. The sample may be a melanoma sample such as a melanomas will be superficial spreading melanoma, nodular melanoma, lentigo maligna melanoma, acral lentiginous melanoma, unclassifiable or other (spitzoid/desmoplastic/nevoid/spindle cell) melanoma. The sample may be normal skin, a benign nevi, a melanoma-in-situs (MIS), or a high-grade dysplastic nevi (HGDN).

Biological samples may be obtained using any of a number of methods in the art. Examples of biological samples comprising potential melanocytic lesions include those obtained from excised skin biopsies, such as punch biopsies, shave biopsies, fine needle aspirates (FNA), or surgical excisions; or biopsy from non-cutaneous tissues such as lymph node tissue, mucosa, conjuctiva, or uvea, other embodiments. The biological sample can be obtained by shaving, waxing, or stripping the region of interest on the skin. A non-limiting example of a product for stripping skin for RNA recovery is the EGIR™ tape strip product (DermTech International, La Jolla, Calif., see also, Wachsman et al., 2011, Brit. J. Derm. 164 797-806). Representative biopsy techniques include, but are not limited to, excisional biopsy, incisional biopsy, needle biopsy, surgical biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally contains a suspension of cells from within the tumor mass. The biological sample may be a microdissected sample, such as a PALM-laser (Carl Zeiss MicroImaging GmbH, Germany) capture microdissected sample.

A sample may also be a sample of muscosal surfaces, blood and blood fractions or products (e.g., serum, plasma, platelets, red blood cells, white blood cells, circulating tumor cells isolated from blood, free DNA isolated from blood, and the like), sputum, lymph and tongue tissue, cultured cells, e.g., primary cultures, explants, and transformed cells, stool, urine, etc. The sample may also be vascular tissue or cells from blood vessels such as microdissected blood vessel cells of endothelial origin. A sample is typically obtained from a eukaryotic organism, most preferably a mammal such as a primate e.g., chimpanzee or human; cow; dog; cat; a rodent, e.g., guinea pig; rat; mouse; rabbit.

A sample can be treated with a fixative such as formaldehyde and embedded in paraffin (FFPE) and sectioned for use in the methods of the invention. Alternatively, fresh or frozen tissue may be used. These cells may be fixed, e.g., in alcoholic solutions such as 100% ethanol or 3:1 methanol:acetic acid. Nuclei can also be extracted from thick sections of paraffin-embedded specimens to reduce truncation artifacts and eliminate extraneous embedded material. Typically, biological samples, once obtained, are harvested and processed prior to hybridization using standard methods known in the art. Such processing typically includes protease treatment and additional fixation in an aldehyde solution such as formaldehyde.

### 5.3. Techniques for Measuring Methylation

A variety of methylation analysis procedures are known in the art and may be used to practice the invention. These assays allow for determination of the methylation state of one or a plurality of CpG sites within a tissue sample. In addition, these methods may be used for absolute or relative quantification of methylated nucleic acids. Another embodiment of the invention are methods of detecting melanoma based on the differentially methylated sites found in tissue analysis described herein, and not differentially methylated in cultured melanocytes and/or melanoma cell lines. Such methylation assays involve, among other techniques, two major steps. The first step is a methylation specific reaction or separation, such as (i) bisulfate treatment, (ii) methylation specific binding, or (iii) methylation specific restriction enzymes. The second major step involves (i) amplification and detection, or (ii) direct detection, by a variety of methods such as (a) PCR (sequence-specific amplification) such as Taqman®, (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

Additionally, restriction enzyme digestion of PCR products amplified from bisulfite-converted DNA may be used, e.g., the method described by Sadri & Hornsby (1996, Nucl. Acids Res. 24:5058-5059), or COBRA (Combined Bisulfite Restriction Analysis) (Xiong & Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Frommer et al., 1992, Proc. Nat. Acad. Sci. USA, 89, 1827-1831). PCR amplification of the bisulfite converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

5.3.1. Methylation-Specific PCR (MSP)

Methylation-Specific PCR (MSP) allows for assessing the methylation status of virtually any group of CpG sites within a CpG island, independent of the use of methylation-sensitive restriction enzymes (Herman et al., 1996, Proc. Nat. Acad. Sci. USA, 93, 9821-9826; U.S. Pat. Nos. 5,786,146, 6,017,704, 6,200,756, 6,265,171 (Herman & Baylin) U.S. Pat. Pub. No. 2010/0144836 (Van Engeland et al.); which are hereby incorporated by reference in their entirety). Briefly, DNA is modified by sodium bisulfite converting unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes. The ColoSure™ test is a commercially available test for colon cancer based on the MSP technology and measurement of methylation of the vimentin gene (Itzkowitz et al., 2007, Clin Gastroenterol. Hepatol. 5(1), 111-117). Alternatively, one may use quantitative multiplexed methylation specific PCR (QM-PCR), as described by Fackler et al. Fackler et al., 2004, Cancer Res. 64(13) 4442-4452; or Fackler et al., 2006, Clin. Cancer Res. 12(11 Pt 1) 3306-3310.

5.3.2. MethyLight and Heavy Methyl Methods

The MethyLight and Heavy Methyl assays are a high-throughput quantitative methylation assay that utilizes fluorescence-based real-time PCR (Taq Mang) technology that requires no further manipulations after the PCR step (Eads, C. A. et al., 2000, Nucleic Acid Res. 28, e 32; Cottrell et al., 2007, J. Urology 177, 1753, U.S. Pat. Nos. 6,331,393 (Laird et al.), the contents of which are hereby incorporated by reference in their entirety). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both. The MethyLight assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites. Typical reagents (e.g., as might be found in a typical MethyLight-based kit) for MethyLight analysis may include, but are not limited to: PCR primers for specific gene (or methylation-altered DNA sequence or CpG island); TaqMan° probes; optimized PCR buffers and deoxynucleotides; and Taq polymerase. The MethyLight technology is used for the commercially available tests for lung cancer (epi proLung BL Reflex Assay); colon cancer (epi proColon assay and mSEPT9 assay) (Epigenomics, Berlin, Germany) PCT Pub. No. WO 2003/064701 (Schweikhardt and Sledziewski), the contents of which is hereby incorporated by reference in its entirety.

Quantitative MethyLight uses bisulfite to convert genomic DNA and the methylated sites are amplified using PCR with methylation independent primers. Detection probes specific for the methylated and unmethylated sites with two different fluorophores provides simultaneous quantitative measurement of the methylation. The Heavy Methyl technique begins with bisulfate conversion of DNA. Next specific blockers prevent the amplification of unmethylated DNA. Methylated genomic DNA does not bind the blockers and their sequences will be amplified. The amplified sequences are detected with a methylation specific probe. (Cottrell et al., 2004, Nuc. Acids Res. 32, e10, the contents of which is hereby incorporated by reference in its entirety).

The Ms-SNuPE technique is a quantitative method for assessing methylation differences at specific CpG sites based on bisulfite treatment of DNA, followed by single-nucleotide primer extension (Gonzalgo & Jones, 1997, Nucleic Acids Res. 25, 2529-2531). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites. Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfate conversion reagents may include: DNA denaturation buffer; sulfonation buffer; DNA recovery regents or kit (e.g., precipitation, ultrafiltration, affinity column); desulfonation buffer; and DNA recovery components.

5.3.3. Differential Binding-Based Methylation Detection Methods

For identification of differentially methylated regions, one approach is to capture methylated DNA. This approach uses a protein, in which the methyl binding domain of MBD2 is fused to the Fc fragment of an antibody (MBD-FC) (Gebhard et al., 2006, Cancer Res. 66:6118-6128; and PCT Pub. No. WO 2006/056480 A2 (Relhi), the contents of which are hereby incorporated by reference in their entirety). This fusion protein has several advantages over conventional methylation specific antibodies. The MBD FC has a higher affinity to methylated DNA and it binds double stranded DNA. Most importantly the two proteins differ in the way they bind DNA. Methylation specific antibodies bind DNA stochastically, which means that only a binary answer can be obtained. The methyl binding domain of MBD-FC, on the other hand, binds DNA molecules regardless of their methylation status. The strength of this protein—DNA interaction is defined by the level of DNA methylation. After binding genomic DNA, eluate solutions of increasing salt concentrations can be used to fractionate non-methylated and methylated DNA allowing for a more controlled separation (Gebhard et al., 2006, Nucleic Acids Res. 34 e82). Consequently this method, called Methyl-CpG immunoprecipitation (MCIP), not only enriches, but also fractionates genomic DNA according to methylation level, which is particularly helpful when the unmethylated DNA fraction should be investigated as well.

Alternatively, one may use 5-methyl cytidine antibodies to bind and precipitate methylated DNA. Antibodies are available from Abcam (Cambridge, Mass.), Diagenode (Sparta, N.J.) or Eurogentec (c/o AnaSpec, Fremont, Calif.). Once the methylated fragments have been separated they may be sequenced using microarray based techniques such as methylated CpG-island recovery assay (MIRA) or methylated DNA immunoprecipitation (MeDIP) (Pelizzola et al., 2008, Genome Res. 18, 1652-1659; O'Geen et al., 2006, BioTechniques 41(5), 577-580, Weber et al., 2005, Nat. Genet. 37, 853-862; Horak and Snyder, 2002, Methods Enzymol., 350, 469-83; Lieb, 2003, Methods Mol. Biol., 224, 99-109). Another technique is methyl-CpG binding domain column/segregation of partly melted molecules (MBD/SPM, Shiraishi et al., 1999, Proc. Natl. Acad. Sci. USA 96(6):2913-2918).

5.3.4. Methylation Specific Restriction Enzymatic Methods

For example, there are methyl-sensitive enzymes that preferentially or substantially cleave or digest at their DNA recognition sequence if it is non-methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. Methyl-sensitive enzymes that digest unmethylated DNA suitable for use in methods of the technology include, but are not limited to, HpalI, HhaI, MaelI, BstUI and AciI. An enzyme that can be used is HpalI that cuts only the unmethylated sequence CCGG. Another enzyme that can be used is Hhal that cuts only the unmethylated sequence GCGC. Both enzymes are available from New England BioLabs®, Inc. Combinations of two or more methyl-sensitive enzymes that digest only unmethylated DNA can also be used. Suitable enzymes that digest only methylated DNA include, but are not limited to, Dpnl, which only cuts at fully methylated 5′-GATC sequences, and McrBC, an endonuclease, which cuts DNA containing modified cytosines (5-methylcytosine or 5-hydroxymethylcytosine or N4-methylcytosine) and cuts at recognition site 5′ . . . Pu^{m}C(N₄₀₋₃₀₀₀)Pu^{m}C . . . 3′ (New England BioLabs, Inc., Beverly, Mass.). Cleavage methods and procedures for selected restriction enzymes for cutting DNA at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Sambrook et al. (See Sambrook et al. Molecular Biology: A Laboratory Approach, Cold Spring Harbor, N.Y. 1989) provide a general description of methods for using restriction enzymes and other enzymes.

The MCA technique is a method that can be used to screen for altered methylation patterns in genomic DNA, and to isolate specific sequences associated with these changes (Toyota et al., 1999, Cancer Res. 59, 2307-2312, U.S. Pat. No. 7,700,324 (Issa et al.) the contents of which are hereby incorporated by reference in their entirety). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

5.3.5. Methylation-Sensitive High Resolution Melting (HRM)

Recently, Wojdacz et al. reported methylation-sensitive high resolution melting as a technique to assess methylation. (Wojdacz and Dobrovic, 2007, Nuc. Acids Res. 35(6) e41; Wojdacz et al. 2008, Nat. Prot. 3(12) 1903-1908; Balic et al., 2009 J. Mol. Diagn. 11 102-108; and US Pat. Pub. No. 2009/0155791 (Wojdacz et al.), the contents of which are hereby incorporated by reference in their entirety). A variety of commercially available real time PCR machines have HRM systems including the Roche LightCycler480, Corbett Research RotorGene6000, and the Applied Biosystems 7500. HRM may also be combined with other amplification techniques such as pyrosequencing as described by Candiloro et al. (Candiloro et al., 2011, Epigenetics 6(4) 500-507). Any of SEQ ID NO 1-353, or portions thereof, may be used in a HRM assay.

5.3.6. Mass Spectroscopic Detection Methods

Another method for analyzing methylation sites is a primer extension assay, including an optimized PCR amplification reaction that produces amplified targets for analysis using mass spectrometry. The assay can also be done in multiplex. Mass spectrometry is a particularly effective method for the detection of polynucleotides associated with the differentially methylated regulatory elements. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. This method is described in detail in PCT Pub. No. WO 2005/012578A1 (Beaulieu et al.) which is hereby incorporated by reference in its entirety. For methylation analysis, the assay can be adopted to detect bisulfate introduced methylation dependent C to T sequence changes. These methods are particularly useful for performing multiplexed amplification reactions and multiplexed primer extension reactions (e.g., multiplexed homogeneous primer mass extension (hME) assays) in a single well to further increase the throughput and reduce the cost per reaction for primer extension reactions.

For a review of mass spectrometry methods using Sequenom® standard iPLEX™ assay and MassARRAY® technology, see Jurinke et al., 2004, Mol. Biotechnol. 26, 147-164. For methods of detecting and quantifying target nucleic acids using cleavable detector probes that are cleaved during the amplification process and detected by mass spectrometry, see PCT Pub. Nos. WO 2006/031745 (Van Der Boom and Boecker); WO 2009/073251 A1(Van Den Boom et al.); WO 2009/114543 A2 (Oeth et al.); and WO 2010/033639 A2 (Ehrich et al.); which are hereby incorporated by reference in their entirety.

5.3.7. Additional Methods for Methylation Analysis

Other methods for DNA methylation analysis include restriction landmark genomic scanning (RLGS, Costello et al., 2002, Meth. Mol. Biol., 200, 53-70), methylation-sensitive-representational difference analysis (MS-RDA, Ushijima and Yamashita, 2009, Methods Mol. Biol. 507, 117-130). Comprehensive high-throughput arrays for relative methylation (CHARM) techniques are described in WO 2009/021141 (Feinberg and Irizarry). The Roche® NimbleGen® microarrays including the Chromatin Immunoprecipitation-on-chip (ChIP-chip) or methylated DNA immunoprecipitation-on-chip (MeDIP-chip). These tools have been used for a variety of cancer applications including melanoma, liver cancer and lung cancer (Koga et al., 2009, Genome Res., 19, 1462-1470; Acevedo et al., 2008, Cancer Res., 68, 2641-2651; Rauch et al., 2008, Proc. Nat. Acad. Sci. USA, 105, 252-257). Others have reported bisulfate conversion, padlock probe hybridization, circularization, amplification and next generation or multiplexed sequencing for high throughput detection of methylation (Deng et al., 2009, Nat. Biotechnol. 27, 353-360; Ball et al., 2009, Nat. Biotechnol. 27, 361-368; U.S. Pat. No. 7,611,869 (Fan)). As an alternative to bisulfate oxidation, Bayeyt et al. have reported selective oxidants that oxidize 5-methylcytosine, without reacting with thymidine, which are followed by PCR or pyrosequencing (WO 2009/049916 (Bayeyt et al.). These references for these techniques are hereby incorporated by reference in their entirety.

5.3.8. Polynucleotide Sequence Amplification and Determination

Following reaction or separation of nucleic acid in a methylation specific manner, the nucleic acid may be subjected to sequence-based analysis. Furthermore, once it is determined that one particular melanoma genomic sequence is hypermethylated or hypomethylated compared to the benign counterpart, the amount of this genomic sequence can be determined Subsequently, this amount can be compared to a standard control value and serve as an indication for the melanoma. In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits of the invention may use any nucleic acid amplification or detection methods known to one skilled in the art, such as those described in U.S. Pat. Nos. 5,525,462 (Takarada et al.); 6,114,117 (Hepp et al.); 6,127,120 (Graham et al.); 6,344,317 (Urnovitz); 6,448,001 (Oku); 6,528,632 (Catanzariti et al.); and PCT Pub. No. WO 2005/111209 (Nakajima et al.); all of which are incorporated herein by reference in their entirety.

In some embodiments, the nucleic acids are amplified by PCR amplification using methodologies known to one skilled in the art. One skilled in the art will recognize, however, that amplification can be accomplished by any known method, such as ligase chain reaction (LCR), Qβ-replicase amplification, rolling circle amplification, transcription amplification, self-sustained sequence replication, nucleic acid sequence-based amplification (NASBA), each of which provides sufficient amplification. Branched-DNA technology may also be used to qualitatively demonstrate the presence of a sequence of the technology, which represents a particular methylation pattern, or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis); which are incorporated herein by reference in their entirety. PCR reagents and protocols are also available from commercial vendors, such as Roche Molecular Systems. PCR may be carried out as an automated process with a thermostable enzyme. In this process, the temperature of the reaction mixture is cycled through a denaturing region, a primer annealing region, and an extension reaction region automatically. Machines specifically adapted for this purpose are commercially available.

Amplified sequences may also be measured using invasive cleavage reactions such as the Invader® technology (Zou et al., 2010, Association of Clinical Chemistry (AACC) poster presentation on Jul. 28, 2010, “Sensitive Quantification of Methylated Markers with a Novel Methylation Specific Technology,” available at www.exactsciences.com; and U.S. Pat. No. 7,011,944 (Prudent et al.) which are incorporated herein by reference in their entirety).

5.3.9. High Throughput and Single Molecule Sequencing Technology

Suitable next generation sequencing technologies are widely available. Examples include the 454 Life Sciences platform (Roche, Branford, Conn.) (Margulies et al. 2005 Nature, 437, 376-380); 111 umina's Genome Analyzer, GoldenGate Methylation Assay, or Infinium Methylation Assays, i.e., Infinium HumanMethylation 27K BeadArray or VeraCode GoldenGate methylation array (Illumina, San Diego, Calif.; Bibkova et al., 2006, Genome Res. 16, 383-393; U.S. Pat. Nos. 6,306,597 and 7,598,035 (Macevicz); 7,232,656 (Balasubramanian et al.)); or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al.); or the Helicos True Single Molecule DNA sequencing technology (Harris et al., 2008 Science, 320, 106-109; U.S. Pat. Nos. 7,037,687 and 7,645,596 (Williams et al.); 7,169,560 (Lapidus et al.); 7,769,400 (Harris)), the single molecule, real-time (SMRT™) technology of Pacific Biosciences, and sequencing (Soni and Meller, 2007, Clin. Chem. 53, 1996-2001) which are incorporated herein by reference in their entirety. These systems allow the sequencing of many nucleic acid molecules isolated from a specimen at high orders of multiplexing in a parallel fashion (Dear, 2003, Brief Funct. Genomic Proteomic, 1(4), 397-416 and McCaughan and Dear, 2010, J. Pathol., 220, 297-306). Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. Machines for pyrosequencing and methylation specific reagents are available from Qiagen, Inc. (Valencia, Calif.). See also Tost and Gut, 2007, Nat. Prot. 2 2265-2275. An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., 2003, J. Biotech. 102, 117-124). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing or detection, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each other for energy transfer to occur successfully. Bailey et al. recently reported a highly sensitive (15 pg methylated DNA) method using quantum dots to detect methylation status using fluorescence resonance energy transfer (MS-qFRET) (Bailey et al. 2009, Genome Res. 19(8), 1455-1461, which is incorporated herein by reference in its entirety).

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., Braslaysky et al., PNAS 100(7): 3960-3964 (2003); U.S. Pat. No. 7,297,518 (Quake et al.) which are incorporated herein by reference in their entirety). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer-released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

The technology may be practiced with digital PCR. Digital PCR was developed by Kalinina and colleagues (Kalinina et al., 1997, Nucleic Acids Res. 25; 1999-2004) and further developed by Vogelstein and Kinzler (1999, Proc. Natl. Acad. Sci. U.S.A. 96; 9236-9241). The application of digital PCR is described by Cantor et al. (PCT Pub. Nos. WO 2005/023091A2 (Cantor et al.); WO 2007/092473 A2, (Quake et al.)), which are hereby incorporated by reference in their entirety. Digital PCR takes advantage of nucleic acid (DNA, cDNA or RNA) amplification on a single molecule level, and offers a highly sensitive method for quantifying low copy number nucleic acid. Fluidigm® Corporation offers systems for the digital analysis of nucleic acids.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in PCT Pub. No. WO 2009/091934 (Cantor).

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected.

A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

The invention encompasses any method known in the art for enhancing the sensitivity of the detectable signal in such assays, including, but not limited to, the use of cyclic probe technology (Bakkaoui et al., 1996, BioTechniques 20: 240-8, which is incorporated herein by reference in its entirety); and the use of branched probes (Urdea et al., 1993, Clin. Chem. 39, 725-6; which is incorporated herein by reference in its entirety). The hybridization complexes are detected according to well-known techniques in the art.

Reverse transcribed or amplified nucleic acids may be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like. Examples of capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotinistreptavidin, folic acid/folate binding protein, vitamin B 12/intrinsic factor, chemical reactive group/complementary chemical reactive group (e.g., sulfhydryl/maleimide, sulfhydry/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

### 5.4. Additional Methods

5.4.1. Antibody Staining/Detection

In some embodiments, the invention may encompass detecting and/or quantitating using antibodies either alone or in conjunction with measurement of methylation levels. Antibodies are already used in current practice in the classification and/or diagnosis of melanocytic lesions (Alonso et al., 2004, Am. J. Pathol. 164(1) 193-203; Ivan & Prieto, 2010, Future Oncol. 6(7), 1163-1175; Linos et al., 2011, Biomarkers Med. 5(3) 333-360; and Rothberg et al., 2009 J. Nat. Canc. Inst. 101(7) 452-474, the contents of which are hereby incorporated by reference in their entireties). Examples of antibodies that are used include HMB45/gp100 (Abcam; AbD Serotec; BioGenex, San Ramon, Calif.; Biocare Medical, Concord, Calif.); MART-1/Melan-A (Abcam; AbD Serotec; BioGenex; Thermo Scientific Pierce Abs., Rockford, Ill.); Microphthalmia transcription factor/MITF-1 (Invitrogen); NKI/C3 (Melanoma Associated Antigen 100+/7 kDa)(Abcam; Thermo Scientific Pierce Abs.); p75NTR/neurotrophin receptor (Abcam; AbD Serotec; Promega, Madison, Wis.); S100 (Abcam; AbD Serotec, Raleigh, N.C.; BioGenex); Tyrosinase (Abcam; AbD Serotec; Thermo Scientific Pierce Abs.). In one embodiment a cocktail of S100, HMB-45 and MART-1/Melan-A is used. Antibodies may also be used to detect the gene products of the methylated genes described herein. Specifically, genes hypomethylated would be expected to show over-expression and genes hypermethylated would be expected to show under-expression. Staining markers of tumor vascular formation may also be used in conjunction with the present invention (Bhati et al., 2008, Am. J. Pathol. 172(5), 1381-1390, including Table 1 on page 1387, the contents of which are incorporated herein by reference in their entirety).

Antibody reagents can be used in assays to detect expression levels of in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., 1996, Curr. Opin. Biotechnol., 7, 60-65. The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., 1997, Electrophoresis, 18, 2184-2193; Bao, 1997, J. Chromatogr. B. Biomed. Sci., 699, 463-480. Liposome immunoassays, such as flow-injection liposome immunoassays and liposome immunosensors, are also suitable for use in the present invention. See, e.g., Rongen et al., 1997, J. Immunol. Methods, 204, 105-133. In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.) and can be performed using a Behring Nephelometer Analyzer (Fink et al., 1989, J. Clin. Chem. Clin. Biochem., 27, 261-276).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 ¹²⁵I can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-/3-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot. The antibodies may be in an array one or more antibodies, single or double stranded nucleic acids, proteins, peptides or fragments thereof, amino acid probes, or phage display libraries. Many protein/antibody arrays are described in the art. These include, for example, arrays produced by Ciphergen Biosystems (Fremont, Calif.), Packard BioScience Company (Meriden Conn.), Zyomyx (Hayward, Calif.) and Phylos (Lexington, Mass.). Examples of such arrays are described in the following patents: U.S. Pat. Nos. 6,225,047 (Hutchens and Yip); 6,537,749 (Kuimelis and Wagner); and 6,329,209 (Wagner et al.), all of which are incorporated herein by reference in their entirety.

5.4.2. Fluorescence in situ Hybridization (FISH) and Comparative Genomic Hybridization (CGH)

In some embodiments, the invention may further encompass detecting and/or quantitating using fluorescence in situ hybridization (FISH) in a sample, preferably a tissue sample, obtained from a subject in accordance with the methods of the invention. FISH is a common methodology used in the art, especially in the detection of specific chromosomal aberrations in tumor cells, for example, to aid in diagnosis and tumor staging. As applied in the methods of the invention, it can be used in conjunction with detecting methylation. For reviews of FISH methodology, see, e.g., Weier et al., 2002, Expert Rev. Mol. Diagn. 2 (2): 109-119; Trask et al., 1991, Trends Genet. 7 (5): 149-154; and Tkachuk et al., 1991, Genet. Anal. Tech. Appl. 8: 676-74; U.S. Pat. No. 6,174,681 (Halling et al.); for multi-color FISH specific to melanoma, see Gerami et al., 2009, Am. J. Surg. Pathol. 33(8) 1146-1156; and PCT Pub. No. WO 2007/028031 A2 (Bastian et al.); all of which are incorporated herein by reference in their entirety. Alternatively, comparative genomic hybridization (CGH) also may be used as part of the methods disclosed herein. Specifically, Bastian et al. describe CGH as a means to find patterns of chromosomal aberrations associated with melanoma (Bastian et al., 2003, Am. J. Pathol. 163(5) 1765-1770).

In alternative embodiments, the invention encompasses use of additional melanoma specific gene expression and/or antibody assays either in situ, i.e., directly upon tissue sections (fixed and/or frozen) of patient tissue obtained from biopsies or resections, such that no nucleic acid purification is necessary; or based on extracted and/or amplified nucleic acids. Targets for such assays are disclosed in Haqq et al. 2005, Proc. Nat. Acad. Sci. USA, 102(17), 6092-6097; Riker et al., 2008, BMC Med. Genomics, 1, 13, pub. 28 Apr. 2008; Hoek et al., 2004, Can. Res. 64, 5270-5282; PCT Pub. Nos. WO 2008/030986 and WO 2009/111661(Kashani-Sabet & Haqq); U.S. Pat. No. 7,247,426 (Yakhini et al.), all of which are incorporated herein by reference in their entirety. Several researchers have reported the use of microRNAs (miRNA) for cancer or melanoma detection. These methods could be used in combination with the methylation methods described herein (see Mueller et al., 2009, J. Invest. Dermatol., 129, 1740-1751; Leidinger et al., 2010, BMC Cancer, 10, 262; U.S. Pat. Pub. 2009/0220969 (Chiang and Shi); PCT Pub. No. WO 2010/068473 (Reynolds and Siva); which are hereby incorporated by reference in their entirety). Alternatively, the methylated nucleic acids may be detected in blood either as free DNA or in circulating tumor cells. For in situ procedures see, e.g., Nuovo, G. J., 1992, PCR In Situ Hybridization: Protocols And Applications, Raven Press, NY, which is incorporated herein by reference in its entirety.

Methods for making nucleic acid microarrays are known to the skilled artisan and are described, for example, in Lockhart et al., 1996, Nat. Biotech. 14, 1675-1680, 1996 Schena et al., 1996, Proc. Natl. Acad. Sci. USA, 93, 10614-10619, U.S. Pat. No. 5,837,832 (Chee et al.) and PCT Pub. No. WO 00/56934 (Englert et al.), herein incorporated by reference. To produce a nucleic acid microarray, oligonucleotides may be synthesized or bound to the surface of a substrate using a chemical coupling procedure and an ink jet application apparatus, as described U.S. Pat. No. 6,015,880 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

The measurement of differentially methylated elements associated with melanoma may alone, or in conjunction with other melanoma detection tools discussed above (antibody staining, PCR, CGH, FISH) may have several other non-limiting uses. Amongst these uses are: (i) reclassifying specimens that were indeterminate or difficult to identify in a pathology laboratory; (ii) deciding to follow up with a lymph node examination and/or PET/CAT/MRI or other imaging methods; (iii) determining the frequency of follow up visits; or (iv) initiating other investigatory analysis such as a blood draw and evaluation for circulating tumor cells. Furthermore, the differentially methylated elements associated with melanoma may help to determine which patients would benefit from adjuvant treatment after surgical resection.

### 5.5. Compositions and Kits

The invention provides compositions and kits measuring methylation or polypeptides or polynucleotides regulated by the differentially methylated elements described herein using DNA methylation specific assays, antibodies specific for the polypeptides or nucleic acids specific for the polynucleotides. Kits for carrying out the diagnostic assays of the invention typically include, in suitable container means, (i) a reagent for methylation specific reaction or separation, (ii) a probe that comprises an antibody or nucleic acid sequence that specifically binds to the marker polypeptides or polynucleotides of the invention, (iii) a label for detecting the presence of the probe and (iv) instructions for how to measure the level of methylation (or polypeptide or polynucleotide). The kits may include several antibodies or polynucleotide sequences encoding polypeptides of the invention, e.g., a a first antibody and/or second and/or third and/or additional antibodies that recognize a protein encoded by a gene differentially methylated in melanoma. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

### 5.6. In Vivo Imaging

The various markers of the invention also provide reagents for in vivo imaging such as, for instance, the imaging of metastasis of melanoma to regional lymph nodes using labeled reagents that detect (i) DNA methylation associated with melanoma, (ii) a polypeptide or polynucleotide regulated by the differentially methylated elements. In vivo imaging techniques may be used, for example, as guides for surgical resection or to detect the distant spread of melanoma. For in vivo imaging purposes, reagents that detect the presence of these proteins or genes, such as antibodies, may be labeled with a positron-emitting isotope (e.g., 18F) for positron emission tomography (PET), gamma-ray isotope (e.g., 99 mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound. Fluorodeoxyglucose (FDG)-PET metabolic uptake alone or in combination with MRI is particularly useful.

Furthermore, such reagents may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. Nos. 4,452,720 (Harada et al.); 5,227,487 (Haugland and Whitaker); and 5,543,295 (Bronstein et al.). Other fluorescent labels suitable for use in the practice of this invention include a fluorescein dye. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. Nos. 4,439,356 (Khanna and Colvin); 5,066,580 (Lee), 5,750,409 (Hermann et al.); and 6,008,379 (Benson et al.). The kits may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, and other rhodamine dyes. Other rhodamine dyes can be found, for example, in U.S. Pat. Nos. 5,936,087 (Benson et al.), 6,025,505 (Lee et al.); 6,080,852 (Lee et al.). The kits may include a cyanine dye, such as, for example, Cy3, Cy3B, Cy3.5, Cy5, Cy5.5, Cy7. Phosphorescent compounds including porphyrins, phthalocyanines, polyaromatic compounds such as pyrenes, anthracenes and acenaphthenes, and so forth, may also be used.

### 5.7. Methods to Identify Compounds

A variety of methods may be used to identify compounds that modulate DNA methylation and prevent or treat melanoma progression. Typically, an assay that provides a readily measured parameter is adapted to be performed in the wells of multi-well plates in order to facilitate the screening of members of a library of test compounds as described herein. Thus, in one embodiment, an appropriate number of cells can be plated into the cells of a multi-well plate, and the effect of a test compound on the expression of a gene differentially methylated in melanoma can be determined. The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to modulate the expression of gene differentially methylated in melanoma. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175 (Rutter and Santi), Furka, 1991, Int. J. Pept. Prot. Res., 37:487-493; and Houghton et al., 1991, Nature, 354:84-88). Other chemistries for generating chemical diversity libraries can also be used. Such chemistries include, but are not limited to: U.S. Pat. Nos. 6,075,121 (Bartlett et al.) peptoids; 6,060,596 (Lerner et al.) encoded peptides; 5,858,670 (Lam et al.) random bio-oligomers; 5,288,514 (Ellman) benzodiazepines; 5,539,083 (Cook et al.) peptide nucleic acid libraries; 5,593,853 (Chen and Radmer) carbohydrate libraries; 5,569,588 (Ashby and Rine) isoprenoids; 5,549,974 (Holmes) thiazolidinones and metathiazanones; 5,525,735 (Takarada et al.) and 5,519,134 (Acevado and Hebert) pyrrolidines; 5,506,337 (Summerton and Weller) morpholino compounds; 5,288,514 (Ellman) benzodiazepines; diversomers such as hydantoins, benzodiazepines and dipeptides (Hobbs et al., 1993, Proc. Nat. Acad. Sci. USA, 90, 6909-6913), vinylogous polypeptides (Hagihara et al., 1992, J. Amer. Chem. Soc., 114, 6568), nonpeptidal peptidomimetics with glucose scaffolding (Hirschmann et al., 1992, J. Amer. Chem. Soc., 114, 9217-9218), analogous organic syntheses of small compound libraries (Chen et al., 1994, J. Amer. Chem. Soc., 116:2661 (1994)), oligocarbamates (Cho et al., 1993, Science, 261, 1303 (1993)), and/or peptidyl phosphonates (Campbell et al., 1994, J. Org. Chem., 59:658), nucleic acid libraries (see Ausubel, Berger and Sambrook, all supra); antibody libraries (see, e.g., Vaughn et al., 1996, Nat. Biotech., 14(3):309-314, carbohydrate libraries, e.g., Liang et al., 1996, Science, 274:1520-1522, small organic molecule libraries (see, e.g., benzodiazepines, Baum, 1993, C&EN, January 18, page 33. Devices for the preparation of combinatorial libraries are commercially available (see, e.g., 357 MPS, 390 MPS, Advanced Chem Tech, Louisville Ky., Symphony, Rainin, Woburn, Mass., 433 A Applied Biosystems, Foster City, Calif., 9050 Plus, Millipore, Bedford, Mass.). In addition, numerous combinatorial libraries are themselves commercially available (see, e.g., ComGenex (Princeton, N.J.), Asinex (Moscow, RU), Tripos, Inc. (St. Louis, Mo.), ChemStar, Ltd., (Moscow, RU), 3D Pharmaceuticals (Exton, Pa.), Martek Biosciences (Columbia, Md.), etc.).

Methylation modifiers are known and have been the basis for several approved drugs. Major classes of enzymes are DNA methyl transferases (DNMTs), histone deacetylases (HDACs), histone methyl transferases (HMTs), and histone acetylases (HATs). DNMT inhibitors azacitidine (Vidaza®) and decitabine have been approved for myelodysplastic syndromes (for a review see Musolino et al., 2010, Eur. J. Haematol. 84, 463-473; Issa, 2010, Hematol. Oncol. Clin. North Am. 24(2), 317-330; Howell et al., 2009, Cancer Control, 16(3) 200-218; which are hereby incorporated by reference in their entirety). HDAC inhibitor, vorinostat (Zolinza®, SAHA) has been approved by FDA for treating cutaneous T-cell lymphoma (CTCL) for patients with progressive, persistent, or recurrent disease (Marks and Breslow, 2007, Nat. Biotech. 25(1), 84-90). Specific examples of compound libraries include: DNA methyl transferase (DNMT) inhibitor libraries available from Chem Div (San Diego, Calif.); cyclic peptides (Nauman et al., 2008, ChemBioChem 9, 194-197); natural product DNMT libraries (Medina-Franco et al, 2010, Mol. Divers., Springer, published online 10 Aug. 2010); HDAC inhibitors from a cyclic α3β-tetrapeptide library (Olsen and Ghadiri, 2009, J. Med. Chem. 52(23), 7836-7846); HDAC inhibitors from chlamydocin (Nishino et al., 2006, Amer. Peptide Symp. 9(7), 393-394).

### 5.8. Methods of Inhibition Using Nucleic Acids

A variety of nucleic acids, such as antisense nucleic acids, siRNAs or ribozymes, may be used to inhibit the function of the markers of this invention. Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

The following Examples further illustrate the invention and are not intended to limit the scope of the invention.

### 6. EXAMPLES

### 6.1. Materials and Methods

Patients and Tissues

Retrospective clinic-based series of primary formalin-fixed, paraffin-embedded (FFPE) invasive cutaneous melanomas (n=22) or melanocytic nevi (n=27) were obtained from the Pathology Archives at UNC. Collection of tissues and associated patient information was approved by the Institutional Review Board at UNC. An honest broker searched the Pathology Laboratory Database at UNC-Chapel Hill and retrieved specimens collected after Jan. 1, 2001; all specimens were de-identified. All common histologic subtypes of primary cutaneous melanomas were included. Nevi were melanocytic and cutaneous, came from patients without melanoma, and included benign common melanocytic nevi, including intradermal, compound, congenital pattern and dysplastic nevi.

Medical Record Information

The UNC melanoma database manager extracted demographic and clinical information from the medical chart, including age, sex, anatomic sites of nevi and melanomas, and Breslow depth and Clark level of melanomas.

Standardized Pathology Review and Enrichment of Melanoma or Nevi

Five μm-thick tissue sections were cut from each block containing melanoma or nevus and were mounted on uncoated glass slides. A hematoxylin and eosin (H&E) slide of each melanoma or nevus specimen was reviewed by an expert dermatopathologist to confirm diagnosis, classify histologic subtype, and score standard histopathology features (histologic subtype, thickness, ulceration, solar elastosis, etc). In addition, the pathologist reviewed each tissue for histologic parameters that could affect assay performance and quality such as formalin-fixation adequacy, tissue size, percent tumor, and percent necrosis. To selectively isolate melanoma or nevi away from surrounding normal skin, H&E slides were used as guides for manual dissection of melanoma or nevus cells from each tissue section.

Cell Lines and Peripheral Blood Leukocytes

The Mel-505 melanoma and MCF-7 breast tumor cell lines were used to establish assay conditions and to assess assay reproducibility and the effects of formalin-fixation and contamination by non-melanocytic cells on methylation profiles. Cell lines were grown in RPMI medium with 10% fetal bovine serum and harvested while in log growth phase. Cells were pelleted and divided into two portions. One portion was used for DNA extraction (non-fixed) and the other pellet was fixed in buffered formalin, embedded in paraffin, and sections were cut from the paraffin blocks and were mounted on uncoated glass slides. Mixtures of DNA obtained from peripheral blood leukocytes (PBL) and the Mel-505 cell line in varying proportions were used to evaluate the effect of contamination of the methylation profile of the Mel-505 melanoma cell line by ‘non-melanocytic’ PBL cells.

Normal Skin

FFPE normal skin tissue was obtained from breast reduction specimens under IRB approval.

### 6.2. DNA Preparation

DNA was prepared from formalin-fixed nevi, melanoma, or normal skin tissues, or cell line pellets as previously published (Thomas et al., 2007, Cancer Epidemiol Biomarkers Prey. 16, 991-977). DNA was purified from non-fixed cell lines or peripheral blood leukocytes using the FlexiGene DNA according to the manufacturer's instructions (Qiagen, Valencia, Calif.).

### 6.3. Bisuifite Treatment of DNA

Sodium bisulfate modification of DNA obtained from FFPE or non-fixed cells was performed using the EZ DNA Methylation Gold kit (Zymo Research, Orange, Calif.). Approximately 500-1000 ng DNA from each tissue specimen was mixed with 130 μl of CT Conversion Reagent in a PCR tube and cycled in a thermal cycler at 98° C. for 10 minutes, 64° C. for 2.5 hours, and stored at 4° C. for up to 20 hours. The sample was then mixed with 600 μl M-binding buffer and spun through the Zymo-Spin IC column for 30 seconds (≧10,000×g). The column was washed with 100 μl of M-Wash buffer, spun, and incubated in 200 μl of M-Desulphonation buffer for 15-20 minutes. The column was then spun for 30 seconds (at ≧10,000×g), washed twice with 200 μl M-Wash buffer, and spun at top speed. The sample was eluted from the column with 10 μl M-Elution buffer and stored in a −20° C. freezer prior to use in the Illumina GoldenGate Methylation assay. After bisulfate treatment, DNA quantity and concentration were measured by a Nanodrop spectrophotometer, and DNA concentration adjusted to 50-60 ng/μl.

### 6.4. Illumina GoldenGate Cancer Panel I Methylation Analysis

Array-based DNA methylation profiling was accomplished using the Illumina GoldenGate Cancer Panel I methylation bead array (Illumina, San Diego, Calif.) to simultaneously interrogate 1505 CpG loci associated with 807 cancer-related genes. Bead arrays were run in the Mammalian Genotyping Core laboratory at the University of North Carolina. The Illumina GoldenGate methylation assay was performed as described previously (Bibikova et al., 2006, Genome Res., 16, 383-393). Two allele-specific oligonucleotides (ASO) and 1 locus-specific oligo (LSO) are designed to interrogate each CpG site, with the LSO containing a sequence which corresponds to a specific address on the BeadArray. Bisulfite-converted DNAs were biotinylated and bound to paramagnetic particles, hybridized to ASO and LSO probes, and the hybridized ASO oligos were extended in a methylation-specific fashion, then ligated to the LSO probe to create amplifiable templates. The joining of two fragments to create a PCR template provides an added level of locus specificity. The PCR that followed used 2 fluorescently-labeled (Cy3, Cy5) and biotinylated universal PCR primers corresponding to the ASO sequences (P1, P2) and a common P3 primer that binds to the LSO sequence. Labeled amplicons were bound to paramagnetic particles and denatured, then after filtering out the biotinylated strands, the fluor-labeled strands were hybridized to the Sentrix BeadArray under a temperature gradient, and imaged using the BeadArray Scanner (Illumina) Methylation status of the interrogated CpG sites was determined by comparing the ratio of the fluorescent signal from the methylated allele to the sum from the fluorescent signals of both methylated and unmethylated alleles. Controls for methylation status used on each bead array included the Zymo Universal Methylated DNA Standard as the positive, fully-methylated control, and a GenomePlex (Sigma) whole genome amplified (WGA) DNA used as the negative, unmethylated control.

### 6.5. Bioinformatics and Statistical Analysis

The data were assembled using the GenomeStudio Methylation software from Illumina (San Diego, Calif.). All array data points were represented by fluorescent signals from both methylated (Cy5) and unmethylated (Cy3) alleles. Background intensity computed from the negative control was subtracted from each data point. The methylation level of individual interrogated CpG sites was determined by the β-value, defined as the ratio of fluorescent signal from the methylated allele to the sum of the fluorescent signals of both the methylated and unmethylated alleles and calculated as β=max(Cy5,0)/(|Cy5|+|Cy3|+100). β values ranged from 0 in the case of completely unmethylated to 1 in the case of fully methylated DNA. The BeadStudio Methylation Module software (Illumina) was used to create scatter plots to examine the relationship between cell line replicates and between FFPE and non-fixed samples. The correlation coefficient, R², was calculated for each comparison.

For studies of melanomas and nevi, average methylation β values were derived from the multiple β values calculated for each CpG site within the melanoma (n=22) or nevus (n=27) groups. Prior to clustering or further statistical analysis, filtering was performed to remove a total of 478 probes that corresponded to 68 CpG sites on the X chromosome and 410 that were reported to contain a single nucleotide polymorphism or repeat within the recognition sequence thus making the probes unreliable in at least some samples (Byun et al., 2009, Hum. Mol. Genet. 18, 4808-4817). In addition, a detection p-value computed by GenomeStudio and representing the probability that the signal from a given CpG locus is distinguishable from the negative controls was used as a metric for quality control for sample performance. β values with a detection p-value greater than 10⁻⁵ were considered unreliable and set to be missing (Marsit et al, 2009, Carcinogenesis, 30, 416-422). Two nevus samples with more than 25% missing β values and 39 CpG loci with more than 20% missing samples were excluded from analysis. The final data contained 988 CpG loci in 646 genes and 49 samples (22 melanomas and 27 moles).

All subsequent statistical analyses were carried out using the R package (http://www.r-project.org/). For exploratory/visualization purposes, unsupervised hierarchical clustering using the Euclidean metric and complete linkage was performed. To adjust for age or gender effect, a linear model was fitted to the logit transformed β-values using age and gender as covariates in comparing the methylation levels between melanomas and moles at each locus. Bonferroni correction was used to adjust for multiple comparisons, i.e., significant loci were selected with p-value≦0.05/988=5.06×10⁻⁵, with an additional filter of mean adjusted β-value difference 0.2 between melanomas and moles to be clinically significant. In addition, the area under the receiver operating characteristics curve (AUC) was computed to summarize the accuracy of correctly classifying melanomas and moles using these significant loci. The Prediction Analysis of Microarrays (PAM) approach (Tibshirani et al. 2002, Proc. Nat. Acad. Sci. USA, 99, 6567-6572) was carried out to assess the classification of melanoma and nevus samples by the method of nearest shrunken centroids.

Gene Ontology Analysis

The DAVID Bioinformatics Resources 6.7 Functional Annotation Tool (http://david.abcc.ncifcrf.gov/home.jsp) was used to perform gene-GO term enrichment analysis to identify the most relevant GO terms associated with the genes found to be differentially methylated between nevi and malignant melanomas. Gene function was also investigated using GeneCards (http://www.genecards.org/).

### 6.6. Results

Optimization and Validation of Illumina Methylation Array in Cell Lines

We optimized conditions for performance of the Illumina GoldenGate Methylation Cancer Panel I array, which is designed to detect methylation at 1505 CpG sites in the promoters and regulatory regions of 807 cancer related genes. We also evaluated array reproducibility, and the impact of formalin fixation and intermixture of melanocytic with non-melanocytic DNA on methylation profiles. In testing a range of bisulfate-treated DNA quantities from 25 to 500 ng, we determined that a minimum of 200 ng non-fixed DNA or 250 ng of formalin-fixed DNA was needed to successfully perform array profiling, and that sufficient DNA was recoverable from the majority of FFPE melanoma or nevus tissues.

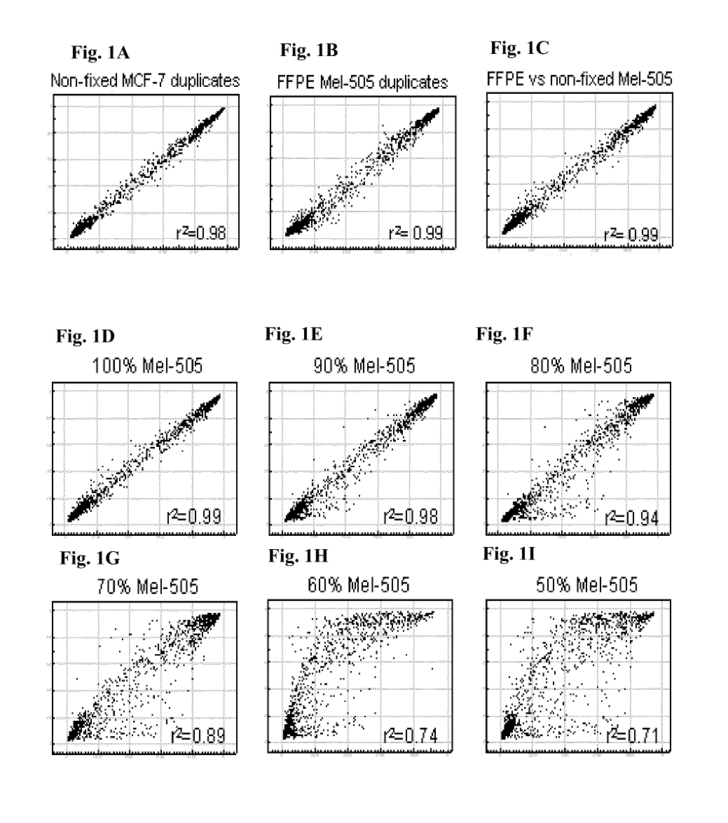

We found very high reproducibility between non-fixed cell lines and the same lines which had undergone the FFPE process. Cell lines were pelleted, formalin-fixed, and paraffin-embedded just as tissue is in the clinical setting to create FFPE-processed equivalents for cell lines. Shown in FIGS. 1A-1C are replicate methylation array profiles of non-formalin-fixed MCF-7 breast tumor cell DNA, formalin-fixed DNA from the Mel-505 melanoma cell line, as well as methylation profiles from non-fixed versus FFPE Mel-505 DNA. Each of these array replicates produced was highly reproducible, showing r² values of ≧0.98. We optimized the Illumina GoldenGate Methylation assay using 250-500 ng, and tested assay performance on matched pairs of frozen and/or FFPE cell line DNA. Using ≧250 ng DNA, methylation profiles were compared and showed very high correlation between frozen duplicates of 8 cell line DNAs (r²=0.98), 20 matched FFPE and frozen cell line DNAs (r²=0.98), and 14 FFPE duplicate DNA samples (r²=0.97). The FFPE tissues produced methylation profiles very similar to those from matched frozen specimens, and that 250 ng or more of FFPE DNA provides suitable template for methylation profiling.

We conducted experiments to gauge the proportion of melanoma cell line MeI-505 DNA that must be present in a tumor/normal DNA mixture in order for the melanoma methylation profile to be evident. In FIGS. 1D-1I, the Mel-505 cell line DNA was diluted with increasing proportions (from 0 to 50%) of DNA from normal peripheral blood leukocytes (PBLs) (90% Mel-505/10% PBL, 80% Mel-505/20% PBL, 70% Mel-505/30% PBL, 60% Mel-505/40% PBL, 50% Mel-505/50% PBL), and each mixture was plotted against the profile for pure (100%) Mel-505 cell line DNA. The Mel-505 cell line profile was evident even after dilution with up to 30% PBL DNA (70% Mel-505/30% PBL mixture) (r²=0.89), indicating that a moderate level of contamination of melanocytic cells by normal DNA will not significantly disrupt the melanoma methylation pattern. This result provides a guideline for estimating the necessary purity of tumor DNA to achieve methylation array results that are representative of melanocytic target DNA.

Characteristics of Patients with Benign Nevi or Malignant Melanoma

Illumina methylation array analysis was performed on 27 FFPE benign nevi, 22 FFPE primary malignant melanomas and 9 FFPE lymph node metastatic melanomas. The patient characteristics as well as histologic and clinical features of these tissues are detailed in Table 1 below. The mean age of nevus patients (29 years) was significantly less than melanoma patients (61 years; p<0.0001). Among patients with nevi, 83% were younger than 40 yrs, whereas only 27% of melanoma patients were younger than 40 yrs. Forty-one percent of nevus patients and 50% of melanoma patients were male. The anatomic site of nevi differed significantly from that of melanomas (p=0.1300), with nevi occurring predominantly on the head and neck (HN) (35%) or trunk (52%), and melanomas occurring mostly on either the trunk (36%) or an extremity (41%). Among nevi, 38% were classified histologically as intradermal melanocytic nevi, 31% were described as compound melanocytic nevi, and 21% were identified as compound melanocytic nevi with congenital pattern. Only 7% of nevi were classified as being compound dysplastic nevi with slight atypia. Among melanomas, 50% were of the superficial spreading histologic type, 14% were lentigo maligna, 14% were acral lentiginous, 9% were nodular, and 9% were spindle cell melanoma. The melanomas consisted mostly of deeper lesions, with 32% having a Breslow depth of ≦1.5 mm, and 68% having Breslow depth of >1.5 mm.

TABLE 1
Clinical and histologic characteristics of 27 non-malignant nevi
and 22 primary cutaneous malignant melanomas and 9 lymph node
metastatic melanomas evaluated for DNA promoter methylation

| | | | | | | Breslow | |
|---|---|---|---|---|---|---|---|
| | | Histologic | Age | | | depth | Presence of |
| No | Lesion | Type/Features | yrs | Sex | Site | (mm) | Lymphocytes |
| 001 | Melanoma | SSM | 89 | Male | extremity | 4.6 | absent |
| 002 | Melanoma | SSM | 33 | Male | trunk | 0.82 | 1-2 |
| 003 | Melanoma | SSM | 81 | female | HN | 3.65 | absent |
| 004 | Melanoma | SSM | 38 | female | trunk | 5.7 | absent |
| 005 | Melanoma | SC | 76 | Male | extremity | 1.3 | 1-2 |
| 006 | Melanoma | NM | 26 | Male | trunk | 1.0 | 3 |
| 007 | Melanoma | SSM | 43 | Male | trunk | 0.59 | 3 |
| 009 | Melanoma | SSM | 35 | Male | trunk | 1.3 | 3 |
| 010 | melanoma | SSM | 78 | Male | extremity | 4.55 | absent |
| 011 | melanoma | SSM | 71 | female | extremity | 3.5 | absent |
| 013 | melanoma | LMM | 82 | female | HN | 1.78 | 1-2 |
| 014 | melanoma | LMM | 83 | female | HN | 3.65 | absent |
| 016 | melanoma | SSM | 70 | Male | extremity | 0.93 | 1-2 |
| 017 | melanoma | SSM | 76 | Male | trunk | 1.25 | 1-2 |
| 019 | melanoma | NM | 68 | female | trunk | 2.6 | absent |
| 021 | melanoma | SC | 47 | female | HN | 10.0 | absent |
| 022 | melanoma | ALM | 84 | female | extremity | 7.1 | absent to |
| | | | | | | | minimal |
| 117 | melanoma | ALM | 31 | female | extremity | 5.4 | absent to |
| | | | | | | | minimal |
| 124 | melanoma | LMM | 67 | female | HN | 5.0 | 1-2 |
| 126 | melanoma | ALM | 69 | Male | trunk | 5.25 | absent |
| 503 | melanoma | SSM | 36 | female | extremity | 4.6 | 1-2 |
| 504 | melanoma | UNCL | 49 | Male | extremity | 4.35 | absent |
| 475 | nevus | compound | 18 | Male | HN | na | absent |
| | | dysplastic nevus | | | | | |
| | | w/slight atypia | | | | | |
| 476 | nevus | compound nevus | 38 | Female | HN | na | absent |
| 477 | nevus | compound nevus | 48 | Female | extremity | na | absent |
| 478 | nevus | compound nevus | 22 | Female | extremity | na | absent |
| 479 | nevus | compound nevus | 34 | Male | HN | na | absent |
| 480 | nevus | compound nevus | 27 | Male | HN | na | absent |
| 481 | nevus | compound nevus | 21 | Female | extremity | na | absent |
| 482 | nevus | compound nevus | 25 | Male | trunk | na | absent |
| 483 | nevus | compound nevus | 13 | Male | trunk | na | absent |
| 484 | nevus | intradermal nevus | 32 | Female | HN | na | absent |
| 485 | nevus | intradermal nevus | 21 | Female | HN | na | absent |
| 486 | nevus | intradermal nevus | 41 | Female | HN | na | absent |
| 487 | nevus | intradermal nevus | 26 | Female | trunk | na | absent |
| 488 | nevus | intradermal nevus | 89 | Female | trunk | na | absent |
| 489 | nevus | intradermal nevus | 13 | Female | HN | na | absent |
| 490 | nevus | intradermal nevus | 26 | Female | extremity | na | absent |
| 492 | nevus | intradermal nevus | 20 | Female | trunk | na | absent |
| 493 | nevus | intradermal nevus | 15 | Female | trunk | na | absent |
| 494 | nevus | compound nevus | 33 | Female | trunk | na | absent |
| 495 | nevus | compound nevus w/ | 9 | Male | HN | na | absent |
| | | congenital pattern | | | | | |
| 496 | nevus | compound nevus | 43 | Male | trunk | na | absent |
| 497 | nevus | compound nevus w/ | 23 | Male | trunk | na | absent |
| | | congenital pattern | | | | | |
| 498 | nevus | compound nevus w/ | 18 | Female | trunk | na | absent |
| | | congenital pattern | | | | | |
| 499 | nevus | compound nevus w/ | 66 | Male | HN | na | absent |
| | | congenital pattern | | | | | |
| 500 | nevus | compound cutaneous | 22 | Female | trunk | na | absent |
| 501 | nevus | compound nevus w/ | 13 | Female | trunk | na | absent |
| | | congenital pattern | | | | | |
| 502 | nevus | compound nevus w/ | 11 | Male | trunk | na | absent |
| | | congenital pattern | | | | | |
| 029 | melanoma | metastasis | 83 | Male | cervical | na | |
| 030 | melanoma | metastasis | 82 | male | cervical | na | |
| 049 | melanoma | metastasis | 73 | male | axillary | na | |
| 061 | melanoma | metastasis | 80 | female | lymph | na | |
| | | | | | node | | |
| 107 | melanoma | metastasis | 47 | male | cervical | na | |
| 114 | melanoma | metastasis | 62 | female | axillary | na | |
| 116 | melanoma | metastasis | 91 | female | inguinal | na | |
| 119 | melanoma | metastasis | 31 | male | inguinal | na | |
| 122 | melanoma | metastasis | 22 | female | axillary | na | |

### 6.7. Comparison of Methylation Profiles in Benign Nevi and Malignant Melanomas

We performed Illumina GoldenGate Cancer Panel I methylation profiling to evaluate promoter methylation patterns in 27 benign nevi and 22 primary melanomas. Illumina methylation array results were subjected to filtering to remove 68 probes that corresponded to CpG sites on the X chromosome and 410 probes that were reported to contain a SNP or repeat (Byun et al, 2009), thus making them unreliable in some samples. Additionally, β values with a detection p-value greater than 10⁻⁵ were considered unreliable and set as missing data points (Marsit et al, 2009); using this criterium, two nevus samples with more than 25% missing β values as well as 39 CpG loci with β values missing in more than 20% missing samples were excluded from analysis. The final data set consisted of 988 CpG loci within 646 genes in 49 specimens (22 melanomas and 27 moles).

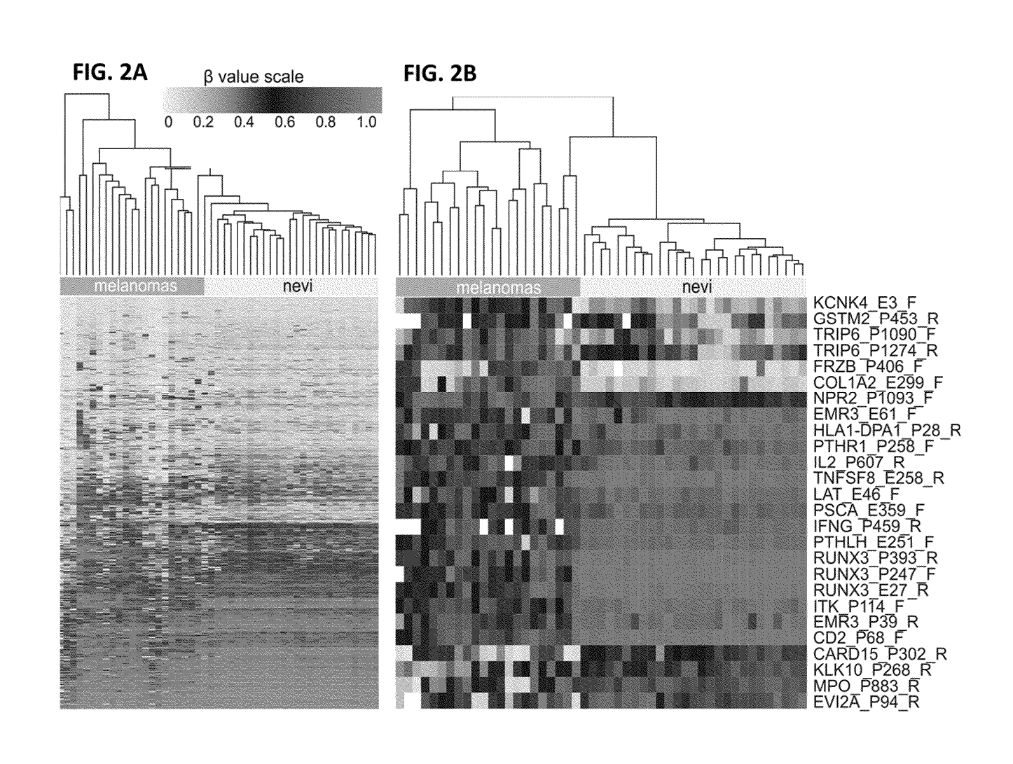

Unsupervised hierarchical clustering was used to compare methylation patterns at 988 CpG loci in benign nevi and malignant melanomas. Clustering produced a clear separation of melanomas from benign nevi, with two major clusters of nevi and at least four clusters of melanomas identified, suggesting that the methylation signature of melanomas is fundamentally distinct from that of nevi. Using class comparison analyses, 75 CpG sites in 63 genes were identified that differed significantly (with P values of ≦0.05) between nevi and melanomas after Bonferroni correction for multiple comparisons; a list of these 75 loci is provided in Table 2. After further adjustment for patient age and sex, we identified a total of 29 CpG loci in 23 genes that differed significantly between melanomas and nevi; these included 22 CpG loci that were significantly hypomethylated and 7 CpG loci that were significantly hypermethylated in melanoma. The heatmap based on supervised clustering of the 29 differentially methylated CpG loci in nevi and melanomas is shown in FIG. 2. The loci that significantly distinguished melanomas from nevi based on methylation were KCNK4, GSTM2, TRIP6 (2 sites), FRZB, COL1A2, NPR2, which showed hypermethylation, and CARD15/NOD2, KLK10, MPO, EVI2A, EMR3 (2 sites), HLA-DPA1, PTHR1, IL2, TNFSF8, LAT, PSCA, IFNG, PTHLH, three sites in RUNX3 (3 sites), ITK, CD2, OSM (2 sites), and CCL3, which showed hypomethylation in melanomas compared with nevi.

TABLE 2
75 CpG sites from the Illumina GoldenGate Methylation Cancer Panel I array that show
significant differences in methylation between melanomas and benign nevi after
Bonferroni correction for multiple comparisons

| TargetID | Raw_p | Bonferroni_p | FDR_p |
|---|---|---|---|
| RUNX3_P393_R | 4.02E−14 | 3.98E−11 | 1.48E−11 |
| CD2_P68_F | 8.05E−14 | 7.95E−11 | 1.48E−11 |
| MPO_P883_R | 8.05E−14 | 7.95E−11 | 1.48E−11 |
| RUNX3_E27_R | 8.05E−14 | 7.95E−11 | 1.48E−11 |
| RUNX3_P247_F | 8.96E−14 | 8.86E−11 | 1.48E−11 |
| OSM_P188_F | 1.61E−13 | 1.59E−10 | 1.99E−11 |
| TNFSF8_E258_R | 2.82E−13 | 2.78E−10 | 3.09E−11 |
| PTHLH_E251_F | 4.83E−13 | 4.77E−10 | 4.77E−11 |
| ITK_E166_R | 2.70E−12 | 2.66E−09 | 2.22E−10 |
| PECAM1_P135_F | 3.68E−11 | 3.64E−08 | 2.27E−09 |
| CCL3_E53_R | 4.88E−11 | 4.82E−08 | 2.68E−09 |
| EVI2A_E420_F | 4.88E−11 | 4.82E−08 | 2.68E−09 |
| ITK_P114_F | 1.09E−10 | 1.08E−07 | 4.90E−09 |
| LAT_E46_F | 1.41E−10 | 1.39E−07 | 5.81E−09 |
| EVI2A_P94_R | 9.23E−10 | 9.12E−07 | 3.04E−08 |
| IL2_P607_R | 2.05E−09 | 2.03E−06 | 6.34E−08 |
| TDG_E129_F | 3.23E−09 | 3.19E−06 | 9.37E−08 |
| IFNG_P459_R | 6.90E−09 | 6.82E−06 | 1.57E−07 |
| GABRA5_P1016_F | 1.22E−08 | 0.000012048 | 2.68E−07 |
| EMR3_P39_R | 1.75E−08 | 1.72E−05 | 3.52E−07 |
| EMR3_E61_F | 2.08E−08 | 2.06E−05 | 4.11E−07 |
| DSG1_P159_R | 2.94E−08 | 2.90E−05 | 5.59E−07 |
| HLA−DPA1_P28_R | 3.48E−08 | 3.44E−05 | 6.38E−07 |
| OSM_P34_F | 4.58E−08 | 0.000045277 | 8.23E−07 |
| ALOX12_E85_R | 4.87E−08 | 4.81E−05 | 8.29E−07 |
| DES_E228_R | 4.87E−08 | 4.81E−05 | 8.29E−07 |
| PTK7_E317_F | 1.09E−07 | 0.000107353 | 1.60E−06 |
| KCNK4_E3_F | 1.27E−07 | 0.000125429 | 1.72E−06 |
| MMP10_E136_R | 1.27E−07 | 0.000125429 | 1.72E−06 |
| KLK10_P268_R | 1.48E−07 | 0.000146312 | 1.95E−06 |
| SNURF_P2_R | 1.48E−07 | 0.000146312 | 1.95E−06 |
| COL1A2_E299_F | 2.01E−07 | 0.000198158 | 2.48E−06 |
| MMP2_P303_R | 2.70E−07 | 0.00026676 | 3.21E−06 |
| FRZB_P406_F | 3.10E−07 | 0.000306716 | 3.59E−06 |
| CASP8_E474_F | 4.17E−07 | 0.000412183 | 4.74E−06 |
| GSTM2_P453_R | 4.40E−07 | 0.000434777 | 4.94E−06 |
| THBS2_P605_R | 4.85E−07 | 0.000479408 | 5.33E−06 |
| EPHA2_P203_F | 5.54E−07 | 0.000547104 | 5.82E−06 |
| GNMT_P197_F | 5.54E−07 | 0.000547104 | 5.82E−06 |
| PTHR1_P258_F | 5.54E−07 | 0.000547104 | 5.82E−06 |
| PSCA_E359_F | 1.26E−06 | 0.001240291 | 1.22E−05 |
| CARD15_P302_R | 1.63E−06 | 0.001613456 | 1.5079E−05 |
| DSG1_E292_F | 2.06E−06 | 0.002037049 | 1.85E−05 |
| IPF1_P750_F | 2.11E−06 | 0.002089181 | 1.85E−05 |
| MUSK_P308_F | 2.11E−06 | 0.002089181 | 1.85E−05 |
| SNURF_E256_R | 2.11E−06 | 0.002089181 | 1.85E−05 |
| ARHGDIB_P148_R | 2.40E−06 | 0.002373277 | 2.05E−05 |
| COL1A1_P117_R | 2.40E−06 | 0.002373277 | 2.05E−05 |
| TRIP6_P1274_R | 2.40E−06 | 0.002373277 | 2.05E−05 |
| MEST_P62_R | 3.50E−06 | 0.003456272 | 2.86E−05 |
| SHB_P691_R | 3.96E−06 | 0.003909276 | 3.18E−05 |
| SYK_P584_F | 3.96E−06 | 0.003909276 | 3.18E−05 |
| SNURF_P78_F | 5.05E−06 | 0.004985595 | 3.96E−05 |
| CDH13_P88_F | 5.79E−06 | 0.005720768 | 4.47E−05 |
| TNFSF8_P184_F | 7.21E−06 | 0.007126004 | 5.48E−05 |
| BMPR1A_E88_F | 8.11E−06 | 0.008011205 | 6.07E−05 |
| OPCML_P71_F | 8.37E−06 | 0.008269514 | 6.22E−05 |
| HBII−52_P563_F | 9.11E−06 | 0.008997683 | 6.57E−05 |
| PWCR1_P357_F | 9.11E−06 | 0.008997683 | 6.57E−05 |
| TRIP6_P1090_F | 9.11E−06 | 0.008997683 | 6.57E−05 |
| CD86_P3_F | 1.02E−05 | 0.010095984 | 7.2633E−05 |
| HOXA11_P698_F | 1.02E−05 | 0.010095984 | 7.2633E−05 |
| NEFL_E23_R | 1.15E−05 | 0.011317697 | 8.08E−05 |
| PTK6_E50_F | 1.28E−05 | 0.012675435 | 8.56E−05 |
| ZIM2_P22_F | 1.28E−05 | 0.012675435 | 8.56E−05 |
| SEMA3B_E96_F | 1.44E−05 | 0.014183028 | 9.52E−05 |
| ALOX12_P223_R | 1.60E−05 | 0.01585551 | 0.000105 |
| NPR2_P1093_F | 1.60E−05 | 0.01585551 | 0.000105 |
| LOX_P313_R | 1.64E−05 | 0.016180651 | 0.00010645 |
| MST1R_P87_R | 2.00E−05 | 0.019762343 | 0.0001275 |
| SERPINA5_E69_F | 2.00E−05 | 0.019762343 | 0.0001275 |
| TNFRSF10D_E27_F | 3.08E−05 | 0.030382223 | 0.00018989 |
| PGR_E183_R | 4.21E−05 | 0.041578421 | 0.00024897 |
| RARA_E128_R | 4.21E−05 | 0.041578421 | 0.00024897 |
| HPN_P374_R | 4.40E−05 | 0.043487012 | 0.00025885 |

29 bolded loci were still significant after adjustment for age and sex.

### 6.8. PAM Analysis to Identify CpG Loci Predictive of Melanoma

From among the 29 CpG sites that significantly distinguished melanomas from benign nevi, we selected a panel of markers for systematic testing in prediction models. Prediction Analysis for Microarray (PAM) was carried out to assess the classification of melanoma and nevus samples by the method of nearest shrunken centroids. The PAM algorithm automatically identifies CpG loci that contribute most to the melanoma classification. Using 10-fold cross-validation to train the classifier, the optimal shrinkage threshold was chosen to be 4.28 with 12 CpG loci required for optimal classification. This approach yielded a zero cross-validation error, with no misclassification. The 12 CpG loci identified by PAM analysis that provided the most accurate prediction of melanoma were: RUNX3_P393_R, RUNX3_P247_F, RUNX3_E27_R, COL1A2_E299_F, MPO_P883_R, TNFSF8_E258_R, CD2_P68_F, EVI2A_P94_R, OSM_P168_F, ITKP114_F, FRZB_P406_F, ITK_E166_R. All but one locus (ITK_E166_R) exhibited mean β differences between melanomas and nevi of ≧0.2.

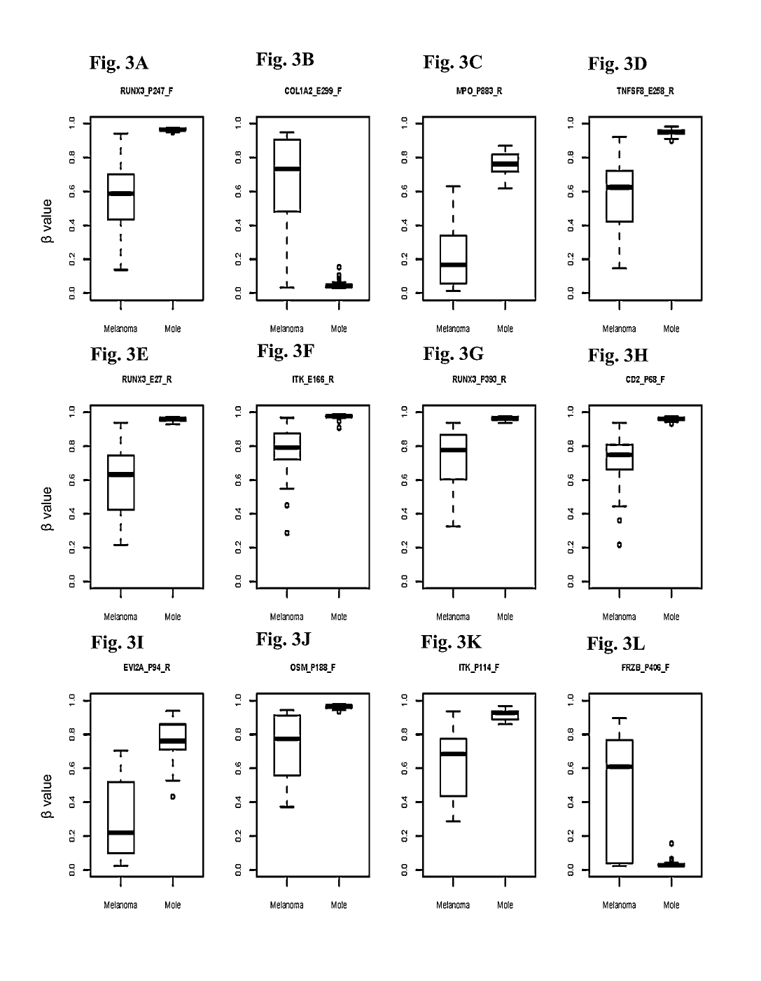

The box plots shown in FIGS. 3A-3L display the mean, range, and standard deviation of β values in nevi and melanomas for the 12 CpG sites that are highly predictive of melanoma as determined by PAM analysis. For most CpG loci showing hypomethylation in melanomas compared with benign nevi, mean methylation β values were very high (nearly 1.0), indicating that these CpG sites were uniformly highly methylated in nevi, however, methylation was lost to varying degrees in primary melanomas. Among the CpG loci exhibiting hypermethylation in melanomas, FRZB_P406_F and COL1A2_E299_F, were poorly methylated in nevi, having mean β values near 0.1, but showed considerably higher methylation in many melanomas, with mean β values between 0.6 and 0.7.

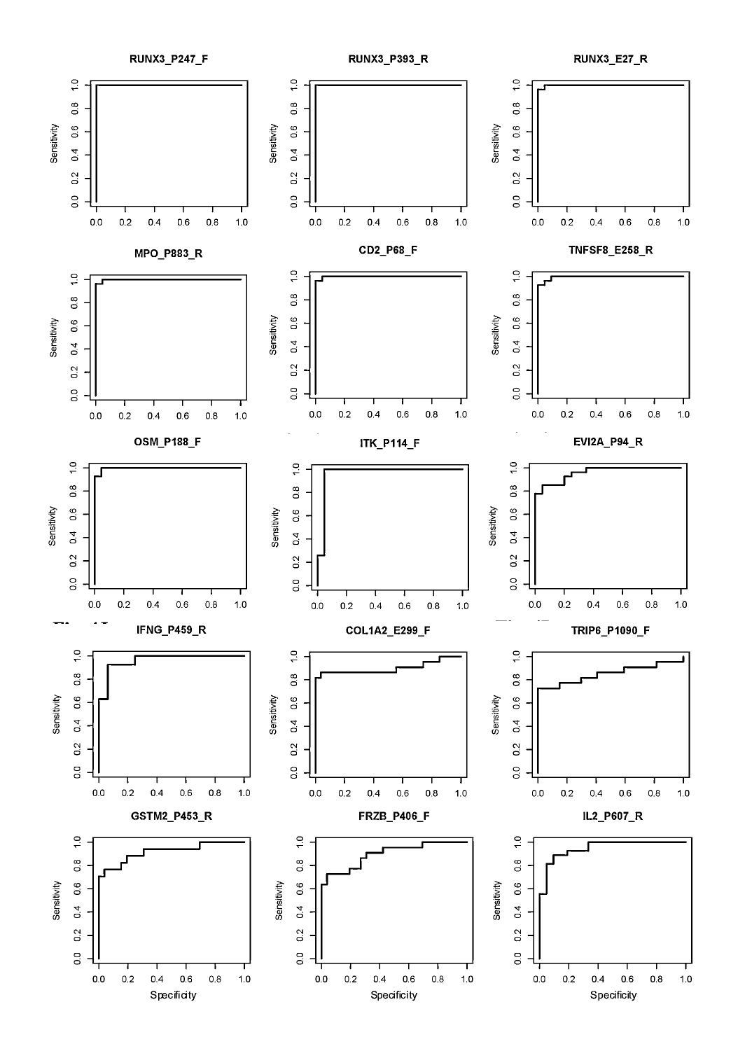

Sensitivity analysis conducted using Receiver Operator Characteristic (ROC) curves are shown in FIGS. 4A-4O which plot the sensitivity versus the specificity of the 12 CpG loci identified by PAM analysis. The area under the curve (AUC) ranged from 0.89 to 0.90 for the 2 hypermethylated loci, and from 0.96 to 1.00 for the 10 hypomethylated loci. In particular, two of the RUNX3 probes (RUNX3_P247_F and RUNX3_P393_R) exhibited both 100% sensitivity and 100% specificity in identifying melanomas. The sensitivity, specificity and AUC for all 29 CpG loci that differed significantly after adjustment between melanomas and nevi, including the 12 predictive loci identified by PAM analysis, are shown in Table 3A. Data on sequences showing differences in methylation levels (β values) may be found in Table 6 for a combined analysis where metastases were included with melanomas. Descriptions of sequences, methylation sites from the Illumina array and gene names may be found in Table 4A and 4B for the melanoma vs. benign nevi comparison. Data for the metastases vs. benign nevi comparison may be found in Table 5A and 5B (Section 6.10). Some additional specific sequences methylated in the metastatic samples may be found in Tables 7A and 7B. Specific sequences and methylation sites for other CpG probes may be obtained from the gene list for the Illumina GoldenGate Cancer Panel 1.

To assess the possibility that methylation differences between melanomas and nevi could result in part from contamination by non-melanocytic DNA, e.g., lymphocytic infiltration of the melanoma specimens or contamination of small melanocytic specimens by normal surrounding skin, the study pathologist estimated the degree of lymphocytic infiltration in melanocytic specimens (Table 1). In addition, we compared the mean methylation 13 profiles in 4 peripheral blood leukocyte (PBL) samples and 2 normal skin specimens with those of nevi and melanomas (data not shown). Significant lymphocytic presence was noted in only 2 melanomas and none of the nevi, making it unlikely that differential methylation involving immune loci was related to the infiltration by tumor-associated lymphocytes. Methylation profiles of PBL samples showed comparable levels of methylation among the 4 specimens at individual CpG loci.

### 6.9. Functions of Genes Differentially Methylated in Melanomas and Nevi

We explored the major functions of the 23 genes (with 29 CpG sites) that most significantly distinguished melanomas from benign nevi. Table 3B provides gene functional information obtained through gene ontology searches using the DAVID Bioinformatics Resources 6.7 (http://david.abcc.ncifcrf.gov/home.jsp) and the human gene database, GeneCards (http://www.genecards.org). Details on the mean β in nevi and melanomas, mean β differences, adjusted p-values, and AUC (and the sensitivity and specificity of melanoma prediction) for each gene are presented in Table 3A. While the number of genes identified was too small to fully evaluate functional pathways, it was of interest that half (13 of 23) possessed immune response or inflammation pathway functions, including roles in T-cell signaling and/or natural killer cell cytotoxicity (IFNG, IL2, ITK, LAT, CD2, CCL3, TNFSF8, HLA-DPA1), myeloid-myeloid cell interactions (EMR3), neutrophil microbicidal activity (MPO), innate immunity (CARD15/NOD2), and NF-κB activation (TRIP6, OSM, CARD15/NOD2). Three genes are involved in thyroid (TRIP6) or parathyroid (PTHLH, PTHR1) hormonal regulation. Several other genes have well-characterized roles in cancer cell growth, cell adhesion, or apoptosis (RUNX3, FRZB, TNFSF8, KLK10, PSCA, OSM, COL1A2). The 3 CpG sites located within the RUNX3 gene all exhibited significantly lower methylation in melanomas compared with nevi even though RUNX3 has been considered a tumor suppressor gene and might be expected to display promoter hypermethylation, rather than hypomethylation, in malignancy (Kitago et al., 2009, Clin. Cancer Res. 15, 2988-2994). However, more recent studies suggest that RUNX3 may have both tumor suppressor and oncogenic functions depending on the cellular context (Chuang and Ito, 2010, Oncogene 29, 2605-2615).

TABLE 3A
Twenty-nine CpG loci exhibiting significant promoter methylation
differences between melanomas and benign nevi

| | | | | | | | | |
|---|---|---|---|---|---|---|---|---|
| | | Nevus | Melanoma | | | | | |
| Gene | CpG/ | mean | mean | | Mean β | | | |
| Symbol | Probe | β | β | P value | Difference | AUC | Skin | PBL |

Hypermethylated in melanomas compared with nevi (n = 7)

| | | | | | | | | |
|---|---|---|---|---|---|---|---|---|
| COL1A2 | E299_F | 0.0386 | 0.5093 | 4.1 × 10⁻⁵ | +0.4707 | 0.9007 | U | U |
| FRZB | P406_F | 0.0255 | 0.2831 | 1.4 × 10⁻² | +0.2576 | 0.8986 | U | U |
| GSTM2 | P453_R | 0.1548 | 0.6087 | 6.3 × 10⁻³ | +0.4539 | 0.9186 | P | M |
| KCNK4 | E3_F | 0.0646 | 0.4014 | 2.6 × 10⁻³ | +0.3369 | 0.9057 | U | M |
| NPR2 | P1093_F | 0.5459 | 0.8224 | 1.8 × 10⁻² | +0.2765 | 0.8434 | P | M |
| TRIP6 | P1090_F | 0.0619 | 0.5741 | 6.3 × 10⁻⁵ | +0.5121 | 0.8518 | U | M |
| TRIP6 | P1274_R | 0.1584 | 0.6660 | 2.7 × 10⁻³ | +0.5076 | 0.8704 | U | M |

Hypomethylated in melanomas compared with nevi (n = 22)

| | | | | | | | | |
|---|---|---|---|---|---|---|---|---|
| CCL3 | E53_R | 0.9227 | 0.7180 | 5.7 × 10⁻⁵ | −0.2047 | 0.9714 | P | M |
| CARD15 | P302_R | 0.5146 | 0.0962 | 3.1 × 10⁻² | −0.4184 | 0.8754 | | |
| EV12A | P94_R | 0.7358 | 0.2121 | 1.3 × 10⁻³ | −0.5237 | 0.9592 | M | U |
| HLA- | P28_R | 0.8886 | 0.5277 | 3.3 × 10⁻² | −0.3609 | 0.9191 | | U |
| IFNG | P459_R | 0.9150 | 0.6334 | 7.9 × 10⁻⁹ | −0.2915 | 0.9630 | M | M |
| ITK | P114_F | 0.9289 | 0.6480 | 2.7 × 10⁻⁶ | −0.2809 | 0.9663 | M | M |
| ITK | E166_R | | | | | | | |
| LAT | E46_F | 0.8780 | 0.4948 | 1.8 × 10⁻² | −0.3832 | 0.9646 | P | U |
| IL2 | P607_R | 0.8922 | 0.6022 | 9.0 × 10⁻³ | −0.2900 | 0.9489 | | M |
| CD2 | P68_F | 0.9620 | 0.7382 | 1.3 × 10⁻⁷ | −0.2238 | 0.9983 | M | U |
| MPO | P883_R | 0.7713 | 0.1750 | 2.4 × 10⁻⁶ | −0.5963 | 0.9983 | P/U | U |
| EMR3 | E61_F | 0.9019 | 0.4205 | 1.3 × 10⁻³ | −0.4814 | 0.9242 | M | P |
| EMR3 | P39_R | 0.9210 | 0.6379 | 2.0 × 10⁻³ | −0.2831 | 0.9259 | M | P |
| OSM | P188_F | 0.9560 | 0.7516 | 3.6 × 10⁻⁶ | −0.2044 | 0.9966 | | |
| OSM | P34_F | 0.9000 | 0.6988 | 3.0 × 10⁻² | −0.2008 | 0.9206 | U | U |
| TNFSF8 | E258_R | 0.9552 | 0.6155 | 1.6 × 10⁻⁷ | −0.3517 | 0.9949 | M | U |
| PTHLH | E251_R | 0.9074 | 0.5488 | 5.8 × 10⁻⁶ | −0.3586 | 0.9933 | | |
| PTHR1 | P258_F | 0.8128 | 0.5253 | 4.5 × 10⁻³ | −0.2875 | 0.8889 | | M |
| RUNX3 | P393_R | 0.9595 | 0.6912 | 3.3 × 10⁻⁸ | −0.2684 | 1.0000 | M | M |
| RUNX3 | E27_R | 0.9550 | 0.6341 | 6.5 × 10⁻⁸ | −0.3209 | 0.9983 | M | M |
| RUNX3 | P247_F | 0.9599 | 0.6005 | 1.1 × 10⁻⁸ | −0.3594 | 1.0000 | M | M |
| PSCA | E359_F | 0.8366 | 0.6169 | 5.2 × 10⁻³ | −0.4105 | 0.8788 | | U |
| KLK10 | P268_R | 0.6305 | 0.2200 | 4.4 × 10⁻² | −0.3397 | 0.9040 | | U |

The 29 CpG loci/genes shown were found to exhibit significantly different methylation between melanomas and nevi after adjustment for age, sex, and Bonferroni correction for multiple comparisons. These loci, with the exception of TK_E166_R, also had mean methylation β value differences between nevi and melanomas of ≧0.2. All loci except ITK_E166_R exhibited. Probes were ranked by significance (adjusted P value) within each of the hypermethylated and hypomethylated groups. P value, nevus mean β, and melanoma mean β were each adjusted for age, sex, multiple comparisons using Bonferroni correction. AUC; area under the ROC curve. Methylation status in normal skin and peripheral blood leukocytes (U; unmethylated (~0.0-0.3), PM; partially methylated (~0.3-0.7), M; highly methylated (~0.8-1.0)).

TABLE 3B
The function/pathway description for the twenty-nine CpG loci

| | |
|---|---|
| Gene Symbol | Function/Pathway Description |

Hypermethylated in melanomas compared with nevi (n = 7)

| | |
|---|---|
| COL1A2 | extracellular matrix, cell commun, focal adhesion |
| FRZB | regulator of Wnt signaling; cell growth & differentiation |
| GSTM2 | carcinogen & oxidative metabolism |
| KCNK4 | potassium ion transport |
| NPR2 | receptor for several small natriuretic peptides |
| TRIP6 | +reg cell migration, release of cytopasmic NF-kB |
| TRIP6 | +reg cell migration, release of cytopasmic NF-kB |
| CCL3 | chemokine activity, immune response, upreg in tumors |
| CARD15 | Immune response to LPS, resulting in NF-kB activation |
| EV12A | Viral insertion site Evi12 mapped to NF1 gene region and |
| | noncoding region of GNN |
| HLA-DPA1 | cell adhesion, antigen presentation, immune response |
| IFNG | NK cell-mediated cytotox, T cell receptor signaling |
| ITK | T cell receptor proliferation & differentiation |
| ITK | T cell receptor proliferation & differentiation |
| LAT | NK cell-mediated cytotox, T cell receptor signaling |
| IL2 | Cytokine that regulates T-cell proliferation |
| CD2 | Mediates adhesion to T cells |
| MPO | Neutrophil oxidative metabolism, anti-apoptotic |
| EMR3 | granulocyte marker, involved in myeloid—myeloid |
| | interactions during immune responses |
| EMR3 | granulocyte marker, involved in myeloid—myeloid |
| | interactions during immune responses |
| OSM | reg cell growth & cytokine production, Jak/STAT pathway |
| OSM | reg cell growth & cytokine production, Jak/STAT pathway |
| TNFSF8 | cytokine, induces T-cell proliferation, pro-apoptosis |
| PTHLH | parathyroid hormone signaling |
| PTHR1 | parathyroid hormone signaling |
| RUNX3 | regulator of cell proliferation, pro-apoptosis |
| RUNX3 | regulator of cell proliferation, pro-apoptosis |
| RUNX3 | regulator of cell proliferation, pro-apoptosis |
| PSCA | membrane antigen, apoptosis, up- or downregulated in cancer |
| KLK10 | secreted serine protease, tumor suppressor |

TABLE 4A
Table 4A shows the accession numbers; specific single CpG coordinate; presence
or absence of CpG islands; specific sequences used in the Illumina GoldenGate array
experiments; and the synonyms for the genes hypomethylated in melanoma. All Accession
numbers and location are based on Ref. Seq. version 36.1.

| Probe_ID | Gid | Accession | Gene_ID | Chrm | CpG_Coor | Dist_to_TSS | CpG_isl |
|---|---|---|---|---|---|---|---|
| ARHGDIB_P148_R | 56676392 | NM_001175.4 | 397 | 12 | 15005977 | −148 | N |
| BMPR1A_E88_F | 41349436 | NM_004329.2 | 657 | 10 | 88506464 | 88 | Y |
| CARD15_P302_R | 11545911 | NM_022162.1 | 64127 | 16 | 49288249 | −302 | N |
| CASP8_E474_F | 73623018 | NM_001228.3 | 841 | 2 | 201806900 | 474 | N |
| CCL3_E53_R | 4506842 | NM_002983.1 | 6348 | 17 | 31441547 | 53 | N |
| CD2_P68_F | 31542293 | NM_001767.2 | 914 | 1 | 117098557 | −68 | N |
| CD86_P3_F | 29029570 | NM_006889.2 | 942 | 3 | 123256908 | −3 | N |
| COL1A1_P117_R | 14719826 | NM_000088.2 | 1277 | 17 | 45634109 | −117 | Y |
| DSG1_E292_F | 4503400 | NM_001942.1 | 1828 | 18 | 27152342 | 292 | N |
| DSG1_P159_R | 4503400 | NM_001942.1 | 1828 | 18 | 27151891 | −159 | N |
| EMR3_E61_F | 23397638 | NM_152939.1 | 84658 | 19 | 14646749 | 61 | N |
| EMR3_P39_R | 23397638 | NM_152939.1 | 84658 | 19 | 14646849 | −39 | N |
| EVI2A_E420_F | 51511748 | NM_001003927.1 | 2123 | 17 | 26672423 | 420 | N |
| EVI2A_P94_R | 51511748 | NM_001003927.1 | 2123 | 17 | 26672937 | −94 | N |
| GABRA5_P1016_F | 6031207 | NM_000810.2 | 2558 | 15 | 24741680 | −1016 | N |
| HBII-52_P563_F | 29171307 | NR_001291.1 | 338433 | 15 | 22966406 | −563 | Y |
| HLA-DPA1_P28_R | 24797073 | NM_033554.2 | 3113 | 6 | 33149384 | −28 | N |
| IFNG_P459_R | 56786137 | NM_000619.2 | 3458 | 12 | 66840247 | −459 | N |
| 1L2_P607_R | 28178860 | NM_000586.2 | 3558 | 4 | 123597937 | −607 | N |
| ITK_E166_R | 21614549 | NM_005546.3 | 3702 | 5 | 156540651 | 166 | N |
| ITK_P114_F | 21614549 | NM_005546.3 | 3702 | 5 | 156540371 | −114 | N |
| KLK10_P268_R | 22208981 | NM_002776.3 | 5655 | 19 | 56215362 | −268 | N |
| LAT_E46_F | 62739153 | NM_014387.3 | 27040 | 16 | 28903694 | 46 | N |
| MMP10_E136_R | 4505204 | NM_002425.1 | 4319 | 11 | 102156418 | 136 | N |
| MMP2_P303_R | 75905807 | NM_004530.2 | 4313 | 16 | 54070286 | −303 | Y |
| MPO_P883_R | 4557758 | NM_000250.1 | 4353 | 17 | 53714178 | −883 | N |
| MUSK_P308_F | 5031926 | NM_005592.1 | 4593 | 9 | 112470652 | −308 | N |
| OPCML_P71_F | 59939898 | NM_002545.3 | 4978 | 11 | 132907684 | −71 | N |
| OSM_P188_F | 28178862 | NM_020530.3 | 5008 | 22 | 28993028 | −188 | Y |
| OSM_P34_F | 28178862 | NM_020530.3 | 5008 | 22 | 28992874 | −34 | N |
| PECAM1_P135_F | 21314616 | NM_000442.2 | 5175 | 17 | 59817858 | −135 | Y |
| PGR_E183_R | 31981491 | NM_000926.2 | 5241 | 11 | 100506282 | 183 | N |
| PSCA_E359_F | 29893565 | NM_005672.2 | 8000 | 8 | 143759274 | 359 | N |
| PTHLH_E251_F | 39995088 | NM_198964.1 | 5744 | 12 | 28015932 | 251 | N |
| PTHR1_P258_F | 39995096 | NM_000316.2 | 5745 | 3 | 46893982 | −258 | N |
| PTK6_E50_F | 27886594 | NM_005975.2 | 5753 | 20 | 61639101 | 50 | Y |
| PTK7_E317_F | 27886610 | NM_002821.3 | 5754 | 6 | 43152324 | 317 | Y |
| PWCR1_P357_F | 29171309 | NR_001290.1 | 63968 | 15 | 22847360 | −357 | N |
| RUNX3_E27_R | 72534651 | NM_001031680.1 | 864 | 1 | 25164035 | 27 | N |
| RUNX3_P247_F | 72534651 | NM_001031680.1 | 864 | 1 | 25164309 | −247 | Y |
| RUNX3_P393_R | 72534651 | NM_001031680.1 | 864 | 1 | 25164455 | −393 | Y |
| SEMA3B_E96_F | 54607087 | NM_004636.2 | 7869 | 3 | 50280140 | 96 | N |
| SERPINA5_E69_F | 34147643 | NM_000624.3 | 5104 | 14 | 94117633 | 69 | N |
| SHB_P691_R | 4506934 | NM_003028.1 | 6461 | 9 | 38059901 | −691 | Y |
| SNURF_E256_R | 29540557 | NM_005678.3 | 8926 | 15 | 22751484 | 256 | Y |
| SNURF_P2_R | 29540557 | NM_005678.3 | 8926 | 15 | 22751226 | −2 | Y |
| SNURF_P78_F | 29540557 | NM_005678.3 | 8926 | 15 | 22751150 | −78 | Y |
| SYK_P584_F | 34147655 | NM_003177.3 | 6850 | 9 | 92603307 | −584 | N |
| TDG_E129_F | 56549140 | NM_001008411.1 | 6996 | 12 | 102883876 | 129 | Y |
| THBS2_P605_R | 40317627 | NM_003247.2 | 7058 | 6 | 169396667 | −605 | N |
| TNFSF8_E258_R | 24119162 | NM_001244.2 | 944 | 9 | 116732333 | 258 | N |
| TNFSF8_P184_F | 24119162 | NM_001244.2 | 944 | 9 | 116732775 | −184 | Y |
| ZIM2_P22_F | 33354272 | NM_015363.3 | 23619 | 19 | 62043909 | −22 | Y |

| | SEQ | |
|---|---|---|
| Probe_ID | ID | Input_Sequence |
| ARHGDIB_P148_R | 1 | GCACATGTGCGAGCATGACAGCCCGTGTGA[CG]TGGAGATGCATGAATGTACACGCAAGA |
| BMPR1A_E88_F | 2 | AGGAGGGAGGAGGGCCAAGGG[CG]GGCAGGAAGGCTTAGGCTCG |
| CARD15_P302_R | 3 | AGAGCTCCGAGTCACGTGGCTTGGG[CG]GGCCTCCCCTTCCTGGTGTCCA |
| CASP8_E474_F | 4 | CCTTGCCCAGAGGCTGCGGGCTG[CG]GGTCAAGACATCAGTAGAAGGAGG |
| CCL3_E53_R | 5 | AGCAGGTGACGGAATGTGGGCT[CG]AGTGTCAGCAGAGCCAAGAAAGGACTG |
| CD2_P68_F | 6 | TGTAAAGAGAGGCACGTGGTTAAGCTCT[CG]GGGTGTGGACTCCACCAGTC |
| CD86_P3_F | 7 | AAGTTAGCTGGGTAGGTATACAGTCATTGC[CG]AGGAAGGCTTGCACAGGGTG |
| COL1A1_P117_R | 8 | CGTGCCCCAGCCAATCAGAGCTGCCTGGCC[CG]GCCCCCAATTTGGGAGTTGG |
| DSG1_E292_F | 9 | GAGTGGATTCTGGTAAAAGTCCTTCATAAT[CG]TGCCCATTGTAAACAAGTGAAAACTTT |
| DSG1_P159_R | 10 | CCCATCACCTGTATAACCCT[CG]GTATTTCTGTTCACTTTAAGAGCCTGCCAC |
| EMR3_E61_F | 11 | AGCAAACTGCTTCCCCTCTTT[CG]CCATCAGACTCATGGTTCTGCTTTTCGTTT |
| EMR3_P39_R | 12 | GGGATGATTGAGTTGGTAAACCCTAA[CG]AGGAAATGCCCTGAAAGTTACATCAC |
| EVI2A_E420_F | 13 | AGGAAACCAAACTTAGATCCTT[CG]TAATCCTAATTTAAAACTCCATGGCGATGG |
| EVI2A_P94_R | 14 | CATGACAGGAGGCTTTGTAGAACCAATCCC[CG]CCTCCAGAGCAGGGAGGGTTTT |
| GABRA5_P1016_F | 15 | TGGTAGAGAAATGAAAGCACCACAGTGTGG[CG]GCTCTGGGAGTGCACTGGC |
| HBII-52_P563_F | 16 | GCCCAGGGGCAGGCTATGTGACTGCC[CG]GTCTGCAGCTGTAAGTGGTTTCT |
| HLA-DPA1_P28_R | 17 | GGAACAGTGATGAGGAACTGAGGC[CG]AGTGGAGGCAGATGAGACTGA |
| IFNG_P459_R | 18 | TGCAAATGACCAGAAAGCAAGGAAAGAATG[CG]GTTAAAAGAACAATTTGGTGAGG |
| IL2_P607_R | 19 | CACCTGGGACACTATGAATGTAACAATAAT[CG]TTATGAAATATGATCTTGTTTTTAGTC |
| ITK_E166_R | 20 | TCTCCCTCGAACTTTAAAGTC[CG]CTTCTTTGTGTTAACCAAAGCCAGCCT |
| ITK_P114_F | 21 | GTGAATTTTGAAAGGATGTGGTTT[CG]GCCTTTGACATCAGAGGAGAAGCTC |
| KLK10_P268_R | 22 | AACAGAAACAAGGAAAAAGGGAAACCCA[CG]CCCACTCTGTGGCCGTGAGTGA |
| LAT_E46_F | 23 | GGGTCCTGGATATGGAGGCCA[CG]GCTGCCAGCTGGCAGGTGGC |
| MMP10_E136_R | 24 | GAGCTGGCCAGTAGCTGCAATAGATGCCAC[CG]TTAATTACCTGGGCAAGATCCTTGT |
| MMP2_P303_R | 25 | CCGGCGTCCCTCCTAGTAGTAC[CG]CTGCTCTCTAACCTCAGGACGTCAAGG |
| MPO_P883_R | 26 | GGACAGGAAATCTGGCTGGAGAC[CG]TTGGGCTTCACAGGAAGGAG |
| MUSK_P308_F | 27 | GGAGAGGTGGGGTGCTGAATT[CG]AAGGTCAGGACACCTATACCTCTGGG |
| OPCML_P71_F | 28 | CAGAGCAGTCCTCCAAGGCA[CG]CATTGGCTCCACTCTCCTGAGCGACGG |
| OSM_P188_F | 29 | CGCTCCTCCTCCTGTTTTCTT[CG]AATTCGTTCTTCGAGGTCAGCCCTAC |
| OSM_P34_F | 30 | CAGGCTGGCAGCCACTTTATGCC[CG]CTGGGGCGATTGGCCAACACCTCATGA |
| PECAM1_P135_F | 31 | CAAGGCACAAGTGACATTTGCCTTGG[CG]TTCTTGACCCTCCCTCTGTCTCGC |
| PGR_E183_R | 32 | GAAGTTTGGATGTTGTGTGCCACACTT[CG]ATTTGTCTTAAGGAATGTGTTCC |
| PSCA_E359_F | 33 | TCCTAGGGGGCAGGTAGACAGACTGA[CG]GATGGATGGGCAGAGATGC |
| PTHLH_E251_F | 34 | CCTCAGTTCATTACTGTAAACCC[CG]TACCTTAAAAGACTCGGCTTCTTCTCAC |
| PTHR1_P258_F | 35 | GGCAAGGAGAGGACTATTGAGGCACACACA[CG]TGTCTGGCAGCCTGAGTGGG |
| PTK6_E50_F | 36 | GGCCCAGGTGAGCCTGGTCC[CG]GGACACCATGGCGGGCGGGCGCAGC |
| PTK7_E317_F | 37 | GGGGGCACAGAGCTTGGGAAGCG[CG]GGAGTCCCGTGGGCAAAAG |
| PWCR1_P357_F | 38 | GGAGAAGTTGTCATGGGAGGCCAGC[CG]CCTGCTGGCAAGGAAGATGG |
| RUNX3_E27_R | 39 | CGGCAGCCAGGGTGGAGGAGCTC[CG]AAGCTGACAGAGCAGAGTGGGCC |
| RUNX3_P247_F | 40 | CGGCCTTGGCTCATTGGCTGGGCCG[CG]GTCACCTGGGCCGTGATGTCACGGCC |
| RUNX3_P393_R | 41 | TTTTATTTGTGAGGCTGGCCTCAGCACG[CG]GCCCAAGAAACAGAACTGAAAGCGG |
| SEMA3B_E96_F | 42 | GAGAGATGCTGCTGCGGAAGTCCT[CG]GTGGAGTGTGAGAAGGCAGC |
| SERPINA5_E69_F | 43 | CCCAGGGCTTGAGGGCATGTGAGG[CG]AGGAGAGGATGGACTCTAGAG |
| SHB_P691_R | 44 | GGTGGGAGCCGGGCCCAGCACCAATC[CG]AGAGCAAGGCTAGGGGAGGTC |
| SNURF_E256_R | 45 | AGGCTTGCTGTTGTGCCGTTCTGCCC[CG]ATGGTATCCTGTCCGCTCGCATTGGGGCG |
| SNURF_P2_R | 46 | AGCCTGCCGCTGCTGCAGCGAGTCTGG[CG]CAGAGTGGAGCGGCCGCCGGAGATGCC |
| SNURF_P78_F | 47 | CCTGCACTGCGGCAAACAAGCACGCCTGCG[CG]GCCGCAGAGGCAGGCTGGCG |
| SYK_P584_F | 48 | TTTATTTGGTTGTGGACGTCAGAGC[CG]TCATGGTAAGAAGGAAGCAAAGCCTT |
| TDG_E129_F | 49 | GGGGTTGTCTTACCGCAGTGAGTACCA[CG]CGGTACTACAGAGACCGGCTGCCC |
| THBS2_P605_R | 50 | AACCTGACGTGCAGGCACAGAGCAAGGACT[CG]AGAGAACGAGAAGCAGTGGCAGCAGCT |
| TNFSF8_E258_R | 51 | CCCCAGGTGGCTGGCCACGGAGCC[CG]CCGGCACATGCATGGCTGTGTCTC |
| TNFSF8_P184_F | 52 | CACACACAAAGCAACTTCTGTTT[CG]TTTAGACTCTGCCACAAAACGCCTTC |
| ZIM2_P22_F | 53 | GCAGCTGCCCAGACTTCTGCAC[CG]AGGTGCAGCTCGACGCCTCCTTGTCA |

| Probe_ID | Synonym | cg_no |
|---|---|---|
| ARHGDIB_P148_R | D4, GDIA2, GDID4, LYGDI, Ly-GDI, RAP1GN1 | cg15450139 |
| BMPR1A_E88_F | ALK3, CD292, ACVRLK3 | cg14602437 |
| CARD15_P302_R | CD, ACUG, BLAU, IBD1, NOD2, NOD2B, PSORAS1 | cg23486288 |
| CASP8_E474_F | CAP4, MACH, MCH5, FLICE, MGC78473 | cg05776114 |
| CCL3_E53_R | MIP1A, SCYA3, G0S19-1, LD78ALPHA, MIP-1-alpha | cg21335375 |
| CD2_P68_F | T11, SRBC | cg20405187 |
| CD86_P3_F | B70, B7-2, LAB72, CD28LG2, MGC34413 | cg01878435 |
| COL1A1_P117_R | OI4, aa 694-711 | cg10100754 |
| DSG1_E292_F | DG1, DSG, CDHF4 | cg20099449 |
| DSG1_P159_R | DG1, DSG, CDHF4 | cg13834042 |
| EMR3_E61_F | . | cg15552238 |
| EMR3_P39_R | . | cg15746620 |
| EVI2A_E420_F | EVDA, EVI2 | cg14414427 |
| EVI2A_P94_R | EVDA, EVI2 | cg23352695 |
| GABRA5_P1016_F | . | cg02225257 |
| HBII-52_P563_F | RNHBII52 | cg21361081 |
| HLA-DPA1_P28_R | HLADP, HLASB, HLA-DP1A | cg13031167 |
| IFNG_P459_R | IFG, IFI | cg03628117 |
| IL2_P607_R | IL-2, TCGF, lymphokine | cg24372185 |
| ITK_E166_R | EMT, LYK, PSCTK2, MGC126257, MGC126258 | cg09489988 |
| ITK_P114_F | EMT, LYK, PSCTK2, MGC126257, MGC126258 | cg18953183 |
| KLK10_P268_R | NES1, PRSSL1 | cg06130787 |
| LAT_E46_F | LAT1, pp36 | cg03108875 |
| MMP10_E136_R | SL-2, STMY2 | cg02061229 |
| MMP2_P303_R | CLG4, MONA, CLG4A, TBE-1, MMP-II | cg20640526 |
| MPO_P883_R | . | cg24997501 |
| MUSK_P308_F | MGC126323, MGC126324 | cg22051739 |
| OPCML_P71_F | OPCM, OBCAM | cg00738841 |
| OSM_P188_F | MGC20461 | cg04546763 |
| OSM_P34_F | MGC20461 | cg10467217 |
| PECAM1_P135_F | CD31, PECAM-1 | cg05359956 |
| PGR_E183_R | PR, NR3C3 | cg24886336 |
| PSCA_E359_F | PRO232 | cg20546389 |
| PTHLH_E251_F | HHM, PLP, PTHR, PTHRP, MGC14611 | cg01333011 |
| PTHR1_P258_F | PTHR, MGC138426, MGC138452 | cg13804333 |
| PTK6_E50_F | BRK | cg03004675 |
| PTK7_E317_F | CCK4 | cg21726633 |
| PWCR1_P357_F | PET1, HBII-85 | cg07197644 |
| RUNX3_E27_R | AML2, CBFA3, PEBP2aC | cg21368948 |
| RUNX3_P247_F | AML2, CBFA3, PEBP2aC | cg10672665 |
| RUNX3_P393_R | AML2, CBFA3, PEBP2aC | cg12607238 |
| SEMA3B_E96_F | SemA, SEMA5, SEMAA, semaV, LUCA-1, FLJ34863 | cg25047248 |
| SERPINA5_E69_F | PCI, PAI3, PROCI, PLANH3 | cg08764227 |
| SHB_P691_R | RP11-3J10.8 | cg19574087 |
| SNURF_E256_R | . | cg07995992 |
| SNURF_P2_R | . | cg17916021 |
| SNURF_P78_F | . | cg15999943 |
| SYK_P584_F | . | cg06713470 |
| TDG_E129_F | . | cg09857351 |
| THBS2_P605_R | TSP2 | cg24654845 |
| TNFSF8_E258_R | CD153, CD30L, CD30LG | cg09980061 |
| TNFSF8_P184_F | CD153, CD30L, CD30LG | cg19343707 |
| ZIM2_P22_F | ZNF656 | cg01034638 |

TABLE 4B
Table 4B shows the accession numbers; specific single CpG coordinate; presence
or absence of CpG islands; specific sequences used in the Illumina GoldenGate array
experiments; and the synonyms for the genes hypermethylated in melanoma. All Accession
numbers and location are based on Ref. Seq. version 36.1.

| Probe_ID | Gid | Accession | Gene_ID | Chrm | CpG_Coor | Dis_to_TSS | CpG_isl |
|---|---|---|---|---|---|---|---|
| ALOX12_E85_R | 4502050 | NM_000697.1 | 239 | 17 | 6840213 | 85 | Y |
| ALOX12_P223_R | 4502050 | NM_000697.1 | 239 | 17 | 6839905 | −223 | Y |
| CDH13_P88_F | 61676095 | NM_001257.3 | 1012 | 16 | 81217991 | −88 | Y |
| COL1A2_E299_F | 48762933 | NM_000089.3 | 1278 | 7 | 93862108 | 299 | Y |
| DES_E228_R | 55749931 | NM_001927.3 | 1674 | 2 | 219991571 | 228 | Y |
| EPHA2_P203_F | 32967310 | NM_004431.2 | 1969 | 1 | 16355354 | −203 | Y |
| FRZB_P406_F | 38455387 | NM_001463.2 | 2487 | 2 | 183440149 | −406 | Y |
| GNMT_P197_F | 54792737 | NM_018960.4 | 27232 | 6 | 43036281 | −197 | Y |
| GSTM2_P453_R | 23065549 | NM_000848.2 | 2946 | 1 | 110011761 | −453 | N |
| HOXA11_P698_F | 24497552 | NM_005523.4 | 3207 | 7 | 27192053 | −698 | Y |
| HPN_P374_R | 33695154 | NM_182983.1 | 3249 | 19 | 40222876 | −374 | N |
| IPF1_P750_F | 4557672 | NM_000209.1 | 3651 | 13 | 27391427 | −750 | Y |
| KCNK4_E3_F | 15718764 | NM_016611.2 | 50801 | 11 | 63815454 | 3 | Y |
| LOX_P313_R | 21264603 | NM_002317.3 | 4015 | 5 | 121442166 | −313 | Y |
| MEST_P62_R | 29294638 | NM_002402.2 | 4232 | 7 | 129913220 | −62 | Y |
| MST1R_P87_R | 4505264 | NM_002447.1 | 4486 | 3 | 49916161 | −87 | Y |
| NEFL_E23_R | 5453761 | NM_006158.1 | 4747 | 8 | 24869923 | 23 | Y |
| NPR2_P1093_F | 73915098 | NM_003995.3 | 4882 | 9 | 35781313 | −1093 | Y |
| RARA_E128_R | 75812906 | NM_000964.2 | 5914 | 17 | 35719100 | 128 | N |
| TNFRSF10D_E27_F | 42544227 | NM_003840.3 | 8793 | 8 | 23077458 | 27 | Y |
| TRIP6_P1090_F | 23308730 | NM_003302.1 | 7205 | 7 | 100301891 | −1090 | Y |
| TRIP6_P1274_R | 23308730 | NM_003302.1 | 7205 | 7 | 100301707 | −1274 | Y |

| | SEQ | |
|---|---|---|
| Probe_ID | ID | Input_Sequence |
| ALOX12_E85_R | 54 | GGGGCCTGGCTCTTCTCCGGGT[CG]TACAACCGCGTGCAGCTTTGGCTGGTCGG |
| ALOX12_P223_R | 55 | CCGTTGGCCTCACCCTGGCT[CG]GGCCCCTTTATCATCCTGCAGCTACG |
| CDH13_P88_F | 56 | CCGTATCTGCCATGCAAAACGAGGGAG[CG]TTAGGAAGGAATCCGTCTTGTAA |
| COL1A2_E299_F | 57 | ACCCTAGGGCCAGGGAAACTTTTGC[CG]TATAAATAGGGCAGATCCGGGCTTT |
| DES_E228_R | 58 | GGCTCTAAGGGCTCCTCCAGCT[CG]GTGACGTCCCGCGTGTACCAGGTGTC |
| EPHA2_P203_F | 59 | TCCAAAGTTTGAGCGTCTCAAAG[CG]CCAGCGCCCCTACGGATTAGCCC |
| FRZB_P406_F | 60 | GGGACGTCTGTGCCTCTGCCCGGG[CG]GCTCTGCACTTTCCTACCTCCCGC |
| GNMT_P197_F | 61 | GGGATTGCACAGAGGGCTGGGTC[CG]CAGGCTGGCTAAAAGGACCTAGCCC |
| GSTM2_P453_R | 62 | CCTTGCCTGTGTTGTCCTTCCCA[CG]TTAGGTCTGTCATGCCACGTATGTCCGCAG |
| HOXA11_P698_F | 63 | TCATTCATGGTCACTTCCGAAG[CG]CTTTAGTGCCTTCCGTCCCTAAACC |
| HPN_P374_R | 64 | CTCCTTGCTGATTTGCACACATTGGC[CG]CTTCAGACACGCACTTCTGGGGCCA |
| IPF1_P750_F | 65 | CCTCGCTGTATTGGGAAGCTACGTTC[CG]GGCTGGCCAAATGGGCCC |
| KCNK4_E3_F | 66 | GAGATGCCAGATTAGCGTGGTGCCTGTC[CG]GAGAGACGGGCCAGCTGATG |
| LOX_P313_R | 67 | AGGCGAAGGCAGCCAGGCCATGGGG[CG]ACGCCAAAATATGCACGAAGAAAAATG |
| MEST_P62_R | 68 | GCCGGAGGCTATTGTCGAAGCCA[CG]GCCTGCCATTTCATACCCTTTGCAA |
| MST1R_P87_R | 69 | GGACTGGGCCAAATTTAAGCAGCGGTCC[CG]ACAGCCCCAAGATAGCGGACCCCCGCC |
| NEFL_E23_R | 70 | CGCCGCTTGTAGGAGGTCGAGTAGTA[CG]GCTCGTAGCTGAAGGAACTCATG |
| NPR2_P1093_F | 71 | AGGACAAACCCTGGGGTCGCTGG[CG]TGTGTGAGATGGAAATGGA |
| RARA_E128_R | 72 | CCCTTCCCAATTCTTTGGC[CG]CCTTTGACCCCGGCCTCTGCTTCTGA |
| TNFRSF10D_E27_F | 73 | CAGAAATCGTCCCCGTAGTTTGTG[CG]CGTGCAAAGGTTCTCGCAGCTACACTGCCA |
| TRIP6_P1090_F | 74 | AAGGGGACTTTGTGAACAGTGGG[CG]GGGAGACGCAGAGGCAGAGG |
| TRIP6_P1274_R | 75 | CTTGGGCATGGTGCCCGCTTGGCATAG[CG]CCCGGCTCCGGATCTTCCTGTGCCT |

| Probe_ID | Synonym | cg_no |
|---|---|---|
| ALOX12_E85_R | LOG12 | cg05878700 |
| ALOX12_P223_R | LOG12 | cg22819332 |
| CDH13_P88_F | CDHH | cg08977371 |
| COL1A2_E299_F | OI4 | cg22877867 |
| DES_E228_R | CSM1, CSM2, CMD1I, FLI12025, FLI39719, FLI41013, FLI41793 | cg21174728 |
| EPHA2_P203_F | ECK | cg15146752 |
| FRZB_P406_F | FRE, FZRB, hFIZ, FRITZ, FRP-3, FRZB1, SFRP3, SRFP3, FRZB-1, | cg25188149 |
| | FRZB-PEN | |
| GNMT_P197_F | . | cg04013093 |
| GSTM2_P453_R | GST4, GSTM, GTHMUS, GSTM2-2 | cg11063364 |
| HOXA11_P698_F | HOX1, HOX1I | cg17466857 |
| HPN_P374_R | TMPRSS1 | cg03537100 |
| IPF1_P750_F | IUF1, PDX1, IDX-1, MODY4, PDX-1, STF-1 | cg14584091 |
| KCNK4_E3_F | TRAAK, DKFZP566E164 | cg01352108 |
| LOX_P313_R | MGC105112 | cg08623535 |
| MEST_P62_R | PEG1, MGC8703, MGC111102, DKFZp686L18234 | cg07409197 |
| MST1R_P87_R | RON, PTK8, CDw136 | cg01709977 |
| NEFL_E23_R | NFL, NF-L, NF68, CMT1F, CMT2E | cg00987688 |
| NPR2_P1093_F | AMDM, NPRB, ANPRB, GUC2B, NPRBi, GUCY2B | cg17151902 |
| RARA_E128_R | RAR, NR1B1 | cg00848035 |
| TNFRSF10D_E27_F | DCR2, CD264, TRUNDD, TRAILR4 | cg01031400 |
| TRIP6_P1090_F | OIP1, ZRP-1, MGC3837, MGC4423, MGC10556, MGC10558, MGC29959 | cg09357642 |
| TRIP6_P1274_R | OIP1, ZRP-1, MGC3837, MGC4423, MGC10556, MGC10558, MGC29959 | cg06647679 |

### 6.10. Methylation Profiles for Metastastic Melanoma Samples

Using the methods described above, the methylation data for nine melanoma metastases was compared with the benign moles. Eighteen more Genes/CpG sites were found to be significant in this comparison with nine additional hypomethylated and nine hypermethylated genes. The metastases sample descriptions may be found in Table 1. For results of metastases vs. benign nevi see Table 5A and 5B below. For results of combined melanomas and metastases vs. benign nevi see Table 6A and 6B below. For gene descriptions and methylated sequences of the 18 significant additional genes see Table 7A and Table 7B.

TABLE 5A
shows the methylation sites, methylation levels, β values for benign
nevi and metastatic melanomas and difference in β values for
genes hypermethylated in melanoma metastasis.

| TargetID | Met β ave | Nevi β ave | Met vs Nevi Diff. |
|---|---|---|---|
| ALOX12_E85_R | 0.79 | 0.33 | 0.46 |
| ALOX12_P223_R | 0.69 | 0.41 | 0.29 |
| ASCL2_E76_R | 0.32 | 0.11 | 0.21 |
| ASCL2_P360_F | 0.40 | 0.10 | 0.29 |
| AXIN1_P995_R | 0.68 | 0.31 | 0.37 |
| AXL_P223_R | 0.47 | 0.22 | 0.25 |
| BCR_P346_F | 0.60 | 0.25 | 0.35 |
| CALCA_E174_R | 0.53 | 0.15 | 0.38 |
| CCNA1_E7_F | 0.26 | 0.06 | 0.20 |
| CD9_P504_F | 0.31 | 0.04 | 0.27 |
| CD9_P585_R | 0.59 | 0.19 | 0.40 |
| CDH11_E102_R | 0.31 | 0.04 | 0.28 |
| CFTR_P372_R | 0.55 | 0.35 | 0.20 |
| COL1A2_E299_F | 0.58 | 0.05 | 0.53 |
| CTSL_P264_R | 0.43 | 0.18 | 0.26 |
| DDIT3_P1313_R | 0.60 | 0.14 | 0.46 |
| DES_E228_R | 0.29 | 0.06 | 0.23 |
| DIO3_E230_R | 0.73 | 0.51 | 0.22 |
| DLK1_E227_R | 0.33 | 0.09 | 0.24 |
| DNAJC15_P65_F | 0.74 | 0.53 | 0.21 |
| DSC2_E90_F | 0.55 | 0.13 | 0.42 |
| EPHA2_P203_F | 0.54 | 0.16 | 0.38 |
| EPHA2_P340_R | 0.31 | 0.09 | 0.22 |
| EPHA5_P66_F | 0.56 | 0.29 | 0.27 |
| ER_seq_a1_S60_F | 0.34 | 0.12 | 0.23 |
| ESR2_E66_F | 0.34 | 0.04 | 0.30 |
| FASTK_P598_R | 0.63 | 0.39 | 0.24 |
| FGF1_E5_F | 0.77 | 0.53 | 0.24 |
| FGF1_P357_R | 0.75 | 0.43 | 0.32 |
| FRK_P258_F | 0.76 | 0.43 | 0.33 |
| FRK_P36_F | 0.74 | 0.40 | 0.34 |
| FRZB_E186_R | 0.60 | 0.24 | 0.35 |
| FRZB_P406_F | 0.47 | 0.04 | 0.44 |
| FZD9_E458_F | 0.47 | 0.25 | 0.22 |
| GNMT_E126_F | 0.24 | 0.03 | 0.21 |
| GNMT_P197_F | 0.47 | 0.19 | 0.28 |
| GRB7_E71_R | 0.50 | 0.28 | 0.22 |
| GSTM2_P453_R | 0.58 | 0.21 | 0.37 |
| HFE_E273_R | 0.36 | 0.10 | 0.26 |
| HOXA5_E187_F | 0.82 | 0.58 | 0.24 |
| HOXA5_P1324_F | 0.57 | 0.34 | 0.23 |
| HOXA9_E252_R | 0.71 | 0.27 | 0.43 |
| HS3ST2_E145_R | 0.41 | 0.06 | 0.35 |
| IGF1_E394_F | 0.74 | 0.34 | 0.40 |
| IGF2AS_P203_F | 0.45 | 0.20 | 0.25 |
| IGFBP5_P9_R | 0.36 | 0.14 | 0.21 |
| IHH_E186_F | 0.29 | 0.06 | 0.24 |
| IL17RB_E164_R | 0.26 | 0.06 | 0.20 |
| IPF1_P750_F | 0.64 | 0.38 | 0.26 |
| KCNK4_E3_F | 0.43 | 0.09 | 0.34 |
| LIG3_P622_R | 0.57 | 0.32 | 0.25 |
| LOX_P313_R | 0.47 | 0.09 | 0.39 |
| LYN_P241_F | 0.30 | 0.06 | 0.24 |
| MAP3K8_P1036_F | 0.77 | 0.28 | 0.49 |
| MC2R_P1025_F | 0.47 | 0.22 | 0.25 |
| MOS_E60_R | 0.33 | 0.13 | 0.20 |
| MST1R_E42_R | 0.83 | 0.62 | 0.21 |
| MST1R_P87_R | 0.83 | 0.38 | 0.46 |
| MT1A_E13_R | 0.39 | 0.17 | 0.22 |
| MYOD1_E156_F | 0.45 | 0.04 | 0.41 |
| NEFL_E23_R | 0.50 | 0.24 | 0.26 |
| NEO1_P1067_F | 0.34 | 0.06 | 0.28 |
| NPR2_P1093_F | 0.88 | 0.57 | 0.31 |
| NPR2_P618_F | 0.30 | 0.08 | 0.22 |
| OGG1_E400_F | 0.45 | 0.06 | 0.39 |
| p16_seq_47_S188_R | 0.24 | 0.04 | 0.20 |
| PAX6_P1121_F | 0.34 | 0.10 | 0.24 |
| PENK_P447_R | 0.33 | 0.09 | 0.24 |
| PGF_P320_F | 0.36 | 0.06 | 0.29 |
| PYCARD_P393_F | 0.28 | 0.08 | 0.21 |
| RARA_E128_R | 0.36 | 0.11 | 0.25 |
| RARA_P176_R | 0.65 | 0.37 | 0.28 |
| RARB_P60_F | 0.40 | 0.12 | 0.28 |
| RARRES1_P426_R | 0.66 | 0.42 | 0.24 |
| RIPK3_P124_F | 0.51 | 0.27 | 0.24 |
| S100A4_E315_F | 0.38 | 0.11 | 0.28 |
| SEMA3A_P658_R | 0.51 | 0.30 | 0.21 |
| SEPT5_P441_F | 0.40 | 0.14 | 0.26 |
| SEPT5_P464_R | 0.59 | 0.30 | 0.28 |
| SEPT9_P58_R | 0.62 | 0.18 | 0.44 |
| SOX17_P287_R | 0.49 | 0.23 | 0.26 |
| SOX17_P303_F | 0.39 | 0.17 | 0.23 |
| SOX2_P546_F | 0.39 | 0.10 | 0.29 |
| TAL1_E122_F | 0.35 | 0.14 | 0.20 |
| TGFB2_E226_R | 0.50 | 0.29 | 0.21 |
| TGFBI_P173_F | 0.45 | 0.22 | 0.24 |
| TNFRSF10C_P7_F | 0.38 | 0.14 | 0.24 |
| TNFRSF10D_E27_F | 0.69 | 0.42 | 0.27 |
| TNK1_P221_F | 0.51 | 0.15 | 0.36 |
| TRIP6_P1090_F | 0.64 | 0.11 | 0.53 |
| TRIP6_P1274_R | 0.69 | 0.22 | 0.47 |

TABLE 5B
shows the methylation sites, methylation levels, β values for
benign nevi and metastatic melanomas and difference in β
values for genes hypomethylated in melanoma metastasis.

| TargetID | Met β ave | Nevi β ave | Met vs Nevi |
|---|---|---|---|
| AFF3_P122_F | 0.71 | 0.98 | −0.26 |
| ATP10A_P524_R | 0.59 | 0.83 | −0.24 |
| BCL3_E71_F | 0.21 | 0.42 | −0.21 |
| CAPG_E228_F | 0.52 | 0.75 | −0.23 |
| CASP8_E474_F | 0.40 | 0.75 | −0.36 |
| CCL3_E53_R | 0.62 | 0.93 | −0.31 |
| CD2_P68_F | 0.57 | 0.96 | −0.39 |
| CD34_P780_R | 0.58 | 0.88 | −0.30 |
| CD86_P3_F | 0.41 | 0.76 | −0.35 |
| COL1A1_P117_R | 0.31 | 0.68 | −0.36 |
| DLC1_P695_F | 0.70 | 0.94 | −0.24 |
| DNASE1L1_P39_R | 0.26 | 0.54 | −0.28 |
| EMR3_E61_F | 0.50 | 0.89 | −0.39 |
| EMR3_P39_R | 0.47 | 0.90 | −0.43 |
| EVI2A_E420_F | 0.65 | 0.97 | −0.32 |
| EVI2A_P94_R | 0.33 | 0.77 | −0.44 |
| GUCY2D_P48_R | 0.26 | 0.49 | −0.22 |
| HLA−DOA_P191_R | 0.60 | 0.81 | −0.21 |
| HLA−DPA1_P28_R | 0.42 | 0.89 | −0.47 |
| HLA−DPB1_E2_R | 0.31 | 0.71 | −0.41 |
| HLA−DRA_P77_R | 0.13 | 0.41 | −0.29 |
| IFNG_P459_R | 0.62 | 0.90 | −0.28 |
| IL1B_P829_F | 0.52 | 0.73 | −0.21 |
| IL2_P607_R | 0.68 | 0.89 | −0.22 |
| ITK_E166_R | 0.71 | 0.97 | −0.27 |
| ITK_P114_F | 0.63 | 0.92 | −0.29 |
| KLK10_P268_R | 0.18 | 0.67 | −0.49 |
| KRT1_P798_R | 0.64 | 0.85 | −0.22 |
| LAT_E46_F | 0.34 | 0.89 | −0.56 |
| LTA_P214_R | 0.65 | 0.94 | −0.30 |
| LTB4R_P163_F | 0.76 | 0.96 | −0.20 |
| MMP10_E136_R | 0.70 | 0.91 | −0.21 |
| MMP2_P197_F | 0.18 | 0.64 | −0.46 |
| MMP2_P303_R | 0.31 | 0.83 | −0.51 |
| MMP7_E59_F | 0.40 | 0.61 | −0.21 |
| MPO_P883_R | 0.16 | 0.76 | −0.60 |
| MT1A_P600_F | 0.68 | 0.95 | −0.27 |
| MUSK_P308_F | 0.69 | 0.91 | −0.22 |
| NOTCH4_P938_F | 0.65 | 0.94 | −0.29 |
| OPCML_P71_F | 0.27 | 0.71 | −0.44 |
| OSM_P188_F | 0.61 | 0.96 | −0.36 |
| OSM_P34_F | 0.55 | 0.91 | −0.36 |
| PECAM1_P135_F | 0.71 | 0.94 | −0.23 |
| PLAU_P176_R | 0.27 | 0.49 | −0.22 |
| POMC_P400_R | 0.60 | 0.87 | −0.27 |
| PSCA_E359_F | 0.52 | 0.85 | −0.33 |
| PTHLH_E251_F | 0.58 | 0.91 | −0.33 |
| PTHR1_P258_F | 0.47 | 0.83 | −0.36 |
| PTK6_E50_F | 0.36 | 0.61 | −0.25 |
| PTPN6_E171_R | 0.51 | 0.90 | −0.39 |
| PTPN6_P282_R | 0.71 | 0.95 | −0.23 |
| RUNX3_E27_R | 0.58 | 0.96 | −0.37 |
| RUNX3_P247_F | 0.60 | 0.96 | −0.36 |
| RUNX3_P393_R | 0.72 | 0.96 | −0.24 |
| S100A4_P194_R | 0.70 | 0.90 | −0.20 |
| SEMA3B_E96_F | 0.21 | 0.68 | −0.47 |
| SEMA3B_P110_R | 0.25 | 0.69 | −0.44 |
| SEMA3C_P642_F | 0.45 | 0.70 | −0.25 |
| SERPINA5_P156_F | 0.25 | 0.47 | −0.22 |
| SHB_P691_R | 0.33 | 0.80 | −0.47 |
| SLC14A1_E295_F | 0.70 | 0.92 | −0.22 |
| SNURF_E256_R | 0.64 | 0.85 | −0.21 |
| SPDEF_E116_R | 0.40 | 0.70 | −0.31 |
| SPI1_P48_F | 0.74 | 0.97 | −0.23 |
| TDGF1_E53_R | 0.59 | 0.82 | −0.22 |
| THBS2_P605_R | 0.46 | 0.93 | −0.46 |
| TIE1_E66_R | 0.73 | 0.96 | −0.23 |
| TNFSF10_E53_F | 0.36 | 0.67 | −0.30 |
| TNFSF10_P2_R | 0.55 | 0.91 | −0.35 |
| TNFSF8_E258_R | 0.59 | 0.95 | −0.36 |
| TNFSF8_P184_F | 0.18 | 0.50 | −0.32 |
| VAMP8_P114_F | 0.31 | 0.67 | −0.37 |
| ZAP70_P220_R | 0.63 | 0.89 | −0.26 |

TABLE 6
shows the methylation sites, Raw p values, Bonferroni corrections, methylation levels, β values for
benign nevi and combined melanomas and metastatic melanomas and difference in β values.
A positive meandif shows hypomethylation in melanoma and a negative meandif is hypomethylation
in melanoma.

| TargetID | Raw_p | Bonferroni_p | FDR_p | Mel_Mean | Mol_Mean | Meandif |
|---|---|---|---|---|---|---|
| ACTG2_P346_F | 1.82E−09 | 1.73E−06 | 2.83E−08 | 0.762 | 0.915 | 0.154 |
| ACVR1_E328_R | 7.08E−06 | 0.00671794 | 4.1E−05 | 0.763 | 0.891 | 0.127 |
| AFF3_P122_F | 5.59E−13 | 5.31E−10 | 2.53E−11 | 0.815 | 0.977 | 0.162 |
| AGXT_E115_R | 1.58E−09 | 1.50E−06 | 2.58E−08 | 0.921 | 0.969 | 0.049 |
| ALOX12_E85_R | 5.65E−10 | 5.36E−07 | 1.12E−08 | 0.775 | 0.322 | −0.452 |
| ALOX12_P223_R | 4.33E−06 | 0.00411105 | 2.69E−05 | 0.718 | 0.411 | −0.307 |
| APBA2_P227_F | 8.17E−11 | 7.76E−08 | 2.22E−09 | 0.918 | 0.981 | 0.063 |
| APBA2_P305_R | 3.94E−06 | 0.0037405 | 2.52E−05 | 0.845 | 0.932 | 0.087 |
| ARHGAP9_P260_F | 5.23E−05 | 0.04967831 | 0.000248 | 0.828 | 0.945 | 0.117 |
| ARHGDIB_P148_R | 7.36E−08 | 6.9888E−05 | 6.59E−07 | 0.406 | 0.613 | 0.207 |
| B3GALT5_P330_F | 4.10E−08 | 3.8934E−05 | 4.01E−07 | 0.821 | 0.956 | 0.135 |
| BCL3_E71_F | 6.56E−08 | 6.2267E−05 | 5.99E−07 | 0.217 | 0.415 | 0.199 |
| BLK_P668_R | 1.12E−12 | 1.07E−09 | 4.84E−11 | 0.857 | 0.971 | 0.114 |
| BMPR1A_E88_F | 2.78E−07 | 0.00026391 | 2.26E−06 | 0.537 | 0.766 | 0.229 |
| BMPR2_P1271_F | 9.25E−06 | 0.00877848 | 5.19E−05 | 0.031 | 0.065 | 0.034 |
| C4B_P191_F | 7.36E−08 | 6.9888E−05 | 6.59E−07 | 0.923 | 0.976 | 0.053 |
| CARD15_P302_R | 5.18E−06 | 0.00491922 | 3.13E−05 | 0.250 | 0.537 | 0.286 |
| CASP8_E474_F | 5.47E−09 | 5.19E−06 | 7.53E−08 | 0.390 | 0.750 | 0.360 |
| CCL3_E53_R | 2.07E−13 | 1.96E−10 | 1.09E−11 | 0.678 | 0.927 | 0.249 |
| CD1A_P414_R | 2.24E−08 | 2.1257E−05 | 2.42E−07 | 0.894 | 0.977 | 0.084 |
| CD2_P68_F | 1.52E−16 | 1.45E−13 | 2.89E−14 | 0.669 | 0.960 | 0.291 |
| CD34_P339_R | 7.60E−10 | 7.21E−07 | 1.44E−08 | 0.753 | 0.909 | 0.156 |
| CD34_P780_R | 3.96E−06 | 0.00375531 | 2.52E−05 | 0.668 | 0.880 | 0.212 |
| CD86_P3_F | 1.16E−06 | 0.00110267 | 8.17E−06 | 0.421 | 0.757 | 0.336 |
| CDH11_E102_R | 6.19E−06 | 0.00587433 | 3.65E−05 | 0.351 | 0.036 | −0.315 |
| CDH13_P88_F | 4.64E−06 | 0.00440444 | 2.86E−05 | 0.666 | 0.367 | −0.299 |
| CDH17_P376_F | 5.84E−08 | 5.5435E−05 | 5.38E−07 | 0.906 | 0.959 | 0.053 |
| COL1A1_P117_R | 1.05E−08 | 9.99E−06 | 1.31E−07 | 0.342 | 0.677 | 0.335 |
| COL1A2_E299_F | 8.13E−09 | 7.71E−06 | 1.06E−07 | 0.614 | 0.051 | −0.563 |
| COMT_E401_F | 2.8E−05 | 0.02660434 | 0.000142 | 0.122 | 0.232 | 0.111 |
| CSF2_E248_R | 4.11E−12 | 3.90E−09 | 1.56E−10 | 0.880 | 0.969 | 0.088 |
| CSF3_E242_R | 3.18E−09 | 3.02E−06 | 4.65E−08 | 0.914 | 0.970 | 0.056 |
| CSF3_P309_R | 4.83E−10 | 4.58E−07 | 9.96E−09 | 0.775 | 0.907 | 0.132 |
| DES_E228_R | 3.03E−10 | 2.87E−07 | 6.68E−09 | 0.283 | 0.063 | −0.220 |
| DES_P1006_R | 4.79E−09 | 4.54E−06 | 6.78E−08 | 0.740 | 0.887 | 0.148 |
| DLC1_P695_F | 2.70E−12 | 2.56E−09 | 1.07E−10 | 0.731 | 0.941 | 0.210 |
| DMP1_P134_F | 7.13E−09 | 6.77E−06 | 9.53E−08 | 0.824 | 0.940 | 0.116 |
| DSC2_E90_F | 4.33E−06 | 0.00411105 | 2.69E−05 | 0.400 | 0.129 | −0.271 |
| DSG1_E292_F | 1.73E−06 | 0.00163847 | 1.18E−05 | 0.683 | 0.896 | 0.213 |
| DSG1_P159_R | 3.04E−05 | 0.02880779 | 0.000151 | 0.394 | 0.663 | 0.270 |
| EGF_P242_R | 2.59E−10 | 2.45E−07 | 5.98E−09 | 0.843 | 0.951 | 0.108 |
| EGR4_E70_F | 1.05E−06 | 0.00099955 | 7.57E−06 | 0.223 | 0.366 | 0.143 |
| EMR3_E61_F | 8.82E−10 | 8.37E−07 | 1.61E−08 | 0.501 | 0.889 | 0.388 |
| EMR3_P39_R | 1.08E−10 | 1.03E−07 | 2.78E−09 | 0.604 | 0.900 | 0.296 |
| EPHA2_P203_F | 4.10E−08 | 3.8934E−05 | 4.01E−07 | 0.515 | 0.162 | −0.353 |
| EPHA2_P340_R | 1.71E−06 | 0.00162373 | 1.18E−05 | 0.335 | 0.090 | −0.245 |
| EPHB4_P313_R | 5.65E−06 | 0.00536546 | 3.38E−05 | 0.071 | 0.184 | 0.113 |
| EPHX1_P22_F | 4.07E−11 | 3.87E−08 | 1.25E−09 | 0.897 | 0.968 | 0.071 |
| ERBB3_E331_F | 3.84E−05 | 0.03647948 | 0.000189 | 0.056 | 0.081 | 0.025 |
| EVI2A_E420_F | 9.23E−14 | 8.76E−11 | 6.26E−12 | 0.727 | 0.966 | 0.239 |
| EVI2A_P94_R | 1.79E−11 | 1.70E−08 | 6.30E−10 | 0.311 | 0.764 | 0.453 |
| FER_E119_F | 2.39E−05 | 0.02266018 | 0.000122 | 0.062 | 0.149 | 0.087 |
| FGF6_P139_R | 4.23E−05 | 0.0401189 | 0.000205 | 0.799 | 0.948 | 0.148 |
| FGF7_P610_F | 4.16E−05 | 0.03943251 | 0.000202 | 0.898 | 0.951 | 0.053 |
| FGF9_P1404_F | 5.67E−06 | 0.00537696 | 3.38E−05 | 0.103 | 0.170 | 0.068 |
| FGFR1_E317_F | 3.96E−06 | 0.00375531 | 2.52E−05 | 0.063 | 0.109 | 0.045 |
| FGR_P39_F | 8.77E−06 | 0.00832709 | 4.96E−05 | 0.942 | 0.973 | 0.031 |
| FOSL2_E384_R | 2.24E−08 | 2.1257E−05 | 2.42E−07 | 0.892 | 0.953 | 0.061 |
| FRZB_P406_F | 1.62E−09 | 1.53E−06 | 2.60E−08 | 0.481 | 0.036 | −0.445 |
| FZD9_P175_F | 7.07E−07 | 0.00067093 | 5.24E−06 | 0.138 | 0.201 | 0.063 |
| GABRA5_P1016_F | 7.60E−10 | 7.21E−07 | 1.44E−08 | 0.763 | 0.955 | 0.192 |
| GNMT_P197_F | 2.78E−07 | 0.00026391 | 2.26E−06 | 0.483 | 0.189 | −0.294 |
| GPR116_E328_R | 2.50E−07 | 0.00023715 | 2.06E−06 | 0.896 | 0.968 | 0.072 |
| GPR116_P850_F | 1.79E−08 | 1.6964E−05 | 2.07E−07 | 0.868 | 0.934 | 0.067 |
| GSTM2_P453_R | 7.94E−10 | 7.53E−07 | 1.48E−08 | 0.577 | 0.202 | −0.374 |
| HBII-52_P563_F | 6.76E−06 | 0.00641451 | 3.94E−05 | 0.579 | 0.865 | 0.286 |
| HBII-52_P659_F | 2.35E−06 | 0.00222931 | 1.57E−05 | 0.827 | 0.957 | 0.130 |
| HGF_P1293_R | 5.21E−07 | 0.00049439 | 3.96E−06 | 0.911 | 0.968 | 0.057 |
| HLA-DPA1_P28_R | 4.83E−10 | 4.58E−07 | 9.96E−09 | 0.516 | 0.884 | 0.367 |
| HLA-DPB1_P540_F | 1.20E−08 | 1.1358E−05 | 1.48E−07 | 0.948 | 0.980 | 0.032 |
| HOXA11_P698_F | 1.56E−05 | 0.01478451 | 8.31E−05 | 0.863 | 0.674 | −0.189 |
| HOXA9_E252_R | 7.38E−07 | 0.00070047 | 5.43E−06 | 0.732 | 0.288 | −0.444 |
| HTR2A_E10_R | 4.74E−06 | 0.00449816 | 2.88E−05 | 0.849 | 0.946 | 0.096 |
| IAPP_E280_F | 1.34E−08 | 1.2717E−05 | 1.63E−07 | 0.837 | 0.952 | 0.115 |
| ICAM1_E242_F | 1.72E−05 | 0.0163513 | 9.08E−05 | 0.048 | 0.090 | 0.043 |
| IFNG_P459_R | 2.36E−11 | 2.24E−08 | 8.01E−10 | 0.641 | 0.898 | 0.257 |
| IGF1_E394_F | 2.46E−07 | 0.00023381 | 2.05E−06 | 0.645 | 0.343 | −0.302 |
| IGF2AS_E4_F | 1.05E−06 | 0.00099955 | 7.57E−06 | 0.164 | 0.311 | 0.147 |
| IL10_P348_F | 9.23E−14 | 8.76E−11 | 6.26E−12 | 0.945 | 0.982 | 0.037 |
| IL12B_E25_F | 3.72E−06 | 0.00353231 | 2.42E−05 | 0.896 | 0.948 | 0.052 |
| IL12B_P1453_F | 2.2E−05 | 0.02089918 | 0.000114 | 0.758 | 0.874 | 0.115 |
| IL13_E75_R | 1.88E−10 | 1.78E−07 | 4.45E−09 | 0.931 | 0.980 | 0.049 |
| IL2_P607_R | 3.68E−11 | 3.49E−08 | 1.20E−09 | 0.585 | 0.893 | 0.308 |
| IPF1_P750_F | 1.05E−06 | 0.00099955 | 7.57E−06 | 0.700 | 0.372 | −0.328 |
| ITK_E166_R | 1.06E−14 | 1.00E−11 | 1.00E−12 | 0.754 | 0.974 | 0.221 |
| ITK_P114_F | 2.07E−13 | 1.96E−10 | 1.09E−11 | 0.624 | 0.919 | 0.295 |
| JAG1_P66_F | 2.2E−05 | 0.02089918 | 0.000114 | 0.064 | 0.113 | 0.049 |
| KCNK4_E3_F | 3.03E−10 | 2.87E−07 | 6.68E−09 | 0.457 | 0.093 | −0.364 |
| KLK10_P268_R | 3.54E−10 | 3.36E−07 | 7.64E−09 | 0.246 | 0.664 | 0.418 |
| KLK11_P1290_F | 2.86E−08 | 2.7146E−05 | 2.92E−07 | 0.827 | 0.944 | 0.118 |
| KRT1_P798_R | 1.09E−09 | 1.03E−06 | 1.88E−08 | 0.663 | 0.851 | 0.188 |
| LAT_E46_F | 5.00E−13 | 4.74E−10 | 2.50E−11 | 0.487 | 0.893 | 0.406 |
| LCK_E28_F | 2.84E−14 | 2.70E−11 | 2.25E−12 | 0.798 | 0.956 | 0.159 |
| LMO2_E148_F | 1.22E−13 | 1.15E−10 | 7.22E−12 | 0.895 | 0.977 | 0.082 |
| LOX_P313_R | 4.02E−07 | 0.00038107 | 3.15E−06 | 0.524 | 0.085 | −0.439 |
| LTA_P214_R | 1.35E−10 | 1.28E−07 | 3.38E−09 | 0.723 | 0.943 | 0.220 |
| LTB4R_P163_F | 3.43E−15 | 3.25E−12 | 4.07E−13 | 0.818 | 0.962 | 0.144 |
| MAP3K8_P1036_F | 1.16E−06 | 0.00110267 | 8.17E−06 | 0.605 | 0.277 | −0.327 |
| MAPK9_P1175_F | 3.89E−05 | 0.03695243 | 0.00019 | 0.919 | 0.963 | 0.044 |
| MAS1_P469_R | 3.66E−08 | 3.4691E−05 | 3.69E−07 | 0.904 | 0.962 | 0.058 |
| MAS1_P657_R | 5.20E−08 | 4.9315E−05 | 4.98E−07 | 0.923 | 0.975 | 0.053 |
| MEST_P62_R | 1.04E−05 | 0.00988532 | 5.68E−05 | 0.586 | 0.287 | −0.299 |
| MMP10_E136_R | 1.18E−09 | 1.12E−06 | 2.00E−08 | 0.690 | 0.914 | 0.223 |
| MMP19_E274_R | 2.74E−06 | 0.00260103 | 1.81E−05 | 0.839 | 0.934 | 0.095 |
| MMP2_P197_F | 1.81E−07 | 0.00017139 | 1.54E−06 | 0.296 | 0.648 | 0.352 |
| MMP2_P303_R | 1.02E−09 | 9.70E−07 | 1.80E−08 | 0.431 | 0.829 | 0.398 |
| MMP7_P613_F | 4.86E−11 | 4.61E−08 | 1.40E−09 | 0.885 | 0.958 | 0.073 |
| MMP9_P237_R | 1.7E−05 | 0.01609838 | 8.99E−05 | 0.075 | 0.148 | 0.073 |
| MPL_P62_F | 1.25E−09 | 1.19E−06 | 2.08E−08 | 0.828 | 0.950 | 0.122 |
| MPO_P883_R | 1.52E−16 | 1.45E−13 | 2.89E−14 | 0.209 | 0.762 | 0.553 |
| MSH3_E3_F | 1.16E−07 | 0.00011008 | 1.00E−06 | 0.772 | 0.875 | 0.103 |
| MSH3_P13_R | 2.39E−05 | 0.02266018 | 0.000122 | 0.546 | 0.690 | 0.144 |
| MST1R_P87_R | 5.84E−08 | 5.5435E−05 | 5.38E−07 | 0.704 | 0.369 | −0.335 |
| MT1A_P600_F | 1.28E−06 | 0.00121572 | 8.94E−06 | 0.736 | 0.954 | 0.218 |
| MUSK_P308_F | 2.21E−08 | 2.1012E−05 | 2.42E−07 | 0.662 | 0.908 | 0.246 |
| MYOD1_E156_F | 1.56E−06 | 0.00148455 | 1.08E−05 | 0.277 | 0.043 | −0.234 |
| NEFL_E23_R | 1.04E−05 | 0.00988532 | 5.68E−05 | 0.499 | 0.243 | −0.256 |
| NOS2A_E117_R | 5.04E−12 | 4.79E−09 | 1.84E−10 | 0.849 | 0.962 | 0.113 |
| NOTCH4_P938_F | 1.75E−08 | 1.6587E−05 | 2.05E−07 | 0.732 | 0.936 | 0.204 |
| NPR2 P1093_F | 7.55E−08 | 7.1693E−05 | 6.70E−07 | 0.817 | 0.578 | −0.239 |
| OPCML_P71_F | 4.10E−07 | 0.00038891 | 3.19E−06 | 0.278 | 0.711 | 0.432 |
| OSM_P188_F | 3.05E−16 | 2.89E−13 | 4.82E−14 | 0.696 | 0.963 | 0.267 |
| OSM_P34_F | 1.08E−10 | 1.03E−07 | 2.78E−09 | 0.630 | 0.913 | 0.283 |
| PDGFA_P78_F | 3.04E−05 | 0.02880779 | 0.000151 | 0.104 | 0.170 | 0.065 |
| PDGFRA_E125_F | 1.2E−05 | 0.01141812 | 6.49E−05 | 0.776 | 0.928 | 0.152 |
| PECAM1_P135_F | 1.22E−13 | 1.15E−10 | 7.22E−12 | 0.722 | 0.938 | 0.217 |
| PGR_E183_R | 5.23E−05 | 0.04967831 | 0.000248 | 0.665 | 0.840 | 0.175 |
| PIK3R1_P307_F | 1.1E−05 | 0.01046542 | 5.98E−05 | 0.907 | 0.955 | 0.047 |
| PLA2G2A_E268_F | 5.84E−08 | 5.5435E−05 | 5.38E−07 | 0.721 | 0.899 | 0.178 |
| PLG_E406_F | 4.86E−11 | 4.61E−08 | 1.40E−09 | 0.810 | 0.947 | 0.137 |
| PMP22_P975_F | 5.91E−09 | 5.61E−06 | 8.01E−08 | 0.783 | 0.952 | 0.169 |
| PRDM2_P1340_R | 4.69E−05 | 0.04451017 | 0.000225 | 0.914 | 0.960 | 0.047 |
| PROM1_P44_R | 1.81E−10 | 1.72E−07 | 4.40E−09 | 0.834 | 0.954 | 0.120 |
| PSCA_E359_F | 2.24E−08 | 2.1257E−05 | 2.42E−07 | 0.600 | 0.847 | 0.247 |
| PTHLH_E251_F | 2.70E−12 | 2.56E−09 | 1.07E−10 | 0.613 | 0.909 | 0.296 |
| PTHLH_P757_F | 1.49E−14 | 1.41E−11 | 1.28E−12 | 0.844 | 0.955 | 0.111 |
| PTHR1_E36_R | 1.01E−08 | 9.63E−06 | 1.28E−07 | 0.924 | 0.966 | 0.042 |
| PTHR1_P258_F | 8.13E−09 | 7.71E−06 | 1.06E−07 | 0.540 | 0.831 | 0.291 |
| PTK6_E50_F | 1.88E−06 | 0.00178615 | 1.28E−05 | 0.302 | 0.617 | 0.314 |
| PTK7_E317_F | 2.86E−08 | 2.7146E−05 | 2.92E−07 | 0.424 | 0.668 | 0.245 |
| PTPN6_E171_R | 3.02E−05 | 0.02869854 | 0.000151 | 0.670 | 0.898 | 0.227 |
| PTPN6_P282_R | 1.91E−05 | 0.01814096 | 9.97E−05 | 0.855 | 0.946 | 0.091 |
| PWCR1_E81_R | 2.77E−09 | 2.63E−06 | 4.11E−08 | 0.858 | 0.974 | 0.116 |
| PWCR1_P357_F | 3.30E−06 | 0.00312863 | 2.16E−05 | 0.663 | 0.858 | 0.194 |
| PYCARD_P393_F | 1.16E−06 | 0.00110267 | 8.17E−06 | 0.287 | 0.077 | −0.210 |
| RARA_E128_R | 4.33E−06 | 0.00411105 | 2.69E−05 | 0.368 | 0.108 | −0.261 |
| RIPK3_P124_F | 8.47E−06 | 0.00803373 | 4.81E−05 | 0.613 | 0.272 | −0.341 |
| RUNX3_E27_R | 1.06E−14 | 1.00E−11 | 1.00E−12 | 0.611 | 0.958 | 0.346 |
| RUNX3_P247_F | 1.43E−16 | 1.35E−13 | 2.89E−14 | 0.584 | 0.965 | 0.380 |
| RUNX3_P393_R | 1.52E−16 | 1.45E−13 | 2.89E−14 | 0.705 | 0.963 | 0.257 |
| S100A4_E315_F | 9.56E−06 | 0.009075 | 5.31E−05 | 0.377 | 0.105 | −0.272 |
| SEMA3B_E96_F | 4.62E−08 | 4.3835E−05 | 4.47E−07 | 0.333 | 0.685 | 0.351 |
| SERPINA5_E69_F | 7.38E−06 | 0.00700089 | 4.22E−05 | 0.595 | 0.787 | 0.191 |
| SFTPA1_P421_F | 9.00E−10 | 8.54E−07 | 1.61E−08 | 0.806 | 0.940 | 0.134 |
| SFTPB_P689_R | 2.97E−07 | 0.00028206 | 2.37E−06 | 0.758 | 0.885 | 0.127 |
| SFTPD_E169_F | 9.26E−09 | 8.78E−06 | 1.19E−07 | 0.781 | 0.936 | 0.155 |
| SHB_P691_R | 1.36E−08 | 1.2898E−05 | 1.63E−07 | 0.430 | 0.805 | 0.376 |
| SLC14A1_E295_F | 2.10E−09 | 1.99E−06 | 3.21E−08 | 0.729 | 0.917 | 0.188 |
| SLC22A2_E271_R | 2.85E−08 | 2.7048E−05 | 2.92E−07 | 0.926 | 0.976 | 0.050 |
| SLC22A3_P634_F | 4.74E−06 | 0.00449816 | 2.88E−05 | 0.636 | 0.816 | 0.181 |
| SNCG_P98_R | 9.56E−06 | 0.009075 | 5.31E−05 | 0.707 | 0.866 | 0.158 |
| SNRPN_SEQ_18_S99_F | 4.22E−06 | 0.00400335 | 2.67E−05 | 0.642 | 0.792 | 0.150 |
| SNURF_E256_R | 2.85E−08 | 2.7048E−05 | 2.92E−07 | 0.591 | 0.849 | 0.258 |
| SNURF_P2_R | 4.18E−09 | 3.97E−06 | 6.01E−08 | 0.412 | 0.613 | 0.200 |
| SNURF_P78_F | 2.74E−06 | 0.00260103 | 1.81E−05 | 0.636 | 0.805 | 0.169 |
| SOD3_P225_F | 3.96E−11 | 3.76E−08 | 1.25E−09 | 0.946 | 0.980 | 0.033 |
| SPI1_E205_F | 6.76E−06 | 0.00641451 | 3.94E−05 | 0.543 | 0.715 | 0.171 |
| SPI1_P48_F | 7.62E−17 | 7.23E−14 | 2.89E−14 | 0.797 | 0.969 | 0.172 |
| STAT5A_E42_F | 9.26E−08 | 8.7841E−05 | 8.06E−07 | 0.093 | 0.205 | 0.112 |
| SYK_P584_F | 4.24E−07 | 0.00040208 | 3.27E−06 | 0.707 | 0.896 | 0.189 |
| TDGF1_E53_R | 3.09E−07 | 0.00029349 | 2.45E−06 | 0.627 | 0.815 | 0.189 |
| TDG_E129_F | 5.84E−08 | 5.5435E−05 | 5.38E−07 | 0.638 | 0.818 | 0.180 |
| TEK_P526_F | 2.27E−06 | 0.00215777 | 1.53E−05 | 0.695 | 0.856 | 0.162 |
| TFF2_P557_R | 2.53E−08 | 2.4032E−05 | 2.70E−07 | 0.910 | 0.974 | 0.065 |
| THBS2_P605_R | 1.95E−08 | 1.8488E−05 | 2.23E−07 | 0.573 | 0.944 | 0.370 |
| THPO_E483_F | 5.47E−09 | 5.19E−06 | 7.53E−08 | 0.915 | 0.976 | 0.061 |
| TIE1_E66_R | 5.59E−13 | 5.31E−10 | 2.53E−11 | 0.774 | 0.957 | 0.183 |
| TIMP3_P690_R | 5.21E−07 | 0.00049439 | 3.96E−06 | 0.961 | 0.982 | 0.021 |
| TJP2_P518_F | 2.59E−05 | 0.02455862 | 0.000131 | 0.176 | 0.335 | 0.159 |
| TNFRSF10D_E27_F | 1.04E−05 | 0.00988532 | 5.68E−05 | 0.721 | 0.411 | −0.309 |
| TNFSF10_E53_F | 1.87E−05 | 0.01775313 | 9.81E−05 | 0.374 | 0.669 | 0.294 |
| TNFSF8_E258_R | 5.33E−16 | 5.06E−13 | 7.23E−14 | 0.585 | 0.950 | 0.365 |
| TNFSF8_P184_F | 4.10E−08 | 3.8934E−05 | 4.01E−07 | 0.225 | 0.497 | 0.272 |
| TRAF4_P372_F | 1.98E−08 | 1.8786E−05 | 2.24E−07 | 0.142 | 0.314 | 0.172 |
| TRIP6_P1090_F | 9.26E−08 | 8.7841E−05 | 8.06E−07 | 0.598 | 0.116 | −0.482 |
| TRIP6_P1274_R | 1.54E−08 | 1.4634E−05 | 1.83E−07 | 0.652 | 0.224 | −0.428 |
| TRPM5_E87_F | 5.79E−11 | 5.50E−08 | 1.62E−09 | 0.790 | 0.938 | 0.148 |
| UGT1A1_E11_F | 6.39E−07 | 0.00060637 | 4.77E−06 | 0.932 | 0.976 | 0.045 |
| UGT1A1_P315_R | 6.19E−06 | 0.00587433 | 3.65E−05 | 0.699 | 0.852 | 0.153 |
| UGT1A1_P564_R | 3.84E−05 | 0.03647948 | 0.000189 | 0.942 | 0.980 | 0.039 |
| USP29_P282_R | 7.38E−06 | 0.00700089 | 4.22E−05 | 0.845 | 0.954 | 0.109 |
| VAMP8_P114_F | 4.49E−05 | 0.04260645 | 0.000216 | 0.398 | 0.673 | 0.275 |
| VAV2_P1182_F | 2.91E−07 | 0.00027589 | 2.34E−06 | 0.035 | 0.060 | 0.025 |
| WNT8B_E487_F | 5.02E−10 | 4.76E−07 | 1.01E−08 | 0.767 | 0.924 | 0.156 |
| WNT8B_P216_R | 2.12E−07 | 0.00020104 | 1.78E−06 | 0.920 | 0.954 | 0.034 |
| XPC_P226_R | 5.77E−07 | 0.0005477 | 4.35E−06 | 0.731 | 0.865 | 0.134 |
| ZAP70_P220_R | 1.32E−05 | 0.01249291 | 7.06E−05 | 0.728 | 0.894 | 0.166 |
| ZIM2_P22_F | 2.01E−07 | 0.00019112 | 1.71E−06 | 0.536 | 0.721 | 0.186 |
| ZIM3_E203_F | 2.77E−09 | 2.63E−06 | 4.11E−08 | 0.916 | 0.979 | 0.063 |
| ZNFN1A1_P179_F | 1.64E−09 | 1.56E−06 | 2.60E−08 | 0.933 | 0.980 | 0.048 |

TABLE 7A
Table 7A shows the accession numbers; specific single CpG coordinate; presence
or absence of CpG islands; specific sequences used in the Illumina GoldenGate
array experiments; and the synonyms for additional genes hypomethylated in
melanoma metastasis.
All Accession numbers and location are based on Ref. Seq. version 36.1.

| Probe_ID | Gid | Accession | Gene_ID | Chrm | CpG_Coor | Dis_to_TSS | CpG I |
|---|---|---|---|---|---|---|---|
| CD34_P780_R | 68342037 | NM_001025109.1 | 947 | 1 | 206152086 | −780 | N |
| DLC1_P695_F | 33188432 | NM_182643.1 | 10395 | 8 | 13417461 | −695 | N |
| LTA_P214_R | 6806892 | NM_000595.2 | 4049 | 6 | 31647858 | −214 | N |
| MMP2_P197_F | 75905807 | NM_004530.2 | 4313 | 16 | 54070392 | −197 | Y |
| MT1A_P600_F | 71274112 | NM_005946.2 | 4489 | 16 | 55229479 | −600 | Y |
| NOTCH4_P938_F | 55770875 | NM_004557.3 | 4855 | 6 | 32300760 | −938 | N |
| PTPN6_E171_R | 34328901 | NM_080548.2 | 5777 | 12 | 6926172 | 171 | Y |
| TNFSF10_E53_F | 23510439 | NM_003810.2 | 8743 | 3 | 173723910 | 53 | N |
| VAMP8_P114_F | 14043025 | NM_003761.2 | 8673 | 2 | 85658114 | −114 | N |

| Probe_ID | SEQ ID | Input_Sequence |
|---|---|---|
| CD34_P780_R | 76 | GGCAGCCTAGTCTTGGGGACGTAGAGA[CG]GGAGAAAGGAGAAGCCAGCCT |
| DLC1_P695_F | 77 | ACAACTGCTTCCATCTAGCATGGCAG[CG]TTCCTGAATCACATCTCTAAAGCCGCT |
| LTA_P214_R | 78 | CCTTTCCCAGAACTCAGT[CG]CCTGAACCCCCAGCCTGTGGTTCTC |
| MMP2_P197_F | 79 | GCGAGAGAGGCAAGTGGGGTGA[CG]AGGTCGTGCACTGAGGGTG |
| MT1A_P600_F | 80 | AGAGTGAGAGGCCGACCCGTGTTCC[CG]TGTTACTGTGTACGGAGTAGTGG |
| NOTCH4_P938_F | 81 | CCTGAGAGCCTTCCCCTAC[CG]GGGAATATACTTCACCAGCACCACTTT |
| PTPN6_E171_R | 82 | GAGATGCTGTCCCGTGGGTAAGTCC[CG]GGCACCATCGGGGTCCCAGTCT |
| TNFSF10_E53_F | 83 | GACTGCTGTAAGTCAGCCAGGCAGC[CG]GTCACTGAAGCCCTTCCTTCTCTATT |
| VAMP8_P114_F | 84 | CACTGGGAGGACAGTGAAGAATGCC[CG]CCTACCTGGGGAAACCTGAGT |

| Probe_ID | Synonym | cg_no |
|---|---|---|
| CD34_P780_R | . | cg14637677 |
| DLC1_P695_F | HP, ARHGAP7, STARD12, FLJ21120, p122-RhoGAP | cg00933411 |
| LTA_P214_R | LT, TNFB, TNFSF1 | cg20798246 |
| MMP2_P197_F | CLG4, MONA, CLG4A, TBE-1, MMP-II | cg20597545 |
| MT1A_P600_F | MT1, MTC, MT1S, MGC32848 | cg10731123 |
| NOTCH4_P938_F | INT3, NOTCH3, MGC74442 | cg05166027 |
| PTPN6_E171_R | HCP, HCPH, SHP1, SHP-1, HPTP1C, PTP-1C, SHP-1L, SH-PTP1 | cg00788854 |
| TNFSF10_E53_F | TL2, APO2L, CD253, TRAIL, Apo-2L | cg16555388 |
| VAMP8_P114_F | EDB, VAMP5 | cg17641218 |

TABLE 7B
Table 7B shows the accession numbers; specific single CpG
coordinate; presence or absence of CpG islands; specific
sequences used in the Illumina GoldenGate array experiments;
and the synonyms for additional genes hypermethylated in
melanoma metastasis. All Accession numbers and location are
based on Ref. Seq. version 36.1.

| Probe_ID | Gid | Accession | Ge_ID | Chrm | CpG_Coor | Dis_to_TS | CpG_i |
|---|---|---|---|---|---|---|---|
| IGF1_E394_F | 19923111 | NM_000618.2 | 3479 | 12 | 101398060 | 394 | N |
| HOXA9_E252_R | 24497558 | NM_002142.3 | 3205 | 7 | 27171422 | 252 | Y |
| MAP3K8_P1036_F | 22035597 | NM_005204.2 | 1326 | 10 | 30761836 | −1036 | Y |
| PYCARD_P393_F | 22035619 | NM_145182.1 | 29108 | 16 | 31122145 | −393 | N |
| MYOD1_E156_F | 23111008 | NM_002478.3 | 4654 | 11 | 17697891 | 156 | Y |
| DSC2_E90_F | 40806177 | NM_024422.2 | 1824 | 18 | 26936285 | 90 | Y |
| CDH11_E102_R | 16306531 | NM_001797.2 | 1009 | 16 | 63713318 | 102 | Y |
| RIPK3_P124_F | 40254843 | NM_006871.2 | 11035 | 14 | 23879137 | −124 | N |
| S100A4_E315_F | 9845515 | NM_019554.1 | 6275 | 1 | 151784591 | 315 | N |

| Probe_ID | SEQ ID | Input_Sequence |
|---|---|---|
| IGF1_E394_F | 85 | TGTGCAAATGCATCCATCTCCC[CG]AGCTATTTTTCAGATTCCACAGAATTGCA |
| HOXA9_E252_R | 86 | TGGGTTCCACGAGGCGCCAAACACCGT[CG]CCTTGGACTGGAAGCTGCACG |
| MAP3K8_P1036_F | 87 | ACCTGGGCACTGGGAAGAATAGGG[CG]TGGACTTGGAGTGTGACCG |
| PYCARD_P393_F | 88 | CCAGCATAACATGGCCAACC[CG]ATGGCTCCCGAAACCTTGCCAGATGC |
| MYOD1_E156_F | 89 | TGGGCGAAGCCAGGACCGTGCCG[CG]CCACCGCCAGGATATGGAGCTACTGTC |
| DSC2_E90_F | 90 | CTGCGCAAGGTGTTTCTCACCAG[CG]GACGCCACCTATAAGGCCCATCTC |
| CDH11_E102_R | 91 | GAGGGTGGACGCAACCTCCGAGC[CG]CCAGTCCCTGGCGCAGGGCAAGCG |
| RIPK3_P124_F | 92 | AAAGCTAGTGCCTTTCTCCTTGACTAG[CG]TTTCCTGAGCACCTGCCGCAGCC |
| S100A4_E315_F | 93 | CATACCAACACGTACTATAGCAACAG[CG]TGTGCAAGCCCACATCTCAGAAGCA |

| Probe_ID | Synonym | cg_no |
|---|---|---|
| IGF1_E394_F | IGFI | cg17084217 |
| HOXA9_E252_R | HOX1, ABD-B, HOX1G, HOX1.7, MGC1934 | cg10604830 |
| MAP3K8_P1036_F | COT, EST, ESTF, TPL2, Tpl-2, c-COT, FLJ10486 | cg21555918 |
| PYCARD_P393_F | ASC, TMS1, CARDS, MGC10332 | cg23185156 |
| MYOD1_E156_F | PUM, MYF3, MYOD | cg20325846 |
| DSC2_E90_F | DG2, DSC3, CDHF2, DGII/III, DKFZp686I11137 | cg08156793 |
| CDH11_E102_R | OB, CAD11, CDHOB, OSF-4 | cg05318914 |
| RIPK3_P124_F | RIP3, RIP3 beta, RIP3 gamma | cg13583230 |
| S100A4_E315_F | 42A, 18A2, CAPL, MTS1, P9KA, PEL98 | cg22502265 |

The results above were confirmed in a second sample set. Specifically, sample set #2, an independent set of 25 melanomas and 29 nevi underwent DNA methylation profiling using the Illumina GoldenGate Cancer Panel I and passed filtering criteria. The melanomas were of a variety of histologic subtypes and ranged in Breslow thickness from 0.42 to 10.75 mm. The majority of nevi (21 of 29) had varying degree of histologic atypia. Of the panel of 22 genes identified through analysis of the initial sample set, 14 were also statistically significant for differential methylation in an independent data set including dysplastic nevi after adjustment for age, sex and multiple comparisons. In order to identify and account for potential confounders in studying methylation differences between melanomas and nevi, host factors such as age, sex, anatomic site, and solar elastosis (sun damage to the surrounding lesional skin) were examined. These host factors were not associated with differential methylation at the 26 loci in the marker panel.

The 14 genes were CARD15, CD2, EMR3 (2 CpG loci), EVI2A, FRZB, HLA-DPA1, IFNG, IL2, ITK, LAT, MPO, PTHLH, RUNX3 (3 CpG loci), and TNFSF8. It should be noted that the FRZB_E186 CpG locus rather than FRZB_P406 was significantly differentially methylated in sample set #2. The AUC's for CpG sites within these genes remained high in sample set #2, ranging from 0.79 to 0.97. See Conway et al., 2011, Pigment Cell Melanoma Res. 24 352-360, and supplemental materials, the contents of which are hereby incorporated by reference.

Additional confirmation of the methylation specific markers is found in Table 8 below that shows 168 CpG sites that distinguish melanomas from benign nevi after Bonferroni correction.

TABLE 8

| | | | | | Mole | Mel | |
|---|---|---|---|---|---|---|---|
| | | | | | Mean | Mean | Mean |
| Target ID | Raw_p | Bonferroni_p | FDR_p | AUC | β | β | Δβ |
| ACTG2_P346_F | 4.42E−07 | 0.000434634 | 3.62E−06 | 0.780 | 0.921 | 0.819 | 0.101 |
| AFF3_P122_F | 4.63E−13 | 4.56E−10 | 1.57E−11 | 0.883 | 0.963 | 0.882 | 0.080 |
| ALOX12_E85_R | 2.83E−09 | 2.78E−06 | 3.98E−08 | 0.824 | 0.325 | 0.651 | −0.327 |
| APBA2_P227_F | 9.39E−07 | 0.000923948 | 7.22E−06 | 0.774 | 0.971 | 0.937 | 0.034 |
| APOA1_P261_F | 8.02E−10 | 7.89E−07 | 1.27E−08 | 0.837 | 0.932 | 0.796 | 0.136 |
| AREG_P217_R | 3.20E−05 | 0.031506185 | 0.000169388 | 0.734 | 0.184 | 0.130 | 0.054 |
| ATP10A_P524_R | 3.43E−06 | 0.003377711 | 2.27E−05 | 0.778 | 0.814 | 0.633 | 0.182 |
| B3GALT5_P330_F | 8.65E−10 | 8.51E−07 | 1.35E−08 | 0.833 | 0.957 | 0.867 | 0.090 |
| BCL3_E71_F | 7.62E−06 | 0.007494111 | 4.54E−05 | 0.750 | 0.459 | 0.314 | 0.144 |
| BLK_P668_R | 1.22E−18 | 1.20E−15 | 1.32E−16 | 0.944 | 0.963 | 0.834 | 0.129 |
| BMP4_P199_R | 4.83E−05 | 0.047524599 | 0.000240023 | 0.731 | 0.622 | 0.753 | −0.131 |
| BMPR1A_E88_F | 2.00E−06 | 0.001968243 | 1.42E−05 | 0.765 | 0.817 | 0.627 | 0.190 |
| C4B_P191_F | 9.65E−06 | 0.00949199 | 5.65E−05 | 0.748 | 0.975 | 0.951 | 0.024 |
| CARD15_P302_R | 1.16E−09 | 1.15E−06 | 1.74E−08 | 0.833 | 0.489 | 0.211 | 0.278 |
| CASP8_E474_F | 6.64E−06 | 0.006538478 | 4.01E−05 | 0.752 | 0.780 | 0.554 | 0.226 |
| CCL3_E53_R | 1.00E−14 | 9.86E−12 | 4.33E−13 | 0.904 | 0.928 | 0.770 | 0.158 |
| CD1A_P414_R | 5.58E−07 | 0.000548982 | 4.46E−06 | 0.778 | 0.949 | 0.878 | 0.071 |
| CD2_P68_F | 1.78E−17 | 1.75E−14 | 1.17E−15 | 0.933 | 0.927 | 0.728 | 0.198 |
| CD34_P339_R | 2.67E−06 | 0.002628299 | 1.82E−05 | 0.762 | 0.916 | 0.808 | 0.108 |
| CD34_P780_R | 3.39E−08 | 3.34E−05 | 4.07E−07 | 0.804 | 0.837 | 0.653 | 0.184 |
| CD86_P3_F | 1.67E−08 | 1.64E−05 | 2.13E−07 | 0.810 | 0.772 | 0.489 | 0.283 |
| COL1A1_P117_R | 3.50E−11 | 3.44E−08 | 6.75E−10 | 0.856 | 0.694 | 0.359 | 0.335 |
| COL1A2_E299_F | 1.93E−06 | 0.001897802 | 1.40E−05 | 0.765 | 0.066 | 0.280 | −0.214 |
| COMT_E401_F | 2.44E−07 | 0.000239712 | 2.24E−06 | 0.786 | 0.314 | 0.187 | 0.128 |
| CRK_P721_F | 7.01E−06 | 0.006895396 | 4.20E−05 | 0.753 | 0.483 | 0.290 | 0.194 |
| CSF2_E248_R | 5.55E−08 | 5.47E−05 | 6.28E−07 | 0.801 | 0.946 | 0.879 | 0.068 |
| CSF3_E242_R | 2.98E−07 | 0.000292827 | 2.69E−06 | 0.784 | 0.959 | 0.904 | 0.055 |
| CSF3_P309_R | 2.54E−07 | 0.000249538 | 2.31E−06 | 0.785 | 0.887 | 0.783 | 0.105 |
| DAB2IP_E18_R | 4.04E−05 | 0.039721765 | 0.000206884 | 0.732 | 0.162 | 0.100 | 0.062 |
| DES_P1006_R | 7.68E−08 | 7.55E−05 | 8.39E−07 | 0.796 | 0.905 | 0.801 | 0.105 |
| DLC1_P695_F | 1.89E−12 | 1.86E−09 | 5.04E−11 | 0.875 | 0.941 | 0.804 | 0.137 |
| DMP1_P134_F | 8.35E−08 | 8.22E−05 | 8.74E−07 | 0.796 | 0.912 | 0.821 | 0.091 |
| DSG1_E292_F | 9.97E−09 | 9.81E−06 | 1.31E−07 | 0.816 | 0.897 | 0.742 | 0.154 |
| DSG1_P159_R | 9.57E−10 | 9.42E−07 | 1.45E−08 | 0.832 | 0.697 | 0.398 | 0.299 |
| DSP_P36_F | 7.03E−07 | 0.000691676 | 5.53E−06 | 0.775 | 0.212 | 0.125 | 0.088 |
| EGF_P242_R | 1.91E−16 | 1.88E−13 | 1.11E−14 | 0.923 | 0.955 | 0.864 | 0.091 |
| EMR3_E61_F | 1.49E−18 | 1.46E−15 | 1.33E−16 | 0.943 | 0.880 | 0.524 | 0.355 |
| EMR3_P39_R | 6.28E−16 | 6.18E−13 | 3.43E−14 | 0.919 | 0.862 | 0.567 | 0.295 |
| EPHB4_E476_R | 2.07E−07 | 0.00020398 | 1.96E−06 | 0.787 | 0.333 | 0.209 | 0.124 |
| EPHB4_P313_R | 4.14E−08 | 4.08E−05 | 4.86E−07 | 0.818 | 0.269 | 0.114 | 0.154 |
| EPHX1_P22_F | 5.79E−06 | 0.005698994 | 3.56E−05 | 0.753 | 0.959 | 0.914 | 0.045 |
| EVI2A_E420_F | 3.27E−18 | 3.22E−15 | 2.68E−16 | 0.940 | 0.964 | 0.851 | 0.113 |
| EVI2A_P94_R | 9.81E−16 | 9.66E−13 | 5.08E−14 | 0.919 | 0.825 | 0.436 | 0.389 |
| FANCE_P356_R | 3.10E−07 | 0.00030472 | 2.72E−06 | 0.783 | 0.397 | 0.207 | 0.190 |
| FASTK_P257_F | 8.98E−07 | 0.000883867 | 7.01E−06 | 0.774 | 0.114 | 0.066 | 0.048 |
| FER_E119_F | 3.81E−06 | 0.003753345 | 2.47E−05 | 0.759 | 0.210 | 0.122 | 0.087 |
| FGF12_E61_R | 3.95E−06 | 0.003889874 | 2.54E−05 | 0.759 | 0.198 | 0.119 | 0.079 |
| FGF6_E294_F | 1.03E−05 | 0.010164268 | 5.98E−05 | 0.756 | 0.941 | 0.838 | 0.103 |
| FGF6_P139_R | 6.16E−06 | 0.006062049 | 3.74E−05 | 0.759 | 0.947 | 0.820 | 0.127 |
| FGFR1_E317_F | 1.04E−11 | 1.02E−08 | 2.27E−10 | 0.864 | 0.118 | 0.065 | 0.053 |
| FLI1_E29_F | 3.99E−06 | 0.003924016 | 2.55E−05 | 0.759 | 0.132 | 0.084 | 0.047 |
| FOSL2_E384_R | 1.91E−07 | 0.000188082 | 1.83E−06 | 0.788 | 0.943 | 0.891 | 0.052 |
| FRZB_E186_R | 3.43E−09 | 3.38E−06 | 4.69E−08 | 0.823 | 0.251 | 0.617 | −0.366 |
| FRZB_P406_F | 3.36E−07 | 0.000330741 | 2.84E−06 | 0.784 | 0.066 | 0.433 | −0.367 |
| FZD9_P175_F | 1.01E−12 | 9.91E−10 | 2.91E−11 | 0.878 | 0.212 | 0.123 | 0.089 |
| GABRA5_P1016_F | 3.31E−12 | 3.26E−09 | 8.14E−11 | 0.871 | 0.945 | 0.812 | 0.133 |
| GML_P281_R | 3.71E−06 | 0.003652683 | 2.42E−05 | 0.760 | 0.899 | 0.756 | 0.143 |
| GPR116_P850_F | 2.92E−09 | 2.87E−06 | 4.04E−08 | 0.829 | 0.938 | 0.878 | 0.061 |
| HBII-52_P563_F | 9.45E−13 | 9.30E−10 | 2.82E−11 | 0.879 | 0.890 | 0.624 | 0.266 |
| HBII-52_P659_F | 1.15E−07 | 0.00011289 | 1.16E−06 | 0.799 | 0.953 | 0.859 | 0.095 |
| HGF_P1293_R | 1.61E−06 | 0.001580085 | 1.20E−05 | 0.767 | 0.966 | 0.930 | 0.036 |
| HLA-DPA1_P28_R | 2.59E−12 | 2.54E−09 | 6.69E−11 | 0.873 | 0.849 | 0.520 | 0.329 |
| HLA-DPB1_E2_R | 2.70E−12 | 2.66E−09 | 6.82E−11 | 0.886 | 0.666 | 0.376 | 0.290 |
| HLA-DRA_P77_R | 1.23E−06 | 0.00120708 | 9.29E−06 | 0.771 | 0.407 | 0.197 | 0.210 |
| HOXA9_E252_R | 1.98E−06 | 0.001950492 | 1.42E−05 | 0.777 | 0.247 | 0.595 | −0.349 |
| HPN_P374_R | 1.42E−05 | 0.013956409 | 8.02E−05 | 0.755 | 0.525 | 0.669 | −0.144 |
| HTR2A_E10_R | 8.72E−06 | 0.008580868 | 5.17E−05 | 0.749 | 0.944 | 0.882 | 0.062 |
| IAPP_E280_F | 3.02E−08 | 2.97E−05 | 3.72E−07 | 0.813 | 0.943 | 0.873 | 0.070 |
| IFNG_P459_R | 7.75E−23 | 7.63E−20 | 2.54E−20 | 0.985 | 0.843 | 0.529 | 0.314 |
| IL12B_P1453_F | 1.48E−05 | 0.014570463 | 8.33E−05 | 0.744 | 0.877 | 0.781 | 0.097 |
| IL13_E75_R | 2.67E−06 | 0.002628299 | 1.82E−05 | 0.762 | 0.972 | 0.944 | 0.028 |
| IL1B_P582_R | 4.49E−06 | 0.004415524 | 2.83E−05 | 0.759 | 0.918 | 0.813 | 0.105 |
| IL2_P607_R | 3.19E−13 | 3.14E−10 | 1.12E−11 | 0.891 | 0.879 | 0.640 | 0.239 |
| INS_P248_F | 3.00E−07 | 0.000295448 | 2.69E−06 | 0.789 | 0.853 | 0.655 | 0.198 |
| IPF1_P750_F | 2.69E−06 | 0.002643505 | 1.82E−05 | 0.765 | 0.399 | 0.593 | −0.194 |
| ITK_E166_R | 1.35E−18 | 1.32E−15 | 1.32E−16 | 0.943 | 0.951 | 0.762 | 0.188 |
| ITK_P114_F | 4.48E−20 | 4.41E−17 | 6.92E−18 | 0.956 | 0.898 | 0.636 | 0.262 |
| JAG1_P66_F | 3.36E−07 | 0.000330741 | 2.84E−06 | 0.784 | 0.142 | 0.092 | 0.050 |
| KCNK4_E3_F | 1.50E−07 | 0.000147184 | 1.49E−06 | 0.790 | 0.236 | 0.509 | −0.273 |
| KIAA0125_E29_F | 5.03E−05 | 0.049508182 | 0.000248693 | 0.732 | 0.868 | 0.733 | 0.135 |
| KLK10_P268_R | 8.20E−08 | 8.07E−05 | 8.74E−07 | 0.797 | 0.669 | 0.399 | 0.270 |
| KLK11_P103_R | 4.76E−06 | 0.004688393 | 2.95E−05 | 0.757 | 0.746 | 0.528 | 0.218 |
| KLK11_P1290_F | 1.61E−07 | 0.000158742 | 1.59E−06 | 0.791 | 0.926 | 0.837 | 0.089 |
| KRT1_P798_R | 2.94E−17 | 2.89E−14 | 1.81E−15 | 0.939 | 0.841 | 0.604 | 0.237 |
| LAT_E46_F | 8.15E−13 | 8.02E−10 | 2.51E−11 | 0.889 | 0.885 | 0.601 | 0.285 |
| LCK_E28_F | 1.01E−14 | 9.96E−12 | 4.33E−13 | 0.906 | 0.960 | 0.870 | 0.089 |
| LMO2_E148_F | 2.42E−11 | 2.38E−08 | 4.86E−10 | 0.860 | 0.967 | 0.927 | 0.040 |
| LOX_P313_R | 1.89E−05 | 0.018560597 | 0.000104862 | 0.742 | 0.115 | 0.380 | −0.266 |
| LTA_P214_R | 3.43E−11 | 3.38E−08 | 6.75E−10 | 0.858 | 0.944 | 0.833 | 0.111 |
| LTB4R_P163_F | 3.50E−12 | 3.44E−09 | 8.40E−11 | 0.873 | 0.920 | 0.793 | 0.127 |
| MAF_E77_R | 1.02E−05 | 0.010062142 | 5.95E−05 | 0.748 | 0.135 | 0.067 | 0.069 |
| MALT1_P406_R | 3.23E−06 | 0.003180027 | 2.15E−05 | 0.763 | 0.148 | 0.076 | 0.071 |
| MAPK14_P327_R | 4.71E−06 | 0.004635345 | 2.93E−05 | 0.760 | 0.177 | 0.088 | 0.089 |
| MATK_P190_R | 5.05E−05 | 0.049738537 | 0.000248693 | 0.736 | 0.289 | 0.179 | 0.110 |
| MEST_P62_R | 9.33E−06 | 0.009178578 | 5.50E−05 | 0.748 | 0.305 | 0.509 | −0.204 |
| MMP10_E136_R | 3.60E−09 | 3.54E−06 | 4.85E−08 | 0.822 | 0.894 | 0.707 | 0.187 |
| MMP19_E274_R | 1.55E−06 | 0.001522929 | 1.16E−05 | 0.767 | 0.922 | 0.839 | 0.083 |
| MMP2_P197_F | 4.08E−08 | 4.02E−05 | 4.84E−07 | 0.804 | 0.648 | 0.386 | 0.263 |
| MMP2_P303_R | 5.05E−13 | 4.97E−10 | 1.66E−11 | 0.884 | 0.831 | 0.497 | 0.334 |
| MMP9_P237_R | 1.99E−06 | 0.001959709 | 1.42E−05 | 0.768 | 0.200 | 0.106 | 0.094 |
| MOS_P746_F | 1.76E−05 | 0.017361256 | 9.86E−05 | 0.743 | 0.789 | 0.611 | 0.178 |
| MPL_P62_F | 9.37E−07 | 0.000921959 | 7.22E−06 | 0.784 | 0.938 | 0.887 | 0.051 |
| MPO_P883_R | 1.72E−21 | 1.69E−18 | 3.38E−19 | 0.967 | 0.686 | 0.207 | 0.479 |
| MSH3_E3_F | 1.55E−10 | 1.53E−07 | 2.63E−09 | 0.846 | 0.878 | 0.769 | 0.109 |
| MSH3_P13_R | 2.41E−05 | 0.023709722 | 0.000130993 | 0.737 | 0.740 | 0.585 | 0.155 |
| MST1R_P87_R | 3.68E−06 | 0.003620829 | 2.41E−05 | 0.758 | 0.446 | 0.629 | −0.184 |
| MUSK_P308_F | 3.37E−07 | 0.000331991 | 2.84E−06 | 0.784 | 0.917 | 0.790 | 0.127 |
| NDN_P1110_F | 3.12E−07 | 0.000307373 | 2.72E−06 | 0.787 | 0.922 | 0.814 | 0.108 |
| NEFL_E23_R | 1.83E−09 | 1.80E−06 | 2.68E−08 | 0.828 | 0.267 | 0.509 | −0.242 |
| NEU1_P745_F | 4.61E−07 | 0.00045392 | 3.75E−06 | 0.782 | 0.217 | 0.097 | 0.120 |
| NOS2A_E117_R | 1.95E−13 | 1.92E−10 | 7.12E−12 | 0.888 | 0.954 | 0.891 | 0.064 |
| NOTCH4_P938_F | 3.76E−15 | 3.70E−12 | 1.76E−13 | 0.909 | 0.929 | 0.790 | 0.139 |
| NPR2_P1093_F | 2.36E−05 | 0.023191257 | 0.00012956 | 0.740 | 0.680 | 0.787 | −0.107 |
| OPCML_P71_F | 5.89E−13 | 5.79E−10 | 1.87E−11 | 0.885 | 0.747 | 0.332 | 0.415 |
| OSM_P188_F | 1.23E−17 | 1.21E−14 | 8.66E−16 | 0.935 | 0.956 | 0.794 | 0.162 |
| OSM_P34_F | 6.92E−10 | 6.81E−07 | 1.12E−08 | 0.842 | 0.915 | 0.737 | 0.178 |
| PADI4_E24_F | 8.01E−08 | 7.88E−05 | 8.66E−07 | 0.796 | 0.854 | 0.651 | 0.203 |
| PDGFA_P78_F | 5.99E−06 | 0.005898733 | 3.66E−05 | 0.753 | 0.242 | 0.153 | 0.088 |
| PDGFRA_E125_F | 4.52E−08 | 4.44E−05 | 5.23E−07 | 0.804 | 0.869 | 0.660 | 0.209 |
| PECAM1_P135_F | 6.81E−11 | 6.70E−08 | 1.24E−09 | 0.853 | 0.916 | 0.777 | 0.139 |
| PEG3_E496_F | 3.72E−05 | 0.03657662 | 0.000191501 | 0.735 | 0.658 | 0.487 | 0.171 |
| PGR_E183_R | 2.78E−10 | 2.74E−07 | 4.64E−09 | 0.842 | 0.860 | 0.643 | 0.217 |
| PI3_P1394_R | 1.01E−07 | 9.95E−05 | 1.04E−06 | 0.802 | 0.575 | 0.318 | 0.257 |
| PLA2G2A_E268_F | 1.15E−08 | 1.13E−05 | 1.48E−07 | 0.814 | 0.867 | 0.689 | 0.178 |
| PLG_E406_F | 2.24E−14 | 2.21E−11 | 8.83E−13 | 0.900 | 0.962 | 0.887 | 0.075 |
| PMP22_P975_F | 4.60E−06 | 0.004525044 | 2.88E−05 | 0.757 | 0.936 | 0.849 | 0.087 |
| PROM1_P44_R | 5.52E−07 | 0.000542987 | 4.45E−06 | 0.781 | 0.945 | 0.891 | 0.054 |
| PRSS1_E45_R | 2.25E−07 | 0.000221013 | 2.10E−06 | 0.788 | 0.768 | 0.541 | 0.227 |
| PRSS1_P1249_R | 3.47E−05 | 0.034151828 | 0.000181659 | 0.740 | 0.658 | 0.463 | 0.196 |
| PSCA_E359_F | 3.41E−05 | 0.033539227 | 0.000179354 | 0.733 | 0.835 | 0.678 | 0.157 |
| PTHLH_E251_F | 3.95E−19 | 3.88E−16 | 4.85E−17 | 0.948 | 0.883 | 0.669 | 0.214 |
| PTHLH_P757_F | 1.07E−10 | 1.05E−07 | 1.91E−09 | 0.850 | 0.930 | 0.848 | 0.083 |
| PTHR1_P258_F | 2.31E−06 | 0.002277735 | 1.63E−05 | 0.765 | 0.781 | 0.583 | 0.198 |
| PTK6_E50_F | 4.22E−05 | 0.041567996 | 0.000214786 | 0.733 | 0.719 | 0.489 | 0.230 |
| PTK7_E317_F | 1.13E−10 | 1.11E−07 | 1.98E−09 | 0.850 | 0.609 | 0.361 | 0.248 |
| PWCR1_E81_R | 3.96E−18 | 3.90E−15 | 3.00E−16 | 0.939 | 0.974 | 0.884 | 0.090 |
| PWCR1_P357_F | 8.35E−08 | 8.22E−05 | 8.74E−07 | 0.796 | 0.865 | 0.700 | 0.165 |
| PXN_P308_F | 1.22E−05 | 0.011986257 | 6.97E−05 | 0.745 | 0.331 | 0.205 | 0.126 |
| RARA_E128_R | 1.86E−06 | 0.001829761 | 1.36E−05 | 0.766 | 0.102 | 0.296 | −0.195 |
| RARA_P176_R | 1.02E−06 | 0.00100545 | 7.79E−06 | 0.776 | 0.368 | 0.590 | −0.222 |
| RARRES1_P57_R | 4.87E−08 | 4.79E−05 | 5.57E−07 | 0.802 | 0.724 | 0.491 | 0.233 |
| RIPK3_P124_F | 3.30E−07 | 0.000324591 | 2.84E−06 | 0.787 | 0.382 | 0.650 | −0.267 |
| RUNX3_E27_R | 1.17E−21 | 1.15E−18 | 2.87E−19 | 0.967 | 0.935 | 0.622 | 0.313 |
| RUNX3_P247_F | 4.92E−20 | 4.84E−17 | 6.92E−18 | 0.957 | 0.955 | 0.666 | 0.289 |
| RUNX3_P393_R | 4.05E−24 | 3.99E−21 | 2.37E−21 | 0.981 | 0.946 | 0.705 | 0.241 |
| S100A2_P1186_F | 4.37E−05 | 0.042970545 | 0.000220362 | 0.730 | 0.744 | 0.541 | 0.202 |
| S100A4_P194_R | 1.75E−07 | 0.000172513 | 1.71E−06 | 0.790 | 0.871 | 0.698 | 0.173 |
| SEMA3B_E96_F | 1.44E−07 | 0.000141254 | 1.44E−06 | 0.791 | 0.643 | 0.387 | 0.255 |
| SEMA3B_P110_R | 2.26E−05 | 0.022243687 | 0.000124965 | 0.738 | 0.687 | 0.439 | 0.248 |
| SERPINA5_E69_ F | 6.65E−09 | 6.55E−06 | 8.85E−08 | 0.817 | 0.839 | 0.651 | 0.188 |
| SERPINA5_P156_F | 1.66E−06 | 0.0016315 | 1.23E−05 | 0.768 | 0.547 | 0.345 | 0.201 |
| SERPINB2_P939_F | 3.00E−06 | 0.002955566 | 2.02E−05 | 0.790 | 0.954 | 0.917 | 0.037 |
| SFN_P248_F | 1.22E−05 | 0.011986257 | 6.97E−05 | 0.745 | 0.351 | 0.215 | 0.136 |
| SFTPA1_P421_F | 1.93E−11 | 1.90E−08 | 4.04E−10 | 0.868 | 0.928 | 0.823 | 0.106 |
| SFTPB_P689_R | 2.41E−06 | 0.002375967 | 1.69E−05 | 0.778 | 0.889 | 0.821 | 0.068 |
| SFTPD_E169_F | 1.47E−12 | 1.45E−09 | 4.03E−11 | 0.876 | 0.934 | 0.809 | 0.125 |
| SHB_P691_R | 4.09E−06 | 0.004024225 | 2.60E−05 | 0.757 | 0.634 | 0.376 | 0.258 |
| SIN3B_P514_R | 1.80E−07 | 0.000177418 | 1.74E−06 | 0.793 | 0.924 | 0.815 | 0.109 |
| SLC14A1_E295_F | 1.39E−13 | 1.37E−10 | 5.27E−12 | 0.890 | 0.904 | 0.719 | 0.185 |
| SLC22A2_E271_R | 3.11E−07 | 0.000306227 | 2.72E−06 | 0.785 | 0.967 | 0.897 | 0.070 |
| SLC22A3_P634_F | 4.23E−05 | 0.041668393 | 0.000214786 | 0.730 | 0.820 | 0.668 | 0.152 |
| SNRPN_E14_F | 3.56E−05 | 0.035015362 | 0.000185266 | 0.734 | 0.880 | 0.734 | 0.146 |
| SNRPN_P230_R | 9.73E−08 | 9.57E−05 | 1.01E−06 | 0.796 | 0.941 | 0.844 | 0.097 |
| SNRPN_seq_18_S99_F | 1.98E−08 | 1.95E−05 | 2.50E−07 | 0.813 | 0.824 | 0.645 | 0.178 |
| SNURF_P2_R | 6.48E−08 | 6.37E−05 | 7.16E−07 | 0.798 | 0.671 | 0.473 | 0.198 |
| SNURF_P78_F | 4.64E−05 | 0.045690286 | 0.000233114 | 0.729 | 0.815 | 0.642 | 0.173 |
| SPI1_P48_F | 4.49E−12 | 4.42E−09 | 1.05E−10 | 0.869 | 0.961 | 0.856 | 0.105 |
| STAT5A_E42_F | 4.08E−07 | 0.000401853 | 3.38E−06 | 0.781 | 0.356 | 0.212 | 0.144 |
| SYK_P584_F | 4.00E−11 | 3.94E−08 | 7.57E−10 | 0.859 | 0.893 | 0.708 | 0.185 |
| TDG_E129_F | 5.72E−12 | 5.63E−09 | 1.31E−10 | 0.868 | 0.835 | 0.665 | 0.170 |
| TEK_P526_F | 3.11E−08 | 3.06E−05 | 3.77E−07 | 0.804 | 0.846 | 0.716 | 0.131 |
| TFF2_P557_R | 2.57E−09 | 2.53E−06 | 3.66E−08 | 0.825 | 0.967 | 0.923 | 0.043 |
| TGFB3_E58_R | 6.48E−08 | 6.37E−05 | 7.16E−07 | 0.798 | 0.845 | 0.891 | −0.046 |
| THPO_E483_F | 2.34E−07 | 0.000230256 | 2.17E−06 | 0.786 | 0.967 | 0.923 | 0.043 |
| TIE1_E66_R | 4.83E−10 | 4.76E−07 | 7.93E−09 | 0.839 | 0.938 | 0.833 | 0.106 |
| TJP2_P518_F | 1.12E−11 | 1.10E−08 | 2.40E−10 | 0.865 | 0.346 | 0.149 | 0.197 |
| TMEM63A_E63_F | 2.88E−05 | 0.028340214 | 0.000154023 | 0.739 | 0.138 | 0.052 | 0.086 |
| TNFRSF10D_E27_F | 1.24E−05 | 0.012194527 | 7.05E−05 | 0.746 | 0.401 | 0.596 | −0.195 |
| TNFSF10_E53_F | 2.49E−08 | 2.45E−05 | 3.10E−07 | 0.806 | 0.552 | 0.275 | 0.277 |
| TNFSF10_P2_R | 2.38E−05 | 0.023396482 | 0.00012998 | 0.744 | 0.839 | 0.612 | 0.227 |
| TNFSF8_E258_R | 4.81E−24 | 4.74E−21 | 2.37E−21 | 0.981 | 0.929 | 0.593 | 0.336 |
| TNFSF8_P184_F | 2.21E−11 | 2.18E−08 | 4.54E−10 | 0.859 | 0.565 | 0.255 | 0.311 |
| TRAF4_P372_F | 1.55E−10 | 1.53E−07 | 2.63E−09 | 0.846 | 0.313 | 0.163 | 0.150 |
| TRIP6_P1090_F | 2.01E−09 | 1.98E−06 | 2.91E−08 | 0.827 | 0.357 | 0.688 | −0.332 |
| TRIP6_P1274_R | 2.82E−05 | 0.027782809 | 0.000151819 | 0.735 | 0.451 | 0.655 | −0.203 |
| TRPM5_E87_F | 1.45E−14 | 1.43E−11 | 5.94E−13 | 0.902 | 0.935 | 0.794 | 0.140 |
| TSG101_P257_R | 8.97E−10 | 8.83E−07 | 1.38E−08 | 0.846 | 0.400 | 0.188 | 0.212 |
| TWIST1_P44_R | 3.61E−05 | 0.035511764 | 0.000186904 | 0.739 | 0.158 | 0.072 | 0.086 |
| UGT1A1_E11_F | 6.07E−12 | 5.98E−09 | 1.36E−10 | 0.867 | 0.971 | 0.922 | 0.049 |
| UGT1A1_P315_R | 4.08E−11 | 4.01E−08 | 7.57E−10 | 0.857 | 0.875 | 0.706 | 0.169 |
| UGT1A1_P564_R | 3.20E−05 | 0.031506185 | 0.000169388 | 0.734 | 0.967 | 0.920 | 0.047 |
| USP29_P282_R | 3.81E−07 | 0.000374549 | 3.17E−06 | 0.783 | 0.948 | 0.889 | 0.059 |
| VAV1_E9_F | 5.80E−07 | 0.000570634 | 4.60E−06 | 0.777 | 0.420 | 0.229 | 0.191 |
| WNT10B_P993_F | 4.79E−05 | 0.047109973 | 0.000239137 | 0.729 | 0.270 | 0.189 | 0.081 |
| WNT8B_E487_F | 1.27E−15 | 1.25E−12 | 6.25E−14 | 0.926 | 0.897 | 0.769 | 0.128 |
| WNT8B_P216_R | 1.41E−12 | 1.39E−09 | 3.96E−11 | 0.893 | 0.952 | 0.922 | 0.030 |
| WRN_P969_F | 3.19E−06 | 0.00314233 | 2.14E−05 | 0.760 | 0.932 | 0.849 | 0.084 |
| ZIM3_E203_F | 1.73E−06 | 0.001700572 | 1.27E−05 | 0.766 | 0.971 | 0.927 | 0.044 |
| ZNFN1A1_E102_F | 2.69E−06 | 0.002643505 | 1.82E−05 | 0.765 | 0.855 | 0.715 | 0.140 |
| ZNFN1A1_P179_F | 2.60E−05 | 0.025596467 | 0.00014064 | 0.739 | 0.969 | 0.943 | 0.026 |

### 6.11. Comparison of Methylation Profiles in Benign and Dysplastic Nevi, Primary Malignant Melanomas and Metastatic Melanoma

Illumina GoldenGate Cancer Panel I methylation profiling was performed in metastatic melanomas (n=11) to evaluate promoter methylation patterns. Illumina methylation array results were subjected to filtering using the same criterion as in the earlier sets of nevi and melanoma. Using class comparison analyses, promoter methylation patterns of metastatic melanomas were compared to promoter methylation patterns in benign and dysplastic nevi (n=56), and primary melanomas (n=47). Initial results found 91 CpG sites hypermethylated and 72 CpG sites hypomethylated in metastases when compared to nevi. (Table 5A/B) After Bonferroni correction for multiple comparisons, 75 CpG sites were identified that differed significantly (with P values of ≦0.05) between nevi and metastatic melanomas. Comparison of statistically significant sites of nevi and melanoma to nevi and metastases identified 31 overlapping CpG sites. No statistically significant differences in methylation patterns were seen between primary melanomas and metastatic melanomas for the CpG sites identified to define nevi.

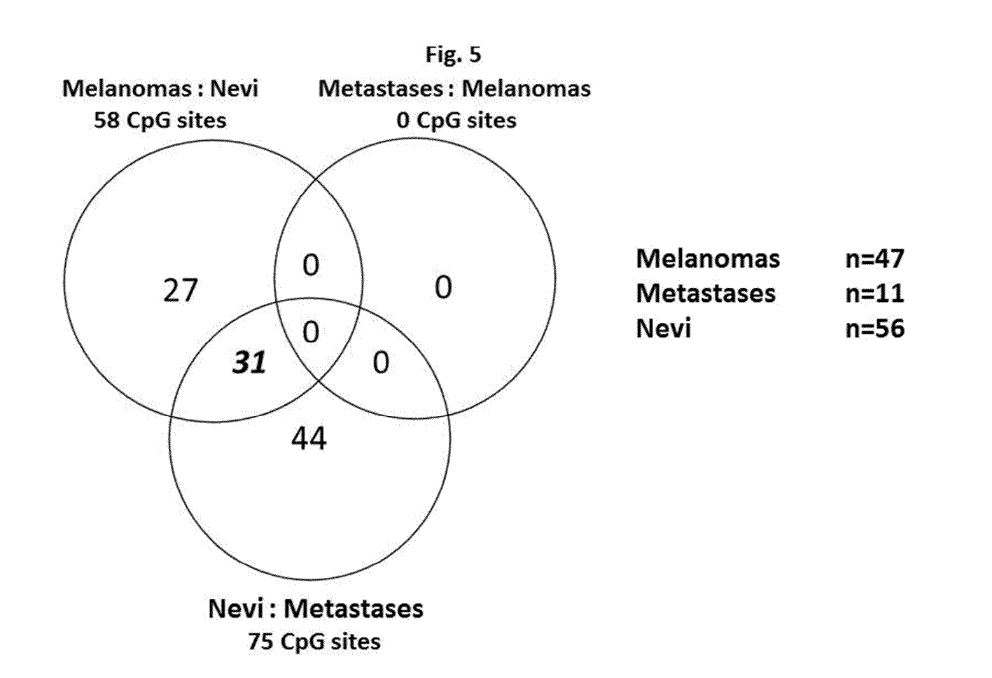

FIG. 5 shows a Venn diagram of CpG sites that statistically significantly distinguish between nevi (dysplastic and non-dysplastic) and primary melanomas or metastases. The number of statistically significant differential CpG sites, after Bonferoni correction for multiple comparisons and adjusting for age and gender, (p≦0.05) are listed for each of the three comparisons. The diagram is based on sample sets of nevi (n=56), melanoma (n=47), and metastases (n=11). 58 CpG sites distinguish between nevi and melanomas. 75 CpG sites distinguish between nevi and metastases. 31 common CpG sites differentiate nevi from either primary melanomas or metastases.

### 6.12. Methylation Markers for Normal Skin

Because normal skin may be a confounding contaminant for mole or melanoma samples, an analysis was undertaken to find methylation markers for normal skin. Using the methods described above, profiling was performed on FFPE normal skin specimens (N=42) discarded from surgeries. Tables 9A-9D below show the results of this analysis.

TABLE 9A
Statistically significant CpGs between skin and melanoma

| | p.val.skin. | q.val.skin. | coef.skin. | mean. | mean. | mean. |
|---|---|---|---|---|---|---|
| ProbeID | v.mela | v.mela | v.mela | β.skin | β.mela | β.diff |
| AATK_E63_R | 1.04E−07 | 1.52E−06 | 1.4214 | 0.695 | 0.904 | −0.209 |
| AATK_P519_R | 5.77E−11 | 2.54E−09 | 1.8072 | 0.609 | 0.904 | −0.295 |
| AATK_P709_R | 8.09E−09 | 1.64E−07 | 1.9841 | 0.288 | 0.730 | −0.442 |
| ALOX12_P223_R | 3.72E−11 | 1.80E−09 | 2.4206 | 0.211 | 0.740 | −0.528 |
| AXL_P223_R | 9.49E−08 | 1.40E−06 | 1.7792 | 0.079 | 0.336 | −0.258 |
| BMP4_P199_R | 3.37E−11 | 1.69E−09 | 2.0563 | 0.395 | 0.831 | −0.435 |
| CALCA_P171_F | 0.000318 | 0.001586 | 0.9918 | 0.254 | 0.477 | −0.223 |
| CAPG_E228_F | 4.94E−06 | 4.44E−05 | 1.5022 | 0.196 | 0.512 | −0.316 |
| CASP10_P334_F | 0.000221 | 0.001143 | 1.1924 | 0.200 | 0.450 | −0.250 |
| CDH13_P88_F | 2.11E−07 | 2.72E−06 | 1.8729 | 0.183 | 0.593 | −0.410 |
| COL1A2_P407_R | 5.62E−08 | 8.79E−07 | 1.7663 | 0.326 | 0.736 | −0.411 |
| CPA4_E20_F | 0.000484 | 0.002202 | 1.0139 | 0.263 | 0.494 | −0.231 |
| CRIP1_P274_F | 3.29E−05 | 0.000227 | 1.3256 | 0.309 | 0.627 | −0.317 |
| CRIP1_P874_R | 2.78E−13 | 5.07E−11 | 2.2923 | 0.082 | 0.465 | −0.383 |
| CSF1R_P73_F | 4.76E−07 | 5.13E−06 | 1.3677 | 0.328 | 0.653 | −0.325 |
| CSF3R_P8_F | 0.003225 | 0.011268 | 0.9969 | 0.439 | 0.677 | −0.238 |
| DDR1_P332_R | 8.60E−12 | 6.26E−10 | 2.4730 | 0.289 | 0.827 | −0.538 |
| EYA4_P794_F | 0.001818 | 0.006894 | 1.1556 | 0.359 | 0.640 | −0.281 |
| FGF9_P862_R | 7.43E−13 | 9.01E−11 | 1.2886 | 0.145 | 0.380 | −0.235 |
| GJB2_P931_R | 5.18E−08 | 8.20E−07 | 1.6722 | 0.376 | 0.762 | −0.386 |
| GRB10_P496_R | 3.76E−05 | 0.000257 | 1.4099 | 0.479 | 0.786 | −0.306 |
| GRB7_E71_R | 5.51E−14 | 1.61E−11 | 2.1378 | 0.129 | 0.553 | −0.424 |
| GRB7_P160_R | 4.87E−07 | 5.15E−06 | 1.6047 | 0.415 | 0.778 | −0.363 |
| HCK_P858_F | 0.000134 | 0.000757 | 1.4883 | 0.379 | 0.715 | −0.335 |
| HOXA9_P303_F | 6.25E−09 | 1.34E−07 | 2.0299 | 0.073 | 0.375 | −0.302 |
| IFNGR2_P377_R | 0.000347 | 0.001693 | 1.3107 | 0.247 | 0.548 | −0.301 |
| IGFBP1_E48_R | 8.48E−10 | 2.42E−08 | 1.9080 | 0.651 | 0.926 | −0.275 |
| IGFBP1_P12_R | 0.000135 | 0.000757 | 1.1764 | 0.645 | 0.853 | −0.208 |
| IL17RB_P788_R | 7.21E−10 | 2.19E−08 | 2.5332 | 0.062 | 0.451 | −0.389 |
| IL1RN_E42_F | 3.57E−07 | 3.99E−06 | 1.1514 | 0.625 | 0.840 | −0.215 |
| IL1RN_P93_R | 2.36E−10 | 7.99E−09 | 1.6854 | 0.379 | 0.765 | −0.386 |
| IPF1_P234_F | 0.000275 | 0.001387 | 1.2457 | 0.312 | 0.604 | −0.293 |
| JAK3_P1075_R | 2.78E−09 | 6.64E−08 | 1.6399 | 0.449 | 0.806 | −0.358 |
| KIAA1804_P689_R | 7.39E−08 | 1.13E−06 | 2.2881 | 0.068 | 0.415 | −0.347 |
| LEFTY2_P561_F | 0.00011 | 0.000644 | 1.0741 | 0.384 | 0.644 | −0.260 |
| LY6G6E_P45_R | 2.24E−10 | 7.78E−09 | 1.7792 | 0.599 | 0.898 | −0.299 |
| MEST_E150_F | 4.74E−05 | 0.000308 | 1.2153 | 0.310 | 0.598 | −0.288 |
| MET_E333_F | 7.83E−06 | 6.63E−05 | 1.4735 | 0.220 | 0.545 | −0.325 |
| MMP7_E59_F | 7.68E−06 | 6.54E−05 | 1.1203 | 0.286 | 0.550 | −0.264 |
| MPO_P883_R | 0.00041 | 0.00192 | −1.0215 | 0.425 | 0.211 | 0.215 |
| MST1R_E42_R | 1.97E−10 | 6.99E−09 | 2.1092 | 0.264 | 0.743 | −0.479 |
| MUC1_E18_R | 1.55E−10 | 6.10E−09 | 1.5059 | 0.553 | 0.847 | −0.294 |
| NBL1_E205_R | 6.49E−07 | 6.66E−06 | 1.4316 | 0.524 | 0.819 | −0.295 |
| NBL1_P24_F | 8.63E−07 | 8.78E−06 | 1.5593 | 0.309 | 0.680 | −0.371 |
| PDGFRA_E125_F | 0.000207 | 0.001086 | 1.2002 | 0.489 | 0.759 | −0.270 |
| PLAU_P176_R | 7.39E−10 | 2.20E−08 | 2.2742 | 0.070 | 0.418 | −0.348 |
| POMC_P400_R | 2.31E−07 | 2.89E−06 | 1.8722 | 0.280 | 0.715 | −0.435 |
| PRSS8_E134_R | 2.13E−13 | 4.42E−11 | 1.9091 | 0.664 | 0.930 | −0.266 |
| PTPN6_E171_R | 3.81E−07 | 4.24E−06 | 1.8056 | 0.314 | 0.727 | −0.413 |
| PTPRO_P371_F | 0.000309 | 0.001544 | 1.2753 | 0.154 | 0.394 | −0.239 |
| RARA_P176_R | 1.53E−07 | 2.06E−06 | 1.9454 | 0.237 | 0.681 | −0.444 |
| SEMA3A_P343_F | 3.62E−05 | 0.000248 | 1.4898 | 0.118 | 0.365 | −0.247 |
| SEMA3B_P110_R | 1.59E−05 | 0.00012 | 1.3407 | 0.121 | 0.343 | −0.222 |
| SERPINE1_E189_R | 4.00E−07 | 4.41E−06 | 1.5515 | 0.179 | 0.500 | −0.321 |
| SHB_P691_R | 9.72E−07 | 9.76E−06 | 1.7027 | 0.097 | 0.366 | −0.270 |
| SNCG_E119_F | 3.29E−11 | 1.69E−09 | 2.1366 | 0.260 | 0.748 | −0.487 |
| SNCG_P53_F | 1.02E−08 | 2.02E−07 | 2.1023 | 0.286 | 0.761 | −0.476 |
| SNCG_P98_R | 0.00054 | 0.002414 | 0.8917 | 0.481 | 0.692 | −0.211 |
| SPDEF_P6_R | 1.39E−09 | 3.67E−08 | 1.8819 | 0.362 | 0.784 | −0.423 |
| SPP1_E140_R | 0.000433 | 0.001999 | 1.0557 | 0.412 | 0.666 | −0.254 |
| STAT5A_P704_R | 6.56E−08 | 1.02E−06 | 1.8785 | 0.199 | 0.618 | −0.419 |
| TAL1_P594_F | 2.35E−05 | 0.00017 | 1.4210 | 0.383 | 0.713 | −0.330 |
| TEK_E75_F | 0.000186 | 0.000996 | 1.1881 | 0.528 | 0.785 | −0.257 |
| TGFB2_E226_R | 1.81E−17 | 8.81E−15 | 3.3352 | 0.150 | 0.831 | −0.681 |
| TGFB3_E58_R | 6.03E−11 | 2.58E−09 | 1.8890 | 0.571 | 0.898 | −0.327 |
| TGFBI_P173_F | 0.000122 | 0.00071 | 1.4164 | 0.116 | 0.346 | −0.230 |
| THBS2_P605_R | 3.27E−05 | 0.000227 | 1.6874 | 0.237 | 0.624 | −0.387 |
| THY1_P149_R | 7.03E−05 | 0.000432 | 1.2327 | 0.149 | 0.374 | −0.225 |
| TNFRSF10A_P171_F | 2.45E−07 | 3.00E−06 | 1.9202 | 0.155 | 0.547 | −0.392 |
| TNFRSF10D_E27_F | 6.47E−18 | 4.71E−15 | 3.1605 | 0.125 | 0.752 | −0.627 |
| TNFRSF10D_P70_F | 5.96E−13 | 8.68E−11 | 2.2537 | 0.193 | 0.678 | −0.485 |
| TNFSF10_E53_F | 1.37E−07 | 1.88E−06 | 1.7039 | 0.108 | 0.395 | −0.288 |
| TNFSF10_P2_R | 9.69E−11 | 3.92E−09 | 2.8088 | 0.150 | 0.742 | −0.591 |
| WNT10B_P823_R | 0.003172 | 0.011235 | 1.1306 | 0.309 | 0.574 | −0.265 |

TABLE 9B
Statistically significant CpGs between skin and moles

| | p.val.skin. | q.val.skin. | coef.skin. | mean. | mean. | mean. |
|---|---|---|---|---|---|---|
| ProbeID | v.mole | v.mole | v.mole | beta.skin | beta.mole | beta.diff |
| AATK_E63_R | 2.17E−08 | 3.44E−07 | 1.3860 | 0.700 | 0.903 | −0.202 |
| AATK_P519_R | 9.76E−09 | 1.69E−07 | 1.5241 | 0.631 | 0.886 | −0.255 |
| AATK_P709_R | 9.26E−08 | 1.20E−06 | 1.6614 | 0.300 | 0.690 | −0.390 |
| ALOX12_P223_R | 5.53E−06 | 3.80E−05 | 1.4178 | 0.183 | 0.475 | −0.292 |
| AXL_P223_R | 9.20E−09 | 1.61E−07 | 1.7461 | 0.070 | 0.299 | −0.229 |
| BMP4_P199_R | 3.90E−06 | 2.91E−05 | 1.4192 | 0.364 | 0.695 | −0.331 |
| CALCA_P171_F | 0.000664 | 0.00212 | 0.9302 | 0.239 | 0.442 | −0.203 |
| CAPG_E228_F | 5.33E−12 | 2.50E−10 | 2.3537 | 0.186 | 0.706 | −0.520 |
| CASP10_P334_F | 2.38E−12 | 1.24E−10 | 1.8892 | 0.171 | 0.566 | −0.395 |
| CDH13_P88_F | 5.60E−05 | 0.000259 | 1.1776 | 0.177 | 0.411 | −0.234 |
| COL1A2_P407_R | 2.09E−11 | 7.80E−10 | 2.0681 | 0.329 | 0.795 | −0.466 |
| CPA4_E20_F | 5.07E−06 | 3.56E−05 | 1.2938 | 0.261 | 0.563 | −0.302 |
| CRIP1_P274_F | 1.44E−09 | 3.32E−08 | 1.8763 | 0.283 | 0.715 | −0.432 |
| CRIP1_P874_R | 1.98E−21 | 4.80E−19 | 2.6804 | 0.080 | 0.560 | −0.479 |
| CSF1R_P73_F | 2.08E−07 | 2.34E−06 | 1.3586 | 0.342 | 0.667 | −0.325 |
| CSF3R_P8_F | 7.06E−10 | 1.71E−08 | 2.0001 | 0.458 | 0.859 | −0.401 |
| DDR1_P332_R | 9.70E−10 | 2.32E−08 | 2.0063 | 0.263 | 0.721 | −0.459 |
| EYA4_P794_F | 0.017419 | 0.03324 | 0.8850 | 0.361 | 0.578 | −0.217 |
| FGF9_P862_R | 2.07E−13 | 1.37E−11 | 1.4261 | 0.137 | 0.397 | −0.260 |
| GJB2_P931_R | 1.48E−07 | 1.84E−06 | 1.6227 | 0.381 | 0.757 | −0.376 |
| GRB10_P496_R | 5.69E−06 | 3.87E−05 | 1.3395 | 0.471 | 0.772 | −0.301 |
| GRB7_E71_R | 5.71E−10 | 1.44E−08 | 1.8195 | 0.107 | 0.404 | −0.297 |
| GRB7_P160_R | 2.68E−10 | 7.23E−09 | 1.8566 | 0.392 | 0.803 | −0.411 |
| HCK_P858_F | 0.000129 | 0.00052 | 1.2238 | 0.346 | 0.637 | −0.291 |
| HOXA9_P303_F | 8.84E−09 | 1.58E−07 | 1.7524 | 0.067 | 0.293 | −0.226 |
| IFNGR2_P377_R | 6.99E−07 | 6.65E−06 | 1.7076 | 0.249 | 0.646 | −0.397 |
| IGFBP1_E48_R | 4.44E−09 | 8.97E−08 | 1.9125 | 0.679 | 0.934 | −0.255 |
| IGFBP1_P12_R | 1.46E−06 | 1.22E−05 | 1.4924 | 0.645 | 0.890 | −0.245 |
| IL17RB_P788_R | 3.67E−20 | 7.63E−18 | 3.3227 | 0.055 | 0.612 | −0.557 |
| IL1RN_E42_F | 6.32E−07 | 6.13E−06 | 1.1331 | 0.630 | 0.841 | −0.211 |
| IL1RN_P93_R | 8.25E−12 | 3.53E−10 | 1.7523 | 0.375 | 0.776 | −0.401 |
| IPF1_P234_F | 0.000167 | 0.000645 | 1.1957 | 0.278 | 0.556 | −0.278 |
| JAK3_P1075_R | 1.54E−10 | 4.58E−09 | 1.7489 | 0.466 | 0.832 | −0.366 |
| KIAA1804_P689_R | 1.43E−10 | 4.33E−09 | 1.8411 | 0.065 | 0.305 | −0.240 |
| LEFTY2_P561_F | 2.55E−07 | 2.81E−06 | 1.2830 | 0.406 | 0.710 | −0.304 |
| LY6G6E_P45_R | 6.01E−08 | 8.31E−07 | 1.3572 | 0.603 | 0.855 | −0.252 |
| MEST_E150_F | 6.78E−05 | 0.000302 | 1.1353 | 0.264 | 0.512 | −0.248 |
| MET_E333_F | 6.15E−12 | 2.80E−10 | 1.9534 | 0.212 | 0.655 | −0.443 |
| MMP7_E59_F | 3.67E−12 | 1.84E−10 | 1.5096 | 0.281 | 0.637 | −0.356 |
| MPO_P883_R | 8.32E−11 | 2.69E−09 | 1.3782 | 0.435 | 0.753 | −0.318 |
| MST1R_E42_R | 6.05E−08 | 8.31E−07 | 1.6534 | 0.243 | 0.624 | −0.381 |
| MUC1_E18_R | 3.57E−09 | 7.52E−08 | 1.1762 | 0.551 | 0.799 | −0.248 |
| NBL1_E205_R | 1.24E−08 | 2.12E−07 | 1.3912 | 0.556 | 0.832 | −0.276 |
| NBL1_P24_F | 4.90E−09 | 9.64E−08 | 1.4422 | 0.308 | 0.653 | −0.345 |
| PDGFRA_E125_F | 3.53E−13 | 2.19E−11 | 2.2452 | 0.499 | 0.903 | −0.404 |
| PLAU_P176_R | 1.04E−14 | 8.44E−13 | 2.4790 | 0.063 | 0.445 | −0.381 |
| POMC_P400_R | 1.58E−11 | 6.23E−10 | 2.1786 | 0.316 | 0.797 | −0.481 |
| PRSS8_E134_R | 4.59E−12 | 2.23E−10 | 1.8324 | 0.645 | 0.918 | −0.273 |
| PTPN6_E171_R | 9.03E−20 | 1.64E−17 | 2.8980 | 0.298 | 0.885 | −0.586 |
| PTPRO_P371_F | 1.20E−05 | 7.21E−05 | 1.3796 | 0.141 | 0.386 | −0.245 |
| RARA_P176_R | 0.001157 | 0.003366 | 1.1595 | 0.197 | 0.429 | −0.232 |
| SEMA3A_P343_F | 2.33E−06 | 1.85E−05 | 1.4008 | 0.103 | 0.311 | −0.207 |
| SEMA3B_P110_R | 3.67E−19 | 5.34E−17 | 2.6197 | 0.120 | 0.651 | −0.531 |
| SERPINE1_E189_R | 1.00E−14 | 8.44E−13 | 2.1014 | 0.164 | 0.611 | −0.447 |
| SHB_P691_R | 1.42E−18 | 1.88E−16 | 3.0713 | 0.099 | 0.695 | −0.596 |
| SNCG_E119_F | 7.68E−11 | 2.54E−09 | 2.0937 | 0.274 | 0.752 | −0.478 |
| SNCG_P53_F | 2.41E−15 | 2.51E−13 | 2.8608 | 0.299 | 0.881 | −0.582 |
| SNCG_P98_R | 6.08E−06 | 4.10E−05 | 1.1626 | 0.528 | 0.777 | −0.249 |
| SPDEF_P6_R | 5.50E−15 | 5.34E−13 | 2.3115 | 0.365 | 0.851 | −0.486 |
| SPP1_E140_R | 2.15E−10 | 5.90E−09 | 1.8149 | 0.433 | 0.822 | −0.388 |
| STAT5A_P704_R | 7.14E−06 | 4.72E−05 | 1.3639 | 0.205 | 0.502 | −0.297 |
| TAL1_P594_F | 0.001295 | 0.00369 | 1.1308 | 0.338 | 0.606 | −0.268 |
| TEK_E75_F | 0.001369 | 0.003848 | 1.0130 | 0.525 | 0.753 | −0.228 |
| TGFB2_E226_R | 0.00013 | 0.00052 | 1.4123 | 0.145 | 0.407 | −0.263 |
| TGFB3_E58_R | 7.06E−07 | 6.68E−06 | 1.3078 | 0.565 | 0.828 | −0.263 |
| TGFBI_P173_F | 1.97E−06 | 1.59E−05 | 1.4111 | 0.101 | 0.313 | −0.211 |
| THBS2_P605_R | 7.83E−15 | 7.13E−13 | 3.1447 | 0.248 | 0.883 | −0.635 |
| THY1_P149_R | 1.98E−07 | 2.28E−06 | 1.3813 | 0.135 | 0.378 | −0.244 |
| TNFRSF10A_P171_F | 5.34E−07 | 5.32E−06 | 1.7166 | 0.129 | 0.442 | −0.313 |
| TNFRSF10D_E27_F | 3.11E−11 | 1.13E−09 | 2.1540 | 0.103 | 0.489 | −0.386 |
| TNFRSF10D_P70_F | 1.89E−22 | 1.37E−19 | 2.6349 | 0.172 | 0.740 | −0.568 |
| TNFSF10_E53_F | 7.09E−26 | 1.03E−22 | 3.2196 | 0.095 | 0.718 | −0.622 |
| TNFSF10_P2_R | 9.29E−22 | 3.38E−19 | 3.6021 | 0.174 | 0.879 | −0.706 |
| WNT10B_P823_R | 8.10E−07 | 7.42E−06 | 1.7577 | 0.301 | 0.710 | −0.409 |

TABLE 9C
Statistically significant CpGs between skin and moles and melanoma

| | p.value. | q.value. | coef. | mean. | mean. | mean. |
|---|---|---|---|---|---|---|
| | skin. | skin. | skin. | beta. | beta. | beta. |
| ProbeID | vs.mole | vs.mole | vs.mole | skin | mole | diff |
| CAPG_E228_F | 5.33E−12 | 2.50E−10 | 2.3537 | 0.186 | 0.706 | −0.520 |
| MPO_P883_R | 8.32E−11 | 2.69E−09 | 1.3782 | 0.435 | 0.753 | −0.318 |
| RARA_P176_R | 0.001157 | 0.003366 | 1.1595 | 0.197 | 0.429 | −0.232 |
| SEMA3B_P110_R | 3.67E−19 | 5.34E−17 | 2.6197 | 0.120 | 0.651 | −0.531 |
| SHB_P691_R | 1.42E−18 | 1.88E−16 | 3.0713 | 0.099 | 0.695 | −0.596 |
| TGFB2_E226_R | 0.00013 | 0.00052 | 1.4123 | 0.145 | 0.407 | −0.263 |
| THBS2_P605_R | 7.83E−15 | 7.13E−13 | 3.1447 | 0.248 | 0.883 | −0.635 |
| TNFRSF10D_E27_F | 3.11E−11 | 1.13E−09 | 2.1540 | 0.103 | 0.489 | −0.386 |
| TNFSF10_E53_F | 7.09E−26 | 1.03E−22 | 3.2196 | 0.095 | 0.718 | −0.622 |
| WNT10B_P823_R | 8.10E−07 | 7.42E−06 | 1.7577 | 0.301 | 0.710 | −0.409 |

TABLE 9D
shows the accession numbers; specific single CpG coordinate; presence or absence
of CpG islands; specific sequences used in the Illumina GoldenGate array experiments; and
the synonyms for genes hypermethylated or hypomethylated in normal skin v. mole and
melanoma analysis. All gene IDs and accession numbers are from Ref Seq. version 36.1.

| Probe_ID | Gid | Accession | Gene_ID | Chrm | CpG_Coor | Dist_to_TSS | CpG_i |
|---|---|---|---|---|---|---|---|
| AATK_E63_R | 89041906 | XM_927215.1 | 9625 | 17 | 76709831 | 63 | N |
| AATK_P519_R | 89041906 | XM_927215.1 | 9625 | 17 | 76710413 | −519 | Y |
| AATK_P709_R | 89041906 | XM_927215.1 | 9625 | 17 | 76710603 | −709 | Y |
| ALOX12_E85_R | 4502050 | NM_000697.1 | 239 | 17 | 6840213 | 85 | Y |
| ALOX12_P223_R | 4502050 | NM_000697.1 | 239 | 17 | 6839905 | −223 | Y |
| ASCL2_P360_F | 42716308 | NM_005170.2 | 430 | 11 | 2249118 | −360 | Y |
| ASCL2_P609_R | 42716308 | NM_005170.2 | 430 | 11 | 2249367 | −609 | Y |
| AXL_P223_R | 21536465 | NM_021913.2 | 558 | 19 | 46416440 | −223 | Y |
| B3GALT5_E246_R | 15451880 | NM_033170.1 | 10317 | 21 | 39951370 | 246 | N |
| BGN_P333_R | 34304351 | NM_001711.3 | 633 | X | 152413272 | −333 | N |
| BLK_P14_F | 33469981 | NM_001715.2 | 640 | 8 | 11388916 | −14 | N |
| BMP4_P123_R | 19528651 | NM_130851.1 | 652 | 14 | 53493485 | −123 | Y |
| BMP4_P199_R | 19528651 | NM_130851.1 | 652 | 14 | 53493561 | −199 | Y |
| CALCA_P171_F | 76880483 | NM_001033952.1 | 796 | 11 | 14950579 | −171 | Y |
| CAPG_E228_F | 63252912 | NM_001747.2 | 822 | 2 | 85490959 | 228 | N |
| CASP10_E139_F | 47078266 | NM_001230.3 | 843 | 2 | 201756239 | 139 | N |
| CASP10_P334_F | 47078266 | NM_001230.3 | 843 | 2 | 201755766 | −334 | N |
| CDH11_E102_R | 16306531 | NM_001797.2 | 1009 | 16 | 63713318 | 102 | Y |
| CDH11_P354_R | 16306531 | NM_001797.2 | 1009 | 16 | 63713774 | −354 | Y |
| CDH13_P88_F | 61676095 | NM_001257.3 | 1012 | 16 | 81217991 | −88 | Y |
| CFTR_P372_R | 6995995 | NM_000492.2 | 1080 | 7 | 116906881 | −372 | Y |
| COL1A2_E299_F | 48762933 | NM_000089.3 | 1278 | 7 | 93862108 | 299 | Y |
| COL1A2_P407_R | 48762933 | NM_000089.3 | 1278 | 7 | 93861402 | −407 | N |
| COL1A2_P48_R | 48762933 | NM_000089.3 | 1278 | 7 | 93861761 | −48 | Y |
| CPA4_E20_F | 61743915 | NM_016352.2 | 51200 | 7 | 129720250 | 20 | N |
| CRIP1_P274_F | 39725694 | NM_001311.3 | 1396 | 14 | 105024320 | −274 | Y |
| CRIP1_P874_R | 39725694 | NM_001311.3 | 1396 | 14 | 105023720 | −874 | Y |
| CSF1R_P73_F | 27262658 | NM_005211.2 | 1436 | 5 | 149473201 | −73 | N |
| CSF3R_P8_F | 27437044 | NM_172313.1 | 1441 | 1 | 36721104 | −8 | N |
| CYP1B1_E83_R | 13325059 | NM_000104.2 | 1545 | 2 | 38156713 | 83 | Y |
| DDR1_P332_R | 38327631 | NM_001954.3 | 780 | 6 | 30959508 | −332 | N |
| DDR2_E331_F | 62420885 | NM_001014796.1 | 4921 | 1 | 160869183 | 331 | N |
| DDR2_P743_R | 62420885 | NM_001014796.1 | 4921 | 1 | 160868109 | −743 | N |
| DSC2_E90_F | 40806177 | NM_024422.2 | 1824 | 18 | 26936285 | 90 | Y |
| ELK3_P514_F | 44955920 | NM_005230.2 | 2004 | 12 | 95111824 | −514 | Y |
| ELL_P693_F | 47078265 | NM_006532.2 | 8178 | 19 | 18494611 | −693 | Y |
| EMR3_E61_F | 23397638 | NM_152939.1 | 84658 | 19 | 14646749 | 61 | N |
| EVI2A_P94_R | 51511748 | NM_001003927.1 | 2123 | 17 | 26672937 | −94 | N |
| EYA4_P794_F | 26667248 | NM_004100.2 | 2070 | 6 | 133603412 | −794 | Y |
| FANCE_P356_R | 66879667 | NM_021922.2 | 2178 | 6 | 35527760 | −356 | Y |
| FGF9_P862_R | 4503706 | NM_002010.1 | 2254 | 13 | 21143013 | −862 | Y |
| FGFR1_P204_F | 13186232 | NM_000604.2 | 2260 | 8 | 38445497 | −204 | Y |
| FLT1_P615_R | 32306519 | NM_002019.2 | 2321 | 13 | 27967847 | −615 | Y |
| FRZB_E186_R | 38455387 | NM_001463.2 | 2487 | 2 | 183439557 | 186 | Y |
| FRZB_P406_F | 38455387 | NM_001463.2 | 2487 | 2 | 183440149 | −406 | Y |
| GFI1_P208_R | 71037376 | NM_005263.2 | 2672 | 1 | 92725229 | −208 | Y |
| GJB2_P791_R | 42558282 | NM_004004.3 | 2706 | 13 | 19665828 | −791 | Y |
| GJB2_P931_R | 42558282 | NM_004004.3 | 2706 | 13 | 19665968 | −931 | Y |
| GNMT_P197_F | 54792737 | NM_018960.4 | 27232 | 6 | 43036281 | −197 | Y |
| GP1BB_P278_R | 9945387 | NM_000407.3 | 2812 | 22 | 18090788 | −278 | Y |
| GRB10_P496_R | 48762696 | NM_001001555.1 | 2887 | 7 | 50829148 | −496 | Y |
| GRB7_E71_R | 71979666 | NM_001030002.1 | 2886 | 17 | 35147784 | 71 | N |
| GRB7_P160_R | 71979666 | NM_001030002.1 | 2886 | 17 | 35147553 | −160 | N |
| GRPR_P200_R | 61677286 | NM_005314.2 | 2925 | X | 16051145 | −200 | N |
| HBII-52_E142_F | 29171307 | NR_001291.1 | 338433 | 15 | 22967111 | 142 | N |
| HBII-52_P563_F | 29171307 | NR_001291.1 | 338433 | 15 | 22966406 | −563 | Y |
| HCK_P858_F | 30795228 | NM_002110.2 | 3055 | 20 | 30102860 | −858 | Y |
| HDAC7A_P344_F | 13259521 | NM_015401.1 | 51564 | 12 | 46479534 | −344 | N |
| HFE_E273_R | 21040354 | NM_139010.1 | 3077 | 6 | 26195700 | 273 | Y |
| HHIP_P578_R | 20143972 | NM_022475.1 | 64399 | 4 | 145786045 | −578 | Y |
| HOXA11_E35_F | 24497552 | NM_005523.4 | 3207 | 7 | 27191320 | 35 | Y |
| HOXA11_P92_R | 24497552 | NM_005523.4 | 3207 | 7 | 27191447 | −92 | Y |
| HOXA9_E252_R | 24497558 | NM_002142.3 | 3205 | 7 | 27171422 | 252 | Y |
| HOXA9_P1141_R | 24497558 | NM_002142.3 | 3205 | 7 | 27172815 | −1141 | Y |
| HOXA9_P303_F | 24497558 | NM_002142.3 | 3205 | 7 | 27171977 | −303 | Y |
| HTR2A_P853_F | 60302916 | NM_000621.2 | 3356 | 13 | 46369029 | −853 | N |
| IFNG_E293_F | 56786137 | NM_000619.2 | 3458 | 12 | 66839495 | 293 | N |
| IFNGR2_P377_R | 47419933 | NM_005534.2 | 3460 | 21 | 33696695 | −377 | Y |
| IGF1_E394_F | 19923111 | NM_000618.2 | 3479 | 12 | 101398060 | 394 | N |
| IGFBP1_E48_R | 61744448 | NM_001013029.1 | 3484 | 7 | 45894532 | 48 | Y |
| IGFBP1_P12_R | 61744448 | NM_001013029.1 | 3484 | 7 | 45894472 | −12 | Y |
| IGFBP5_P9_R | 46094066 | NM_000599.2 | 3488 | 2 | 217268525 | −9 | Y |
| IL17RB_P788_R | 27477073 | NM_018725.2 | 55540 | 3 | 53854824 | 392 | Y |
| IL1RN_E42_F | 27894320 | NM_173843.1 | 3557 | 2 | 113591983 | 42 | N |
| IL1RN_P93_R | 27894320 | NM_173843.1 | 3557 | 2 | 113591848 | −93 | N |
| INSR_P1063_R | 4557883 | NM_000208.1 | 3643 | 19 | 7246074 | −1063 | Y |
| IPF1_P234_F | 4557672 | NM_000209.1 | 3651 | 13 | 27391943 | −234 | Y |
| JAK3_P1075_R | 47157314 | NM_000215.2 | 3718 | 19 | 17820875 | −1075 | N |
| KCNK4_E3_F | 15718764 | NM_016611.2 | 50801 | 11 | 63815454 | 3 | Y |
| KCNK4_P171_R | 15718764 | NM_016611.2 | 50801 | 11 | 63815280 | −171 | N |
| KIAA1804_P689_R | 24308329 | NM_032435.1 | 84451 | 1 | 231529448 | −689 | Y |
| KIT_P367_R | 4557694 | NM_000222.1 | 3815 | 4 | 55218551 | −367 | Y |
| KLK10_P268_R | 22208981 | NM_002776.3 | 5655 | 19 | 56215362 | −268 | N |
| KRAS_E82_F | 34485724 | NM_033360.2 | 3845 | 12 | 25295039 | 82 | Y |
| L1CAM_P19_F | 13435352 | NM_024003.1 | 3897 | X | 152794524 | −19 | Y |
| LEFTY2_P561_F | 27436880 | NM_003240.2 | 7044 | 1 | 224196104 | −561 | N |
| LOX_P313_R | 21264603 | NM_002317.3 | 4015 | 5 | 121442166 | −313 | Y |
| LY6G6E_P45_R | 13236491 | NM_024123.1 | 79136 | 6 | 31789613 | −1499 | N |
| LYN_P241_F | 4505054 | NM_002350.1 | 4067 | 8 | 56954685 | −241 | Y |
| MAGEC3_E307_F | 20162567 | NM_138702.1 | 139081 | X | 140754075 | 307 | N |
| MAGEC3_P903_F | 20162567 | NM_138702.1 | 139081 | X | 140752865 | −903 | N |
| MAP3K1_E81_F | 88983555 | XM_042066.10 | 4214 | 5 | 56146103 | 81 | Y |
| MAP3K1_P7_F | 88983555 | XM_042066.10 | 4214 | 5 | 56146015 | −7 | Y |
| MAP3K8_P1036_F | 22035597 | NM_005204.2 | 1326 | 10 | 30761836 | −1036 | Y |
| MAPK4_E273_R | 6715608 | NM_002747.2 | 5596 | 18 | 46444109 | 273 | N |
| MEST_E150_F | 29294638 | NM_002402.2 | 4232 | 7 | 129913432 | 150 | Y |
| MEST_P4_F | 29294638 | NM_002402.2 | 4232 | 7 | 129913278 | −4 | Y |
| MEST_P62_R | 29294638 | NM_002402.2 | 4232 | 7 | 129913220 | −62 | Y |
| MET_E333_F | 42741654 | NM_000245.2 | 4233 | 7 | 116100028 | 333 | Y |
| MMP7_E59_F | 75709180 | NM_002423.3 | 4316 | 11 | 101906629 | 59 | N |
| MPO_P883_R | 4557758 | NM_000250.1 | 4353 | 17 | 53714178 | −883 | N |
| MST1R_E42_R | 4505264 | NM_002447.1 | 4486 | 3 | 49916032 | 42 | Y |
| MUC1_E18_R | 65301116 | NM_002456.4 | 4582 | 1 | 153429306 | 18 | N |
| NBL1_E205_R | 33519445 | NM_005380.3 | 4681 | 1 | 19842518 | 205 | N |
| NBL1_P24_F | 33519445 | NM_005380.3 | 4681 | 1 | 19842289 | −24 | N |
| NOTCH4_E4_F | 55770875 | NM_004557.3 | 4855 | 6 | 32299818 | 4 | N |
| OPCML_P71_F | 59939898 | NM_002545.3 | 4978 | 11 | 132907684 | −71 | N |
| PARP1_P610_R | 11496989 | NM_001618.2 | 142 | 1 | 224663024 | −610 | Y |
| PDGFRA_E125_F | 61699224 | NM_006206.3 | 5156 | 4 | 54790329 | 125 | N |
| PDGFRB_E195_R | 68216043 | NM_002609.3 | 5159 | 5 | 149515420 | 195 | N |
| PGR_P790_F | 31981491 | NM_000926.2 | 5241 | 11 | 100507255 | −790 | N |
| PI3_P1394_R | 31657130 | NM_002638.2 | 5266 | 20 | 43235518 | −1394 | N |
| PLAU_P176_R | 53729348 | NM_002658.2 | 5328 | 10 | 75340720 | −176 | Y |
| POMC_P400_R | 4505948 | NM_000939.1 | 5443 | 2 | 25245356 | −400 | Y |
| PRSS1_E45_R | 21071011 | NM_002769.2 | 5644 | 7 | 142136949 | 45 | N |
| PRSS1_P1249_R | 21071011 | NM_002769.2 | 5644 | 7 | 142135655 | −1249 | N |
| PRSS8_E134_R | 21536453 | NM_002773.2 | 5652 | 16 | 31054518 | 134 | Y |
| PTHR1_P258_F | 39995096 | NM_000316.2 | 5745 | 3 | 46893982 | −258 | N |
| PTK7_E317_F | 27886610 | NM_002821.3 | 5754 | 6 | 43152324 | 317 | Y |
| PTPN6_E171_R | 34328901 | NM_080548.2 | 5777 | 12 | 6926172 | 171 | Y |
| PTPRO_P371_F | 13677212 | NM_002848.2 | 5800 | 12 | 15366383 | −371 | N |
| RARA_E128_R | 75812906 | NM_000964.2 | 5914 | 17 | 35719100 | 128 | N |
| RARA_P176_R | 75812906 | NM_000964.2 | 5914 | 17 | 35718796 | −176 | N |
| RARB_E114_F | 14916495 | NM_016152.2 | 5915 | 3 | 25444872 | 114 | Y |
| RARB_P60_F | 14916495 | NM_016152.2 | 5915 | 3 | 25444698 | −60 | Y |
| RARRES1_P426_R | 46255042 | NM_206963.1 | 5918 | 3 | 159933395 | −426 | Y |
| RARRES1_P57_R | 46255042 | NM_206963.1 | 5918 | 3 | 159933026 | −57 | Y |
| RBP1_P426_R | 8400726 | NM_002899.2 | 5947 | 3 | 140741606 | −426 | Y |
| RIPK1_P744_R | 57242760 | NM_003804.3 | 8737 | 6 | 3021313 | −744 | N |
| RIPK3_P124_F | 40254843 | NM_006871.2 | 11035 | 14 | 23879137 | −124 | N |
| RUNX3_E27_R | 72534651 | NM_001031680.1 | 864 | 1 | 25164035 | 27 | N |
| RUNX3_P247_F | 72534651 | NM_001031680.1 | 864 | 1 | 25164309 | −247 | Y |
| S100A2_P1186_F | 45269153 | NM_005978.3 | 6273 | 1 | 151806116 | −1186 | N |
| SEMA3A_P343_F | 5174672 | NM_006080.1 | 10371 | 7 | 83662191 | −343 | N |
| SEMA3A_P658_R | 5174672 | NM_006080.1 | 10371 | 7 | 83662506 | −658 | N |
| SEMA3B_E96_F | 54607087 | NM_004636.2 | 7869 | 3 | 50280140 | 96 | N |
| SEMA3B_P110_R | 54607087 | NM_004636.2 | 7869 | 3 | 50279934 | −110 | N |
| SERPINA5_P156_F | 34147643 | NM_000624.3 | 5104 | 14 | 94117408 | −156 | N |
| SERPINE1_E189_R | 10835158 | NM_000602.1 | 5054 | 7 | 100557361 | 189 | Y |
| SHB_P691_R | 4506934 | NM_003028.1 | 6461 | 9 | 38059901 | −691 | Y |
| SNCG_E119_F | 4507112 | NM_003087.1 | 6623 | 10 | 88708514 | 119 | N |
| SNCG_P53_F | 4507112 | NM_003087.1 | 6623 | 10 | 88708342 | −53 | Y |
| SNCG_P98_R | 4507112 | NM_003087.1 | 6623 | 10 | 88708297 | −98 | Y |
| SNURF_E256_R | 29540557 | NM_005678.3 | 8926 | 15 | 22751484 | 256 | Y |
| SPDEF_P6_R | 6912579 | NM_012391.1 | 25803 | 6 | 34632075 | −6 | N |
| SPP1_E140_R | 38146097 | NM_000582.2 | 6696 | 4 | 89115966 | 140 | N |
| STAT5A_P704_R | 21618341 | NM_003152.2 | 6776 | 17 | 37692387 | −704 | N |
| SYBL1_P349_F | 27545446 | NM_005638.3 | 6845 | X | 154763858 | −349 | Y |
| TAL1_E122_F | 4507362 | NM_003189.1 | 6886 | 1 | 47467908 | 122 | Y |
| TAL1_P594_F | 4507362 | NM_003189.1 | 6886 | 1 | 47468624 | −594 | Y |
| TEK_E75_F | 4557868 | NM_000459.1 | 7010 | 9 | 27099516 | 75 | N |
| TFF2_P178_F | 48928025 | NM_005423.3 | 7032 | 21 | 42644354 | −178 | N |
| TGFB2_E226_R | 4507462 | NM_003238.1 | 7042 | 1 | 216586717 | 226 | Y |
| TGFB3_E58_R | 4507464 | NM_003239.1 | 7043 | 14 | 75517184 | 58 | N |
| TGFBI_P173_F | 4507466 | NM_000358.1 | 7045 | 5 | 135392424 | −173 | Y |
| THBS2_P605_R | 40317627 | NM_003247.2 | 7058 | 6 | 169396667 | −605 | N |
| THY1_P149_R | 19923361 | NM_006288.2 | 7070 | 11 | 118799239 | −149 | Y |
| TNFRSF10A_P171_F | 21361085 | NM_003844.2 | 8797 | 8 | 23138755 | 70 | Y |
| TNFRSF10A_P91_F | 21361085 | NM_003844.2 | 8797 | 8 | 23138675 | −10 | Y |
| TNFRSF10C_E109_F | 22547120 | NM_003841.2 | 8794 | 8 | 23016488 | 109 | Y |
| TNFRSF10C_P7_F | 22547120 | NM_003841.2 | 8794 | 8 | 23016372 | −7 | Y |
| TNFRSF10D_E27_F | 42544227 | NM_003840.3 | 8793 | 8 | 23077458 | 27 | Y |
| TNFRSF10D_P70_F | 42544227 | NM_003840.3 | 8793 | 8 | 23077555 | −70 | Y |
| TNFSF10_E53_F | 23510439 | NM_003810.2 | 8743 | 3 | 173723910 | 53 | N |
| TNFSF10_P2_R | 23510439 | NM_003810.2 | 8743 | 3 | 173723965 | −2 | N |
| TNFSF8_E258_R | 24119162 | NM_001244.2 | 944 | 9 | 116732333 | 258 | N |
| TNFSF8_P184_F | 24119162 | NM_001244.2 | 944 | 9 | 116732775 | −184 | Y |
| TNK1_P221_F | 4507610 | NM_003985.1 | 8711 | 17 | 7224913 | −221 | Y |
| TRIM29_P261_F | 17402908 | NM_012101.2 | 23650 | 11 | 119514334 | −261 | N |
| TRIP6_P1090_F | 23308730 | NM_003302.1 | 7205 | 7 | 100301891 | −1090 | Y |
| VAV1_E9_F | 7108366 | NM_005428.2 | 7409 | 19 | 6723731 | 9 | Y |
| WNT10B_P823_R | 16936521 | NM_003394.2 | 7480 | 12 | 47652633 | −823 | Y |

| | SEQ | |
|---|---|---|
| Probe_ID | ID | Input_Sequence |
| AATK_E63_R | 94 | GGGCAGAAGCCAGCTTGATGGCAGACACCT[CG]CCACCAGTAGCAGGCGTGGGAGAGTC |
| AATK_P519_R | 95 | GGGGACGTGCCCAGTGGGTCCT[CG]AAGAAGGCAGGACAGAAGGCGG |
| AATK_P709_R | 96 | ACGGGTGGCCCGTGGCCCAGCAG[CG]GCTCCATGGCCAGCGAGGCGG |
| ALOX12_E85_R | 97 | GGGGCCTGGCTCTTCTCCGGGT[CG]TACAACCGCGTGCAGCTTTGGCTGGTCGG |
| ALOX12_P223_R | 98 | CCGTTGGCCTCACCCTGGCT[CG]GGCCCCTTTATCATCCTGCAGCTACG |
| ASCL2_P360_F | 99 | CCTAGCGCAGCTATGTCCCGAG[CG]CGCCCCCACCTGTGCGTTAATCTACTGG |
| ASCL2_P609_R | 100 | GGGCCTGGAGGTCTGCACCCGAC[CG]CCTTGTGCCAGGACGGTCAGGT |
| AXL_P223_R | 101 | GCCAGTAGCATGCCCCTGCC[CG]TCTGGGTCCCTCTGCGTGTCTCTGCTTGTC |
| B3GALT5_E246_R | 102 | CACACTCCTGGCATCCCAG[CG]TCTCCAGCTTGCATGGCCTGTCACGGTATT |
| BGN_P333_R | 103 | CCATCTCTCTTTCCTCTGCCTGG[CG]AGATGCCAGCCAGCACCTCAGTGTC |
| BLK_P14_F | 104 | GACAAAGCAAAACCAGTGAGGCTGAAAGAA[CG]GCTGCCCTGGTGCACACAGATGG |
| BMP4_P123_R | 105 | CCCGGAAGCCCAGGCAGCGCCCGAGTC[CG]CAGCTGCCGTCGGAGCTGGG |
| BMP4_P199_R | 106 | GGGGCTCACCTGGGGACCACGTG[CG]GAGGTACTAGAAAGCATGCACCGACT |
| CALCA_P171_F | 107 | AGGGGTCCTTTGCCCCTGGGTTG[CG]TCACCCTCATGCTTCCAGAACCTG |
| CAPG_E228_F | 108 | CTTTCTTCCTCCTACCTCTGCTT[CG]TAGGTTCGTCTTCCTTCCAGCCTGC |
| CASP10_E139_F | 109 | TTTGTTTTCAGGCAATTTCCCTGAGAAC[CG]TTTACTTCCAGAAGATTGGTGGAG |
| CASP10_P334_F | 110 | TGTGGACATAAGAAAGGGTTAACATGGC[CG]ACAACTATTTCATGAGCTTTTTGGCTT |
| CDH11_E102_R | 111 | GAGGGTGGACGCAACCTCCGAGC[CG]CCAGTCCCTGGCGCAGGGCAAGCG |
| CDH11_P354_R | 112 | TCAGGGCTCAGATGGAGTCTGGAG[CG]ACTGAAGTTGGGCTCCAGGG |
| CDH13_P88_F | 113 | CCGTATCTGCCATGCAAAACGAGGGAG[CG]TTAGGAAGGAATCCGTCTTGTAA |
| CFTR_P372_R | 114 | TCTAGGAAGCTCTCCGGGGAGC[CG]GTTCTCCCGCCGGTGGCTTCTTCTG |
| COL1A2_E299_F | 115 | ACCCTAGGGCCAGGGAAACTTTTGC[CG]TATAAATAGGGCAGATCCGGGCTTT |
| COL1A2_P407_R | 116 | CAAAGCCTATCCTCCCTGTAGC[CG]GGTGCCAAGCAGCCTCGAGCCTGCTC |
| COL1A2_P48_R | 117 | GACTGGACAGCTCCTGCTTTGATCGC[CG]GAGATCTGCAAATTCTGCCCATGTCGGGG |
| CPA4_E20_F | 118 | CTTGACTCAGCCACTGTATGACTGACTCCC[CG]GGGACATGAGGTGGATACT |
| CRIP1_P274_F | 119 | AGACATCACAGCGCTGGGCTAGGGGCG[CG]GCTTGAACTCGCCTAAAGAGCTG |
| CRIP1_P874_R | 120 | CCTCAACTTTGCAGCGTACTTGGAC[CG]CTCTGGCCGCCCTGGGCGCTACCC |
| CSF1R_P73_F | 121 | TCTAGCAGCTGCCTGTCACAGAGCA[CG]CCGGCCTCAATCCGGGCCTGTGGGC |
| CSF3R_P8_F | 122 | GCTTCTCTCCCCGAGCTCTGT[CG]TTAATGGCTCAGCCTCTGACAGGCCCG |
| CYP1B1_E83_R | 123 | GTTGAGATTGAGACTGGGGGT[CG]GTGAGTGGCGTCAATTCCCATG |
| DDR1_P332_R | 124 | GGCCTGGGCGTCTGGACCCC[CG]GGTCCCTTAGAACGCCCTTCAGA |
| DDR2_E331_F | 125 | GCGTTTTAAGTCAGACAAGGAAGGGAA[CG]TAATGAGGCACCACAGACTCGAGAAAT |
| DDR2_P743_R | 126 | TCCTCCCCTGTTGCCTACC[CG]CCCCTTTCACATGATCTCTGACTATAGCTG |
| DSC2_E90_F | 127 | CTGCGCAAGGTGTTTCTCACCAG[CG]GACGCCACCTATAAGGCCCATCTC |
| ELK3_P514_F | 128 | GGCCGAGGGCTGGCTTTTAAAACAC[CG]AAAACCCAGACAGGAACGGTGTCC |
| ELL_P693_F | 129 | ATCCCCACAGTCCCTGAG[CG]ATGGTGCAGTCCAGCTTCATTTTCCTATT |
| EMR3_E61_F | 130 | AGCAAACTGCTTCCCCTCTTT[CG]CCATCAGACTCATGGTTCTGCTTTTCGTTT |
| EVI2A_P94_R | 131 | CATGACAGGAGGCTTTGTAGAACCAATCCC[CG]CCTCCAGAGCAGGGAGGGTTTT |
| EYA4_P794_F | 132 | TCAGCAATGTGCCTAGAGAAGCTCTGACGC[CG]CCTTGGAAGTAAGTCGTTGCTG |
| FANCE_P356_R | 133 | CATGACAAGCAACATGCCGTCAG[CG]TAAATACAGCGCGGGTCCTCTAGCACA |
| FGF9_P862_R | 134 | GACTCAGGGTTTCTTCCTCC[CG]CCTCTCGCAGTGCATCTTTCATTTGCTTTT |
| FGFR1_P204_F | 135 | CTACAGCCTGGTCTCCTTTGGCGTTTG[CG]CCCCTGCATCTGAGCACGTCCCA |
| FLT1_P615_R | 136 | GAAGTCTAGGAAGGCACCGGAGACCCT[CG]GCACAAGGCACTGAACCTGGAGCG |
| FRZB_E186_R | 137 | CAGGATGGGGCAGGGTGCAGCCG[CG]CAGTGGACGCCAAAAGGCCCGCT |
| FRZB_P406_F | 138 | GGGACGTCTGTGCCTCTGCCCGGG[CG]GCTCTGCACTTTCCTACCTCCCGC |
| GFI1_P208_R | 139 | GAGGTCATACCCAGGCACTGGGTGTTGG[CG]GGAGCAGTAAAGCGCCATAAAAGCACC |
| GJB2_P791_R | 140 | GTGCCAAGGACTAAGGTTGGGGG[CG]GTGGGAGAGACAAGCCTCGTT |
| GJB2_P931_R | 141 | GGAACTGCAAGGAGGTGACTCCTTT[CG]GGGTGAGGAGGCCCAGAC |
| GNMT_P197_F | 142 | GGGATTGCACAGAGGGCTGGGTC[CG]CAGGCTGGCTAAAAGGACCTAGCCC |
| GP1BB_P278_R | 143 | ACACGATGCTCCGTTTTCTTC[CG]TTGTGAATGCCGCGTCCTGTCCTGGTGACA |
| GRB10_P496_R | 144 | TACTCTGTCGTGGGCTGAAGGCACC[CG]GCCTGGGAAAAGGAAACC |
| GRB7_E71_R | 145 | GCCTCTGACTTCTCTGTCCGAAGT[CG]GGACACCCTCCTACCACCTGTAGAG |
| GRB7_P160_R | 146 | GGTACTGTCTGTTCGGCTGTCTTCCC[CG]CCTCTCCCCAGGCACCTGCATC |
| GRPR_P200_R | 147 | CACATGGACACCCTGTGCATCAGTGTG[CG]TTTAATTCAAAGACAGACCTCATTTGATAG |
| HBII-52_E142_F | 148 | GGCCCCCGACGGGGCCACTGTATTT[CG]GGCTGCAGACCTAGAGGCCCTG |
| HBII-52_P563_F | 149 | GCCCAGGGGCAGGCTATGTGACTGCC[CG]GTCTGCAGCTGTAAGTGGTTTCT |
| HCK_P858_F | 150 | TGGTGTCTGAATGGAGCAGGCCTG[CG]GAAGAGAAACCGCTGACCACAGACC |
| HDAC7A_P344_F | 151 | AGCCTCACAGGCCCTCTGGGT[CG]CCACCCTCCCATGCTCTATCCC |
| HFE_E273_R | 152 | TCCTCCTGATGCTTTTGCAGACCG[CG]GTCCTGCAGGGGCGCTTGCTGCGTGAGTCC |
| HHIP_P578_R | 153 | AAACCATCTCAGCCTACTCAA[CG]GCATCTGGGATGTCCCCCTGCCTCTA |
| HOXA11_E35_F | 154 | ACCTTGGGCTCTCCGCAGTAGC[CG]AGCTTAACATGATTCTCCACTGCAGCTGCC |
| HOXA11_P92_R | 155 | CAGGGAGGTGCTGGTCATGTGACC[CG]ATGTTGAAATTGACAAGCTGCTAGCT |
| HOXA9_E252_R | 156 | TGGGTTCCACGAGGCGCCAAACACCGT[CG]CCTTGGACTGGAAGCTGCACG |
| HOXA9_P1141_R | 157 | CTACAAGTGGCATGAATGGAAGGCAAGTT[CG]GTTTGGGAAAAGGCAGCCTC |
| HOXA9_P303_F | 158 | CCCCATACACACACTTCTTAAG[CG]GACTATTTTATATCACAATTAATCACGCCA |
| HTR2A_P853_F | 159 | CCTGTTGGCTTCCTCTGGCACGGCT[CG]GCTGGGTTCCTCCCTCCCTGTGCGG |
| IFNG_E293_F | 160 | AGCCTATCAGAGATGCTACAGCAAGT[CG]ATATTCAGTCATTTTCAACCACAAA |
| IFNGR2_P377_R | 161 | CTATGTTGCAAAACCCATTTTTGCTAA[CG]TGTCCAGTGGGCTCCCGGGACGAC |
| IGF1_E394_F | 162 | TGTGCAAATGCATCCATCTCCC[CG]AGCTATTTTTCAGATTCCACAGAATTGCA |
| IGFBP1_E48_R | 163 | ATTTTGAACACTCAGCTCCTAGCGTG[CG]GCGCTGCCAATCATTAACCTCCTGGTGC |
| IGFBP1_P12_R | 164 | CCTCCCACCAGCGGTTTG[CG]TAGGGCCTTGGGTGCACTAGCAAAACAAAC |
| IGFBP5_P9_R | 165 | GAAGTTTCCAAAGAGACTACGGGGCTC[CG]GGAGAGCAGGCGCTTTTAAATAGC |
| IL17RB_P788_R | 166 | CAGCTCCAAATCGCCAGTGCTGA[CG]GCTTCCGCTTTGGGAGCCCCAG |
| IL1RN_E42_F | 167 | GAGGGACTGTGGCCCAGGTACTGCC[CG]GGTGCTACTTTATGGGCAGCAGCT |
| IL1RN_P93_R | 168 | CATCAAGTCAGCCATCAGC[CG]GCCCATCTCCTCATGCTGGCCAAC |
| INSR_P1063_R | 169 | GACGCTTCTGAAAGGGCAAAGACGA[CG]CCAAAGAAGACGCCGGAGACCTC |
| IPF1_P234_F | 170 | CCATTTTGGGGAGCACCGCCAGCTGCC[CG]TTCAGGAGTGTGCAGCAAACTCAGCTG |
| JAK3_P1075_R | 171 | GGACAGGCACAGACTGGAACTTGGACC[CG]AGGCAGGACAGGGAGCTGGC |
| KCNK4_E3_F | 172 | GAGATGCCAGATTAGCGTGGTGCCTGTC[CG]GAGAGACGGGCCAGCTGATG |
| KCNK4_P171_R | 173 | AGGTGGGTCCCAACCTCCA[CG]TCGGCCAATTCCAGGTGGCCCC |
| KIAA1804_P689_R | 174 | GCACTGGCCCAGGTCTGGCAC[CG]CGCTACAATTTCTTCTGTAGCCCGTTCTGA |
| KIT_P367_R | 175 | GCGTGGTGCCCAGCTTCACAAAG[CG]AGCGGGCAGCACCTCCTTGGTCCG |
| KLK10_P268_R | 176 | AACAGAAACAAGGAAAAAGGGAAACCCA[CG]CCCACTCTGTGGCCGTGAGTGA |
| KRAS_E82_F | 177 | TCGCTCCCAGTCCGAAATGG[CG]GGGGCCGGGAGTACTGGCCGAGCCGC |
| L1CAM_P19_F | 178 | CAGCACAGCCAGCCGGGCT[CG]GTTCAGGCTCCGGCCGGAGGGG |
| LEFTY2_P561_F | 179 | CCCATGACATCCTCTGTCTAGACA[CG]GTCAGGACACAAATCTGGCAGCTCTACTGT |
| LOX_P313_R | 180 | AGGCGAAGGCAGCCAGGCCATGGGG[CG]ACGCCAAAATATGCACGAAGAAAAATG |
| LY6G6E_P45_R | 181 | AATCTGGGAGAGGTGATCTGCACCC[CG]AGATCCCGGGATTTGTAGAGTT |
| LYN_P241_F | 182 | GGAAAGGAGACGCGAGAGGTGTAGT[CG]ATGTGCCTGCGAAGCCCAGGCT |
| MAGEC3_E307_F | 183 | TCCCTTGGTTGCAGTAGCCTGTGGT[CG]CTCATGTCTGAATCTCCAGGGAA |
| MAGEC3_P903_F | 184 | TGCAGCCTGAGTTAGACTTCTGCAACGTCC[CG]TGAGGTGGGATCAGGAATG |
| MAP3K1_E81_F | 185 | CTGCAGGGAAGAAGGACGTGCGG[CG]AGAAGCATCGGATTCGGGG |
| MAP3K1_P7_F | 186 | GTAGAGTCCAGGGACTAGGAGGACTCACAA[CG]CAGCGATGGGCAGCCAGGCCCTG |
| MAP3K8_P1036_F | 187 | ACCTGGGCACTGGGAAGAATAGGG[CG]TGGACTTGGAGTGTGACCG |
| MAPK4_E273_R | 188 | CCCTCCCAATGCAGGTTAAGA[CG]ACAGCCTGCGCCCCCAACTAGC |
| MEST_E150_F | 189 | TCAGGAAGCGCATGCGCAACCGGTTCTC[CG]AAACATGGAGTCCTGTAGGCAAGG |
| MEST_P4_F | 190 | GCTGACGCCTGGCAGGGAGAAGG[CG]GCAGCACATGCTGGGCTCGGG |
| MEST_P62_R | 191 | GCCGGAGGCTATTGTCGAAGCCA[CG]GCCTGCCATTTCATACCCTTTGCAA |
| MET_E333_F | 192 | GGAAACTGAAGAGACGTGGCCACGG[CG]AGGACGAAACTAGAATGGGG |
| MMP7_E59_F | 193 | CAGGCACACAGCACACAGCA[CG]GTGAGTCGCATAGCTGCCGTCCAGAGAC |
| MPO_P883_R | 194 | GGACAGGAAATCTGGCTGGAGAC[CG]TTGGGCTTCACAGGAAGGAG |
| MST1R_E42_R | 195 | AGCAGCAACAGGAAGGACTGAGGCAGCGG[CG]GGAGGAGCTCCATCGAGGC |
| MUC1_E18_R | 196 | GGAGGGGGCAGAACAGATTCAGGCAGG[CG]CTGGCTGCTTGAGAGGTG |
| NBL1_E205_R | 197 | AAATCCCCAAGTCCTACAAT[CG]TGTCCCAGTGGTGTCCCTGGGCCAC |
| NBL1_P24_F | 198 | GAATTCCGGGCAGAGGGAAGGG[CG]CAGGCAACAGCTAGGAGGCGCAGATGC |
| NOTCH4_E4_F | 199 | CCTCGGCCTGCTGCAAGCCTCA[CG]TCTGAGCTGTTTCCTGAGTCACACAATGTC |
| OPCML_P71_F | 200 | CAGAGCAGTCCTCCAAGGCA[CG]CATTGGCTCCACTCTCCTGAGCGACGG |
| PARP1_P610_R | 201 | TCCGGGAAGCGCAGGCCCCCGCCT[CG]GGAATATAGTTGATTGGCCCGA |
| PDGFRA_E125_F | 202 | GTGTGGGACATTCATTGCGGAATAACAT[CG]GAGGAGAAGGTAAGGGAA |
| PDGFRB_E195_R | 203 | AAGCATCCTTCGGGAGGAGCAGAGC[CG]CCAGAGGGGCCGCCCTGG |
| PGR_P790_F | 204 | CACTAGCAGTTATTCCACATTTC[CG]CCTAAATCTCCCAGCAGCCACTAATAT |
| PI3_P1394_R | 205 | AAAGGCTTCCACAGTCTGACATT[CG]TTTATGTCTCCCTCAGTTTCAGGCTTGG |
| PLAU_P176_R | 206 | TCTCGATTCCTCAGTCCAGA[CG]CTGTTGGGTCCCCTCCGCTGGAGATC |
| POMC_P400_R | 207 | TGGTTCGCATTTGGCGGTAAATATCAC[CG]TCTGCACACGGGGAGGCCTCC |
| PRSS1_E45_R | 208 | CTGATCCTTACCTTTGTGGCAGCTGCT[CG]TGAGTATCATGCCCTGCCTCAGGCCC |
| PRSS1_P1249_R | 209 | TAGCCCCCTGGCCAGGTC[CG]ATTTCAACACCAAGTTTCTGAGCTTTT |
| PRSS8_E134_R | 210 | GGGAGACGCCTGGAGTATCCGAAG[CG]AGCAGTGTGGACGAGTCACCAGCACCG |
| PTHR1_P258_F | 211 | GGCAAGGAGAGGACTATTGAGGCACACACA[CG]TGTCTGGCAGCCTGAGTGGG |
| PTK7_E317_F | 212 | GGGGGCACAGAGCTTGGGAAGCG[CG]GGAGTCCCGTGGGCAAAAG |
| PTPN6_E171_R | 213 | GAGATGCTGTCCCGTGGGTAAGTCC[CG]GGCACCATCGGGGTCCCAGTCT |
| PTPRO_P371_F | 214 | TGAGAGGGAACTGGGATCTGG[CG]CCTGGATTGCTCAAGAGAGGTC |
| RARA_E128_R | 215 | CCCTTCCCAATTCTTTGGC[CG]CCTTTGACCCCGGCCTCTGCTTCTGA |
| RARA_P176_R | 216 | GAACTGTTCCTGTCCCCAGC[CG]ATGACCAGACGCCCATCTTTCTTC |
| RARB_E114_F | 217 | GAGGACTGGGATGCCGAGAACG[CG]AGCGATCCGAGCAGGGTTTGTC |
| RARB_P60_F | 218 | CTAGTTGGGTCATTTGAAGGTTAGCAGCC[CG]GGTAGGGTTCACCGAAAGTTCA |
| RARRES1_P426_R | 219 | CGGAGAAAGGGGCAGGCCGCAG[CG]GGCATTGATGGGGCTCCT |
| RARRES1_P57_R | 220 | CCAGGGCGAAGGTCTGTAGCGAGCC[CG]GGTCCCCATGGGGCCACTCC |
| RBP1_P426_R | 221 | GAAAGCTGGGAGGTTCAACTACGGG[CG]AGAAAATTGGGGCACTTTCCACG |
| RIPK1_P744_R | 222 | CCCCTGTGTGAGCTACTGCCTGCCTC[CG]GTGCTCTGTTTCTGTCCCTAGAGTTCTTTT |
| RIPK3_P124_F | 223 | AAAGCTAGTGCCTTTCTCCTTGACTAG[CG]TTTCCTGAGCACCTGCCGCAGCC |
| RUNX3_E27_R | 224 | CGGCAGCCAGGGTGGAGGAGCTC[CG]AAGCTGACAGAGCAGAGTGGGCC |
| RUNX3_P247_F | 225 | CGGCCTTGGCTCATTGGCTGGGCCG[CG]GTCACCTGGGCCGTGATGTCACGGCC |
| S100A2_P1186_F | 226 | TCTACACCTTGGCACAGCCAC[CG]AGTGTCCCTTGCTCCCCTCAGTACTT |
| SEMA3A_P343_F | 227 | CCTTTTATCTAAGCTCCTCTGATAGC[CG]GTGGCAGTCTCTAATCCTGCTCCCTGCTTC |
| SEMA3A_P658_R | 228 | GAGATTAGAGCCGGGAGCAGAACCCTCAGG[CG]TGCCTGTGAAAGGCATGTAGCTATAA |
| SEMA3B_E96_F | 229 | GAGAGATGCTGCTGCGGAAGTCCT[CG]GTGGAGTGTGAGAAGGCAGC |
| SEMA3B_P110_R | 230 | CTTGTGCCCATTCCACTCC[CG]CCTGGCTGCCGTCTCCAGCTGGTCCC |
| SERPINA5_P156_F | 231 | GCGTCTGCAGGCAGGCCTGCTGGC[CG]GAAACCTGCCAGGAAAGGAAG |
| SERPINE1_E189_R | 232 | CGCTATTCCTCTATTTTCTTTTCCT[CG]GACCTGCAGCCTTGGGTCGACCCTGC |
| SHB_P691_R | 233 | GGTGGGAGCCGGGCCCAGCACCAATC[CG]AGAGCAAGGCTAGGGGAGGTC |
| SNCG_E119_F | 234 | GGAAAAGACCAAGCAGGGGGTGA[CG]GAAGCAGCTGAGAAGACCAAGGAG |
| SNCG_P53_F | 235 | CGTCAATAGGAGGCATCGGGGACAGC[CG]CTGCGGCAGCACTCGAGCCAGCTCAAG |
| SNCG_P98_R | 236 | GCTGGCTGGGCTCCAGCTGGCCTC[CG]CATCAATATTTCATCGGCGTCAATAGGA |
| SNURF_E256_R | 237 | AGGCTTGCTGTTGTGCCGTTCTGCCC[CG]ATGGTATCCTGTCCGCTCGCATTGGGGCG |
| SPDEF_P6_R | 238 | TGTGCTGGGAGGAAGTCAGACAGCCG[CG]AGATGAAGAGTTGGCCAGGGC |
| SPP1_E140_R | 239 | AGTTGCAGCCTTCTCAGCCAAA[CG]CCGACCAAGGTACAGCTTCAGTTTGCTACT |
| STAT5A_P704_R | 240 | CAGCCACCGACAGGCTGCATGA[CG]GTGGCAAAGTCACTTCCCCTCTCTG |
| SYBL1_P349_F | 241 | ATTTTGTCTGTGAGGAAACGGG[CG]ACGCTGCCTACTGAGACTAAGCAGGA |
| TAL1_E122_F | 242 | CCGACAGGCTGTCTGGAACATTTT[CG]AACCCTCCAACTGGGATCGGTCTGGTT |
| TAL1_P594_F | 243 | TCACACATCGAAGTCTTGGATTAACTG[CG]AAGGCCTCCTTCTATTTGCCGCGGCTT |
| TEK_E75_F | 244 | GTAGGACGATGCTAATGGAAAGTCACAAAC[CG]CTGGGTTTTTGAAAGGATC |
| TFF2_P178_F | 245 | GCCAGGGTGACTCTCTCCCTGCT[CG]GTGATACCTCTTCCTGCCCTGGACAGA |
| TGFB2_E226_R | 246 | TTTCTGATCCTGCATCTGGTCACGGT[CG]CGCTCAGCCTGTCTACCTGCAGCACACT |
| TGFB3_E58_R | 247 | CAGGAAGCGCTGGCAACCCTGAGGA[CG]AAGAAGCGGACTGTGTGCCTT |
| TGFBI_P173_F | 248 | ACTGAGCACGGGCACAGTGCGGGAG[CG]GGTGGGTGCCCAGGGCAG |
| THBS2_P605_R | 249 | AACCTGACGTGCAGGCACAGAGCAAGGACT[CG]AGAGAACGAGAAGCAGTGGCAGCAGCT |
| THY1_P149_R | 250 | GGAAGGAAGAGAAGGCGGTCC[CG]CATTGGTGTGAGAGTGGCAGG |
| TNFRSF10A_P171_F | 251 | TCGTTTTGCCACTTGGTCCCAG[CG]CCAGGCTTCTCGGTCGGGAGTTGACCT |
| TNFRSF10A_P91_F | 252 | TTCCTCTGTGACCGCCCTTGC[CG]CTCTCAGCTTCTGTTCCTCAACCAC |
| TNFRSF10C_E109_F | 253 | AGGGGTGAAGGAGCGCTTCCTAC[CG]TTAGGGAACTCTGGGGACAG |
| TNFRSF10C_P7_F | 254 | GGGTATAAATTCAGAGGCGCTGCGCTC[CG]ATTCTGGCAGTGCAGCTGTGGG |
| TNFRSF10D_E27_F | 255 | CAGAAATCGTCCCCGTAGTTTGTG[CG]CGTGCAAAGGTTCTCGCAGCTACACTGCCA |
| TNFRSF10D_P70_F | 256 | CGTGGTCAGTTGTACTCCCTTCC[CG]CAGTCACTTCCAGGCACTCAGGCTGG |
| TNFSF10_E53_F | 257 | GACTGCTGTAAGTCAGCCAGGCAGC[CG]GTCACTGAAGCCCTTCCTTCTCTATT |
| TNFSF10_P2_R | 258 | TCTTTTATAGTCAGTGAGGAAATGAAAG[CG]AATGAGTTGTTTTTCTGGGT |
| TNFSF8_E258_R | 259 | CCCCAGGTGGCTGGCCACGGAGCC[CG]CCGGCACATGCATGGCTGTGTCTC |
| TNFSF8_P184_F | 260 | CACACACAAAGCAACTTCTGTTT[CG]TTTAGACTCTGCCACAAAACGCCTTC |
| TNK1_P221_F | 261 | GGCTGGAAAGACGTGAAGGAAGA[CG]AGCAGAGGAGAAGGGAAGG |
| TRIM29_P261_F | 262 | GCACTTGCTTCTCATCCGGGGAG[CG]GGGAGTCTCCGTCTTCACAAGTGGGCA |
| TRIP6_P1090_F | 263 | AAGGGGACTTTGTGAACAGTGGG[CG]GGGAGACGCAGAGGCAGAGG |
| VAV1_E9_F | 264 | AAAGAAGAGGAAGTGGTAGCACTAGCTGT[CG]CTCCACAGGCGAGCAGGGCAGGCG |
| WNT10B_P823_R | 265 | CTTGGGGTGCACAGGCAAAGGCAAAC[CG]CCTTAGGGAGACCCAGTGGCAGCG |

| Probe_ID | Synonym | cg_no |
|---|---|---|
| AATK_E63_R | . | cg05292376 |
| AATK_P519_R | . | cg17279079 |
| AATK_P709_R | . | cg02979355 |
| ALOX12_E85_R | LOG12 | cg05878700 |
| ALOX12_P223_R | LOG12 | cg22819332 |
| ASCL2_P360_F | ASH2, HASH2, MASH2 | cg15376678 |
| ASCL2_P609_R | ASH2, HASH2, MASH2 | cg00868120 |
| AXL_P223_R | UFO | cg09524393 |
| B3GALT5_E246_R | B3T5, GLCT5, B3GalTx, B3GalT-V, beta3Gal-T5 | cg11479877 |
| BGN_P333_R | PGI, DSPG1, PG-S1, SLRR1A | cg04929865 |
| BLK_P14_F | MGC10442 | cg22826986 |
| BMP4_P123_R | ZYME, BMP2B, BMP2B1 | cg26240298 |
| BMP4_P199_R | ZYME, BMP2B, BMP2B1 | cg09229893 |
| CALCA_P171_F | CT, KC, CGRP, CALC1, CGRP1, CGRP-I, MGC126648 | cg24117998 |
| CAPG_E228_F | MCP, AFCP | cg13268943 |
| CASP10_E139_F | MCH4, ALPS2, FLICE2 | cg20209903 |
| CASP10_P334_F | MCH4, ALPS2, FLICE2 | cg13782463 |
| CDH11_E102_R | OB, CAD11, CDHOB, OSF-4 | cg05318914 |
| CDH11_P354_R | OB, CAD11, CDHOB, OSF-4 | cg13126606 |
| CDH13_P88_F | CDHH | cg08977371 |
| CFTR_P372_R | CF, MRP7, ABC35, ABCC7, TNR-CFTR, dJ760C5.1 | cg24329417 |
| COL1A2_E299_F | OI4 | cg22877867 |
| COL1A2_P407_R | OI4 | cg16337370 |
| COL1A2_P48_R | OI4 | cg26942275 |
| CPA4_E20_F | CPA3 | cg01796223 |
| CRIP1_P274_F | CRHP, CRIP, CRP1 | cg05417129 |
| CRIP1_P874_R | CRHP, CRIP, CRP1 | cg03324382 |
| CSF1R_P73_F | FMS, CSFR, FIM2, C-FMS, CD115 | cg01875467 |
| CSF3R_P8_F | CD114, GCSFR | cg00474419 |
| CYP1B1_E83_R | CP1B, GLC3A | cg09991178 |
| DDR1_P332_R | CAK, DDR, NEP, PTK3, RTK6, TRKE, CD167, EDDR1, | cg02680487 |
| | MCK10, NTRK4, PTK3A | |
| DDR2_E331_F | TKT, NTRKR3, TYRO10 | cg22740835 |
| DDR2_P743_R | TKT, NTRKR3, TYRO10 | cg23028772 |
| DSC2_E90_F | DG2, DSC3, CDHF2, DGII/III, DKFZp686I11137 | cg08156793 |
| ELK3_P514_F | ERP, NET, SAP2 | cg11467837 |
| ELL_P693_F | Men, ELL1, C19orf17, ELL_HUMAN, DKFZp434I1916 | cg09597048 |
| EMR3_E61_F | . | cg15552238 |
| EVI2A_P94_R | EVDA, EVI2 | cg23352695 |
| EYA4_P794_F | CMD1J, DFNA10 | cg24842760 |
| FANCE_P356_R | FAE, FACE | cg04035266 |
| FGF9_P862_R | GAF, HBFG-9, MGC119914, MGC119915 | cg02259997 |
| FGFR1_P204_F | H2, H3, H4, H5, CEK, FLG, FLT2, KAL2, BFGFR, | cg20658205 |
| | C-FGR, CD331, N-SAM | |
| FLT1_P615_R | FLT, VEGFR1 | cg26282369 |
| FRZB_E186_R | FRE, FZRB, hFIZ, FRITZ, FRP-3, FRZB1, SFRP3, | cg01872931 |
| | SRFP3, FRZB-1, FRZB-PEN | |
| FRZB_P406_F | FRE, FZRB, hFIZ, FRITZ, FRP-3, FRZB1, SFRP3, | cg25188149 |
| | SRFP3, FRZB-1, FRZB-PEN | |
| GFI1_P208_R | ZNF163 | cg20125091 |
| GJB2_P791_R | HID, KID, PPK, CX26, DFNA3, DFNB1, NSRD1 | cg20193013 |
| GJB2_P931_R | HID, KID, PPK, CX26, DFNA3, DFNB1, NSRD1 | cg09195389 |
| GNMT_P197_F | . | cg04013093 |
| GP1BB_P278_R | CD42c | cg19755554 |
| GRB10_P496_R | RSS, IRBP, MEG1, GRB-IR, KIAA0207 | cg19392396 |
| GRB7_E71_R | . | cg23836594 |
| GRB7_P160_R | . | cg08284496 |
| GRPR_P200_R | . | cg26196133 |
| HBII-52_E142_F | RNHBII52 | cg24301180 |
| HBII-52_P563_F | RNHBII52 | cg21361081 |
| HCK_P858_F | JTK9 | cg04775393 |
| HDAC7A_P344_F | HDAC7, DKFZP586J0917 | cg25755806 |
| HFE_E273_R | HH, HFE1, HLA-H, MGC103790, dJ221C16.10.1 | cg13740565 |
| HHIP_P578_R | HIP, FLI20992, FU90230 | cg02524475 |
| HOXA11_E35_F | HOX1, HOX11 | cg08479590 |
| HOXA11_P92_R | HOX1, HOX11 | cg18977999 |
| HOXA9_E252_R | HOX1, ABD-B, HOX1G, HOX1.7, MGC1934 | cg10604830 |
| HOXA9_P1141_R | HOX1, ABD-B, HOX1G, HOX1.7, MGC1934 | cg15262939 |
| HOXA9_P303_F | HOX1, ABD-B, HOX1G, HOX1.7, MGC1934 | cg03715906 |
| HTR2A_P853_F | HTR2, 5-HT2A | cg15268261 |
| IFNG_E293_F | IFG, IFI | cg23001963 |
| IFNGR2_P377_R | AF-1, IFGR2, IFNGT1 | cg21449657 |
| IGF1_E394_F | IGFI | cg17084217 |
| IGFBP1_E48_R | AFBP, IBP1, PP12, IGF-BP25, hIGFBP-1 | cg20666158 |
| IGFBP1_P12_R | AFBP, IBP1, PP12, IGF-BP25, hIGFBP-1 | cg00110785 |
| IGFBP5_P9_R | IBP5 | cg20419545 |
| IL17RB_P788_R | CRL4, EV127, IL17BR, IL17RH1, MGC5245 | cg16868427 |
| IL1RN_E42_F | IRAP, IL1F3, IL1RA, IL-1ra3, ICIL-1RA, MGC10430 | cg17669033 |
| IL1RN_P93_R | IRAP, IL1F3, IL1RA, IL-1ra3, ICIL-1RA, MGC10430 | cg14497465 |
| INSR_P1063_R | CD220 | cg00650214 |
| IPF1_P234_F | IUF1, PDX1, IDX-1, MODY4, PDX-1, STF-1 | cg20815612 |
| JAK3_P1075_R | JAKL, LJAK, JAK-3, L-JAK, JAK3_HUMAN | cg05244380 |
| KCNK4_E3_F | TRAAK, DKFZP566E164 | cg01352108 |
| KCNK4_P171_R | TRAAK, DKFZP566E164 | cg25881850 |
| KIAA1804_P689_R | MLK4, dJ862P8.3 | cg09524235 |
| KIT_P367_R | PBT, SCFR, C-Kit, CD117 | cg23927351 |
| KLK10_P268_R | NES1, PRSSL1 | cg06130787 |
| KRAS_E82_F | KRAS1, KRAS2, RASK2, KI-RAS, C-K-RAS, K-RAS2A, | cg26129757 |
| | K-RAS2B, K-RAS4A, K-RAS4B | |
| L1CAM_P19_F | S10, HSAS, MASA, MIC5, SPG1, CAML1, CD171, | cg12024667 |
| | HSAS1, N-CAML1 | |
| LEFTY2_P561_F | EBAF, LEFTA, TGFB4, LEFTYA, MGC46222 | cg22462235 |
| LOX_P313_R | MGC105112 | cg08623535 |
| LY6G6E_P45_R | G6e, C6orf22 | cg26399860 |
| LYN_P241_F | JTK8 | cg04283851 |
| MAGEC3_E307_F | HCA2, MAGEC4, MAGE-C3, MGC119270, MGC119271 | cg02818322 |
| MAGEC3_P903_F | HCA2, MAGEC4, MAGE-C3, MGC119270, MGC119271 | cg22177388 |
| MAP3K1_E81_F | . | cg00468724 |
| MAP3K1_P7_F | . | cg06448700 |
| MAP3K8_P1036_F | COT, EST, ESTF, TPL2, Tpl-2, c-COT, FLJ10486 | cg21555918 |
| MAPK4_E273_R | ERK3, Erk4, PRKM4, p63MAPK | cg21612229 |
| MEST_E150_F | PEG1, MGC8703, MGC111102, DKFZp686L18234 | cg05241978 |
| MEST_P4_F | PEG1, MGC8703, MGC111102, DKFZp686L18234 | cg20632786 |
| MEST_P62_R | PEG1, MGC8703, MGC111102, DKFZp686L18234 | cg07409197 |
| MET_E333_F | HGFR, RCCP2 | cg24548568 |
| MMP7_E59_F | MMP-7, MPSL1, PUMP-1 | cg10521988 |
| MPO_P883_R | . | cg24997501 |
| MST1R_E42_R | RON, PTK8, CDw136 | cg03714052 |
| MUC1_E18_R | EMA, PEM, PUM, MAM6, PEMT, CD227, H23AG, mucin | cg00265953 |
| NBL1_E205_R | NB, DAN, NO3, DAND1, MGC8972, D1S1733E | cg21813747 |
| NBL1_P24_F | NB, DAN, NO3, DAND1, MGC8972, D1S1733E | cg04102045 |
| NOTCH4_E4_F | INT3, NOTCH3, MGC74442 | cg14700707 |
| OPCML_P71_F | OPCM, OBCAM | cg00738841 |
| PARP1_P610_R | PARP, PPOL, ADPRT, ADPRT1, PARP-1, pADPRT-1 | cg17303114 |
| PDGFRA_E125_F | CD140A, PDGFR2, MGC74795 | cg20629161 |
| PDGFRB_E195_R | JTK12, PDGFR, CD140B, PDGFR1, PDGF-R-beta | cg21817429 |
| PGR_P790_F | PR, NR3C3 | cg01987509 |
| PI3_P1394_R | ESI, WAP3, SKALP, WFDC14, MGC13613 | cg18675416 |
| PLAU_P176_R | ATF, UPA, URK, u-PA | cg26457761 |
| POMC_P400_R | MSH, POC, ACTH, CLIP | cg22632966 |
| PRSS1_E45_R | TRP1, TRY1, TRY4, TRYP1, MGC120175 | cg16567953 |
| PRSS1_P1249_R | TRP1, TRY1, TRY4, TRYP1, MGC120175 | cg09471643 |
| PRSS8_E134_R | CAP1, PROSTASIN | cg27436259 |
| PTHR1_P258_F | PTHR, MGC138426, MGC138452 | cg13804333 |
| PTK7_E317_F | CCK4 | cg21726633 |
| PTPN6_E171_R | HCP, HCPH, SHP1, SHP-1, HPTP1C, PTP-1C, | cg00788854 |
| | SHP-1L, SH-PTP1 | |
| PTPRO_P371_F | PTPU2, GLEPP1, PTP-U2 | cg25816184 |
| RARA_E128_R | RAR, NR1B1 | cg00848035 |
| RARA_P176_R | RAR, NR1B1 | cg10363722 |
| RARB_E114_F | HAP, RRB2, NR1B2 | cg14265392 |
| RARB_P60_F | HAP, RRB2, NR1B2 | cg06720425 |
| RARRES1_P426_R | TIG1 | cg13848998 |
| RARRES1_P57_R | TIG1 | cg12199224 |
| RBP1_P426_R | CRBP, RBPC, CRBP1, CRABP-I | cg11986962 |
| RIPK1_P744_R | RIP, FLJ39204 | cg24303123 |
| RIPK3_P124_F | RIP3, RIP3 beta, RIP3 gamma | cg13583230 |
| RUNX3_E27_R | AML2, CBFA3, PEBP2aC | cg21368948 |
| RUNX3_P247_F | AML2, CBFA3, PEBP2aC | cg10672665 |
| S100A2_P1186_F | CAN19, S100L, MGC111539 | cg21074565 |
| SEMA3A_P343_F | SemD, SEMA1, SEMAD, SEMAL, coll-1, Hsema-I, | cg16346212 |
| | SEMAIII, sema III | |
| SEMA3A_P658_R | SemD, SEMA1, SEMAD, SEMAL, coll-1, Hsema-I, | cg00927350 |
| | SEMAIII, sema III | |
| SEMA3B_E96_F | SemA, SEMA5, SEMAA, semaV, LUCA-1, FLJ34863 | cg25047248 |
| SEMA3B_P110_R | SemA, SEMA5, SEMAA, semaV, LUCA-1, FLJ34863 | cg12999941 |
| SERPINA5_P156_F | PCI, PAI3, PROCI, PLANH3 | cg13984563 |
| SERPINE1_E189_R | PAI, PAI1, PAI-1, PLANH1 | cg10678915 |
| SHB_P691_R | RP11-3J10.8 | cg19574087 |
| SNCG_E119_F | SR, BCSG1 | cg26738310 |
| SNCG_P53_F | SR, BCSG1 | cg12027410 |
| SNCG_P98_R | SR, BCSG1 | cg03677069 |
| SNURF_E256_R | . | cg07995992 |
| SPDEF_P6_R | PDEF, bA375E1.3, RP11-375E1_A.3 | cg10159596 |
| SPP1_E140_R | OPN, BNSP, BSPI, ETA-1, MGC110940 | cg20261167 |
| STAT5A_P704_R | MGF, STAT5 | cg09355539 |
| SYBL1_P349_F | VAMP7, VAMP-7, TI-VAMP | cg11419984 |
| TAL1_E122_F | SCL, TCL5, tal-1 | cg00875272 |
| TAL1_P594_F | SCL, TCL5, tal-1 | cg13537642 |
| TEK_E75_F | TIE2, VMCM, TIE-2, VMCM1, CD202B | cg05749772 |
| TFF2_P178_F | SP, SML1 | cg10018784 |
| TGFB2_E226_R | MGC116892, TGF-beta2 | cg20490551 |
| TGFB3_E58_R | FLJ16571, TGF-beta3 | cg17928876 |
| TGFBI_P173_F | CSD, CDB1, CDG2, CSD1, CSD2, CSD3, LCD1, | cg00833799 |
| | BIGH3, CDGG1 | |
| THBS2_P605_R | TSP2 | cg24654845 |
| THY1_P149_R | CD90 | cg18809507 |
| TNFRSF10A_P171_F | DR4, APO2, CD261, MGC9365, TRAILR1, TRAILR-1 | cg00990613 |
| TNFRSF10A_P91_F | DR4, APO2, CD261, MGC9365, TRAILR1, TRAILR-1 | cg25641272 |
| TNFRSF10C_E109_F | LIT, DCR1, TRID, CD263, TRAILR3 | cg05937208 |
| TNFRSF10C_P7_F | LIT, DCR1, TRID, CD263, TRAILR3 | cg23831143 |
| TNFRSF10D_E27_F | DCR2, CD264, TRUNDD, TRAILR4 | cg01031400 |
| TNFRSF10D_P70_F | DCR2, CD264, TRUNDD, TRAILR4 | cg04134048 |
| TNFSF10_E53_F | TL2, APO2L, CD253, TRAIL, Apo-2L | cg16555388 |
| TNFSF10_P2_R | TL2, APO2L, CD253, TRAIL, Apo-2L | cg27433414 |
| TN FSF8_E258_R | CD153, CD30L, CD30LG | cg09980061 |
| TNFSF8_P184_F | CD153, CD30L, CD30LG | cg19343707 |
| TNK1_P221_F | MGC46193 | cg26000767 |
| TRIM29_P261_F | ATDC | cg13907859 |
| TRIP6_P1090_F | OIP1, ZRP-1, MGC3837, MGC4423, MGC10556, | cg09357642 |
| | MGC10558, MGC29959 | |
| VAV1_E9_F | VAV | cg02621492 |
| WNT10B_P823_R | WNT-12 | cg23890019 |

### 6.13. Methylation Subgroup Analysis

Comparisons were also performed to show the relationship between several biological characteristics of the samples and the methylation profile. These methylation profiles may be used as a surrogate for measuring the biological characteristic, e.g., Breslow depth, when the location does not lend itself to such measurement, failure to annotate the sample, drug or treatment selection; selection of an appropriate combination of independent and additive conventional diagnostic markers to be used in conjunction with the methylation markers described in this application; or other reasons.

Specifically, Table 10 lists CpG methylation sites associated with Breslow depth. In addition, analysis to study mitotic rate (Table 11) and ulceration were performed. For ulceration, one methylation correlated significantly, ProbelD MAP3K1_P7_F with a p value of 0.00096. The results for Breslow depth, mitotic rate, and mutations are shown below.

TABLE 10
CpG Methylation sites associated with Breslow depth

| ProbeID | p.value.Breslow | q.value.Breslow | coef.Breslow | mean.beta.adjusted |
|---|---|---|---|---|
| ABCB4_E429_F | 0.000151351 | 0.055067493 | 0.075844142 | 0.951470796 |
| GNG7_E310_R | 0.000360298 | 0.101027535 | 0.064175387 | 0.963251731 |
| HOXA9_E252_R | 0.000587998 | 0.137395588 | 0.289499517 | 0.706184222 |
| HOXA9_P303_F | 4.68E−05 | 0.055067493 | 0.281051363 | 0.373700815 |
| IRAK3_P185_F | 0.000157111 | 0.055067493 | 0.251012726 | 0.373687534 |
| PTK7_E317_F | 0.000114644 | 0.055067493 | 0.16729955 | 0.341920543 |
| RUNX1T1_E145_R | 0.000767285 | 0.153676131 | 0.217039165 | 0.415388499 |

TABLE 11
CpG Methylation sites associated with mitotic rate (MitRate)

| ProbeID | p.value.MitRate | q.value.MitRate | coef.MitRate | mean.beta |
|---|---|---|---|---|
| USP29_P282_R | 0.000859842 | 0.674750416 | 0.102118216 | 0.870617418 |
| SHH_P104_R | 0.006076261 | 0.674750416 | 0.070286692 | 0.095698924 |
| SEMA3A_P658_R | 0.013545534 | 0.702374503 | 0.144593294 | 0.462172928 |
| MMP14_P208_R | 0.017198003 | 0.705774866 | 0.094456225 | 0.180542789 |
| UGT1A7_P751_R | 0.017395592 | 0.705774866 | 0.060682502 | 0.952848615 |
| AATK_P709_R | 0.038655609 | 0.74311075 | 0.091648145 | 0.718552347 |

TABLE 12
Analysis of CpG sites associated with mutation (Mut) for any mutation in BRAF
codon 15, and NRAS codon 61. Nearly all of the mutation samples had mutations at BRAF
V600. Thus, the sites below may be useful to select specific patients for therapy that are
likely to respond because of the presence of BRAF mutations.

| ProbeID | p.value.Mut.Uni | q.value.Mut.Uni | p.value.Mut.Multi | q.value.Mut.Multi |
|---|---|---|---|---|
| CCR5_P630_R | 0.05511171 | 0.492144064 | 0.000689708 | 0.16766822 |
| CD40_E58_R | 0.001758825 | 0.273985791 | 0.000717553 | 0.16766822 |
| DNMT3B_P352_R | 0.001006177 | 0.230434732 | 0.000641606 | 0.16766822 |
| GPX1_P194_F | 0.001592276 | 0.273985791 | 0.000201973 | 0.16766822 |
| KLK10_P268_R | 6.22E−05 | 0.0872197 | 0.000400032 | 0.16766822 |
| P2RX7_E323_R | 0.001008864 | 0.230434732 | 0.000663112 | 0.16766822 |
| SEMA3B_P110_R | 0.001328043 | 0.267401202 | 0.000895024 | 0.240377935 |

TABLE 13
shows the accession numbers; specific single CpG coordinate; presence or absence
of CpG islands; specific sequences used in the Illumina GoldenGate array experiments; and
the synonyms for genes hypermethylated or hypomethylated in the subset analysis. All gene
IDs and accession numbers are from Ref. Seq. version 36.1.

| Probe_ID | Gid | Accession | Gene_ID | CHRM | CpG_Coor | Dist_to_TSS | CpG_i |
|---|---|---|---|---|---|---|---|
| AATK_P709_R | 89041906 | XM_927215.1 | 9625 | 17 | 76710603 | −709 | Y |
| ABCB4_E429_F | 9961251 | NM_018850.1 | 5244 | 7 | 86947255 | 429 | N |
| CD40_E58_R | 23312370 | NM_152854.1 | 958 | 20 | 44180371 | 58 | Y |
| DNMT3B_P352_R | 28559060 | NM_175848.1 | 1789 | 20 | 30813500 | −352 | N |
| GNG7_E310_R | 32698768 | NM_052847.1 | 2788 | 19 | 2603280 | 310 | Y |
| HOXA9_E252_R | 24497558 | NM_002142.3 | 3205 | 7 | 27171422 | 252 | Y |
| HOXA9_P303_F | 24497558 | NM_002142.3 | 3205 | 7 | 27171977 | −303 | Y |
| IRAK3_P185_F | 6005791 | NM_007199.1 | 11213 | 12 | 64869099 | −185 | Y |
| KLK10_P268_R | 22208981 | NM_002776.3 | 5655 | 19 | 56215362 | −268 | N |
| MAP3K1_P7_F | 88983555 | XM_042066.10 | 4214 | 5 | 56146015 | −7 | Y |
| MMP14_P208_R | 13027797 | NM_004995.2 | 4323 | 14 | 22375425 | −208 | N |
| PTK7_E317_F | 27886610 | NM_002821.3 | 5754 | 6 | 43152324 | 317 | Y |
| RUNX1T1_E145_R | 28329418 | NM_175635.1 | 862 | 8 | 93176474 | 145 | N |
| SEMA3A_P658_R | 5174672 | NM_006080.1 | 10371 | 7 | 83662506 | −658 | N |
| SEMA3B_P110_R | 54607087 | NM_004636.2 | 7869 | 3 | 50279934 | −110 | N |
| SHH_P104_R | 21071042 | NM_000193.2 | 6469 | 7 | 1.55E+08 | −104 | Y |
| UGT1A7_P751_R | 41282212 | NM_019077.2 | 54577 | 2 | 2.34E+08 | −751 | N |
| USP29_P282_R | 56790915 | NM_020903.2 | 57663 | 19 | 62323039 | −282 | Y |

| | SEQ | |
|---|---|---|
| Probe_ID | ID | Input_Sequence |
| AATK_P709_R | 266 | ACGGGTGGCCCGTGGCCCAGCAG[CG]GCTCCATGGCCAGCGAGGCGG |
| ABCB4_E429_F | 267 | TTCCTTGGACTTCTCAGTCTATTCT[CG]CCACTTCTGTCATGTCAGTCAGTCACAC |
| CD40_E58_R | 268 | CGGGCGCCCAGTGGTCCTGC[CG]CCTGGTCTCACCTCGCTATGGTTCGTCTGC |
| DNMT3B_P352_R | 269 | CTGCCCTCTCTGAGCCCC[CG]CCTCCAGGCCTGTGTGTGTGTCTCCGTTCG |
| GNG7_E310_R | 270 | AGGCCAGACGCTGAGAGAGAAAAACACTG[CG]TAATCCCACGTATTGTGGAGTCCAAAA |
| HOXA9_E252_R | 271 | TGGGTTCCACGAGGCGCCAAACACCGT[CG]CCTTGGACTGGAAGCTGCACG |
| HOXA9_P303_F | 272 | CCCCATACACACACTTCTTAAG[CG]GACTATTTTATATCACAATTAATCACGCCA |
| IRAK3_P185_F | 273 | CCCCACCGCAGAGGTGTGAAGGGG[CG]CAAAGCCAGCGAAGGGAGAACCCG |
| KLK10_P268_R | 274 | AACAGAAACAAGGAAAAAGGGAAACCCA[CG]CCCACTCTGTGGCCGTGAGTGA |
| MAP3K1_P7_F | 275 | GTAGAGTCCAGGGACTAGGAGGACTCACAA[CG]CAGCGATGGGCAGCCAGGCCCTG |
| MMP14_P208_R | 276 | CTACAGCCCCCTGCTGTCCAT[CG]CGGCCTCAACCCCTGCAGATGGCA |
| PTK7_E317_F | 277 | GGGGGCACAGAGCTTGGGAAGCG[CG]GGAGTCCCGTGGGCAAAAG |
| RUNX1T1_E145_R | 278 | GGATAGCAGAGGTGATGGGAGATAG[CG]TCAAGGCCAGGGGTAGATGCCTC |
| SEMA3A_P658_R | 279 | GAGATTAGAGCCGGGAGCAGAACCCTCAGG[CG]TGCCTGTGAAAGGCATGTAGCTATAA |
| SEMA3B_P110_R | 280 | CTTGTGCCCATTCCACTCC[CG]CCTGGCTGCCGTCTCCAGCTGGTCCC |
| SHH_P104_R | 281 | ATGGCAGGCTGCCGGCCGCTGATAA[CG]GAACACATCGGAGTTGGGTCG |
| UGT1A7_P751_R | 282 | CGCTAAGACCCTTGCTCTCTTTC[CG]TCGAACATGAGATGCCAATTTCTTTCTGGG |
| USP29_P282_R | 283 | TTTCTCTGAACCCTAACTCCTGC[CG]TTACGCCCCACCAGCTCTAGGCC |

| Probe_ID | Synonym | cg_no |
|---|---|---|
| AATK_P709_R | . | cg02979355 |
| ABCB4_E429_F | MDR3, PGY3, ABC21, MDR2/3, PFIC-3 | cg05279864 |
| CD40_E58_R | p50, Bp50, CDW40, MGC9013, TNFRSF5 | cg20698532 |
| DNMT3B_P352_R | ICF, M.HsaIIIB | cg14703690 |
| GNG7_E310_R | FLJ00058 | cg13502721 |
| HOXA9_E252_R | HOX1, ABD-B, HOX1G, HOX1.7, MGC1934 | cg10604830 |
| HOXA9_P303_F | HOX1, ABD-B, HOX1G, HOX1.7, MGC1934 | cg03715906 |
| IRAK3_P185_F | IRAK-M | cg24003063 |
| KLK10_P268_R | NES1, PRSSL1 | cg06130787 |
| MAP3K1_P7_F | . | cg06448700 |
| MMP14_P208_R | MMP-X1, MTMMP1, MT1-MMP | cg01508380 |
| PTK7_E317_F | CCK4 | cg21726633 |
| RUNX1T1_E145_R | CDR, ETO, MTG8, MTG8b, AML1T1, ZMYND2, CBFA2T1, MGC2796 | cg07538339 |
| SEMA3A_P658_R | SemD, SEMA1, SEMAD, SEMAL, coll-1, Hsema-I, SEMAIII, sema III | cg00927350 |
| SEMA3B_P110_R | SemA, SEMA5, SEMAA, semaV, LUCA-1, FLJ34863 | cg12999941 |
| SHH_P104_R | HHG1, HLP3, HPE3, SMMCI | cg06981396 |
| UGT1A7_P751_R | UDPGT, UGT1G, UGT1*7 | cg16671505 |
| USP29_P282_R | HOM-TES-84/86 | cg16675193 |

### 6.14. Methylation Specific PCR Examples

Sodium bisulfite modification and methylation-specific PCR (Method A): Digested DNA (500 ng) is denatured in 0.3 N NaOH at 37° C. for 15 min (Clark et al., 1994, Nucleic Acids Res. 22, 2990-2997). Then, 3.6 N sodium bisulfite (pH 5.0) and 0.6 mM hydroquinone are added, and the sample undergoes 15 cycles of 1) denaturation at 95° C. for 30 s and 2) incubation at 50° C. for 15 min. The sample is desalted with the Wizard DNA Clean-Up system (Promega, Madison, Wis.), and desulfonated in 0.3 N NaOH. DNA was ethanol-precipitated and dissolved in 20 μl of buffer. Methylation-specific PCR (MSP) is performed with a primer set specific to the methylated or unmethylated sequence (M or U set), using 0.5 μl of the sodium-bisulfite-treated DNA (Herman et al., 1996, Proc. Natl. Acad. Sci. USA, 93, 9821-9826). Primers and probes are designed based on the sequences shown in Table 4. the Zymo Universal Methylated DNA Standard is used as the positive, fully-methylated control, and a GenomePlex (Sigma) whole genome amplified (WGA) DNA is used as the negative, unmethylated control.

Sodium Bisulfite DNA Treatment (Method B)

DNA is sodium bisulfite treated using the EZ DNA Methylation-Gold Kit (Zymo Research, cat. #D5005). The DNA sample (˜10-20 ul lysate or 200-500 ng DNA) is mixed with 130 ul of CT Conversion Reagent in a PCR tube and denatured in a thermal cycler at 98° C. for 10 minutes, sodium bisulfite modified at 64° C. for 2.5 hours, and stored at 4° C. for up to 20 hours. The sample is then mixed with 600 ul M-binding buffer and spun through the Zymo-Spin IC column for 30 seconds (>=10,000×g). The column is washed with 100 ul of M-Wash buffer, spun, and incubated in 200 ul of M-Desulphonation buffer for 15-20 minutes. The column is spun for 30 seconds (>=10,000×g), washed twice with 200 ul M-Wash buffer and spun at top speed. Then the sample is eluted from the column with 10 M-Elution buffer and stored in the freezer (−20° C.) prior to use in methylation assays.

Quantitative Real-Time RT-PCR (Method a)

After treatment with DNase I (Invitrogen, Carlsbad, Calif.), cDNA is synthesized from 3 mg of total RNA using Superscript II (Invitrogen). Real-time PCR is performed using SYBR Green PCR Core Reagents (PE Applied Biosystems, Foster City, Calif.) and an iCycler Thermal Cycler (Bio-Rad Laboratories, Hercules, Calif.). Quantitative RT-PCR is also performed using TaqMan probes and instrumentation (Applied Biosystems, Carlsbad, Calif.). The number of molecules of a specific cDNA in a sample is measured by comparing its amplification with that of standard samples containing 10¹ to 10⁶ molecules. The expression levels in each sample are obtained by normalizing the number of its cDNA molecules with that of the GAPDH, actin, or other housekeeping genes.

Methylation-Specific Quantitative PCR (MS-QPCR)

Sodium-bisulfate modified DNA is PCR amplified in a final volume of 20 uL PCR buffer containing 10 mM Tris-HCl (pH8.3), 50 mM KCl, 2.5-4.5 mM MgCl₂, 150-250 nM dNTPs, 0.2-0.4 uM primers, and 0.5 Units of AmpliTaq Gold polymerase (ABI) for an initial denaturation at 95° C. for 10 minutes followed by 45 cycles at 95° C.-15 s, 55-66° C.-30 s, 72° C.-30 s, and a final extension at 72° C. for 7 minutes. Controls used to quantify methylation values include serially diluted methylated/unmethylated DNAs (Zymo) from 100% methylated to 0% methylated for each gene/CpG of interest, no-template control, reference gene (beta-actin) and standard curve of DNA quantity. Reactions are run using SYBR green (Roche) or methylation specific fluorescently labeled probes (ABI) on the ABI 7900HT Fast instrument with software to calculate standard curves and Ct values. Multiplex PCR can be evaluated in the same well for comparison when using fluorescently labeled methylated (FAM) and unmethylated (VIC) TaqMan (ABI) probes using the ABI 7900HT Fast instrument.

TABLE 14
Target CpG Islands and Primers for Methylation Specific QPCR Primers (SEQ ID
Nos. 285-311 (sense), SEQ ID Nos. 312-339 (antisense))

| Target ID | Sense (5′-3′) | Antisense (5′-3′) |
|---|---|---|
| CD40_E58_R(M) | GGGGTAGGGGAGTTAGTAGAGGTTTC | CACTACAAAAACAAACGAACCATAACG |
| CD40_E58_R(U) | GGGGTAGGGGAGTTAGTAGAGGTTTT | CACTACAAAAACAAACAAACCATAACAA |
| COL1A2_E299_F(M) | TAAGAAGTTAGTTTCGTGGTTACGT | ACCCGAATCTACCCTATTTATACGAC |
| COL1A2_E299_F(U) | TAAGAAGTTAGTTTTGTGGTTATGT | ACCCAAATCTACCCTATTTATACAAC |
| DNMT3B_P352_R(M) | GGGGTTTTGTTTTTTTTGAGTTTTC | ACTCCTTCTAAAACCTTTTTCCCGA |
| DNMT3B_P352_R(U) | GGGGTTTTGTTTTTTTTGAGTTTTT | ACTCCTTCTAAAACCTTTTTCCCAA |
| EMR3_P39_R(M) | ATGTAATTTTTAGGGTATTTTTTCG | CGTCAAACTCATAATTCTACTTTTCGT |
| EMR3_P39_R(U) | ATGTAATTTTTAGGGTATTTTTTTG | CATCAAACTCATAATTCTACTTTTCAT |
| FRZB_P406_F(M) | ATTTTATTTTCGGGAAGAGTAGTCG | AAAAACCCCGCAAAACGT |
| FRZB_P406_F(U) | ATTTTATTTTTGGGAAGAGTAGTTG | AAAAAACCCCACAAAAACAT |
| GSTM2_P109_R(M) | TTCGTTTTGGGTTTTTGGGC | AAAAAAACCTTACTACGACCCCGC |
| GSTM2_P109_R(U) | TTTTTTGTTTTGGGTTTTTGGGTG | AAAAAAAACCTTACTACAACCCCAC |
| HOXA9_E252_R(M) | TGTAGTTTTTAGTTTAAGGCGACGG | AAACGCATATACCTACCGTCCGA |
| HOXA9_E252_R(U) | TGTAGTTTTTAGTTTAAGGTGATGG | ACCAAAAACACATATACCTACCATCCAA |
| HOXA9_P303_F(M) | GGGTTTCGTTGGTCGTATTC | CCATATATTTTTATATAAAAAAATCGTA |
| HOXA9_P303_F(U) | AGGGGTTTTGTTGGTTGTATTT | AAACCATATATTTTTATATAAAAAAATCAT |
| ITK_E166_R(M) | TTTTTTTTCGAATTTTAAAGTTCG | AAACTACTCACATACCCCATAACGA |
| ITK_E166_R(U) | TTTTTTTTGAATTTTAAAGTTTG | AAACTACTCACATACCCCATAACAA |
| KCNK4_E3_F(M) | GGGTTTGGGAGATGTTAGATTAGC | ACCAACCTTCTAACCTTAAACCGAA |
| KCNK4_E3_F(U) | GGTTTGGGAGATGTTAGATTAGTGT | ACCAACCTTCTAACCTTAAACCAAA |
| MT1A_E13_R(M) | GGGTTTTATTAAGTTTTTTACGTGCG | AAATCCATTTCGAACCGCGA |
| MT1A_E13_R(U) | TGGGTTTTATTAAGTTTTTTATGTGTG | TTAAAATCCATTTCAAACCACAA |
| PRSS8_E134_R(M) | GCGGAGTTTAGTTAGTGGGC | AAAACTAACCTCTAAAACAAAAAACGA |
| PRSS8_E134_R(U) | TGGTGGAGTTTAGTTAGTGGGTG | CAAAACTAACCTCTAAAACAAAAAACAA |
| RUNX3_E27_R(M) | GAGTTTTTTTATTTTGGTTGTCGA | TATACCCAAAAATTTAAATTCCCG |
| RUNX3_E27_R(U) | GGAGTTTTTTTATTTTGGTTGTTGA | ATACCCAAAAATTTAAATTCCCAAT |
| TNFSF8_E258_R(M) | TAGGGTTGTAGTAAGTATTTAACGG | CAACACCATAATAATAACCACCGTA |
| TNFSF8_E258_R(U) | ATGGATTTAGGGTTGTAGTAAGTATTTAAT | CAACACCATAATAATAACCACCATA |

TABLE 15
Target CpG Islands and Primers for Bisulfite sequencing or MS-HRM and
Reference Primers (SEQ ID Nos. 340-346 (sense), SEQ ID Nos. 347-353 (antisense).

| Target ID | Sense (5′-3′) | Antisense (5′-3′) |
|---|---|---|
| ITK_P114_F | TGAGTTTATAGTTTTTTAAATATTATTTTA | TACTCAAAAACAACTTACCTTCAAC |
| ITK_E166_R | TGTGTTAAGAGGTGATGTTTAAGGT | AACAAATAAAACTACTCACATACCCC |
| ITK_E166_R | ATTAAGAAATTTTAATAAAAGAGAA | TAAAACTACTCACATACCCCATAAC |
| KIT_P405F-P367R | TTTATTGTTTGGGGAGTATTTGGTAGGT | CCACCTTTCCACCCCTAAAATATAAAC |
| KLK10_P268_R | GGAGATTGTAATAAATTAAGGTTAAAAGAG | TAAAACACACACAAAACTCACTCAC |
| MPO_P883_R | TTATTAGAAGTTAAGAAGAAAGGGGAGTG | ATACATCCAACAACCACCCAATAAAC |
| Beta Actin | TGGTGATGGAGGAGGTTTAGTAAGT | AACCAATAAAACCTACTCCTCCCTTAA |

TABLE 16
lists CpG islands for either MS−QPCR
or bisulfate sequencing.
Target ID
CD40_E58_R
COL1A2_E299_F
DNMT3B_P352_R
EMR3_P39_R
FRZB_P406_F
GSTM2_P109_R
HOXA9_E252_R
HOXA9_P303_F
ITK_E166_R
ITK_P114_F
KCNK4_E3_F
KIT_P367_R
KIT_P405_F
KLK10_P268_R
MPO_P883_R
MT1A_E13_R
PRSS8_E134_R
RUNX3_P247_F
RUNX3_E27_R
TNFSF8_E258_R

### 6.15. Dysplastic Nevi vs. Benign Moles

Patients and Tissues

Because dermatologists have difficulty distinguishing between benign moles and dysplastic nevi, an analysis was undertaken to find methylation markers for normal skin. Using the methods described above, profiling was performed on FFPE samples for dysplastic nevi (N=22) and benign non-dysplastic moles (N=34). The results are show below in Table 17.

TABLE 17

| | | | Non−Dyplastic | | Mean |
|---|---|---|---|---|---|
| Target ID | Raw_p | Bonf_p | Mean β | Dyplastic Mean β | Δβ |
| ALPL_P433_F | 1.05E−05 | 0.01523 | 0.346 | 0.651 | −0.305 |
| BCL6_P248_R | 9.53E−06 | 0.01383 | 0.161 | 0.374 | −0.213 |
| BDNF_E19_R | 7.20E−06 | 0.01045 | 0.266 | 0.527 | −0.261 |
| BDNF_P259_R | 4.23E−06 | 0.00614 | 0.324 | 0.548 | −0.224 |
| CD9_P585_R | 7.04E−06 | 0.01021 | 0.225 | 0.440 | −0.215 |
| CEACAM1_P44_R | 2.53E−06 | 0.00367 | 0.375 | 0.652 | −0.277 |
| CSPG2_P82_R | 7.04E−06 | 0.01021 | 0.210 | 0.470 | −0.259 |
| CTSD_P726_F | 5.24E−06 | 0.00761 | 0.420 | 0.678 | −0.258 |
| EFNB3_E17_R | 4.47E−06 | 0.00648 | 0.388 | 0.605 | −0.217 |
| EPHA2_P203_F | 2.64E−05 | 0.03830 | 0.238 | 0.483 | −0.245 |
| ERN1_P809_R | 3.23E−06 | 0.00469 | 0.227 | 0.451 | −0.224 |
| ETV1_P515_F | 1.41E−06 | 0.00205 | 0.142 | 0.358 | −0.216 |
| FANCE_P356_R | 2.33E−06 | 0.00338 | 0.311 | 0.556 | −0.244 |
| FGF2_P229_F | 1.71E−06 | 0.00248 | 0.326 | 0.588 | −0.261 |
| FGF9_P862_R | 3.23E−06 | 0.00469 | 0.317 | 0.534 | −0.217 |
| GAS7_P622_R | 2.17E−06 | 0.00315 | 0.332 | 0.647 | −0.315 |
| GDF10_E39_F | 7.79E−06 | 0.01130 | 0.208 | 0.457 | −0.249 |
| GFI1_E136_F | 2.45E−05 | 0.03556 | 0.186 | 0.406 | −0.221 |
| HDAC9_P137_R | 7.14E−07 | 0.00104 | 0.126 | 0.352 | −0.226 |
| HLA-DQA2_E93_F | 1.24E−05 | 0.01800 | 0.665 | 0.887 | −0.222 |
| HLA-DRA_P132_R | 4.98E−06 | 0.00723 | 0.239 | 0.506 | −0.266 |
| HTR2A_P853_F | 1.97E−06 | 0.00286 | 0.112 | 0.363 | −0.250 |
| IGF2AS_P203_F | 2.36E−05 | 0.03422 | 0.275 | 0.524 | −0.250 |
| IGFBP6_E47_F | 1.97E−06 | 0.00286 | 0.366 | 0.615 | −0.249 |
| IL16_P93_R | 1.96E−05 | 0.02841 | 0.446 | 0.716 | −0.270 |
| IPF1_P234_F | 5.68E−06 | 0.00824 | 0.447 | 0.658 | −0.211 |
| IPF1_P750_F | 1.15E−05 | 0.01667 | 0.416 | 0.660 | −0.244 |
| JUNB_P1149_R | 2.98E−06 | 0.00432 | 0.147 | 0.360 | −0.213 |
| KCNK4_E3_F | 3.80E−06 | 0.00552 | 0.144 | 0.358 | −0.215 |
| MAP3K8_P1036_F | 1.68E−05 | 0.02443 | 0.330 | 0.586 | −0.256 |
| MMP14_P13_F | 2.64E−05 | 0.03830 | 0.199 | 0.473 | −0.274 |
| MT1A_E13_R | 1.41E−06 | 0.00205 | 0.217 | 0.425 | −0.208 |
| NEU1_P745_F | 2.12E−05 | 0.03073 | 0.160 | 0.369 | −0.208 |
| NFKB1_P496_F | 2.71E−06 | 0.00393 | 0.294 | 0.604 | −0.310 |
| NGFB_P13_F | 2.14E−06 | 0.00311 | 0.172 | 0.438 | −0.266 |
| ONECUT2_E96_F | 2.92E−07 | 0.00042 | 0.131 | 0.339 | −0.209 |
| PCTK1_E77_R | 1.30E−06 | 0.00188 | 0.536 | 0.737 | −0.201 |
| PI3_P1394_R | 4.31E−06 | 0.00626 | 0.569 | 0.784 | −0.215 |
| PYCARD_P150_F | 1.19E−06 | 0.00173 | 0.347 | 0.709 | −0.362 |
| RET_seq_54_S260_F | 1.68E−05 | 0.02443 | 0.147 | 0.420 | −0.273 |
| RIPK1_P744_R | 1.34E−05 | 0.01944 | 0.633 | 0.836 | −0.202 |
| S100A4_E315_F | 6.01E−07 | 0.00087 | 0.141 | 0.351 | −0.210 |
| SEPT9_P374_F | 4.23E−07 | 0.00061 | 0.096 | 0.314 | −0.219 |
| TBX1_P885_R | 3.51E−06 | 0.00509 | 0.147 | 0.356 | −0.208 |
| TFF2_P178_F | 6.01E−07 | 0.00087 | 0.540 | 0.816 | −0.275 |
| TRIP6_P1090_F | 2.84E−05 | 0.04124 | 0.171 | 0.384 | −0.213 |
| VAV1_E9_F | 1.96E−05 | 0.02841 | 0.379 | 0.612 | −0.233 |

### 6.16. ITK Staining Experiments

Immunofluorescence Staining for ITK (IL-2 Inducible T-Cell Kinase).

Melanoma cell lines and cultured melanocytes were investigated for the presence of ITK protein using immunohistochemistry (IHC) with an antibody specific for ITK. Approximately fifty percent of 40 melanoma cell lines showed observable staining for ITK while no ITK staining was observed in the cultured primary melanocytes. IHC was also performed on primary melanoma tissue sections from patients.

In the primary tissue sections, the melanoma stained pink for ITK, while the surrounding normal skin does not stain for ITK. No other ITK staining was detected in the surrounding tissue and ITK staining was not detected in the normal melanocytes. Specifically, the section was stained with an antibody to ITK (abcam; 1:3000) with tyramide Cy5 amplification to visualize ITK (pink color). The specimen was also stained with the blue fluorescent stain DAPI (4′,6-diamidino-2-phenylindole) that binds strongly to A-T rich regions in DNA. A few ITK stained cells were seen at the dermal—epidermal junction extending out from the periphery of the tumor, likely representing migrating melanoma cells. These melanoma cells stained strongly for ITK, and the ITK-staining cells at the dermal-epidermal junction decrease in number as the distance increases from the melanoma. These were likely migrating melanoma cells and this information could be used for margin control at the time of surgery.

One of current markers for margin control, used primarily when melanomas are removed by MOHs surgery, is MART1 IHC staining. Alternatively, surgeons remove tissue based on an arbitrary distance from the tumor. MART1 is also expressed normal melanocytes so MART1 IHC staining shows the density and distribution of the melanocytes as an indicator of a clear margin. However, ITK IHC staining is present and then abruptly becomes absent at the edge of the tumor. ITK shows melanoma cells migrating along the basement membrane out from the tumor must be removed. ITK staining looks like it could be a better measure of clear margins.

Dual Fluorescent Immunohistochemistry (IF) and AQUA

Additionally, the ITK levels for three other melanomas and three nevi were studied quantitatively using Dual Fluorescent Immunohistochemistry and Automated Quantitative Analysis (AQUA) technology. Only melanocytic cells were quantitated using an 5100 mask that defines the melanocytic region. To measure ITK levels in melanoma cells (defined by 5100 staining) the consecutive dual fluorescent IHC was carried out in Bond Autostainer (Leica Microsystems Inc., Norwell Mass.). Slides were deparaffinized in Bond dewax solution (AR9222) and hydrated in Bond wash solution (AR9590). Antigen retrieval for ITK and S100 was performed for 30 min at 1000C in Bond-epitope retrieval solution 2 pH9.0 (AR9640). After pretreatment, slides were first incubated with ITK antibody (1:3000) followed with Bond polymer (DS9800); The tyramide Cy5 amplification was used to visualize ITK (PerkinElmer, Boston, Mass.). After completion of ITK staining the 5100 antibody (Abcam 1:3200) was applied, which was detected with A1exa555 labeled goat anti rabbit secondary antibody (Invitrogen, Carlsbad, Calif.). The stained slides were mounted with ProLong Gold antifade reagent (Molecular Probes, Inc. Eugene, Oreg.) containing 4′,6-diamidino-2-phenylindole (DAPI) to define nuclei. All appropriate quality control stains (single and double) were carried out to make sure that there is no cross-reactivity between the antibodies.

Digitization of Slides and AQUA

H&E stained whole tissue sections were digitally imaged (20× objective) using the Aperio ScanScope XT (Aperio Technologies, Vista, Calif.).

Aperio FL/AQUA Image Analysis

Aperio FL (Aperio Inc) with integrated HistoRx AQUA technology (HistoRx, New Haven, Conn.) was used to scan the whole slides at ×20 objective through DAP1, CY3 and CY5 channels to identify nuclei, 5100 (mask) and ITK (target proteins) respectively. In whole tissue sections the 5100 positive areas within the tumor were annotated for each slide manually using positive pan tool; out of the focus or folded tissue areas were marked by negative pan to exclude from analysis. Annotated layers for each slide were submitted for analysis through spectrum software (Aperio Inc.) using AQUA clustering algorithm according to AQUAnalysis™ user guide: Aperio Edition (Rev. 1.0, CDN0044, HistoRx, New Haven, Conn.). Generated AQUA analysis data (summary of the AQUA scores and compartment masking produced by AQUA) was pushed back to spectrum and exported as csv file.

PM2000/AQUA Image Analysis

To validate AQUA scores obtained through Aperio FL, the high resolution acquisition was performed in PM2000 (HistoRx) as well. The same areas, analyzed in Aperio-FL were acquired in PM2000 for scoring the ITK expression in 5100 mask. The marked images were analyzed by AQUA® software version #2.2 using HistoRx AQUA clustering algorithm. Analysis profile and merged images were generated for each slide. Spots, which didn't pass the validation, were excluded from analysis.

The results (Table 18) demonstrated that ITK is observable in the melanomas and lower in the nevi (moles), as denoted by the Aqua Score that measures expression within the melanocytic region and excludes keratinocyte, fibroblast and other non-melanocytic cell staining. Further staining of normal skin section showed no significant ITK expression in melanocytes within the normal skin.

TABLE 18

| | Aperio FL Average of Target |
|---|---|
| Sample | in Tumor Mask AQUA score |
| Melanoma 1 | 418 |
| Melanoma 2 | 262 |
| Melanoma 3 | 268 |
| Melanoma 4 | 325 |
| Nevus (mole) 1 | 147 |
| Nevus (mole) 2 | 191 |
| Nevus (mole) 3 | 34 |
| Normal skin (melanocytes) | 4 |

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

What is claimed is:

1. A method for detecting melanoma in a tissue sample which comprises:
(a) measuring a level of methylation of one or more regulatory elements differentially methylated in melanoma and benign nevi; and
(b) determining whether melanoma is present or absent in the tissue sample.

2. The method of claim 1, wherein the level of methylation is measured at single CpG site resolution.

3. The method of claim 1, wherein the tissue sample is a common nevi sample.

4. The method of claim 1, wherein the tissue sample is a dysplastic nevi sample.

5. The method of claim 1, wherein the tissue sample is a benign atypical nevi sample.

6. The method of claim 1, wherein the tissue sample is a melanocytic lesion of unknown potential.

7. The method of claim 1, wherein the tissue sample is a formalin-fixed, paraffin-embedded sample.

8. The method of claim 1, wherein the tissue sample is a fresh-frozen sample.

9. The method of claim 1, wherein the tissue sample is a fresh tissue sample.

10. The method of claim 1, wherein the tissue sample is a dissected tissue, an excision biopsy, a needle biopsy, a punch biopsy, a shave biopsy, a strip biopsy, or a skin biopsy sample.

11. The method of claim 1, wherein the tissue sample is a lymph node biopsy sample.

12. The method of claim 1, wherein the lymph node biopsy sample is a sentinel lymph node sample.

13. The method of claim 1, wherein the tissue sample is a sample from a cancer metastasis.

14. The method of claim 1, wherein the regulatory elements are regulatory elements associated with immune response/inflammatory pathway genes, hormonal regulation genes, or cell growth/cell adhesion/apoptosis genes.

15. The method of claim 1, wherein the regulatory elements are regulatory elements associated with a gene encoding CARD15, CCL3, CD2, EMR3, EVI2A, FRZB, GSTM2, HLA-DPA1, IFNG, ITK, KCNK4, KLK10, LAT, MPO, NPR2, OSM, PSCA, PTHLH, PTHR1, RUNX3, TNFSF8 or TRIP6.

16. The method of claim 15, wherein hypermethylation of the regulatory elements associated with a gene encoding FRZB, GSTM2, KCNK4, NPR2, or TRIP6 is indicative of melanoma.

17. The method of claim 15, wherein hypomethylation of the regulatory elements associated with a gene encoding CARD15, CCL3, CD2, EMR3, EVI2A, HLA-DPA1, IFNG, ITK, KLK10, LAT, MPO, OSM, PSCA, PTHLH, PTHR1, RUNX3 or TNFSF8 is indicative of melanoma.

18. The method of claim 1, wherein the level of methylation is measured using a bisulfate conversion-based microarray assay.

19. The method of claim 1, wherein the level of methylation is measured using a differential hybridization assay.

20. The method of claim 1, wherein the level of methylation is measured using a methylated DNA immunoprecipitation based assay.

21. The method of claim 1, wherein the level of methylation is measured using a methylated CpG island recovery assay.

22. The method of claim 1, wherein the level of methylation is measured using a methylation specific polymerase chain reaction assay.

23. The method of claim 1, wherein the level of methylation is measured using a methylation sensitive high resolution melting assay.

24. The method of claim 1, wherein the level of methylation is measured using a microarray assay.

25. The method of claim 1, wherein the level of methylation is measured using a pyrosequencing assay.

26. The method of claim 1, wherein the level of methylation is measured using an invasive cleavage amplification assay.

27. The method of claim 1, wherein the level of methylation is measured using a sequencing by ligation based assay.

28. The method of claim 1, wherein the level of methylation is measured using a mass spectrometry assay.

29. The method of claim 1, further comprising evaluating the quality of the sample by measuring the levels of skin specific markers.

30. The method of claim 29, wherein the skin specific markers are measured by antibody staining, differential methylation, expression analysis, or fluorescence in situ hybridization (FISH).

31. The method of claim 1, further comprising staining the tissue sample with one or more antibodies.

32. The method of claim 31, wherein the antibodies are 5100, gp100 (HMB-45 antibody), MART-1/Melan-A, MITF, or tyrosinase antibodies.

33. The method of claim 32, wherein the antibodies are a cocktail of gp100 (HMB-45 antibody), MART-1/Melan-A, and tyrosinase antibodies.

34. The method of claim 1, further comprising fluorescence in situ hybridization (FISH), comparative genomic hybridization (CGH), or gene expression analysis.

35. The method of claim 1, wherein the regulatory element differentially methylated has a sensitivity analysis area under the curve of greater than 0.70.

36. The method of claim 1, wherein the regulatory element differentially methylated has a sensitivity analysis area under the curve of greater than 0.85.

37. The method of claim 1, wherein the regulatory element differentially methylated has a sensitivity analysis area under the curve of greater than 0.98.

38. The method of claim 1, wherein a plurality of regulatory elements differentially methylated are measured, and together they have a sensitivity analysis area under the curve of greater than 0.99.

39. The method of claim 1, wherein the levels of methylation for 4 or more regulatory elements are measured.

40. The method of claim 1, wherein the levels of methylation for 8 or more regulatory elements are measured.

41. The method of claim 1, wherein the levels of methylation for 12 or more regulatory elements are measured.

42. A kit comprising:
(a) at least one reagent selected from the group consisting of:
(i) a nucleic acid probe capable of specifically hybridizing with a regulatory element differentially methylated in melanoma and benign nevi;
(ii) a pair of nucleic acid primers capable of PCR amplification of a regulatory element differentially methylated in melanoma and benign nevi; and
(iii) a methylation specific antibody and a probe capable of specifically hybridizing with a regulatory element differentially methylated in melanoma and benign nevi; and
(b) instructions for use in measuring a level of methylation of at least one regulatory element in a tissue sample from a subject suspected of having melanoma.

43. A method of identifying a compound that prevents or treats melanoma progression, the method comprising the steps of:
(a) contacting a compound with a sample comprising a cell or a tissue;
(b) measuring a level of methylation of one or more regulatory elements differentially methylated in melanoma and benign nevi; and
(c) determining a functional effect of the compound on the level of methylation; thereby identifying a compound that prevents or treats melanoma.


SEQUENCE LISTING (353 sequences)

SEQ ID NO: 1 - DNA, 59 bp, Artificial Sequence
  oligonucleotide primer
gcacatgtgcgagcatgacagcccgtgtgacgtggagatgcatgaatgtacacgcaaga

SEQ ID NO: 2 - DNA, 43 bp, Artificial Sequence
  oligonucleotide primer
aggagggaggagggccaagggcgggcaggaaggcttaggctcg

SEQ ID NO: 3 - DNA, 49 bp, Artificial Sequence
  oligonucleotide primer
agagctccgagtcacgtggcttgggcgggcctccccttcctggtgtcca

SEQ ID NO: 4 - DNA, 49 bp, Artificial Sequence
  oligonucleotide primer
ccttgcccagaggctgcgggctgcgggtcaagacatcagtagaaggagg

SEQ ID NO: 5 - DNA, 51 bp, Artificial Sequence
  oligonucleotide primer
agcaggtgacggaatgtgggctcgagtgtcagcagagccaagaaaggactg

SEQ ID NO: 6 - DNA, 50 bp, Artificial Sequence
  oligonucleotide primer
tgtaaagagaggcacgtggttaagctctcggggtgtggactccaccagtc

SEQ ID NO: 7 - DNA, 52 bp, Artificial Sequence
  oligonucleotide primer
aagttagctgggtaggtatacagtcattgccgaggaaggcttgcacagggtg

SEQ ID NO: 8 - DNA, 52 bp, Artificial Sequence
  oligonucleotide primer
cgtgccccagccaatcagagctgcctggcccggcccccaatttgggagttgg

SEQ ID NO: 9 - DNA, 59 bp, Artificial Sequence
  oligonucleotide primer
gagtggattctggtaaaagtccttcataatcgtgcccattgtaaacaagtgaaaacttt

SEQ ID NO: 10 - DNA, 52 bp, Artificial Sequence
  oligonucleotide primer
cccatcacctgtataaccctcggtatttctgttcactttaagagcctgccac

SEQ ID NO: 11 - DNA, 53 bp, Artificial Sequence
  oligonucleotide primer
agcaaactgcttcccctctttcgccatcagactcatggttctgcttttcgttt

SEQ ID NO: 12 - DNA, 54 bp, Artificial Sequence
  oligonucleotide primer
gggatgattgagttggtaaaccctaacgaggaaatgccctgaaagttacatcac

SEQ ID NO: 13 - DNA, 54 bp, Artificial Sequence
  oligonucleotide primer
aggaaaccaaacttagatccttcgtaatcctaatttaaaactccatggcgatgg

SEQ ID NO: 14 - DNA, 54 bp, Artificial Sequence
  oligonucleotide primer
catgacaggaggctttgtagaaccaatccccgcctccagagcagggagggtttt

SEQ ID NO: 15 - DNA, 51 bp, Artificial Sequence
  oligonucleotide primer
tggtagagaaatgaaagcaccacagtgtggcggctctgggagtgcactggc

SEQ ID NO: 16 - DNA, 51 bp, Artificial Sequence
  oligonucleotide primer
gcccaggggcaggctatgtgactgcccggtctgcagctgtaagtggtttct

SEQ ID NO: 17 - DNA, 47 bp, Artificial Sequence
  oligonucleotide primer
ggaacagtgatgaggaactgaggccgagtggaggcagatgagactga

SEQ ID NO: 18 - DNA, 55 bp, Artificial Sequence
  oligonucleotide primer
tgcaaatgaccagaaagcaaggaaagaatgcggttaaaagaacaatttggtgagg

SEQ ID NO: 19 - DNA, 59 bp, Artificial Sequence
  oligonucleotide primer
cacctgggacactatgaatgtaacaataatcgttatgaaatatgatcttgtttttagtc

SEQ ID NO: 20 - DNA, 50 bp, Artificial Sequence
  oligonucleotide primer
tctccctcgaactttaaagtccgcttctttgtgttaaccaaagccagcct

SEQ ID NO: 21 - DNA, 51 bp, Artificial Sequence
  oligonucleotide primer
gtgaattttgaaaggatgtggtttcggcctttgacatcagaggagaagctc

SEQ ID NO: 22 - DNA, 52 bp, Artificial Sequence
  oligonucleotide primer
aacagaaacaaggaaaaagggaaacccacgcccactctgtggccgtgagtga

SEQ ID NO: 23 - DNA, 43 bp, Artificial Sequence
  oligonucleotide primer
gggtcctggatatggaggccacggctgccagctggcaggtggc

SEQ ID NO: 24 - DNA, 57 bp, Artificial Sequence
  oligonucleotide primer
gagctggccagtagctgcaatagatgccaccgttaattacctgggcaagatccttgt

SEQ ID NO: 25 - DNA, 51 bp, Artificial Sequence
  oligonucleotide primer
ccggcgtccctcctagtagtaccgctgctctctaacctcaggacgtcaagg

SEQ ID NO: 26 - DNA, 45 bp, Artificial Sequence
  oligonucleotide primer
ggacaggaaatctggctggagaccgttgggcttcacaggaaggag

SEQ ID NO: 27 - DNA, 49 bp, Artificial Sequence
  oligonucleotide primer
ggagaggtggggtgctgaattcgaaggtcaggacacctatacctctggg

SEQ ID NO: 28 - DNA, 49 bp, Artificial Sequence
  oligonucleotide primer
cagagcagtcctccaaggcacgcattggctccactctcctgagcgacgg

SEQ ID NO: 29 - DNA, 49 bp, Artificial Sequence
  oligonucleotide primer
cgctcctcctcctgttttcttcgaattcgttcttcgaggtcagccctac

SEQ ID NO: 30 - DNA, 52 bp, Artificial Sequence
  oligonucleotide primer
caggctggcagccactttatgcccgctggggcgattggccaacacctcatga

SEQ ID NO: 31 - DNA, 52 bp, Artificial Sequence
  oligonucleotide primer
caaggcacaagtgacatttgccttggcgttcttgaccctccctctgtctcgc

SEQ ID NO: 32 - DNA, 52 bp, Artificial Sequence
  oligonucleotide primer
gaagtttggatgttgtgtgccacacttcgatttgtcttaaggaatgtgttcc

SEQ ID NO: 33 - DNA, 47 bp, Artificial Sequence
  oligonucleotide primer
tcctagggggcaggtagacagactgacggatggatgggcagagatgc

SEQ ID NO: 34 - DNA, 53 bp, Artificial Sequence
  oligonucleotide primer
cctcagttcattactgtaaaccccgtaccttaaaagactcggcttcttctcac

SEQ ID NO: 35 - DNA, 52 bp, Artificial Sequence
  oligonucleotide primer
ggcaaggagaggactattgaggcacacacacgtgtctggcagcctgagtggg

SEQ ID NO: 36 - DNA, 47 bp, Artificial Sequence
  oligonucleotide primer
ggcccaggtgagcctggtcccgggacaccatggcgggcgggcgcagc

SEQ ID NO: 37 - DNA, 44 bp, Artificial Sequence
  oligonucleotide primer
gggggcacagagcttgggaagcgcgggagtcccgtgggcaaaag

SEQ ID NO: 38 - DNA, 47 bp, Artificial Sequence
  oligonucleotide primer
ggagaagttgtcatgggaggccagccgcctgctggcaaggaagatgg

SEQ ID NO: 39 - DNA, 48 bp, Artificial Sequence
  oligonucleotide primer
cggcagccagggtggaggagctccgaagctgacagagcagagtgggcc

SEQ ID NO: 40 - DNA, 53 bp, Artificial Sequence
  oligonucleotide primer
cggccttggctcattggctgggccgcggtcacctgggccgtgatgtcacggcc

SEQ ID NO: 41 - DNA, 55 bp, Artificial Sequence
  oligonucleotide primer
ttttatttgtgaggctggcctcagcacgcggcccaagaaacagaactgaaagcgg

SEQ ID NO: 42 - DNA, 46 bp, Artificial Sequence
  oligonucleotide primer
gagagatgctgctgcggaagtcctcggtggagtgtgagaaggcagc

SEQ ID NO: 43 - DNA, 47 bp, Artificial Sequence
  oligonucleotide primer
cccagggcttgagggcatgtgaggcgaggagaggatggactctagag

SEQ ID NO: 44 - DNA, 49 bp, Artificial Sequence
  oligonucleotide primer
ggtgggagccgggcccagcaccaatccgagagcaaggctaggggaggtc

SEQ ID NO: 45 - DNA, 57 bp, Artificial Sequence
  oligonucleotide primer
aggcttgctgttgtgccgttctgccccgatggtatcctgtccgctcgcattggggcg

SEQ ID NO: 46 - DNA, 56 bp, Artificial Sequence
  oligonucleotide primer
agcctgccgctgctgcagcgagtctggcgcagagtggagcggccgccggagatgcc

SEQ ID NO: 47 - DNA, 52 bp, Artificial Sequence
  oligonucleotide primer
cctgcactgcggcaaacaagcacgcctgcgcggccgcagaggcaggctggcg

SEQ ID NO: 48 - DNA, 53 bp, Artificial Sequence
  oligonucleotide primer
tttatttggttgtggacgtcagagccgtcatggtaagaaggaagcaaagcctt

SEQ ID NO: 49 - DNA, 53 bp, Artificial Sequence
  oligonucleotide primer
ggggttgtcttaccgcagtgagtaccacgcggtactacagagaccggctgccc

SEQ ID NO: 50 - DNA, 59 bp, Artificial Sequence
  oligonucleotide primer
aacctgacgtgcaggcacagagcaaggactcgagagaacgagaagcagtggcagcagct

SEQ ID NO: 51 - DNA, 50 bp, Artificial Sequence
  oligonucleotide primer
ccccaggtggctggccacggagcccgccggcacatgcatggctgtgtctc

SEQ ID NO: 52 - DNA, 51 bp, Artificial Sequence
  oligonucleotide primer
cacacacaaagcaacttctgtttcgtttagactctgccacaaaacgccttc

SEQ ID NO: 53 - DNA, 50 bp, Artificial Sequence
  oligonucleotide primer
gcagctgcccagacttctgcaccgaggtgcagctcgacgcctccttgtca

SEQ ID NO: 54 - DNA, 53 bp, Artificial Sequence
  oligonucleotide primer
ggggcctggctcttctccgggtcgtacaaccgcgtgcagctttggctggtcgg

SEQ ID NO: 55 - DNA, 48 bp, Artificial Sequence
  oligonucleotide primer
ccgttggcctcaccctggctcgggcccctttatcatcctgcagctacg

SEQ ID NO: 56 - DNA, 52 bp, Artificial Sequence
  oligonucleotide primer
ccgtatctgccatgcaaaacgagggagcgttaggaaggaatccgtcttgtaa

SEQ ID NO: 57 - DNA, 52 bp, Artificial Sequence
  oligonucleotide primer
accctagggccagggaaacttttgccgtataaatagggcagatccgggcttt

SEQ ID NO: 58 - DNA, 50 bp, Artificial Sequence
  oligonucleotide primer
ggctctaagggctcctccagctcggtgacgtcccgcgtgtaccaggtgtc

SEQ ID NO: 59 - DNA, 48 bp, Artificial Sequence
  oligonucleotide primer
tccaaagtttgagcgtctcaaagcgccagcgcccctacggattagccc

SEQ ID NO: 60 - DNA, 50 bp, Artificial Sequence
  oligonucleotide primer
gggacgtctgtgcctctgcccgggcggctctgcactttcctacctcccgc

SEQ ID NO: 61 - DNA, 50 bp, Artificial Sequence
  oligonucleotide primer
gggattgcacagagggctgggtccgcaggctggctaaaaggacctagccc

SEQ ID NO: 62 - DNA, 55 bp, Artificial Sequence
  oligonucleotide primer
ccttgcctgtgttgtccttcccacgttaggtctgtcatgccacgtatgtccgcag

SEQ ID NO: 63 - DNA, 49 bp, Artificial Sequence
  oligonucleotide primer
tcattcatggtcacttccgaagcgctttagtgccttccgtccctaaacc

SEQ ID NO: 64 - DNA, 53 bp, Artificial Sequence
  oligonucleotide primer
ctccttgctgatttgcacacattggccgcttcagacacgcacttctggggcca

SEQ ID NO: 65 - DNA, 46 bp, Artificial Sequence
  oligonucleotide primer
cctcgctgtattgggaagctacgttccgggctggccaaatgggccc

SEQ ID NO: 66 - DNA, 50 bp, Artificial Sequence
  oligonucleotide primer
gagatgccagattagcgtggtgcctgtccggagagacgggccagctgatg

SEQ ID NO: 67 - DNA, 54 bp, Artificial Sequence
  oligonucleotide primer
aggcgaaggcagccaggccatggggcgacgccaaaatatgcacgaagaaaaatg

SEQ ID NO: 68 - DNA, 50 bp, Artificial Sequence
  oligonucleotide primer
gccggaggctattgtcgaagccacggcctgccatttcataccctttgcaa

SEQ ID NO: 69 - DNA, 57 bp, Artificial Sequence
  oligonucleotide primer
ggactgggccaaatttaagcagcggtcccgacagccccaagatagcggacccccgcc

SEQ ID NO: 70 - DNA, 51 bp, Artificial Sequence
  oligonucleotide primer
cgccgcttgtaggaggtcgagtagtacggctcgtagctgaaggaactcatg

SEQ ID NO: 71 - DNA, 44 bp, Artificial Sequence
  oligonucleotide primer
aggacaaaccctggggtcgctggcgtgtgtgagatggaaatgga

SEQ ID NO: 72 - DNA, 47 bp, Artificial Sequence
  oligonucleotide primer
cccttcccaattctttggccgcctttgaccccggcctctgcttctga

SEQ ID NO: 73 - DNA, 56 bp, Artificial Sequence
  oligonucleotide primer
cagaaatcgtccccgtagtttgtgcgcgtgcaaaggttctcgcagctacactgcca

SEQ ID NO: 74 - DNA, 45 bp, Artificial Sequence
  oligonucleotide primer
aaggggactttgtgaacagtgggcggggagacgcagaggcagagg

SEQ ID NO: 75 - DNA, 54 bp, Artificial Sequence
  oligonucleotide primer
cttgggcatggtgcccgcttggcatagcgcccggctccggatcttcctgtgcct

SEQ ID NO: 76 - DNA, 50 bp, Artificial Sequence
  oligonucleotide primer
ggcagcctagtcttggggacgtagagacgggagaaaggagaagccagcct

SEQ ID NO: 77 - DNA, 55 bp, Artificial Sequence
  oligonucleotide primer
acaactgcttccatctagcatggcagcgttcctgaatcacatctctaaagccgct

SEQ ID NO: 78 - DNA, 45 bp, Artificial Sequence
  oligonucleotide primer
cctttcccagaactcagtcgcctgaacccccagcctgtggttctc

SEQ ID NO: 79 - DNA, 43 bp, Artificial Sequence
  oligonucleotide primer
gcgagagaggcaagtggggtgacgaggtcgtgcactgagggtg

SEQ ID NO: 80 - DNA, 50 bp, Artificial Sequence
  oligonucleotide primer
agagtgagaggccgacccgtgttcccgtgttactgtgtacggagtagtgg

SEQ ID NO: 81 - DNA, 48 bp, Artificial Sequence
  oligonucleotide primer
cctgagagccttcccctaccggggaatatacttcaccagcaccacttt

SEQ ID NO: 82 - DNA, 49 bp, Artificial Sequence
  oligonucleotide primer
gagatgctgtcccgtgggtaagtcccgggcaccatcggggtcccagtct

SEQ ID NO: 83 - DNA, 53 bp, Artificial Sequence
  oligonucleotide primer
gactgctgtaagtcagccaggcagccggtcactgaagcccttccttctctatt

SEQ ID NO: 84 - DNA, 48 bp, Artificial Sequence
  oligonucleotide primer
cactgggaggacagtgaagaatgcccgcctacctggggaaacctgagt

SEQ ID NO: 85 - DNA, 53 bp, Artificial Sequence
  oligonucleotide primer
tgtgcaaatgcatccatctccccgagctatttttcagattccacagaattgca

SEQ ID NO: 86 - DNA, 50 bp, Artificial Sequence
  oligonucleotide primer
tgggttccacgaggcgccaaacaccgtcgccttggactggaagctgcacg

SEQ ID NO: 87 - DNA, 45 bp, Artificial Sequence
  oligonucleotide primer
acctgggcactgggaagaatagggcgtggacttggagtgtgaccg

SEQ ID NO: 88 - DNA, 48 bp, Artificial Sequence
  oligonucleotide primer
ccagcataacatggccaacccgatggctcccgaaaccttgccagatgc

SEQ ID NO: 89 - DNA, 52 bp, Artificial Sequence
  oligonucleotide primer
tgggcgaagccaggaccgtgccgcgccaccgccaggatatggagctactgtc

SEQ ID NO: 90 - DNA, 49 bp, Artificial Sequence
  oligonucleotide primer
ctgcgcaaggtgtttctcaccagcggacgccacctataaggcccatctc

SEQ ID NO: 91 - DNA, 49 bp, Artificial Sequence
  oligonucleotide primer
gagggtggacgcaacctccgagccgccagtccctggcgcagggcaagcg

SEQ ID NO: 92 - DNA, 52 bp, Artificial Sequence
  oligonucleotide primer
aaagctagtgcctttctccttgactagcgtttcctgagcacctgccgcagcc

SEQ ID NO: 93 - DNA, 53 bp, Artificial Sequence
  oligonucleotide primer
cataccaacacgtactatagcaacagcgtgtgcaagcccacatctcagaagca

SEQ ID NO: 94 - DNA, 58 bp, Artificial Sequence
  oligonucleotide primer
gggcagaagccagcttgatggcagacacctcgccaccagtagcaggcgtgggagagtc

SEQ ID NO: 95 - DNA, 46 bp, Artificial Sequence
  oligonucleotide primer
ggggacgtgcccagtgggtcctcgaagaaggcaggacagaaggcgg

SEQ ID NO: 96 - DNA, 46 bp, Artificial Sequence
  oligonucleotide primer
acgggtggcccgtggcccagcagcggctccatggccagcgaggcgg

SEQ ID NO: 97 - DNA, 53 bp, Artificial Sequence; identical to SEQ ID NO: 54
  oligonucleotide primer

SEQ ID NO: 98 - DNA, 48 bp, Artificial Sequence; identical to SEQ ID NO: 55
  oligonucleotide primer

SEQ ID NO: 99 - DNA, 52 bp, Artificial Sequence
  oligonucleotide primer
cctagcgcagctatgtcccgagcgcgcccccacctgtgcgttaatctactgg

SEQ ID NO: 100 - DNA, 47 bp, Artificial Sequence
  oligonucleotide primer
gggcctggaggtctgcacccgaccgccttgtgccaggacggtcaggt

SEQ ID NO: 101 - DNA, 52 bp, Artificial Sequence
  oligonucleotide primer
gccagtagcatgcccctgcccgtctgggtccctctgcgtgtctctgcttgtc

SEQ ID NO: 102 - DNA, 51 bp, Artificial Sequence
  oligonucleotide primer
cacactcctggcatcccagcgtctccagcttgcatggcctgtcacggtatt

SEQ ID NO: 103 - DNA, 50 bp, Artificial Sequence
  oligonucleotide primer
ccatctctctttcctctgcctggcgagatgccagccagcacctcagtgtc

SEQ ID NO: 104 - DNA, 55 bp, Artificial Sequence
  oligonucleotide primer
gacaaagcaaaaccagtgaggctgaaagaacggctgccctggtgcacacagatgg

SEQ ID NO: 105 - DNA, 49 bp, Artificial Sequence
  oligonucleotide primer
cccggaagcccaggcagcgcccgagtccgcagctgccgtcggagctggg

SEQ ID NO: 106 - DNA, 51 bp, Artificial Sequence
  oligonucleotide primer
ggggctcacctggggaccacgtgcggaggtactagaaagcatgcaccgact

SEQ ID NO: 107 - DNA, 49 bp, Artificial Sequence
  oligonucleotide primer
aggggtcctttgcccctgggttgcgtcaccctcatgcttccagaacctg

SEQ ID NO: 108 - DNA, 50 bp, Artificial Sequence
  oligonucleotide primer
ctttcttcctcctacctctgcttcgtaggttcgtcttccttccagcctgc

SEQ ID NO: 109 - DNA, 54 bp, Artificial Sequence
  oligonucleotide primer
tttgttttcaggcaatttccctgagaaccgtttacttccagaagattggtggag

SEQ ID NO: 110 - DNA, 57 bp, Artificial Sequence
  oligonucleotide primer
tgtggacataagaaagggttaacatggccgacaactatttcatgagctttttggctt

SEQ ID NO: 111 - DNA, 49 bp, Artificial Sequence; identical to SEQ ID NO: 91
  oligonucleotide primer

SEQ ID NO: 112 - DNA, 46 bp, Artificial Sequence
  oligonucleotide primer
tcagggctcagatggagtctggagcgactgaagttgggctccaggg

SEQ ID NO: 113 - DNA, 52 bp, Artificial Sequence; identical to SEQ ID NO: 56
  oligonucleotide primer

SEQ ID NO: 114 - DNA, 49 bp, Artificial Sequence
  oligonucleotide primer
tctaggaagctctccggggagccggttctcccgccggtggcttcttctg

SEQ ID NO: 115 - DNA, 52 bp, Artificial Sequence; identical to SEQ ID NO: 57
  oligonucleotide primer

SEQ ID NO: 116 - DNA, 50 bp, Artificial Sequence
  oligonucleotide primer
caaagcctatcctccctgtagccgggtgccaagcagcctcgagcctgctc

SEQ ID NO: 117 - DNA, 57 bp, Artificial Sequence
  oligonucleotide primer
gactggacagctcctgctttgatcgccggagatctgcaaattctgcccatgtcgggg

SEQ ID NO: 118 - DNA, 51 bp, Artificial Sequence
  oligonucleotide primer
cttgactcagccactgtatgactgactccccggggacatgaggtggatact

SEQ ID NO: 119 - DNA, 52 bp, Artificial Sequence
  oligonucleotide primer
agacatcacagcgctgggctaggggcgcggcttgaactcgcctaaagagctg

SEQ ID NO: 120 - DNA, 51 bp, Artificial Sequence
  oligonucleotide primer
cctcaactttgcagcgtacttggaccgctctggccgccctgggcgctaccc

SEQ ID NO: 121 - DNA, 52 bp, Artificial Sequence
  oligonucleotide primer
tctagcagctgcctgtcacagagcacgccggcctcaatccgggcctgtgggc

SEQ ID NO: 122 - DNA, 50 bp, Artificial Sequence
  oligonucleotide primer
gcttctctccccgagctctgtcgttaatggctcagcctctgacaggcccg

SEQ ID NO: 123 - DNA, 45 bp, Artificial Sequence
  oligonucleotide primer
gttgagattgagactgggggtcggtgagtggcgtcaattcccatg

SEQ ID NO: 124 - DNA, 45 bp, Artificial Sequence
  oligonucleotide primer
ggcctgggcgtctggacccccgggtcccttagaacgcccttcaga

SEQ ID NO: 125 - DNA, 56 bp, Artificial Sequence
  oligonucleotide primer
gcgttttaagtcagacaaggaagggaacgtaatgaggcaccacagactcgagaaat

SEQ ID NO: 126 - DNA, 51 bp, Artificial Sequence
  oligonucleotide primer
tcctcccctgttgcctacccgcccctttcacatgatctctgactatagctg

SEQ ID NO: 127 - DNA, 49 bp, Artificial Sequence; identical to SEQ ID NO: 90
  oligonucleotide primer

SEQ ID NO: 128 - DNA, 51 bp, Artificial Sequence
  oligonucleotide primer
ggccgagggctggcttttaaaacaccgaaaacccagacaggaacggtgtcc

SEQ ID NO: 129 - DNA, 49 bp, Artificial Sequence
  oligonucleotide primer
atccccacagtccctgagcgatggtgcagtccagcttcattttcctatt

SEQ ID NO: 130 - DNA, 53 bp, Artificial Sequence; identical to SEQ ID NO: 11
  oligonucleotide primer

SEQ ID NO: 131 - DNA, 54 bp, Artificial Sequence; identical to SEQ ID NO: 14
  oligonucleotide primer

SEQ ID NO: 132 - DNA, 54 bp, Artificial Sequence
  oligonucleotide primer
tcagcaatgtgcctagagaagctctgacgccgccttggaagtaagtcgttgctg

SEQ ID NO: 133 - DNA, 52 bp, Artificial Sequence
  oligonucleotide primer
catgacaagcaacatgccgtcagcgtaaatacagcgcgggtcctctagcaca

SEQ ID NO: 134 - DNA, 52 bp, Artificial Sequence
  oligonucleotide primer
gactcagggtttcttcctcccgcctctcgcagtgcatctttcatttgctttt

SEQ ID NO: 135 - DNA, 52 bp, Artificial Sequence
  oligonucleotide primer
ctacagcctggtctcctttggcgtttgcgcccctgcatctgagcacgtccca

SEQ ID NO: 136 - DNA, 53 bp, Artificial Sequence
  oligonucleotide primer
gaagtctaggaaggcaccggagaccctcggcacaaggcactgaacctggagcg

SEQ ID NO: 137 - DNA, 48 bp, Artificial Sequence
  oligonucleotide primer
caggatggggcagggtgcagccgcgcagtggacgccaaaaggcccgct

SEQ ID NO: 138 - DNA, 50 bp, Artificial Sequence; identical to SEQ ID NO: 60
  oligonucleotide primer

SEQ ID NO: 139 - DNA, 57 bp, Artificial Sequence
  oligonucleotide primer
gaggtcatacccaggcactgggtgttggcgggagcagtaaagcgccataaaagcacc

SEQ ID NO: 140 - DNA, 46 bp, Artificial Sequence
  oligonucleotide primer
gtgccaaggactaaggttgggggcggtgggagagacaagcctcgtt

SEQ ID NO: 141 - DNA, 45 bp, Artificial Sequence
  oligonucleotide primer
ggaactgcaaggaggtgactcctttcggggtgaggaggcccagac

SEQ ID NO: 142 - DNA, 50 bp, Artificial Sequence; identical to SEQ ID NO: 61
  oligonucleotide primer

SEQ ID NO: 143 - DNA, 53 bp, Artificial Sequence
  oligonucleotide primer
acacgatgctccgttttcttccgttgtgaatgccgcgtcctgtcctggtgaca

SEQ ID NO: 144 - DNA, 45 bp, Artificial Sequence
  oligonucleotide primer
tactctgtcgtgggctgaaggcacccggcctgggaaaaggaaacc

SEQ ID NO: 145 - DNA, 51 bp, Artificial Sequence
  oligonucleotide primer
gcctctgacttctctgtccgaagtcgggacaccctcctaccacctgtagag

SEQ ID NO: 146 - DNA, 50 bp, Artificial Sequence
  oligonucleotide primer
ggtactgtctgttcggctgtcttccccgcctctccccaggcacctgcatc

SEQ ID NO: 147 - DNA, 59 bp, Artificial Sequence
  oligonucleotide primer
cacatggacaccctgtgcatcagtgtgcgtttaattcaaagacagacctcatttgatag

SEQ ID NO: 148 - DNA, 49 bp, Artificial Sequence
  oligonucleotide primer
ggcccccgacggggccactgtatttcgggctgcagacctagaggccctg

SEQ ID NO: 149 - DNA, 51 bp, Artificial Sequence; identical to SEQ ID NO: 16
  oligonucleotide primer

SEQ ID NO: 150 - DNA, 51 bp, Artificial Sequence
  oligonucleotide primer
tggtgtctgaatggagcaggcctgcggaagagaaaccgctgaccacagacc

SEQ ID NO: 151 - DNA, 45 bp, Artificial Sequence
  oligonucleotide primer
agcctcacaggccctctgggtcgccaccctcccatgctctatccc

SEQ ID NO: 152 - DNA, 56 bp, Artificial Sequence
  oligonucleotide primer
tcctcctgatgcttttgcagaccgcggtcctgcaggggcgcttgctgcgtgagtcc

SEQ ID NO: 153 - DNA, 49 bp, Artificial Sequence
  oligonucleotide primer
aaaccatctcagcctactcaacggcatctgggatgtccccctgcctcta

SEQ ID NO: 154 - DNA, 54 bp, Artificial Sequence
  oligonucleotide primer
accttgggctctccgcagtagccgagcttaacatgattctccactgcagctgcc

SEQ ID NO: 155 - DNA, 52 bp, Artificial Sequence
  oligonucleotide primer
cagggaggtgctggtcatgtgacccgatgttgaaattgacaagctgctagct

SEQ ID NO: 156 - DNA, 50 bp, Artificial Sequence; identical to SEQ ID NO: 86
  oligonucleotide primer

SEQ ID NO: 157 - DNA, 51 bp, Artificial Sequence
  oligonucleotide primer
ctacaagtggcatgaatggaaggcaagttcggtttgggaaaaggcagcctc

SEQ ID NO: 158 - DNA, 54 bp, Artificial Sequence
  oligonucleotide primer
ccccatacacacacttcttaagcggactattttatatcacaattaatcacgcca

SEQ ID NO: 159 - DNA, 52 bp, Artificial Sequence
  oligonucleotide primer
cctgttggcttcctctggcacggctcggctgggttcctccctccctgtgcgg

SEQ ID NO: 160 - DNA, 53 bp, Artificial Sequence
  oligonucleotide primer
agcctatcagagatgctacagcaagtcgatattcagtcattttcaaccacaaa

SEQ ID NO: 161 - DNA, 53 bp, Artificial Sequence
  oligonucleotide primer
ctatgttgcaaaacccatttttgctaacgtgtccagtgggctcccgggacgac

SEQ ID NO: 162 - DNA, 53 bp, Artificial Sequence; identical to SEQ ID NO: 85
  oligonucleotide primer

SEQ ID NO: 163 - DNA, 56 bp, Artificial Sequence
  oligonucleotide primer
attttgaacactcagctcctagcgtgcggcgctgccaatcattaacctcctggtgc

SEQ ID NO: 164 - DNA, 50 bp, Artificial Sequence
  oligonucleotide primer
cctcccaccagcggtttgcgtagggccttgggtgcactagcaaaacaaac

SEQ ID NO: 165 - DNA, 53 bp, Artificial Sequence
  oligonucleotide primer
gaagtttccaaagagactacggggctccgggagagcaggcgcttttaaatagc

SEQ ID NO: 166 - DNA, 47 bp, Artificial Sequence
  oligonucleotide primer
cagctccaaatcgccagtgctgacggcttccgctttgggagccccag

SEQ ID NO: 167 - DNA, 51 bp, Artificial Sequence
  oligonucleotide primer
gagggactgtggcccaggtactgcccgggtgctactttatgggcagcagct

SEQ ID NO: 168 - DNA, 45 bp, Artificial Sequence
  oligonucleotide primer
catcaagtcagccatcagccggcccatctcctcatgctggccaac

SEQ ID NO: 169 - DNA, 50 bp, Artificial Sequence
  oligonucleotide primer
gacgcttctgaaagggcaaagacgacgccaaagaagacgccggagacctc

SEQ ID NO: 170 - DNA, 56 bp, Artificial Sequence
  oligonucleotide primer
ccattttggggagcaccgccagctgcccgttcaggagtgtgcagcaaactcagctg

SEQ ID NO: 171 - DNA, 49 bp, Artificial Sequence
  oligonucleotide primer
ggacaggcacagactggaacttggacccgaggcaggacagggagctggc

SEQ ID NO: 172 - DNA, 50 bp, Artificial Sequence; identical to SEQ ID NO: 66
  oligonucleotide primer

SEQ ID NO: 173 - DNA, 43 bp, Artificial Sequence
  oligonucleotide primer
aggtgggtcccaacctccacgtcggccaattccaggtggcccc

SEQ ID NO: 174 - DNA, 53 bp, Artificial Sequence
  oligonucleotide primer
gcactggcccaggtctggcaccgcgctacaatttcttctgtagcccgttctga

SEQ ID NO: 175 - DNA, 49 bp, Artificial Sequence
  oligonucleotide primer
gcgtggtgcccagcttcacaaagcgagcgggcagcacctccttggtccg

SEQ ID NO: 176 - DNA, 52 bp, Artificial Sequence; identical to SEQ ID NO: 22
  oligonucleotide primer

SEQ ID NO: 177 - DNA, 48 bp, Artificial Sequence
  oligonucleotide primer
tcgctcccagtccgaaatggcgggggccgggagtactggccgagccgc

SEQ ID NO: 178 - DNA, 43 bp, Artificial Sequence
  oligonucleotide primer
cagcacagccagccgggctcggttcaggctccggccggagggg

SEQ ID NO: 179 - DNA, 56 bp, Artificial Sequence
  oligonucleotide primer
cccatgacatcctctgtctagacacggtcaggacacaaatctggcagctctactgt

SEQ ID NO: 180 - DNA, 54 bp, Artificial Sequence; identical to SEQ ID NO: 67
  oligonucleotide primer

SEQ ID NO: 181 - DNA, 49 bp, Artificial Sequence
  oligonucleotide primer
aatctgggagaggtgatctgcaccccgagatcccgggatttgtagagtt

SEQ ID NO: 182 - DNA, 49 bp, Artificial Sequence
  oligonucleotide primer
ggaaaggagacgcgagaggtgtagtcgatgtgcctgcgaagcccaggct

SEQ ID NO: 183 - DNA, 50 bp, Artificial Sequence
  oligonucleotide primer
tcccttggttgcagtagcctgtggtcgctcatgtctgaatctccagggaa

SEQ ID NO: 184 - DNA, 51 bp, Artificial Sequence
  oligonucleotide primer
tgcagcctgagttagacttctgcaacgtcccgtgaggtgggatcaggaatg

SEQ ID NO: 185 - DNA, 44 bp, Artificial Sequence
  oligonucleotide primer
ctgcagggaagaaggacgtgcggcgagaagcatcggattcgggg

SEQ ID NO: 186 - DNA, 55 bp, Artificial Sequence
  oligonucleotide primer
gtagagtccagggactaggaggactcacaacgcagcgatgggcagccaggccctg

SEQ ID NO: 187 - DNA, 45 bp, Artificial Sequence; identical to SEQ ID NO: 87
  oligonucleotide primer

SEQ ID NO: 188 - DNA, 45 bp, Artificial Sequence
  oligonucleotide primer
ccctcccaatgcaggttaagacgacagcctgcgcccccaactagc

SEQ ID NO: 189 - DNA, 54 bp, Artificial Sequence
  oligonucleotide primer
tcaggaagcgcatgcgcaaccggttctccgaaacatggagtcctgtaggcaagg

SEQ ID NO: 190 - DNA, 46 bp, Artificial Sequence
  oligonucleotide primer
gctgacgcctggcagggagaaggcggcagcacatgctgggctcggg

SEQ ID NO: 191 - DNA, 50 bp, Artificial Sequence; identical to SEQ ID NO: 68
  oligonucleotide primer

SEQ ID NO: 192 - DNA, 47 bp, Artificial Sequence
  oligonucleotide primer
ggaaactgaagagacgtggccacggcgaggacgaaactagaatgggg

SEQ ID NO: 193 - DNA, 50 bp, Artificial Sequence
  oligonucleotide primer
caggcacacagcacacagcacggtgagtcgcatagctgccgtccagagac

SEQ ID NO: 194 - DNA, 45 bp, Artificial Sequence; identical to SEQ ID NO: 26
  oligonucleotide primer

SEQ ID NO: 195 - DNA, 50 bp, Artificial Sequence
  oligonucleotide primer
agcagcaacaggaaggactgaggcagcggcgggaggagctccatcgaggc

SEQ ID NO: 196 - DNA, 47 bp, Artificial Sequence
  oligonucleotide primer
ggagggggcagaacagattcaggcaggcgctggctgcttgagaggtg

SEQ ID NO: 197 - DNA, 47 bp, Artificial Sequence
  oligonucleotide primer
aaatccccaagtcctacaatcgtgtcccagtggtgtccctgggccac

SEQ ID NO: 198 - DNA, 51 bp, Artificial Sequence
  oligonucleotide primer
gaattccgggcagagggaagggcgcaggcaacagctaggaggcgcagatgc

SEQ ID NO: 199 - DNA, 54 bp, Artificial Sequence
  oligonucleotide primer
cctcggcctgctgcaagcctcacgtctgagctgtttcctgagtcacacaatgtc

SEQ ID NO: 200 - DNA, 49 bp, Artificial Sequence; identical to SEQ ID NO: 28
  oligonucleotide primer

SEQ ID NO: 201 - DNA, 48 bp, Artificial Sequence
  oligonucleotide primer
tccgggaagcgcaggcccccgcctcgggaatatagttgattggcccga

SEQ ID NO: 202 - DNA, 48 bp, Artificial Sequence
  oligonucleotide primer
gtgtgggacattcattgcggaataacatcggaggagaaggtaagggaa

SEQ ID NO: 203 - DNA, 45 bp, Artificial Sequence
  oligonucleotide primer
aagcatccttcgggaggagcagagccgccagaggggccgccctgg

SEQ ID NO: 204 - DNA, 52 bp, Artificial Sequence
  oligonucleotide primer
cactagcagttattccacatttccgcctaaatctcccagcagccactaatat

SEQ ID NO: 205 - DNA, 53 bp, Artificial Sequence
  oligonucleotide primer
aaaggcttccacagtctgacattcgtttatgtctccctcagtttcaggcttgg

SEQ ID NO: 206 - DNA, 48 bp, Artificial Sequence
  oligonucleotide primer
tctcgattcctcagtccagacgctgttgggtcccctccgctggagatc

SEQ ID NO: 207 - DNA, 50 bp, Artificial Sequence
  oligonucleotide primer
tggttcgcatttggcggtaaatatcaccgtctgcacacggggaggcctcc

SEQ ID NO: 208 - DNA, 55 bp, Artificial Sequence
  oligonucleotide primer
ctgatccttacctttgtggcagctgctcgtgagtatcatgccctgcctcaggccc

SEQ ID NO: 209 - DNA, 47 bp, Artificial Sequence
  oligonucleotide primer
tagccccctggccaggtccgatttcaacaccaagtttctgagctttt

SEQ ID NO: 210 - DNA, 53 bp, Artificial Sequence
  oligonucleotide primer
gggagacgcctggagtatccgaagcgagcagtgtggacgagtcaccagcaccg

SEQ ID NO: 211 - DNA, 52 bp, Artificial Sequence; identical to SEQ ID NO: 35
  oligonucleotide primer

SEQ ID NO: 212 - DNA, 44 bp, Artificial Sequence; identical to SEQ ID NO: 37
  oligonucleotide primer

SEQ ID NO: 213 - DNA, 49 bp, Artificial Sequence; identical to SEQ ID NO: 82
  oligonucleotide primer

SEQ ID NO: 214 - DNA, 45 bp, Artificial Sequence
  oligonucleotide primer
tgagagggaactgggatctggcgcctggattgctcaagagaggtc

SEQ ID NO: 215 - DNA, 47 bp, Artificial Sequence; identical to SEQ ID NO: 72
  oligonucleotide primer

SEQ ID NO: 216 - DNA, 46 bp, Artificial Sequence
  oligonucleotide primer
gaactgttcctgtccccagccgatgaccagacgcccatctttcttc

SEQ ID NO: 217 - DNA, 46 bp, Artificial Sequence
  oligonucleotide primer
gaggactgggatgccgagaacgcgagcgatccgagcagggtttgtc

SEQ ID NO: 218 - DNA, 53 bp, Artificial Sequence
  oligonucleotide primer
ctagttgggtcatttgaaggttagcagcccgggtagggttcaccgaaagttca

SEQ ID NO: 219 - DNA, 42 bp, Artificial Sequence
  oligonucleotide primer
cggagaaaggggcaggccgcagcgggcattgatggggctcct

SEQ ID NO: 220 - DNA, 47 bp, Artificial Sequence
  oligonucleotide primer
ccagggcgaaggtctgtagcgagcccgggtccccatggggccactcc

SEQ ID NO: 221 - DNA, 50 bp, Artificial Sequence
  oligonucleotide primer
gaaagctgggaggttcaactacgggcgagaaaattggggcactttccacg

SEQ ID NO: 222 - DNA, 58 bp, Artificial Sequence
  oligonucleotide primer
cccctgtgtgagctactgcctgcctccggtgctctgtttctgtccctagagttctttt

SEQ ID NO: 223 - DNA, 52 bp, Artificial Sequence; identical to SEQ ID NO: 92
  oligonucleotide primer

SEQ ID NO: 224 - DNA, 48 bp, Artificial Sequence; identical to SEQ ID NO: 39
  oligonucleotide primer

SEQ ID NO: 225 - DNA, 53 bp, Artificial Sequence; identical to SEQ ID NO: 40
  oligonucleotide primer

SEQ ID NO: 226 - DNA, 49 bp, Artificial Sequence
  oligonucleotide primer
tctacaccttggcacagccaccgagtgtcccttgctcccctcagtactt

SEQ ID NO: 227 - DNA, 58 bp, Artificial Sequence
  oligonucleotide primer
ccttttatctaagctcctctgatagccggtggcagtctctaatcctgctccctgcttc

SEQ ID NO: 228 - DNA, 58 bp, Artificial Sequence
  oligonucleotide primer
gagattagagccgggagcagaaccctcaggcgtgcctgtgaaaggcatgtagctataa

SEQ ID NO: 229 - DNA, 46 bp, Artificial Sequence; identical to SEQ ID NO: 42
  oligonucleotide primer

SEQ ID NO: 230 - DNA, 47 bp, Artificial Sequence
  oligonucleotide primer
cttgtgcccattccactcccgcctggctgccgtctccagctggtccc

SEQ ID NO: 231 - DNA, 47 bp, Artificial Sequence
  oligonucleotide primer
gcgtctgcaggcaggcctgctggccggaaacctgccaggaaaggaag

SEQ ID NO: 232 - DNA, 53 bp, Artificial Sequence
  oligonucleotide primer
cgctattcctctattttcttttcctcggacctgcagccttgggtcgaccctgc

SEQ ID NO: 233 - DNA, 49 bp, Artificial Sequence; identical to SEQ ID NO: 44
  oligonucleotide primer

SEQ ID NO: 234 - DNA, 49 bp, Artificial Sequence
  oligonucleotide primer
ggaaaagaccaagcagggggtgacggaagcagctgagaagaccaaggag

SEQ ID NO: 235 - DNA, 55 bp, Artificial Sequence
  oligonucleotide primer
cgtcaataggaggcatcggggacagccgctgcggcagcactcgagccagctcaag

SEQ ID NO: 236 - DNA, 54 bp, Artificial Sequence
  oligonucleotide primer
gctggctgggctccagctggcctccgcatcaatatttcatcggcgtcaatagga

SEQ ID NO: 237 - DNA, 57 bp, Artificial Sequence; identical to SEQ ID NO: 45
  oligonucleotide primer

SEQ ID NO: 238 - DNA, 49 bp, Artificial Sequence
  oligonucleotide primer
tgtgctgggaggaagtcagacagccgcgagatgaagagttggccagggc

SEQ ID NO: 239 - DNA, 54 bp, Artificial Sequence
  oligonucleotide primer
agttgcagccttctcagccaaacgccgaccaaggtacagcttcagtttgctact

SEQ ID NO: 240 - DNA, 49 bp, Artificial Sequence
  oligonucleotide primer
cagccaccgacaggctgcatgacggtggcaaagtcacttcccctctctg

SEQ ID NO: 241 - DNA, 50 bp, Artificial Sequence
  oligonucleotide primer
attttgtctgtgaggaaacgggcgacgctgcctactgagactaagcagga

SEQ ID NO: 242 - DNA, 53 bp, Artificial Sequence
  oligonucleotide primer
ccgacaggctgtctggaacattttcgaaccctccaactgggatcggtctggtt

SEQ ID NO: 243 - DNA, 56 bp, Artificial Sequence
  oligonucleotide primer
tcacacatcgaagtcttggattaactgcgaaggcctccttctatttgccgcggctt

SEQ ID NO: 244 - DNA, 51 bp, Artificial Sequence
  oligonucleotide primer
gtaggacgatgctaatggaaagtcacaaaccgctgggtttttgaaaggatc

SEQ ID NO: 245 - DNA, 52 bp, Artificial Sequence
  oligonucleotide primer
gccagggtgactctctccctgctcggtgatacctcttcctgccctggacaga

SEQ ID NO: 246 - DNA, 56 bp, Artificial Sequence
  oligonucleotide primer
tttctgatcctgcatctggtcacggtcgcgctcagcctgtctacctgcagcacact

SEQ ID NO: 247 - DNA, 48 bp, Artificial Sequence
  oligonucleotide primer
caggaagcgctggcaaccctgaggacgaagaagcggactgtgtgcctt

SEQ ID NO: 248 - DNA, 45 bp, Artificial Sequence
  oligonucleotide primer
actgagcacgggcacagtgcgggagcgggtgggtgcccagggcag

SEQ ID NO: 249 - DNA, 59 bp, Artificial Sequence; identical to SEQ ID NO: 50
  oligonucleotide primer

SEQ ID NO: 250 - DNA, 44 bp, Artificial Sequence
  oligonucleotide primer
ggaaggaagagaaggcggtcccgcattggtgtgagagtggcagg

SEQ ID NO: 251 - DNA, 51 bp, Artificial Sequence
  oligonucleotide primer
tcgttttgccacttggtcccagcgccaggcttctcggtcgggagttgacct

SEQ ID NO: 252 - DNA, 48 bp, Artificial Sequence
  oligonucleotide primer
ttcctctgtgaccgcccttgccgctctcagcttctgttcctcaaccac

SEQ ID NO: 253 - DNA, 45 bp, Artificial Sequence
  oligonucleotide primer
aggggtgaaggagcgcttcctaccgttagggaactctggggacag

SEQ ID NO: 254 - DNA, 51 bp, Artificial Sequence
  oligonucleotide primer
gggtataaattcagaggcgctgcgctccgattctggcagtgcagctgtggg

SEQ ID NO: 255 - DNA, 56 bp, Artificial Sequence; identical to SEQ ID NO: 73
  oligonucleotide primer

SEQ ID NO: 256 - DNA, 51 bp, Artificial Sequence
  oligonucleotide primer
cgtggtcagttgtactcccttcccgcagtcacttccaggcactcaggctgg

SEQ ID NO: 257 - DNA, 53 bp, Artificial Sequence; identical to SEQ ID NO: 83
  oligonucleotide primer

SEQ ID NO: 258 - DNA, 50 bp, Artificial Sequence
  oligonucleotide primer
tcttttatagtcagtgaggaaatgaaagcgaatgagttgtttttctgggt

SEQ ID NO: 259 - DNA, 50 bp, Artificial Sequence; identical to SEQ ID NO: 51
  oligonucleotide primer

SEQ ID NO: 260 - DNA, 51 bp, Artificial Sequence; identical to SEQ ID NO: 52
  oligonucleotide primer

SEQ ID NO: 261 - DNA, 44 bp, Artificial Sequence
  oligonucleotide primer
ggctggaaagacgtgaaggaagacgagcagaggagaagggaagg

SEQ ID NO: 262 - DNA, 52 bp, Artificial Sequence
  oligonucleotide primer
gcacttgcttctcatccggggagcggggagtctccgtcttcacaagtgggca

SEQ ID NO: 263 - DNA, 45 bp, Artificial Sequence; identical to SEQ ID NO: 74
  oligonucleotide primer

SEQ ID NO: 264 - DNA, 55 bp, Artificial Sequence
  oligonucleotide primer
aaagaagaggaagtggtagcactagctgtcgctccacaggcgagcagggcaggcg

SEQ ID NO: 265 - DNA, 52 bp, Artificial Sequence
  oligonucleotide primer
cttggggtgcacaggcaaaggcaaaccgccttagggagacccagtggcagcg

SEQ ID NO: 266 - DNA, 46 bp, Artificial Sequence; identical to SEQ ID NO: 96
  oligonucleotide primer

SEQ ID NO: 267 - DNA, 55 bp, Artificial Sequence
  oligonucleotide primer
ttccttggacttctcagtctattctcgccacttctgtcatgtcagtcagtcacac

SEQ ID NO: 268 - DNA, 52 bp, Artificial Sequence
  oligonucleotide primer
cgggcgcccagtggtcctgccgcctggtctcacctcgctatggttcgtctgc

SEQ ID NO: 269 - DNA, 50 bp, Artificial Sequence
  oligonucleotide primer
ctgccctctctgagcccccgcctccaggcctgtgtgtgtgtctccgttcg

SEQ ID NO: 270 - DNA, 58 bp, Artificial Sequence
  oligonucleotide primer
aggccagacgctgagagagaaaaacactgcgtaatcccacgtattgtggagtccaaaa

SEQ ID NO: 271 - DNA, 50 bp, Artificial Sequence; identical to SEQ ID NO: 86
  oligonucleotide primer

SEQ ID NO: 272 - DNA, 54 bp, Artificial Sequence; identical to SEQ ID NO: 158
  oligonucleotide primer

SEQ ID NO: 273 - DNA, 50 bp, Artificial Sequence
  oligonucleotide primer
ccccaccgcagaggtgtgaaggggcgcaaagccagcgaagggagaacccg

SEQ ID NO: 274 - DNA, 52 bp, Artificial Sequence; identical to SEQ ID NO: 22
  oligonucleotide primer

SEQ ID NO: 275 - DNA, 55 bp, Artificial Sequence; identical to SEQ ID NO: 186
  oligonucleotide primer

SEQ ID NO: 276 - DNA, 47 bp, Artificial Sequence
  oligonucleotide primer
ctacagccccctgctgtccatcgcggcctcaacccctgcagatggca

SEQ ID NO: 277 - DNA, 44 bp, Artificial Sequence; identical to SEQ ID NO: 37
  oligonucleotide primer

SEQ ID NO: 278 - DNA, 50 bp, Artificial Sequence
  oligonucleotide primer
ggatagcagaggtgatgggagatagcgtcaaggccaggggtagatgcctc

SEQ ID NO: 279 - DNA, 58 bp, Artificial Sequence; identical to SEQ ID NO: 228
  oligonucleotide primer

SEQ ID NO: 280 - DNA, 47 bp, Artificial Sequence; identical to SEQ ID NO: 230
  oligonucleotide primer

SEQ ID NO: 281 - DNA, 48 bp, Artificial Sequence
  oligonucleotide primer
atggcaggctgccggccgctgataacggaacacatcggagttgggtcg

SEQ ID NO: 282 - DNA, 55 bp, Artificial Sequence
  oligonucleotide primer
cgctaagacccttgctctctttccgtcgaacatgagatgccaatttctttctggg

SEQ ID NO: 283 - DNA, 48 bp, Artificial Sequence
  oligonucleotide primer
tttctctgaaccctaactcctgccgttacgccccaccagctctaggcc

SEQ ID NO: 284 - DNA, 26 bp, Artificial Sequence
  oligonucleotide primer
ggggtaggggagttagtagaggtttc

SEQ ID NO: 285 - DNA, 26 bp, Artificial Sequence
  oligonucleotide primer
ggggtaggggagttagtagaggtttt

SEQ ID NO: 286 - DNA, 25 bp, Artificial Sequence
  oligonucleotide primer
taagaagttagtttcgtggttacgt

SEQ ID NO: 287 - DNA, 25 bp, Artificial Sequence
  oligonucleotide primer
taagaagttagttttgtggttatgt

SEQ ID NO: 288 - DNA, 25 bp, Artificial Sequence
  oligonucleotide primer
ggggttttgttttttttgagttttc

SEQ ID NO: 289 - DNA, 25 bp, Artificial Sequence
  oligonucleotide primer
ggggttttgttttttttgagttttt

SEQ ID NO: 290 - DNA, 25 bp, Artificial Sequence
  oligonucleotide primer
atgtaatttttagggtattttttcg

SEQ ID NO: 291 - DNA, 25 bp, Artificial Sequence
  oligonucleotide primer
atgtaatttttagggtatttttttg

SEQ ID NO: 292 - DNA, 25 bp, Artificial Sequence
  oligonucleotide primer
attttattttcgggaagagtagtcg

SEQ ID NO: 293 - DNA, 25 bp, Artificial Sequence
  oligonucleotide primer
attttatttttgggaagagtagttg

SEQ ID NO: 294 - DNA, 20 bp, Artificial Sequence
  oligonucleotide primer
ttcgttttgggtttttgggc

SEQ ID NO: 295 - DNA, 24 bp, Artificial Sequence
  oligonucleotide primer
ttttttgttttgggtttttgggtg

SEQ ID NO: 296 - DNA, 25 bp, Artificial Sequence
  oligonucleotide primer
tgtagtttttagtttaaggcgacgg

SEQ ID NO: 297 - DNA, 25 bp, Artificial Sequence
  oligonucleotide primer
tgtagtttttagtttaaggtgatgg

SEQ ID NO: 298 - DNA, 20 bp, Artificial Sequence
  oligonucleotide primer
gggtttcgttggtcgtattc

SEQ ID NO: 299 - DNA, 22 bp, Artificial Sequence
  oligonucleotide primer
aggggttttgttggttgtattt

SEQ ID NO: 300 - DNA, 24 bp, Artificial Sequence
  oligonucleotide primer
ttttttttcgaattttaaagttcg

SEQ ID NO: 301 - DNA, 23 bp, Artificial Sequence
  oligonucleotide primer
ttttttttgaattttaaagtttg

SEQ ID NO: 302 - DNA, 24 bp, Artificial Sequence
  oligonucleotide primer
gggtttgggagatgttagattagc

SEQ ID NO: 303 - DNA, 25 bp, Artificial Sequence
  oligonucleotide primer
ggtttgggagatgttagattagtgt

SEQ ID NO: 304 - DNA, 26 bp, Artificial Sequence
  oligonucleotide primer
gggttttattaagttttttacgtgcg

SEQ ID NO: 305 - DNA, 27 bp, Artificial Sequence
  oligonucleotide primer
tgggttttattaagttttttatgtgtg

SEQ ID NO: 306 - DNA, 20 bp, Artificial Sequence
  oligonucleotide primer
gcggagtttagttagtgggc

SEQ ID NO: 307 - DNA, 23 bp, Artificial Sequence
  oligonucleotide primer
tggtggagtttagttagtgggtg

SEQ ID NO: 308 - DNA, 24 bp, Artificial Sequence
  oligonucleotide primer
gagtttttttattttggttgtcga

SEQ ID NO: 309 - DNA, 25 bp, Artificial Sequence
  oligonucleotide primer
ggagtttttttattttggttgttga

SEQ ID NO: 310 - DNA, 25 bp, Artificial Sequence
  oligonucleotide primer
tagggttgtagtaagtatttaacgg

SEQ ID NO: 311 - DNA, 30 bp, Artificial Sequence
  oligonucleotide primer
atggatttagggttgtagtaagtatttaat

SEQ ID NO: 312 - DNA, 27 bp, Artificial Sequence
  oligonucleotide primer
cactacaaaaacaaacgaaccataacg

SEQ ID NO: 313 - DNA, 28 bp, Artificial Sequence
  oligonucleotide primer
cactacaaaaacaaacaaaccataacaa

SEQ ID NO: 314 - DNA, 26 bp, Artificial Sequence
  oligonucleotide primer
acccgaatctaccctatttatacgac

SEQ ID NO: 315 - DNA, 26 bp, Artificial Sequence
  oligonucleotide primer
acccaaatctaccctatttatacaac

SEQ ID NO: 316 - DNA, 25 bp, Artificial Sequence
  oligonucleotide primer
actccttctaaaacctttttcccga

SEQ ID NO: 317 - DNA, 25 bp, Artificial Sequence
  oligonucleotide primer
actccttctaaaacctttttcccaa

SEQ ID NO: 318 - DNA, 25 bp, Artificial Sequence
  oligonucleotide primer
tcaaactcataattctacttttcgt

SEQ ID NO: 319 - DNA, 27 bp, Artificial Sequence
  oligonucleotide primer
catcaaactcataattctacttttcat

SEQ ID NO: 320 - DNA, 18 bp, Artificial Sequence
  oligonucleotide primer
aaaaaccccgcaaaacgt

SEQ ID NO: 321 - DNA, 20 bp, Artificial Sequence
  oligonucleotide primer
aaaaaaccccacaaaaacat

SEQ ID NO: 322 - DNA, 24 bp, Artificial Sequence
  oligonucleotide primer
aaaaaaaccttactacgaccccgc

SEQ ID NO: 323 - DNA, 25 bp, Artificial Sequence
  oligonucleotide primer
aaaaaaaaccttactacaaccccac

SEQ ID NO: 324 - DNA, 23 bp, Artificial Sequence
  oligonucleotide primer
aaacgcatatacctaccgtccga

SEQ ID NO: 325 - DNA, 28 bp, Artificial Sequence
  oligonucleotide primer
accaaaaacacatatacctaccatccaa

SEQ ID NO: 326 - DNA, 28 bp, Artificial Sequence
  oligonucleotide primer
ccatatatttttatataaaaaaatcgta

SEQ ID NO: 327 - DNA, 30 bp, Artificial Sequence
  oligonucleotide primer
aaaccatatatttttatataaaaaaatcat

SEQ ID NO: 328 - DNA, 25 bp, Artificial Sequence
  oligonucleotide primer
aaactactcacataccccataacga

SEQ ID NO: 329 - DNA, 25 bp, Artificial Sequence
  oligonucleotide primer
aaactactcacataccccataacaa

SEQ ID NO: 330 - DNA, 25 bp, Artificial Sequence
  oligonucleotide primer
accaaccttctaaccttaaaccgaa

SEQ ID NO: 331 - DNA, 25 bp, Artificial Sequence
  oligonucleotide primer
accaaccttctaaccttaaaccaaa

SEQ ID NO: 332 - DNA, 20 bp, Artificial Sequence
  oligonucleotide primer
aaatccatttcgaaccgcga

SEQ ID NO: 333 - DNA, 23 bp, Artificial Sequence
  oligonucleotide primer
ttaaaatccatttcaaaccacaa

SEQ ID NO: 334 - DNA, 27 bp, Artificial Sequence
  oligonucleotide primer
aaaactaacctctaaaacaaaaaacga

SEQ ID NO: 335 - DNA, 28 bp, Artificial Sequence
  oligonucleotide primer
caaaactaacctctaaaacaaaaaacaa

SEQ ID NO: 336 - DNA, 24 bp, Artificial Sequence
  oligonucleotide primer
tatacccaaaaatttaaattcccg

SEQ ID NO: 337 - DNA, 25 bp, Artificial Sequence
  oligonucleotide primer
atacccaaaaatttaaattcccaat

SEQ ID NO: 338 - DNA, 25 bp, Artificial Sequence
  oligonucleotide primer
caacaccataataataaccaccgta

SEQ ID NO: 339 - DNA, 25 bp, Artificial Sequence
  oligonucleotide primer
caacaccataataataaccaccata

SEQ ID NO: 340 - DNA, 30 bp, Artificial Sequence
  oligonucleotide primer
tgagtttatagttttttaaatattatttta

SEQ ID NO: 341 - DNA, 25 bp, Artificial Sequence
  oligonucleotide primer
tgtgttaagaggtgatgtttaaggt

SEQ ID NO: 342 - DNA, 25 bp, Artificial Sequence
  oligonucleotide primer
attaagaaattttaataaaagagaa

SEQ ID NO: 343 - DNA, 28 bp, Artificial Sequence
  oligonucleotide primer
tttattgtttggggagtatttggtaggt

SEQ ID NO: 344 - DNA, 30 bp, Artificial Sequence
  oligonucleotide primer
ggagattgtaataaattaaggttaaaagag

SEQ ID NO: 345 - DNA, 30 bp, Artificial Sequence
  oligonucleotide primer
ttattagaagttaagaagaaaggggagtga

SEQ ID NO: 346 - DNA, 25 bp, Artificial Sequence
  oligonucleotide primer
tggtgatggaggaggtttagtaagt

SEQ ID NO: 347 - DNA, 25 bp, Artificial Sequence
  oligonucleotide primer
tactcaaaaacaacttaccttcaac

SEQ ID NO: 348 - DNA, 26 bp, Artificial Sequence
  oligonucleotide primer
aacaaataaaactactcacatacccc

SEQ ID NO: 349 - DNA, 25 bp, Artificial Sequence
  oligonucleotide primer
taaaactactcacataccccataac

SEQ ID NO: 350 - DNA, 27 bp, Artificial Sequence
  oligonucleotide primer
ccacctttccacccctaaaatataaac

SEQ ID NO: 351 - DNA, 25 bp, Artificial Sequence
  oligonucleotide primer
taaaacacacacaaaactcactcac

SEQ ID NO: 352 - DNA, 25 bp, Artificial Sequence
  oligonucleotide primer
tacatccaacaaccacccaataaac

SEQ ID NO: 353 - DNA, 27 bp, Artificial Sequence
  oligonucleotide primer
aaccaataaaacctactcctcccttaa